(19) United States
(12) Patent Application Publication
(10) Pub. No.: US 2021/0269782 A1
(43) Pub. Date: Sep. 2, 2021

(54) RNA-GUIDED EFFECTOR PROTEINS AND METHODS OF USE THEREOF

(71) Applicant: The Regents of the University of California, Oakland, CA (US)

(72) Inventors: Jennifer A. Doudna, Berkeley, CA (US); David Frank Savage, Berkeley, CA (US); Sean A. Higgins, Berkeley, CA (US); Benjamin L. Oakes, Berkeley, CA (US)

(21) Appl. No.: 17/057,531

(22) PCT Filed: Jun. 25, 2019
(86) PCT No.: PCT/US19/39041

§ 371 Date: Nov. 20, 2020

Related U.S. Application Data
(60) Provisional application No. 62/690,282, filed on Jun. 26, 2018.

(51) Int. Cl. C12N 9/22; C12N 15/11; C07K 19/00

(52) U.S. Cl. CPC C12N 9/22; C12N 2310/20; C07K 19/00; C12N 15/11

(57) ABSTRACT

The present disclosure provides an RNA-guided effector polypeptide having a length that is less than Streptococcus pyogenes Cas9, and that retains the ability, when complexed with a guide RNA, to bind to a target nucleic acid. The present disclosure provides a fusion polypeptide comprising: i) an RNA-guided effector polypeptide of the present disclosure; and ii) a fusion partner. The present disclosure further provides nucleic acids encoding an RNA-guided effector polypeptide, or a fusion polypeptide, of the present disclosure. Methods of using an RNA-guided effector polypeptide, or a fusion polypeptide, of the present disclosure are also provided.

### CROSS-REFERENCE

This application claims the benefit of U.S. Provisional Patent Application No. 62/690,282, filed Jun. 26, 2018, which application is incorporated herein by reference in its entirety.

### STATEMENT REGARDING FEDERALLY SPONSORED RESEARCH

This invention was made with government support under EB018658 awarded by the National Institutes of Health. The government has certain rights in the invention

### INCORPORATION BY REFERENCE OF SEQUENCE LISTING PROVIDED AS A TEXT FILE

A Sequence Listing is provided herewith as a text file, “BERK-383WO_SEQ_LISTING_ST25.txt” created on Jun. 18, 2019 and having a size of 7,871 KB. The contents of the text file are incorporated by reference herein in their entirety.

### INTRODUCTION

Bacterial adaptive immune systems employ CRISPRs (clustered regularly interspaced short palindromic repeats) and CRISPR-associated (Cas) proteins for RNA-guided nucleic acid cleavage. The CRISPR-Cas systems thereby confer adaptive immunity in bacteria and archaea via RNA-guided nucleic acid interference. To provide anti-viral immunity, processed CRISPR array transcripts (crRNAs) assemble with Cas protein-containing surveillance complexes that recognize nucleic acids bearing sequence complementarity to the virus derived segment of the crRNAs, known as the spacer.

Class 2 CRISPR-Cas systems are streamlined versions in which a single Cas protein (an effector protein, e.g., a type V Cas effector protein such as Cpf1) bound to RNA is responsible for binding to and cleavage of a targeted sequence. The programmable nature of these minimal systems has facilitated their use as a versatile technology that continues to revolutionize the field of genome manipulation.

The physical size of CRISPR-Cas9 proteins can complicate their delivery. There is a need in the art for CRISPR-Cas9 proteins that are smaller than those currently in use.

### SUMMARY

The present disclosure provides an RNA-guided effector polypeptide having a length that is less than Streptococcus pyogenes Cas9, and that retains the ability, when complexed with a guide RNA, to bind to a target nucleic acid. The present disclosure provides a fusion polypeptide comprising: i) an RNA-guided effector polypeptide of the present disclosure; and ii) a fusion partner. The present disclosure further provides nucleic acids encoding an RNA-guided effector polypeptide, or a fusion polypeptide, of the present disclosure. Methods of using an RNA-guided effector polypeptide, or a fusion polypeptide, of the present disclosure are also provided.

### BRIEF DESCRIPTION OF THE DRAWINGS

FIG. 1 provide an amino acid sequence of Streptococcus pyogenes Cas9 (Spy Cas9).

FIG. 2A-2B depict start and end sites of deletions of Cas9.

FIG. 3 provides the amino acid sequence of D3CE, an example of an RNA-guided effector polypeptide of the present disclosure.

FIG. 4 provides the amino acid sequence of D4CE, an example of an RNA-guided effector polypeptide of the present disclosure.

FIG. 5A-5B provide an amino acid sequence alignment of D3CE with Spy Cas9.

FIG. 6A-6B provide an amino acid sequence alignment of D4CE with Spy Cas9.

FIGS. 7A and 7B provide amino acid sequences of the catalytic domain of a FokI nuclease (FIG. 7A) and the “Sharkey” FokI variant (FIG. 7B).

FIG. 8 depicts the frequency of highly functional deletion variants across dCas9 sequence. Shown on the graph are the general locations of the A, B, C, and D deletion variants.

FIG. 9 depicts the repression activity of examples of RNA-guided effector polypeptides of the present disclosure, where the RNA-guided effector polypeptides are fused to a transcriptional repressor. Indicated in brackets are the boundaries of the deletion(s).

FIGS. 10A and 10B through FIGS. 22A and 22B provide amino acid sequences of examples of RNA-guided effector polypeptides of the present disclosure.

FIG. 23 provides deletion sites of RNA-guided effector polypeptides of the present disclosure.

FIG. 24A-24E depicts the percentage of mCherry positive mammalian cells in a CRISPR interference assay to assess the function of dCas9 variants with deletion sites in mammalian cells.

FIG. 25 depicts an immunoblotting assay for Flag-tagged MISER-dCas9 or WT-dCas9 KRAB fusion proteins stably expressed in U-251 cells and co-expressing a non-targeting guide (sgNT1).

FIG. 26 depicts an RNP-based gel shift assay to assess in vitro function of variants dCas9, Δ3CE, and Δ4CE.

FIG. 27 depicts an immunoblotting assay of variants Δ3CE, and Δ4CE that programmably bind to DNA and are functional in human cells.

### DEFINITIONS

The terms “polynucleotide” and “nucleic acid,” used interchangeably herein, refer to a polymeric form of nucleotides of any length, either ribonucleotides or deoxyribonucleotides. Thus, terms “polynucleotide” and “nucleic acid” encompass single-stranded DNA; double-stranded DNA; multi-stranded DNA; single-stranded RNA; double-stranded RNA; multi-stranded RNA; genomic DNA; cDNA; DNA-RNA hybrids; and a polymer comprising purine and pyrimidine bases or other natural, chemically or biochemically modified, non-natural, or derivatized nucleotide bases.

The term “oligonucleotide” refers to a polynucleotide of between 4 and 100 nucleotides of single- or double-stranded nucleic acid (e.g., DNA, RNA, or a modified nucleic acid). However, for the purposes of this disclosure, there is no upper limit to the length of an oligonucleotide. Oligonucleotides are also known as “oligomers” or “oligos” and can be isolated from genes, transcribed (in vitro and/or in vivo), or chemically synthesized. The terms “polynucleotide” and “nucleic acid” should be understood to include, as applicable to the embodiments being described, single-stranded (such as sense or antisense) and double-stranded polynucleotides.

By “hybridizable” or “complementary” or “substantially complementary” it is meant that a nucleic acid (e.g. RNA, DNA) comprises a sequence of nucleotides that enables it to non-covalently bind, i.e. form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. Standard Watson-Crick base-pairing includes: adenine/adenosine) (A) pairing with thymidine/thymidine (T), A pairing with uracil/uridine (U), and guanine/guanosine) (G) pairing with cytosine/cytidine (C). In addition, for hybridization between two RNA molecules (e.g., dsRNA), and for hybridization of a DNA molecule with an RNA molecule (e.g., when a DNA target nucleic acid base pairs with a guide RNA, etc.): G can also base pair with U. For example, G/U base-pairing is partially responsible for the degeneracy (i.e., redundancy) of the genetic code in the context of tRNA anti-codon base-pairing with codons in mRNA. Thus, in the context of this disclosure, a G (e.g., of a protein-binding segment (e.g., dsRNA duplex) of a guide RNA molecule; of a target nucleic acid (e.g., target DNA) base pairing with a guide RNA) is considered complementary to both a U and to C. For example, when a G/U base-pair can be made at a given nucleotide position of a protein-binding segment (e.g., dsRNA duplex) of a guide RNA molecule, the position is not considered to be non-complementary, but is instead considered to be complementary.

Hybridization requires that the two nucleic acids contain complementary sequences, although mismatches between bases are possible. The conditions appropriate for hybridization between two nucleic acids depend on the length of the nucleic acids and the degree of complementarity, variables well known in the art. The greater the degree of complementarity between two nucleotide sequences, the greater the value of the melting temperature (Tm) for hybrids of nucleic acids having those sequences. Typically, the length for a hybridizable nucleic acid is 8 nucleotides or more (e.g., 10 nucleotides or more, 12 nucleotides or more, 15 nucleotides or more, 20 nucleotides or more, 22 nucleotides or more, 25 nucleotides or more, or 30 nucleotides or more).

It is understood that the sequence of a polynucleotide need not be 100% complementary to that of its target nucleic acid to be specifically hybridizable. Moreover, a polynucleotide may hybridize over one or more segments such that intervening or adjacent segments are not involved in the hybridization event (e.g., a loop structure or hairpin structure, a ‘bulge’, and the like). A polynucleotide can comprise 60% or more, 65% or more, 70% or more, 75% or more, 80% or more, 85% or more, 90% or more, 95% or more, 98% or more, 99% or more, 99.5% or more, or 100% sequence complementarity to a target region within the target nucleic acid sequence to which it will hybridize. For example, an antisense nucleic acid in which 18 of 20 nucleotides of the antisense compound are complementary to a target region, and would therefore specifically hybridize, would represent 90 percent complementarity. The remaining noncomplementary nucleotides may be clustered or interspersed with complementary nucleotides and need not be contiguous to each other or to complementary nucleotides. Percent complementarity between particular stretches of nucleic acid sequences within nucleic acids can be determined using any convenient method. Example methods include BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), e.g., using default settings, which uses the algorithm of Smith and Waterman (Adv. Appl. Math., 1981, 2, 482-489).

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

“Binding” as used herein (e.g. with reference to an RNA-binding domain of a polypeptide, binding to a target nucleic acid, and the like) refers to a non-covalent interaction between macromolecules (e.g., between a protein and a nucleic acid; between a guide RNA and a target nucleic acid; and the like). While in a state of non-covalent interaction, the macromolecules are said to be “associated” or “interacting” or “binding” (e.g., when a molecule X is said to interact with a molecule Y, it is meant the molecule X binds to molecule Y in a non-covalent manner). Not all components of a binding interaction need be sequence-specific (e.g., contacts with phosphate residues in a DNA backbone), but some portions of a binding interaction may be sequence-specific. Binding interactions are generally characterized by a dissociation constant (K_{d}) of less than 10⁻⁶ M, less than 10⁻⁷ M, less than 10⁻⁸ M, less than 10⁻⁹ M, less than 10⁻¹⁰ M, less than 10⁻¹¹ M, less than 10⁻¹² M, less than 10⁻¹³ M, less than 10⁻¹⁴ M, or less than 10⁻¹⁵ M. “Affinity” refers to the strength of binding, increased binding affinity being correlated with a lower K_{d}.

By “binding domain” it is meant a protein domain that is able to bind non-covalently to another molecule. A binding domain can bind to, for example, an RNA molecule (an RNA-binding domain) and/or a protein molecule (a protein-binding domain). In the case of a protein having a protein-binding domain, it can in some cases bind to itself (to form homodimers, homotrimers, etc.) and/or it can bind to one or more regions of a different protein or proteins.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide containing side chains consisting of asparagine and glutamine; a group of amino acids having aromatic side chains consists of phenylalanine, tyrosine, and tryptophan; a group of amino acids having basic side chains consists of lysine, arginine, and histidine; a group of amino acids having acidic side chains consists of glutamate and aspartate; and a group of amino acids having sulfur containing side chains consists of cysteine and methionine. Exemplary conservative amino acid substitution groups are: valine-leucine-isoleucine, phenylalanine-tyrosine, lysine-arginine, alanine-valine-glycine, and asparagine-glutamine.

A polynucleotide or polypeptide has a certain percent “sequence identity” to another polynucleotide or polypeptide, meaning that, when aligned, that percentage of bases or amino acids are the same, and in the same relative position, when comparing the two sequences. Sequence identity can be determined in a number of different ways. To determine sequence identity, sequences can be aligned using various methods and computer programs (e.g., BLAST, T-COFFEE, MUSCLE, MAFFT, Phyre2, etc.), available over the world wide web at sites including ncbi(dot)nlm(dot)nili(dot)gov/BLAST, ebi(dot)ac(dot)uk/Tools/msa/tcoffee/, ebi(dot)ac(dot)uk/Tools/msa/muscle/, mafft(dot)cbrc(dot)jp/alignment/software/, www(dot)sbg(dot)bio(dot)ic(dot)ac(dot)uk/˜phyre2/. See, e.g., Altschul et al. (1990), J. Mol. Bioi. 215:403-10.

The terms “DNA regulatory sequences,” “control elements,” and “regulatory elements,” used interchangeably herein, refer to transcriptional and translational control sequences, such as promoters, enhancers, polyadenylation signals, terminators, protein degradation signals, and the like, that provide for and/or regulate transcription of a non-coding sequence (e.g., guide RNA) or a coding sequence (e.g., protein coding) and/or regulate translation of an encoded polypeptide.

As used herein, a “promoter sequence” is a DNA regulatory region capable of binding RNA polymerase and initiating transcription of a downstream (3′ direction) coding or non-coding sequence. Eukaryotic promoters will often, but not always, contain “TATA” boxes and “CAT” boxes. Various promoters, including inducible promoters, may be used to drive the various nucleic acids (e.g., vectors) of the present disclosure.

The term “naturally-occurring” or “unmodified” or “wild type” as used herein as applied to a nucleic acid, a polypeptide, a cell, or an organism, refers to a nucleic acid, polypeptide, cell, or organism that is found in nature.

“Recombinant,” as used herein, means that a particular nucleic acid (DNA or RNA) is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides, to provide a synthetic nucleic acid which is capable of being expressed from a recombinant transcriptional unit contained in a cell or in a cell-free transcription and translation system. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions, and may indeed act to modulate production of a desired product by various mechanisms (see “DNA regulatory sequences”, below). Alternatively, DNA sequences encoding RNA (e.g., guide RNA) that is not translated may also be considered recombinant. Thus, e.g., the term “recombinant” nucleic acid refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a codon encoding the same amino acid, a conservative amino acid, or a non-conservative amino acid. Alternatively, it is performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques. When a recombinant polynucleotide encodes a polypeptide, the sequence of the encoded polypeptide can be naturally occurring (“wild type”) or can be a variant (e.g., a mutant) of the naturally occurring sequence. Thus, the term “recombinant” polypeptide does not necessarily refer to a polypeptide whose sequence does not naturally occur. Instead, a “recombinant” polypeptide is encoded by a recombinant DNA sequence, but the sequence of the polypeptide can be naturally occurring (“wild type”) or non-naturally occurring (e.g., a variant, a mutant, etc.). Thus, a “recombinant” polypeptide is the result of human intervention, but may be a naturally occurring amino acid sequence.

A “vector” or “expression vector” is a replicon, such as plasmid, phage, virus, or cosmid, to which another DNA segment, i.e. an “insert”, may be attached so as to bring about the replication of the attached segment in a cell.

An “expression cassette” comprises a DNA coding sequence operably linked to a promoter. “Operably linked” refers to a juxtaposition wherein the components so described are in a relationship permitting them to function in their intended manner. For instance, a promoter is operably linked to a coding sequence if the promoter affects its transcription or expression.

The terms “recombinant expression vector,” or “DNA construct” are used interchangeably herein to refer to a DNA molecule comprising a vector and one insert. Recombinant expression vectors are usually generated for the purpose of expressing and/or propagating the insert(s), or for the construction of other recombinant nucleotide sequences. The insert(s) may or may not be operably linked to a promoter sequence and may or may not be operably linked to DNA regulatory sequences.

Any given component, or combination of components can be unlabeled, or can be detectably labeled with a label moiety. In some cases, when two or more components are labeled, they can be labeled with label moieties that are distinguishable from one another.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “an RNA-guided effector polypeptide” includes a plurality of such RNA-guided effector polypeptides and reference to “the guide RNA” includes reference to one or more guide RNAs and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provide an RNA-guided effector polypeptide having a length that is less than Streptococcus pyogenes Cas9, and that retains the ability, when complexed with a guide RNA, to bind to a target nucleic acid. The present disclosure provides a fusion polypeptide comprising: i) an RNA-guided effector polypeptide of the present disclosure; and ii) a fusion partner. The present disclosure further provides nucleic acids encoding an RNA-guided effector polypeptide, or a fusion polypeptide, of the present disclosure. Methods of using an RNA-guided effector polypeptide, or a fusion polypeptide, of the present disclosure are also provided.

An RNA-guided effector polypeptide of the present disclosure has a length that is less than Streptococcus pyogenes Cas9 (Spy Cas9). An RNA-guided effector polypeptide that has a length that is less than Spy Cas9 is advantageous. Expression vectors comprising a nucleotide sequence encoding a heterologous polypeptide such as Cas9 have size limitations; such expression vectors can more readily accommodate an RNA-guided effector polypeptide that has a length less than the 1,368-amino acid full-length Spy Cas9. Moreover, a smaller RNA-guided effector polypeptide can serve as a scaffold for a fusion polypeptide comprising the RNA-guided effector polypeptide and a fusion partner, where the fusion partner provides a functionality such as nuclease activity, transcriptional control, base editing, and the like. An expression vector can also accommodate a nucleotide sequence encoding such a fusion polypeptide, due to the smaller size of the RNA-guided effector polypeptide.

### RNA-Guided Effector Polypeptides

The present disclosure provides an RNA-guided effector polypeptide, where the RNA-guided effector polypeptide has a length that is less than 1,368 amino acids, and than retains the ability, when complexed with a guide RNA, to bind to a target nucleic acid.

An RNA-guided effector polypeptide of the present disclosure can have a length of from about 700 amino acids to about 1000 amino acids, e.g., from about 700 amino acids (aa) to about 705 aa, from about 705 aa to about 710 aa, from about 710 aa to about 715 aa, from about 715 aa to about 720 aa, from about 720 aa to about 725 aa, from about 725 aa to about 730 aa, from about 730 aa to about 735 aa, from about 735 aa to about 740 aa, from about 740 aa to about 745 aa, from about 745 aa to about 750 aa, from about 750 aa to about 755 aa, from about 755 aa to about 760 aa, from about 760 aa to about 765 aa, from about 765 aa to about 770 aa, from about 770 aa to 7 about 75 aa, from about 775 aa to about 780 aa, from about 780 aa to about 785 aa, from about 85 aa to about 790 aa, from about 790 aa to about 800 aa, from about 800 aa to about 810 aa, from about 810 aa to about 820 aa, from about 820 aa to about 830 aa, from about 830 aa to about 840 aa, from about 840 aa to about 850 aa, from about 850 aa to about 860 aa, from about 860 aa to about 870 aa, from about 870 aa to about 880 aa, from about 880 aa to about 890 aa, from about 890 aa to about 900 aa, from about 900 aa to about 925 aa, from about 925 aa to about 950 aa, from about 950 aa to about 975 aa, from about 975 aa to about 1000 aa.

In some cases, an RNA-guided effector polypeptide of the present disclosure has a length of from about 750 amino acids to about 950 amino acids. In some cases, an RNA-guided effector polypeptide of the present disclosure has a length of from about 800 amino acids to about 950 amino acids. In some cases, an RNA-guided effector polypeptide of the present disclosure has a length of from about 850 amino acids to about 950 amino acids. In some cases, an RNA-guided effector polypeptide of the present disclosure has a length of from about 850 amino acids to about 1000 amino acids. In some cases, an RNA-guided effector polypeptide of the present disclosure has a length of from about 850 amino acids to about 925 amino acids. In some cases, an RNA-guided effector polypeptide of the present disclosure has a length of from about 850 amino acids to about 900 amino acids. In some cases, an RNA-guided effector polypeptide of the present disclosure has a length of from about 900 amino acids to about 1000 amino acids. In some cases, an RNA-guided effector polypeptide of the present disclosure has a length of from about 950 amino acids to about 1000 amino acids.

In some cases, an RNA-guided effector polypeptide of the present disclosure has a length of from about 1000 amino acids to about 1200 amino acids, e.g., from about 100 amino acids (aa) to about 1050 aa, from about 1050 aa to about 1100 aa, from about 1100 aa to about 1150 aa, or from about 1150 aa to about 1200 aa. In some cases, an RNA-guided effector polypeptide of the present disclosure has a length of from about 1200 amino acids to about 1364 amino acids, e.g., from about 1200 amino acids (aa) to about 1210 aa, from about 1210 aa to about 1220 aa, from about 1220 aa to about 1230 aa, from about 1230 aa to about 1240 aa, from about 1240 aa to about 1250 aa, from about 1250 aa to about 1260 aa, from about 1260 aa to about 1270 aa, from about 1270 aa to about 1280 aa, from about 1280 aa to about 1290 aa, from about 1290 aa to about 1300 aa, from about 1300 aa to about 1310 aa, from about 1310 aa to about 1320 aa, from about 1320 aa to about 1330 aa, from about 1330 aa to about 1340 aa, from about 1340 aa to about 1350 aa, or from about 1350 aa to about 1365 aa.

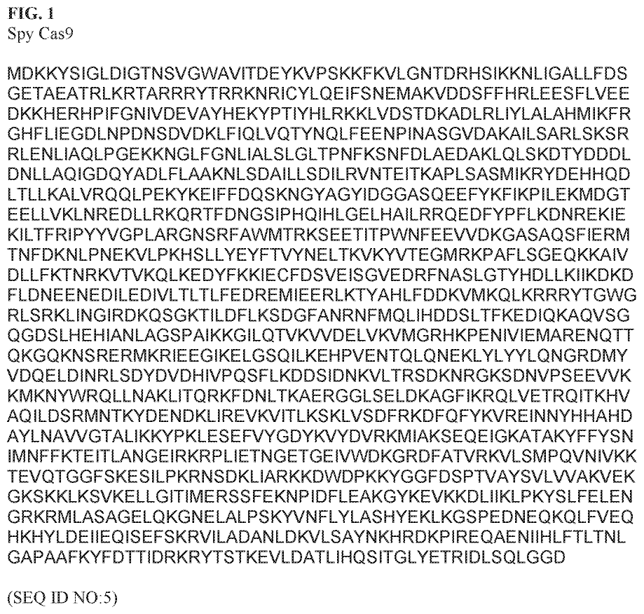

An RNA-guided effector polypeptide of the present disclosure will in some cases exhibit reduced nuclease activity, compared to the nuclease activity exhibited by a Spy Cas9 polypeptide comprising the amino acid sequence depicted in FIG. 1. In some cases, an RNA-guided effector polypeptide of the present disclosure exhibits 50% or less, 40% or less, 30% or less, 25% or less, 20% or less, 15% or less, 10% or less, 5% or less, 2% or less, or 1% or less, of the nuclease activity exhibited by a Spy Cas9 polypeptide comprising the amino acid sequence depicted in FIG. 1. Methods for determining nuclease activity of an RNA-guided effector polypeptide are well known in the art, and any such method can be used to determine whether an RNA-guided effector polypeptide of the present disclosure exhibits reduced nuclease activity, compared to the nuclease activity exhibited by a Spy Cas9 polypeptide comprising the amino acid sequence depicted in FIG. 1. In some cases, an RNA-guided effector polypeptide of the present disclosure does not exhibit detectable nuclease activity.

An RNA-guided effector polypeptide of the present disclosure, when complexed with a guide RNA, retains the ability to bind to a target nucleic acid, where the guide RNA comprises a nucleotide sequence that is complementary to a nucleotide sequence in the target nucleic acid. In some cases, an RNA-guided effector polypeptide of the present disclosure, when complexed with a guide RNA, exhibits at least 50%, at least 60%, at least 70%, at least 80%, at least 90%, at least 95%, or more than 95%, of the binding affinity for a target nucleic acid of a Spy Cas9 polypeptide comprising the amino acid sequence depicted in FIG. 1, when complexed with the same guide RNA, to the same target nucleic acid.

In some cases, an RNA-guided effector polypeptide of the present disclosure comprises 1, 2, 3, or 4 deletions, relative to a Cas9 polypeptide having at least 50%, at least 60%, at least 70%, at least 80%, at least 85%, at least 90%, at least 95%, at least 98%, at least 99%, or 100%, amino acid sequence identity to the Spy Cas9 amino acid sequence depicted in FIG. 1. Each of the 1, 2, 3, or 4 deletions can be from 4 contiguous amino acids to 255 contiguous amino acids in length.

In some cases, RNA-guided effector polypeptide of the present disclosure comprises one or more of:

i) a deletion of from 5 contiguous amino acids to 158 contiguous amino acids of amino acids 166-324 (referred to herein as an “A” deletion);

ii) a deletion of from 30 contiguous amino acids to 255 contiguous amino acids of amino acids 484-739 (referred to herein as a “B” deletion);

iii) a deletion of from 4 contiguous amino acids to 220 contiguous amino acids of amino acids 761-981 (referred to herein as a “C” deletion); and

d) a deletion of from 20 contiguous amino acids to 118 contiguous amino acids of amino acids 994-1112 (referred to herein as a “D” deletion),

where the amino acid numbering is based on the amino acid numbering of the Spy Cas9 amino acid sequence depicted in FIG. 1. An RNA-guided effector polypeptide of the present disclosure can comprise one or more stretches of from 50 to 1000 contiguous amino acids (e.g., from 50 to 75, from 75 to 100, from 100 to 150, from 150 to 200, from 200 to 250, from 250 to 300, from 300 to 350, from 350 to 400, from 400 to 450, from 450 to 500, from 500 to 600, from 600 to 700, from 700 to 800, from 800 to 900, or from 900 to 1000, contiguous amino acids) having at least 50%, at least 60%, at least 70%, at least 80%, at least 85%, at least 90%, at least 95%, at least 98%, at least 99%, or 100%, amino acid sequence identity to corresponding stretches of from 50 to 1000 contiguous amino acids of the Spy Cas9 amino acid sequence depicted in FIG. 1.

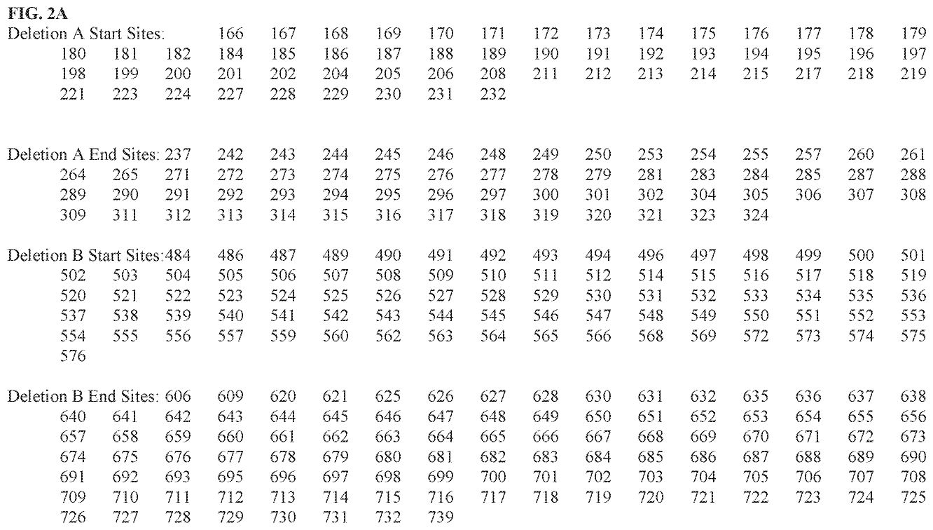

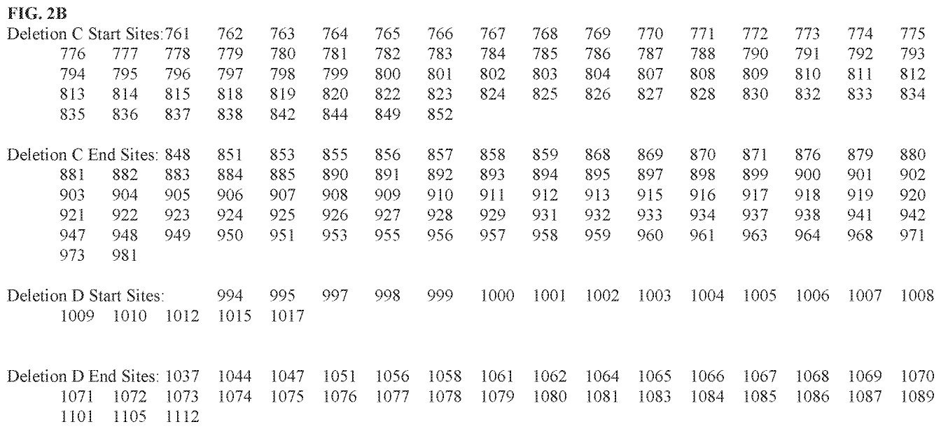

Suitable start and end points for the deletions are depicted in FIGS. 2A and 2B. Any “start” and “end” sites within “A” deletion can be combined to generate an “A” deletion. Any “start” and “end” sites within “B” deletion can be combined to generate a “B” deletion. Any “start” and “end” sites within “C” deletion can be combined to generate a “C” deletion. Any “start” and “end” sites within “D” deletion can be combined to generate a “D” deletion. In some cases, an RNA-guided effector polypeptide of the present disclosure comprises only an “A” deletion. In some cases, an RNA-guided effector polypeptide of the present disclosure comprises only a “B” deletion. In some cases, an RNA-guided effector polypeptide of the present disclosure comprises only a “C” deletion. In some cases, an RNA-guided effector polypeptide of the present disclosure comprises only a “D” deletion. In some cases, an RNA-guided effector polypeptide of the present disclosure comprises only an “A” deletion and a “B” deletion. In some cases, an RNA-guided effector polypeptide of the present disclosure comprises only an “A” deletion and a “C” deletion. In some cases, an RNA-guided effector polypeptide of the present disclosure comprises only an “A” deletion and a “B” deletion. In some cases, an RNA-guided effector polypeptide of the present disclosure comprises only an “A” deletion and a “D” deletion. In some cases, an RNA-guided effector polypeptide of the present disclosure comprises only a “B” deletion and a “C” deletion. In some cases, an RNA-guided effector polypeptide of the present disclosure comprises only a “B” deletion and a “D” deletion. In some cases, an RNA-guided effector polypeptide of the present disclosure comprises only a “C” deletion and a “D” deletion. In some cases, an RNA-guided effector polypeptide of the present disclosure comprises an “A” deletion, a “B” deletion, and a “C” deletion (and not a “D” deletion). In some cases, an RNA-guided effector polypeptide of the present disclosure comprises an “A” deletion, a “B” deletion, and a “D” deletion (and not a “C” deletion). In some cases, an RNA-guided effector polypeptide of the present disclosure comprises an “A” deletion, a “D” deletion, and a “D” deletion (and not a “B” deletion). In some cases, an RNA-guided effector polypeptide of the present disclosure comprises “B” deletion, a “C” deletion, and a “D” deletion (and not an “A” deletion). In some cases, an RNA-guided effector polypeptide of the present disclosure comprises an “A” deletion, a “B” deletion, a “C” deletion, and a “D” deletion.

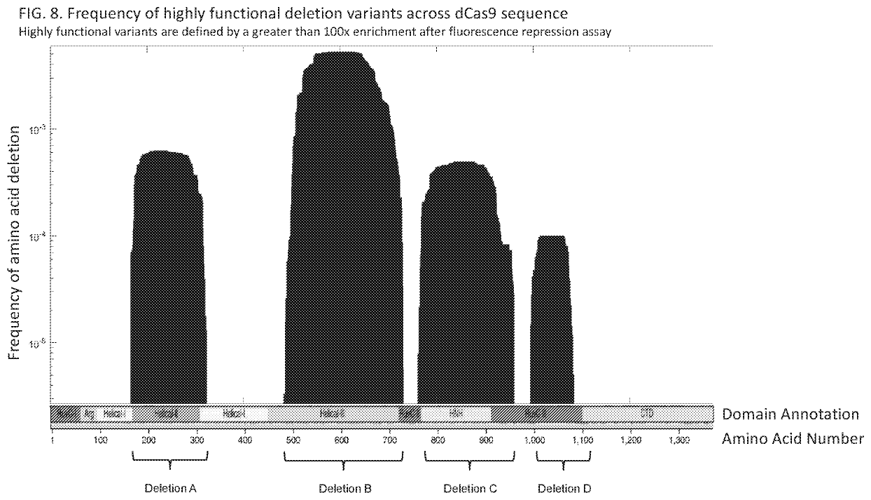

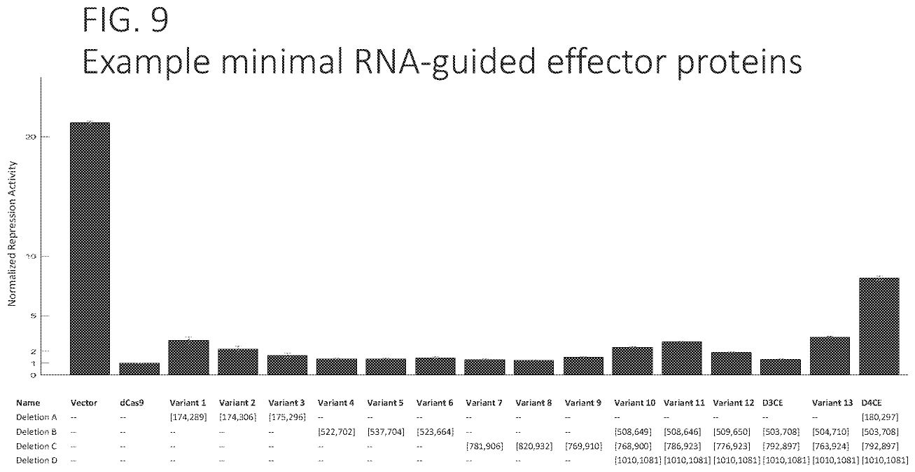

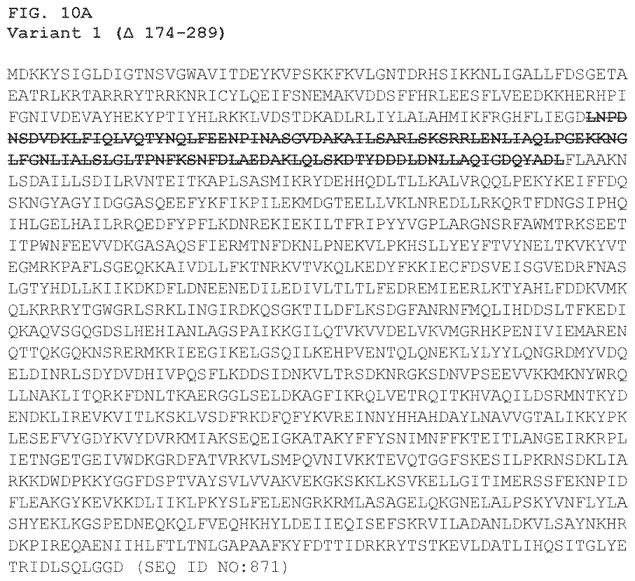

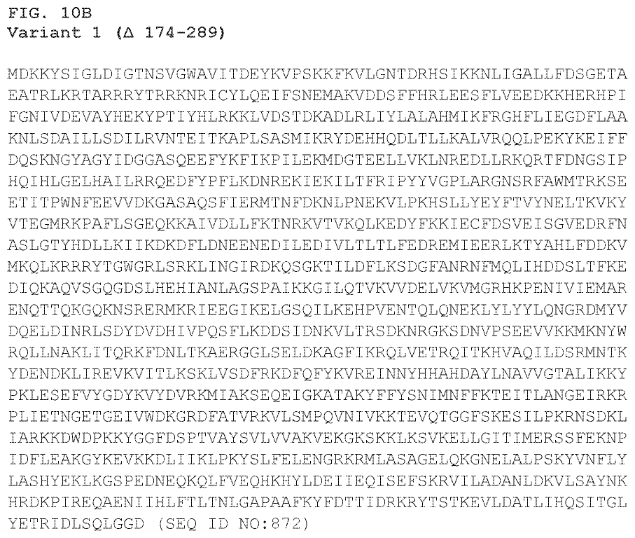

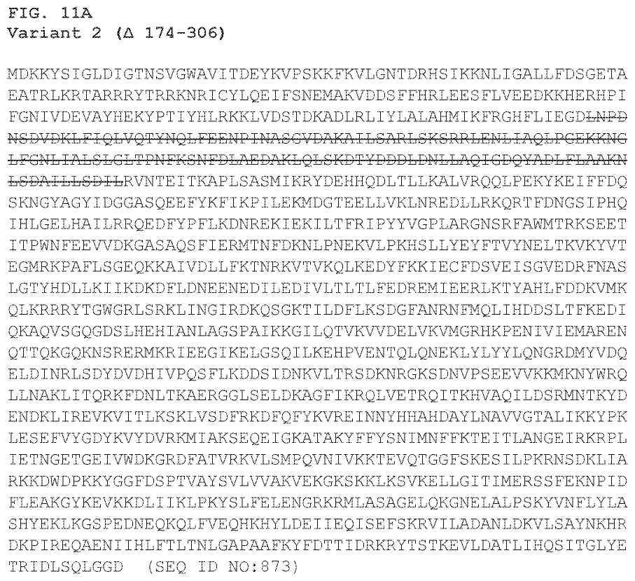

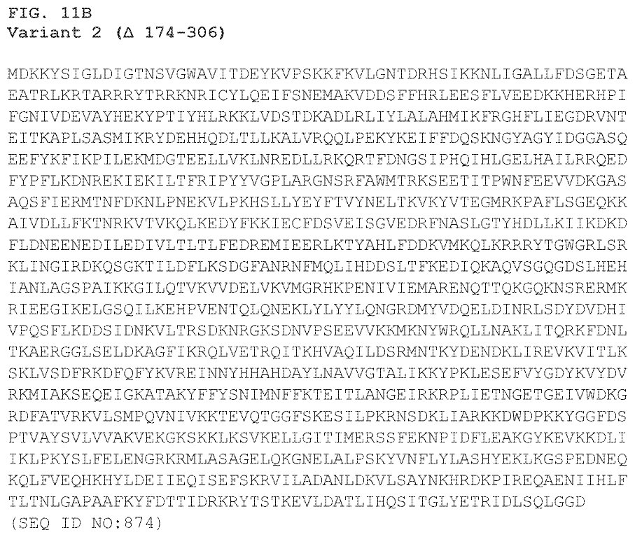

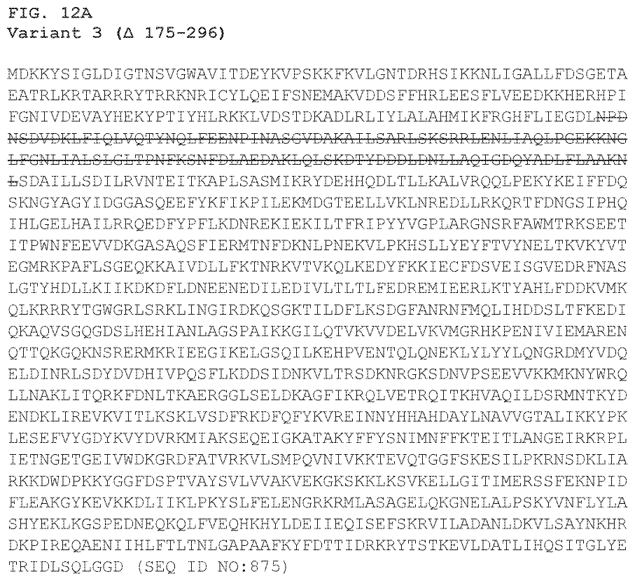

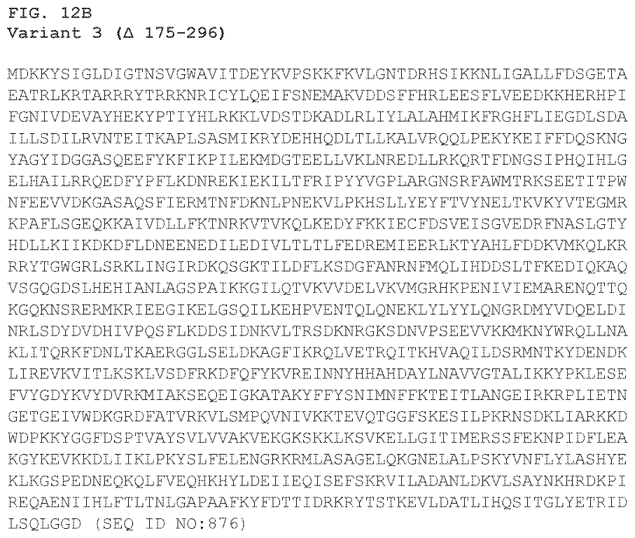

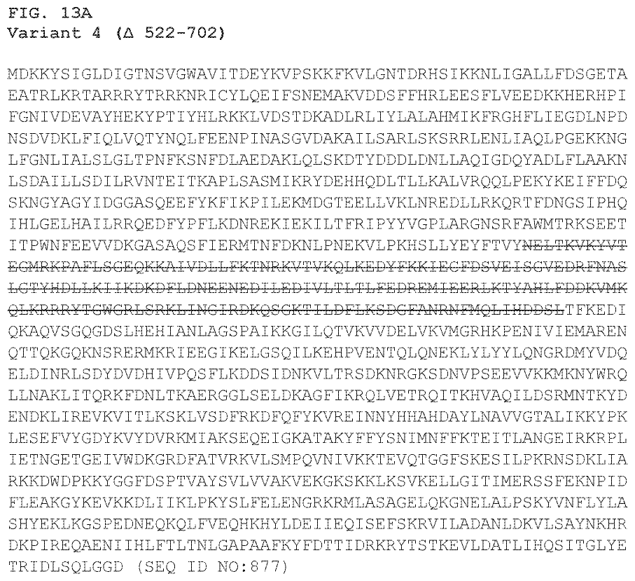

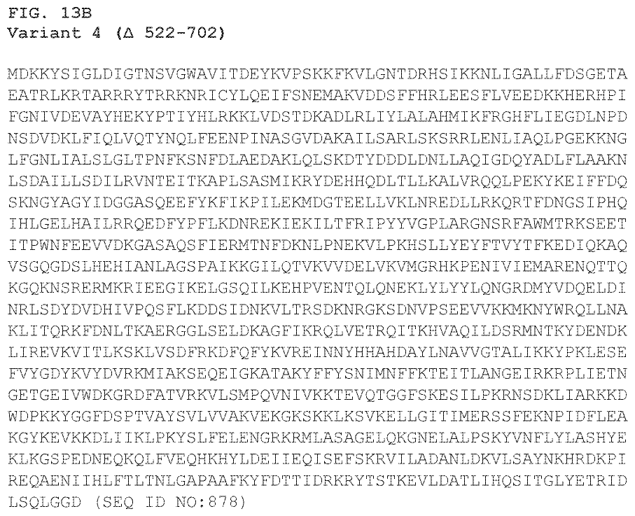

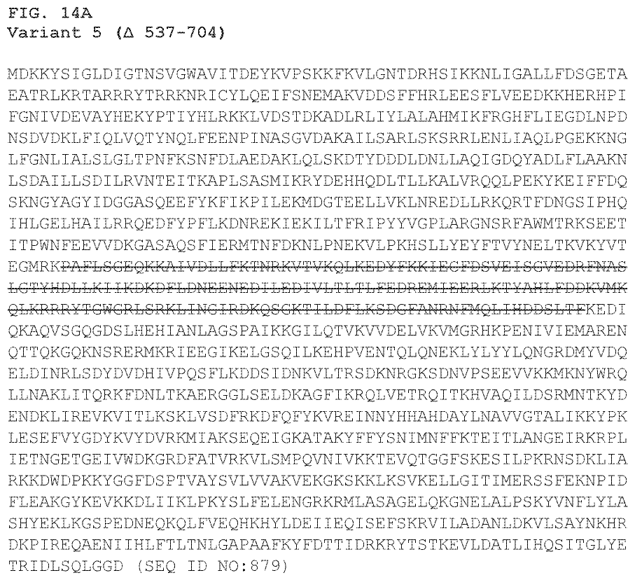

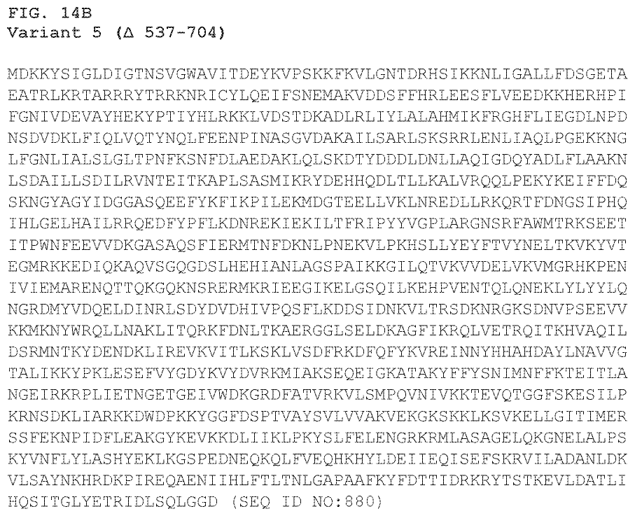

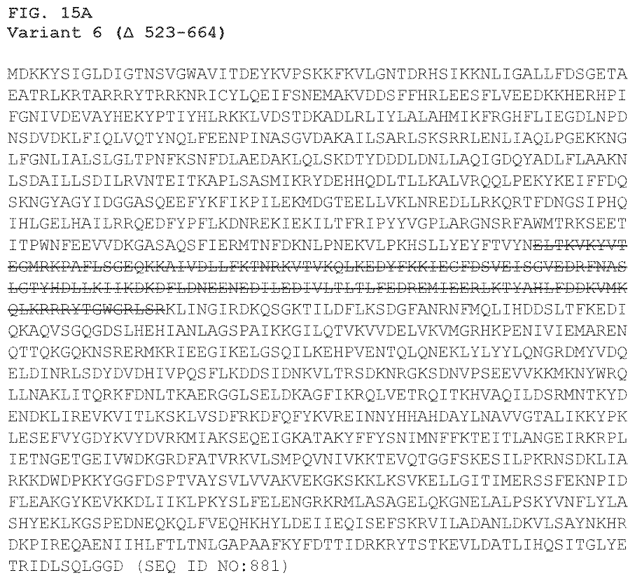

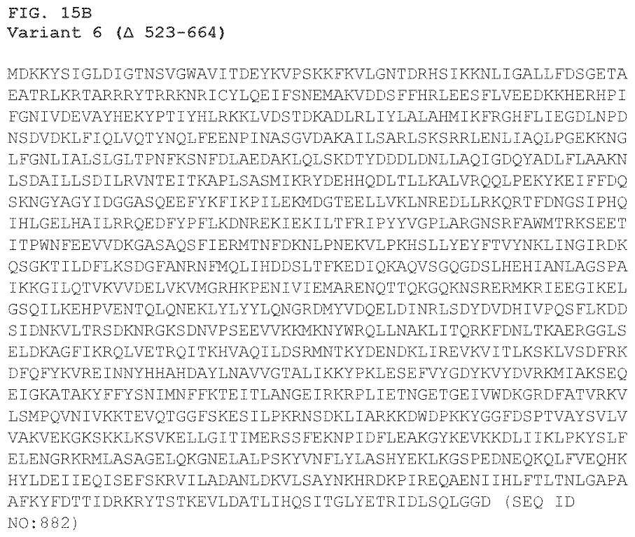

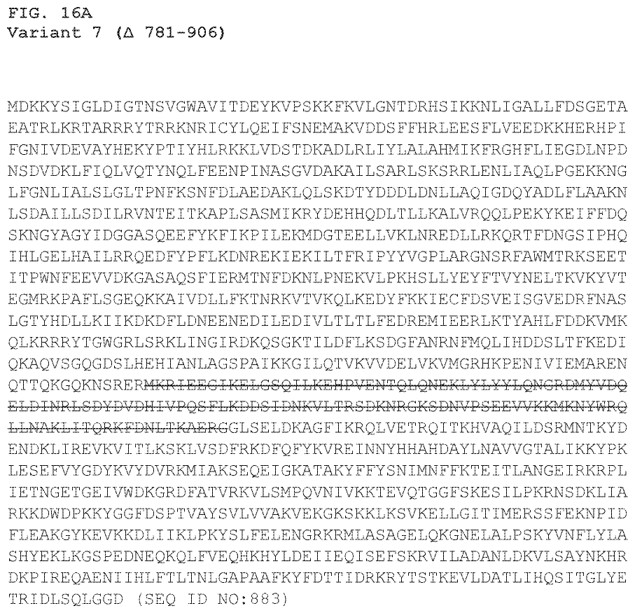

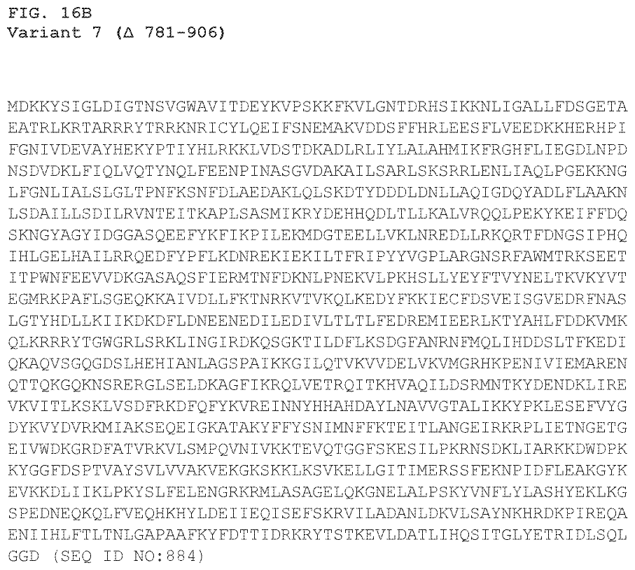

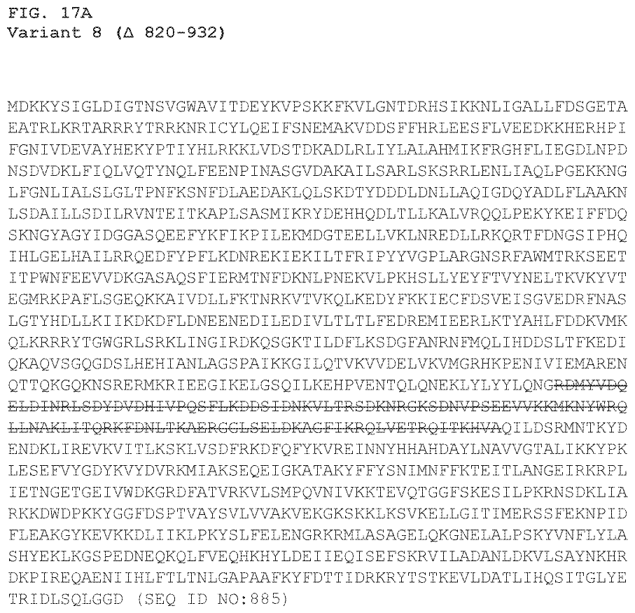

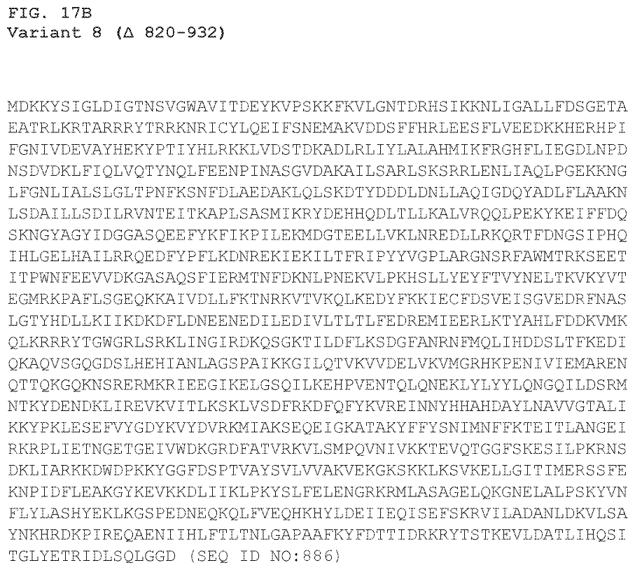

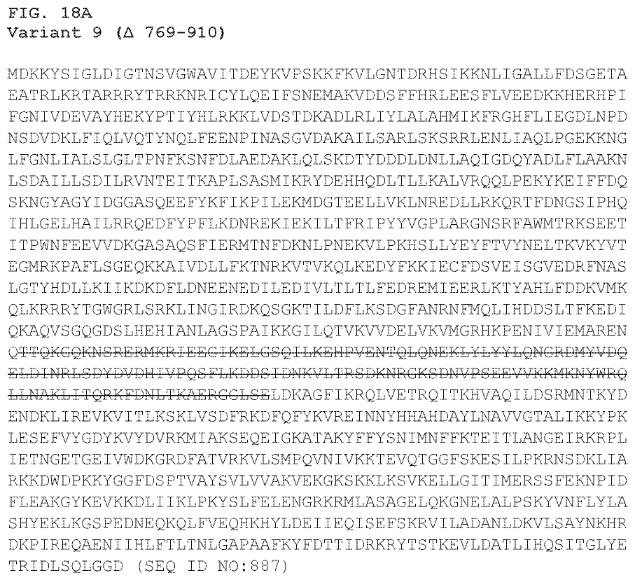

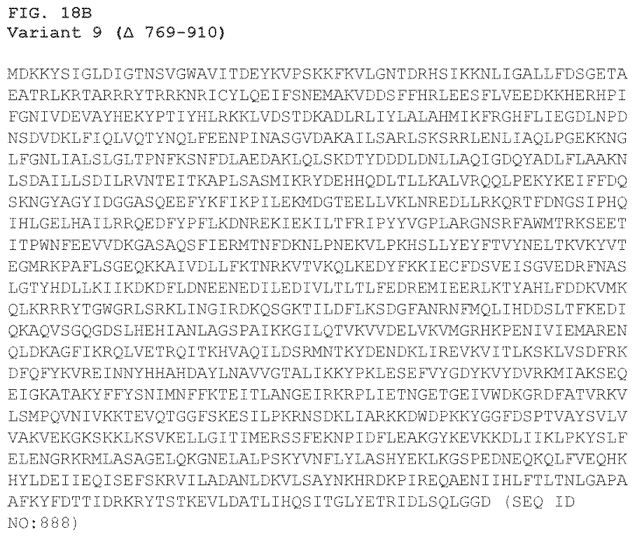

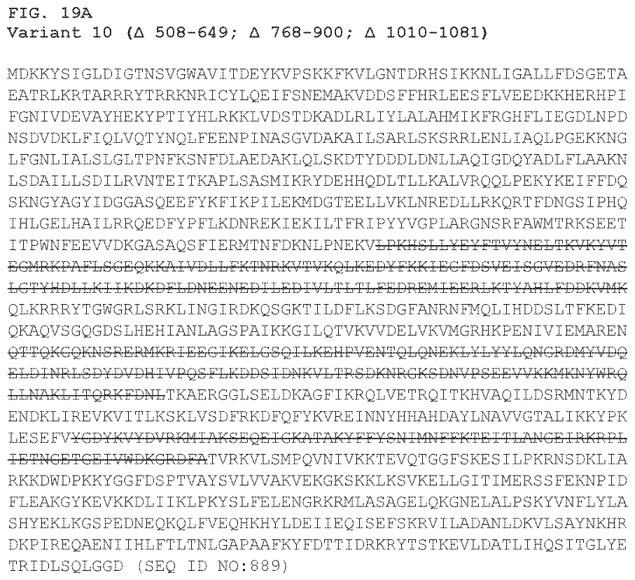

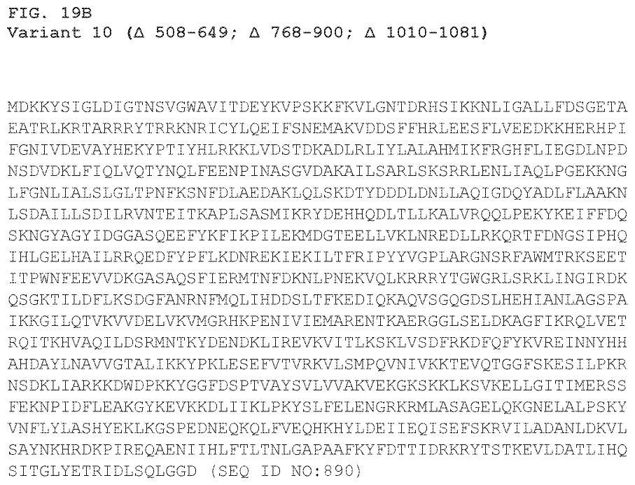

As depicted in FIG. 8, in some cases, an “A” deletion comprises all or a portion of the Helical II domain of Spy Cas9. As depicted in FIG. 8, in some cases, a “B” deletion comprises all or a portion of the Helical III domain of Spy Cas9. As depicted in FIG. 8, in some cases, a “C” deletion comprises all or a portion of the HNH domain of Spy Cas9; and may also comprises a portion of the RuvC III domain of Spy Cas9. As depicted in FIG. 8, in some cases, a “D” deletion comprises a portion of the RuvC III domain of Spy Cas9.

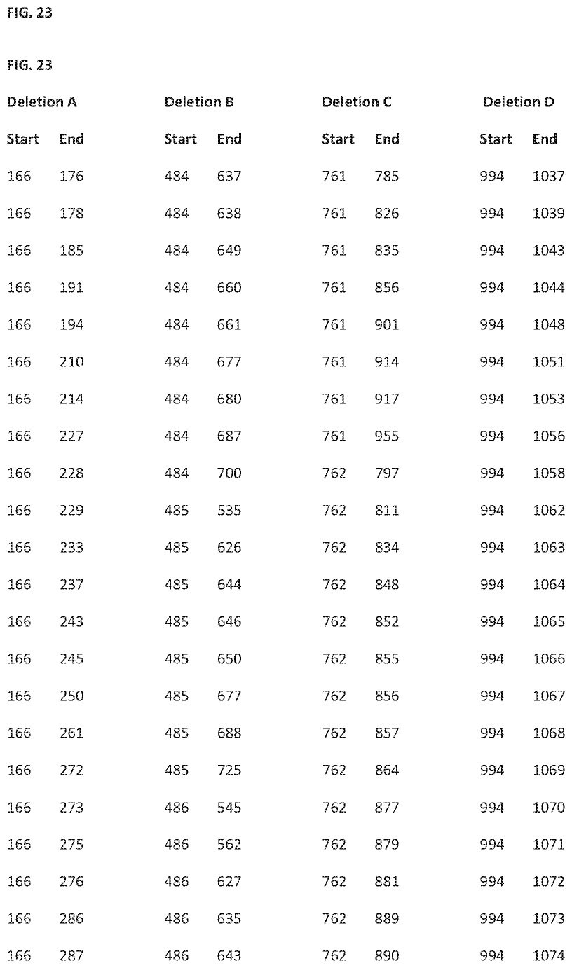

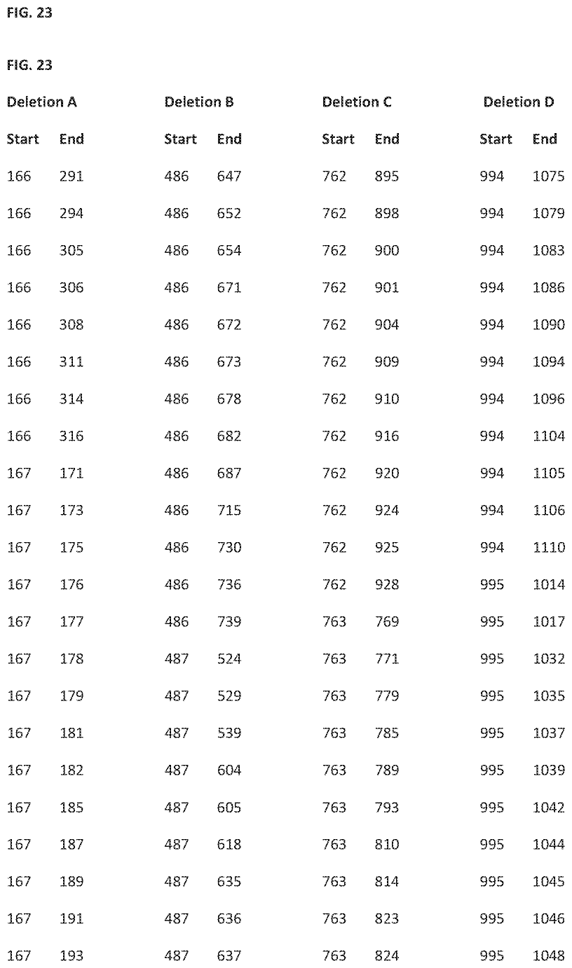

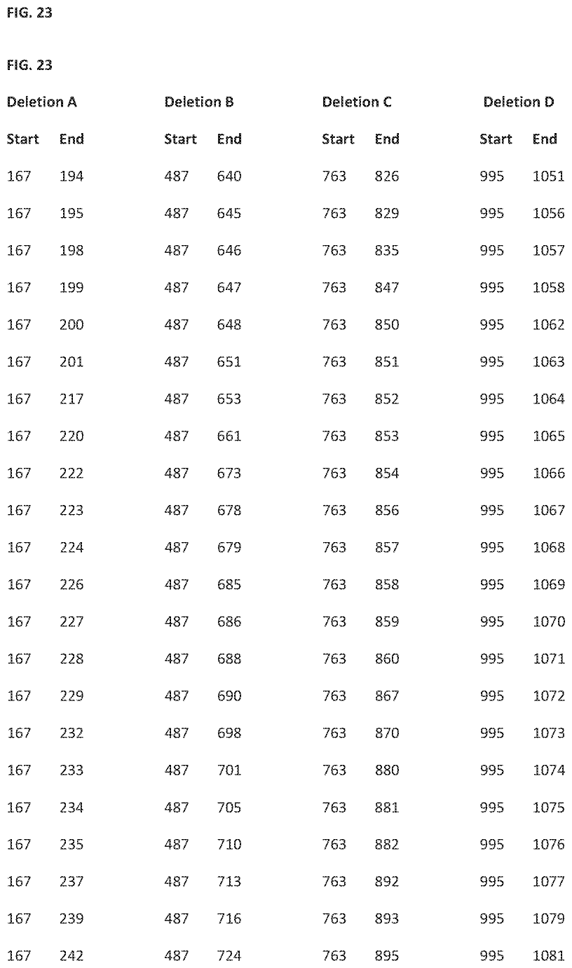

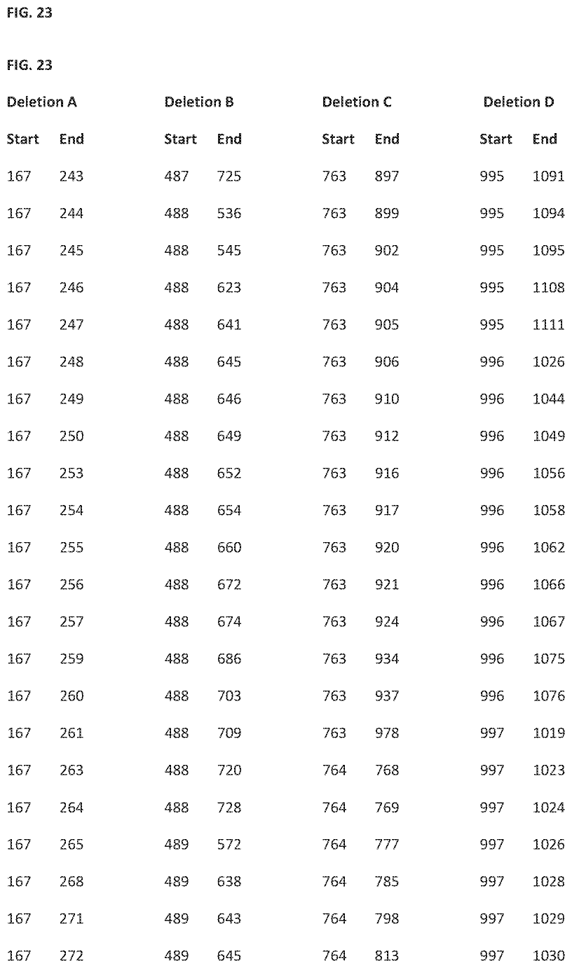

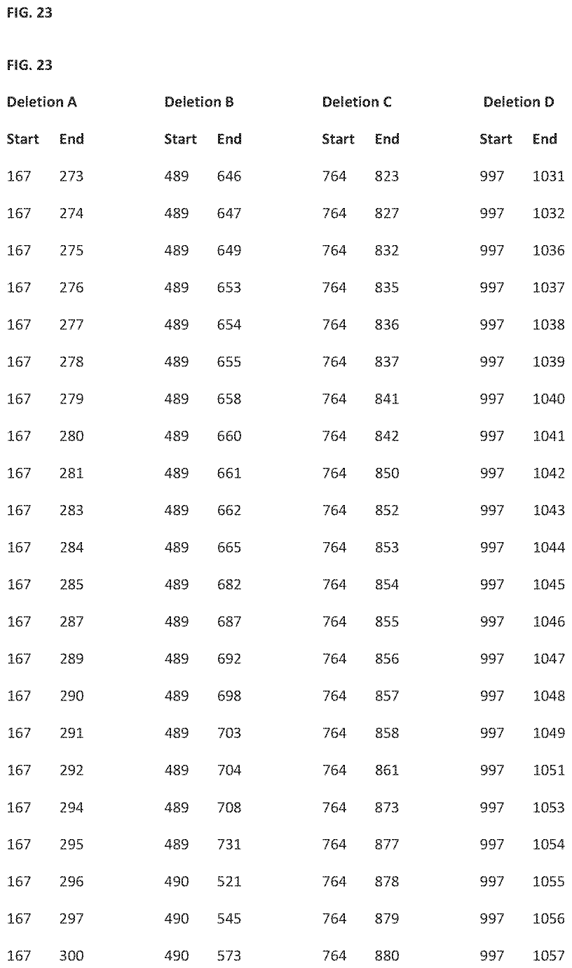

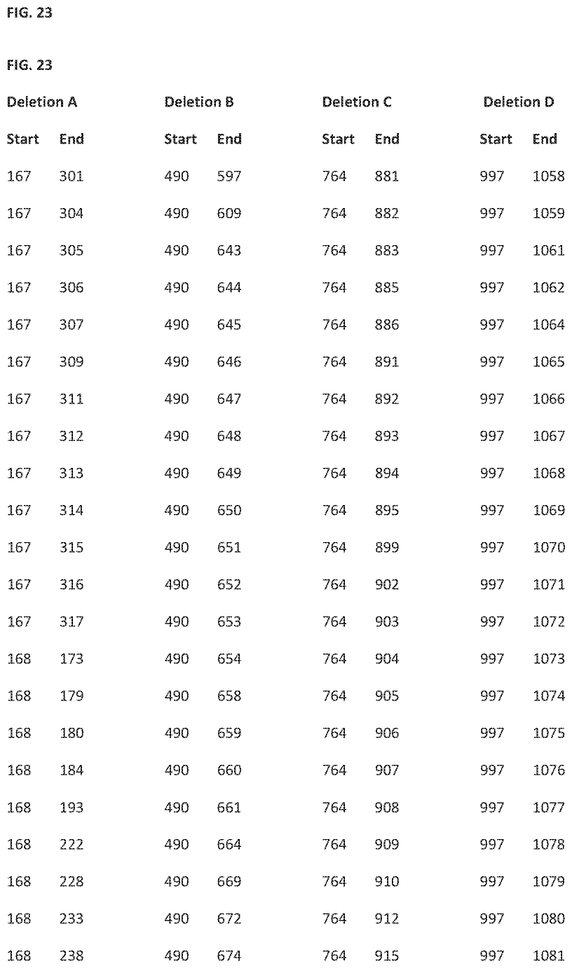

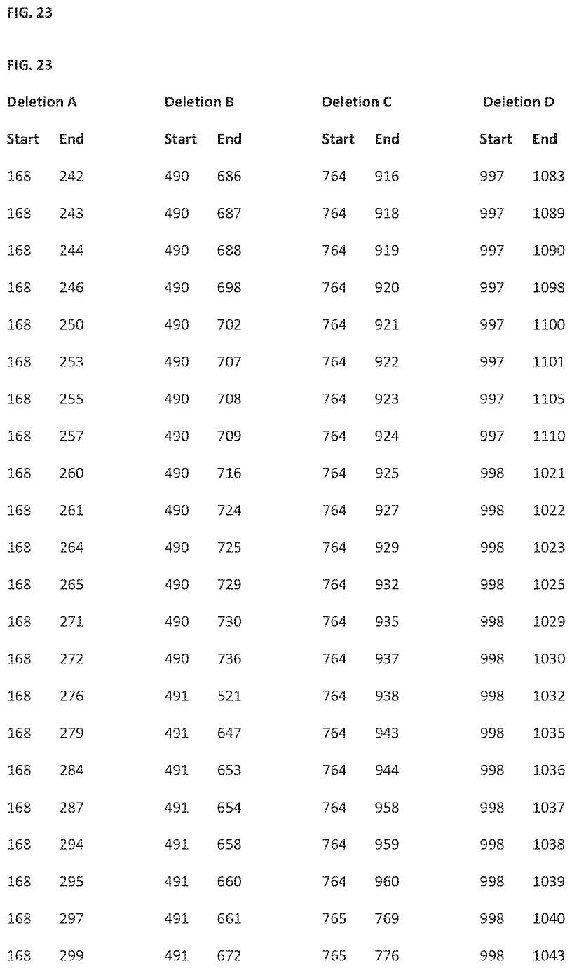

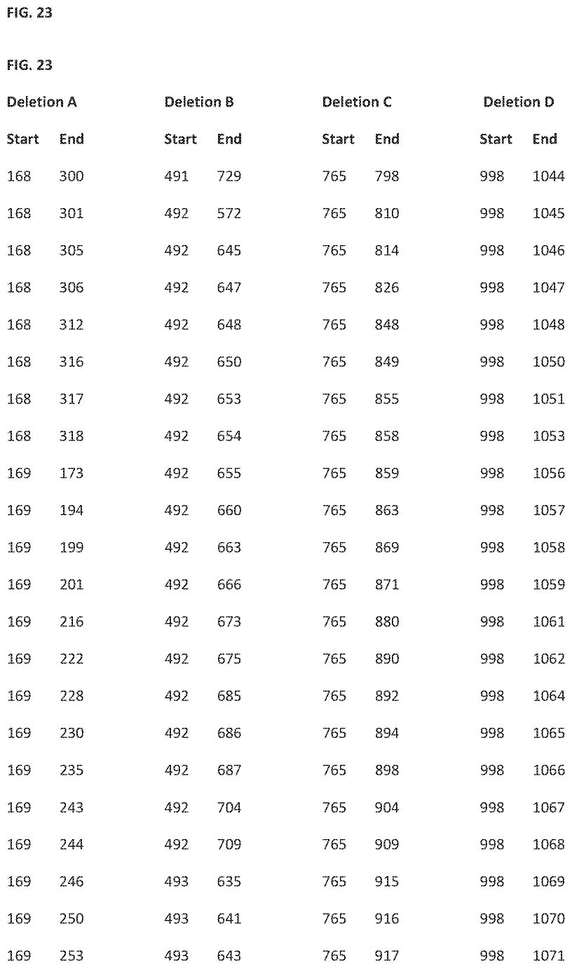

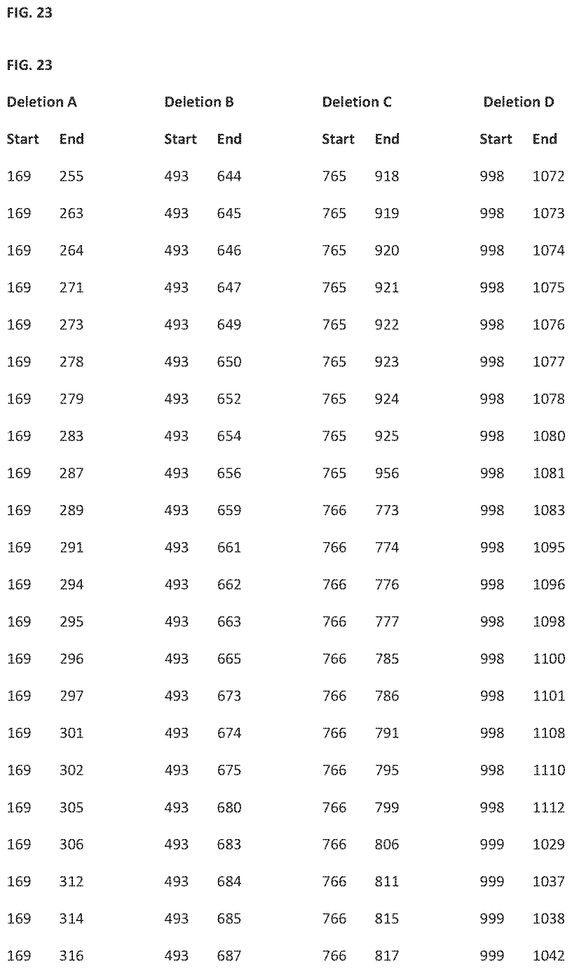

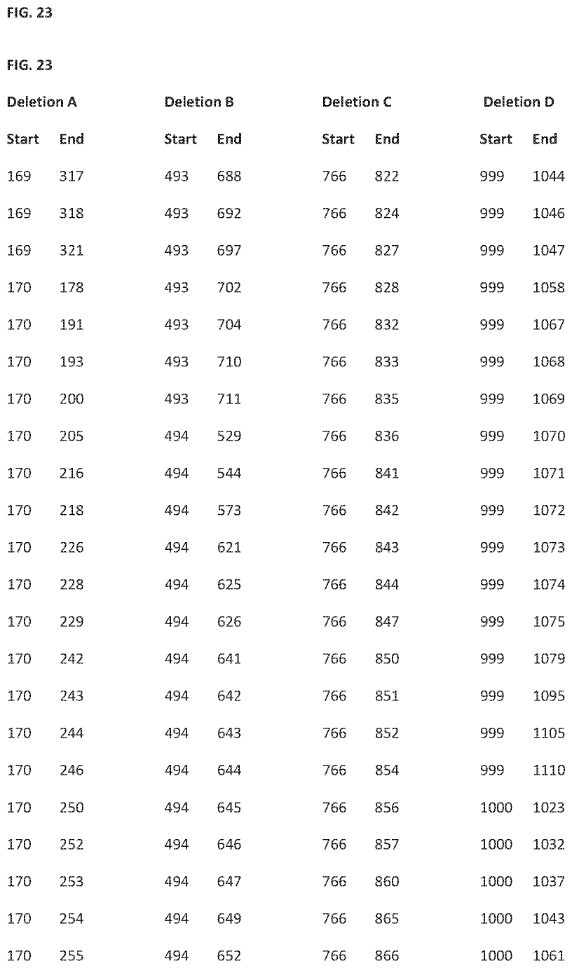

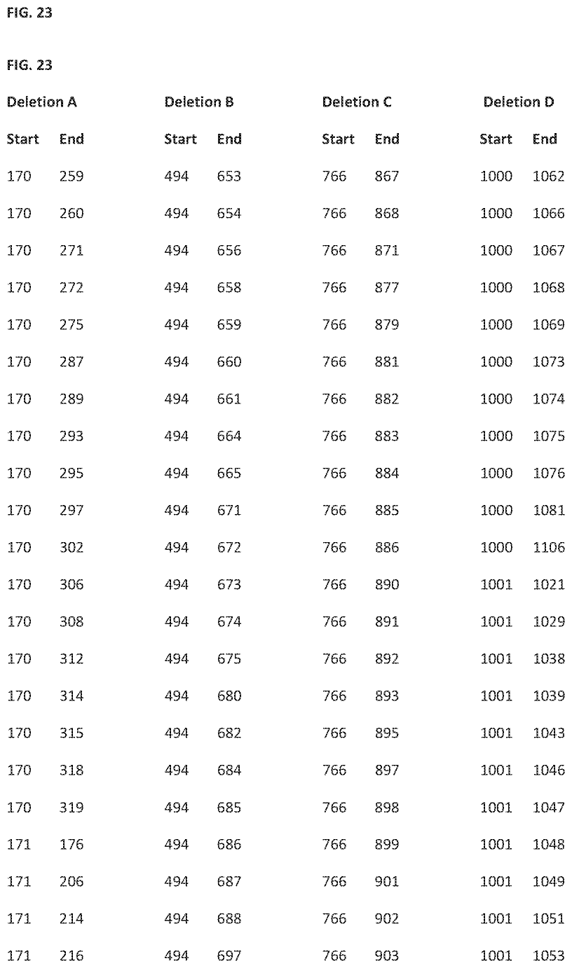

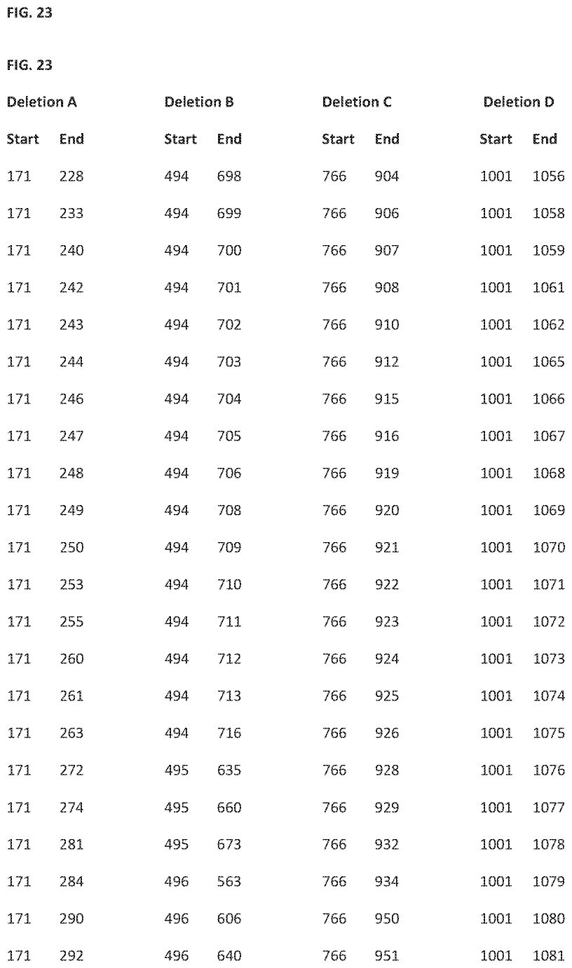

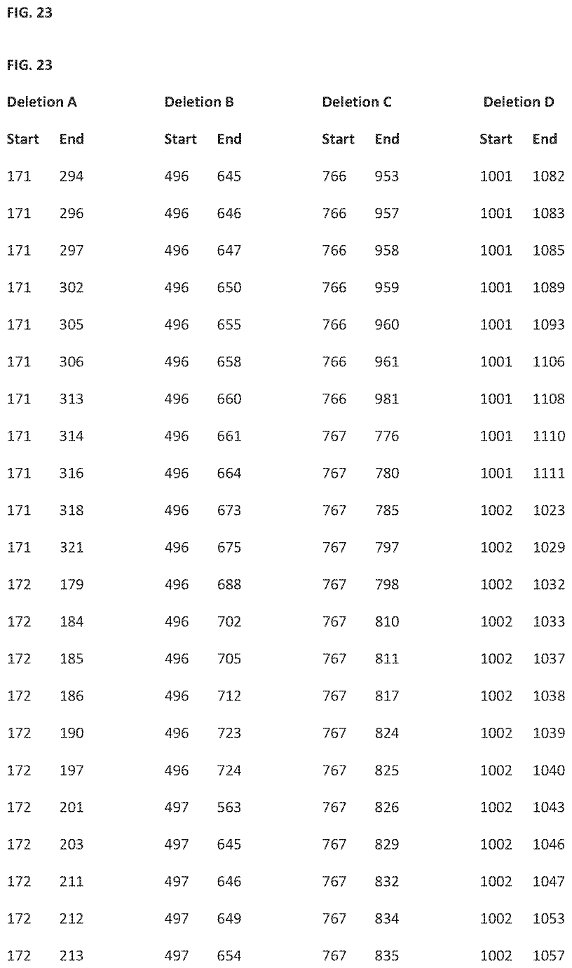

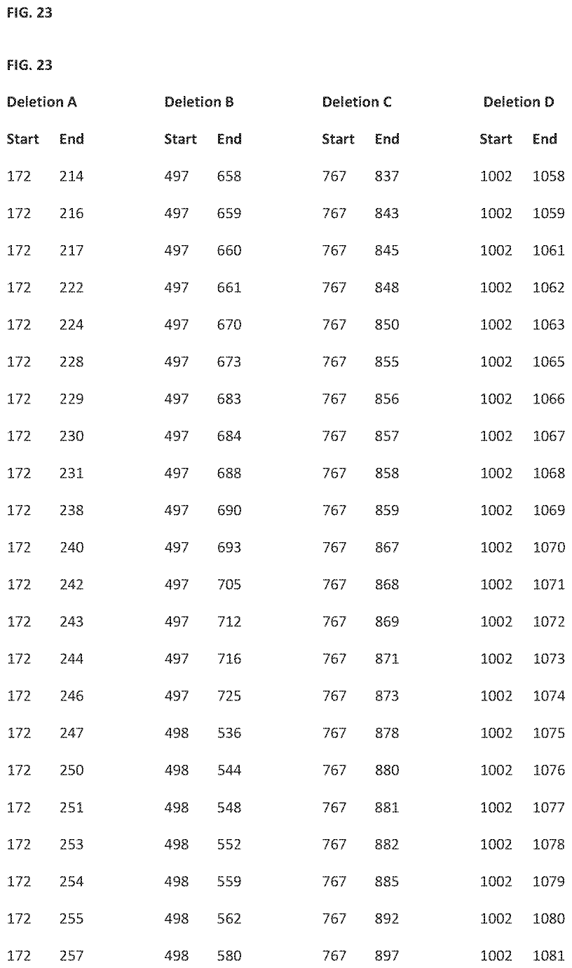

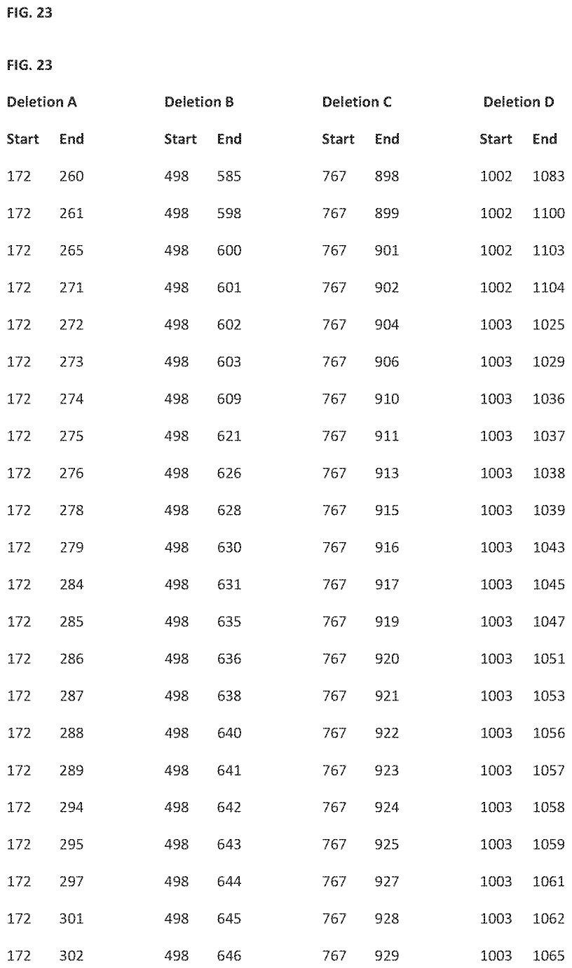

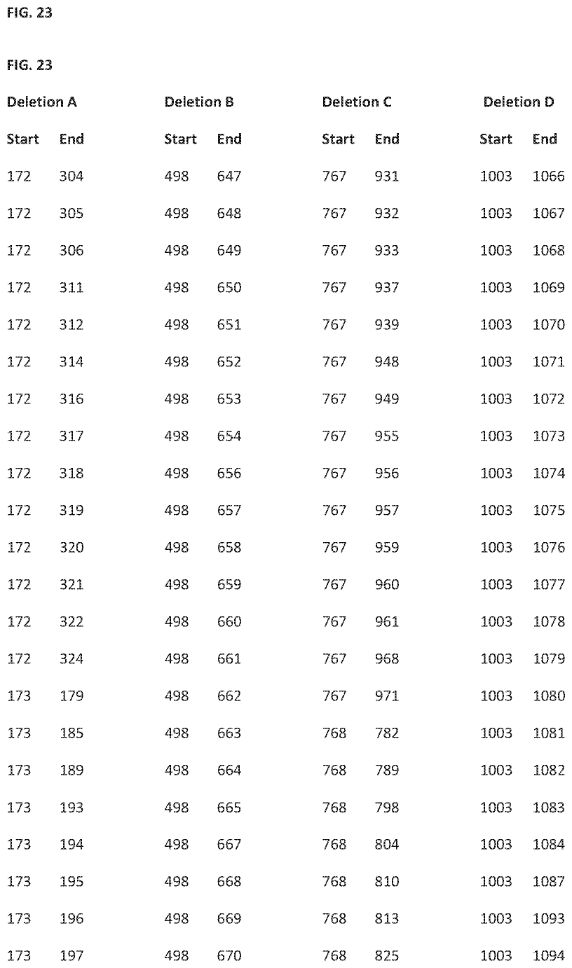

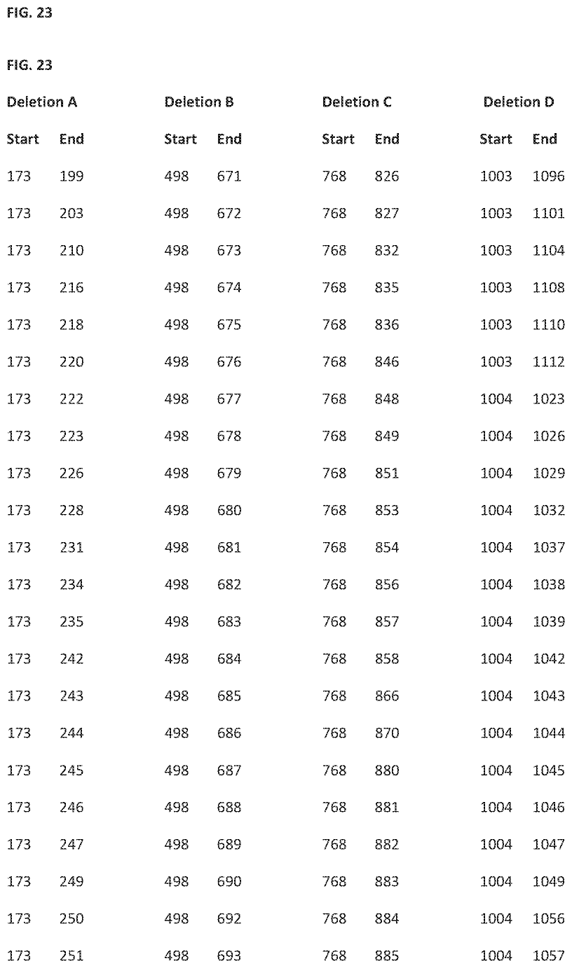

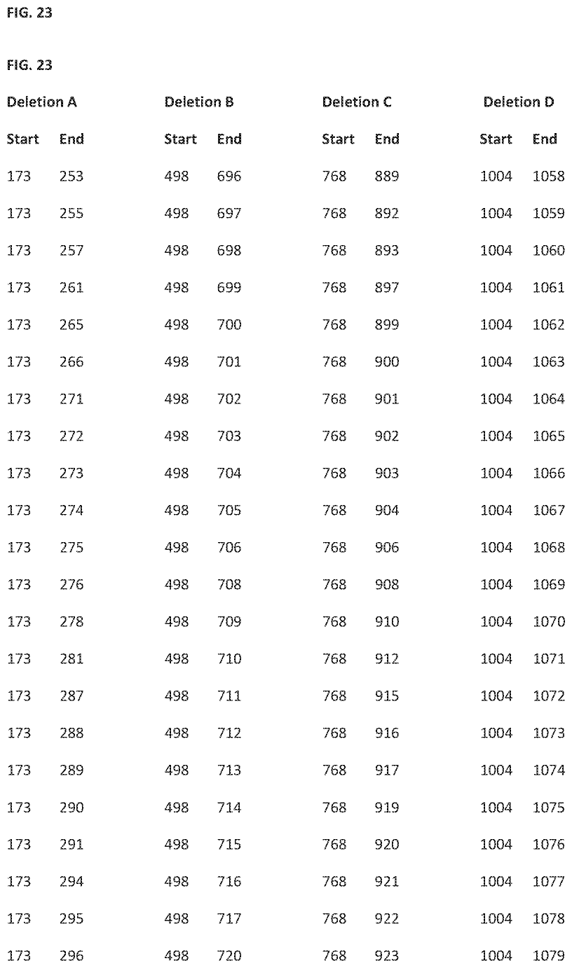

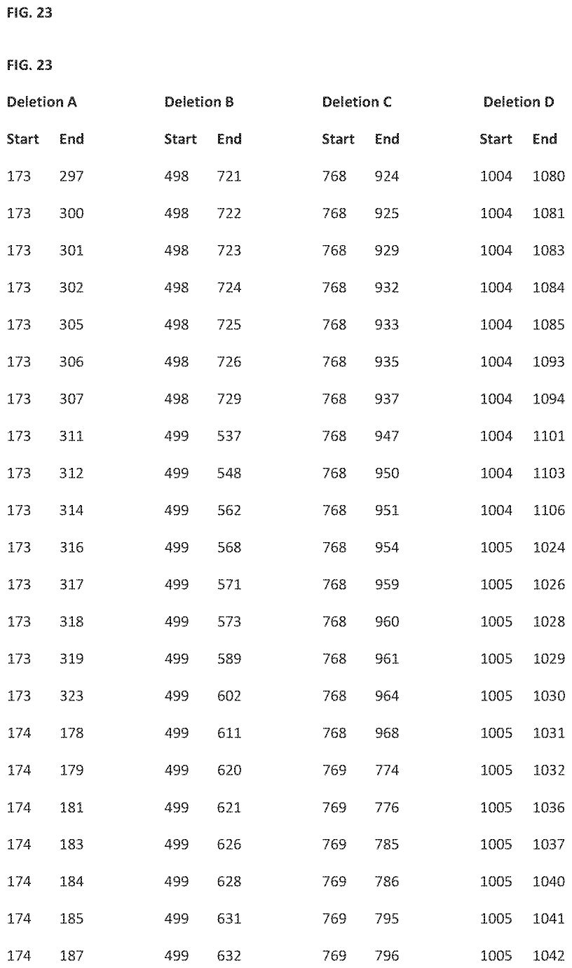

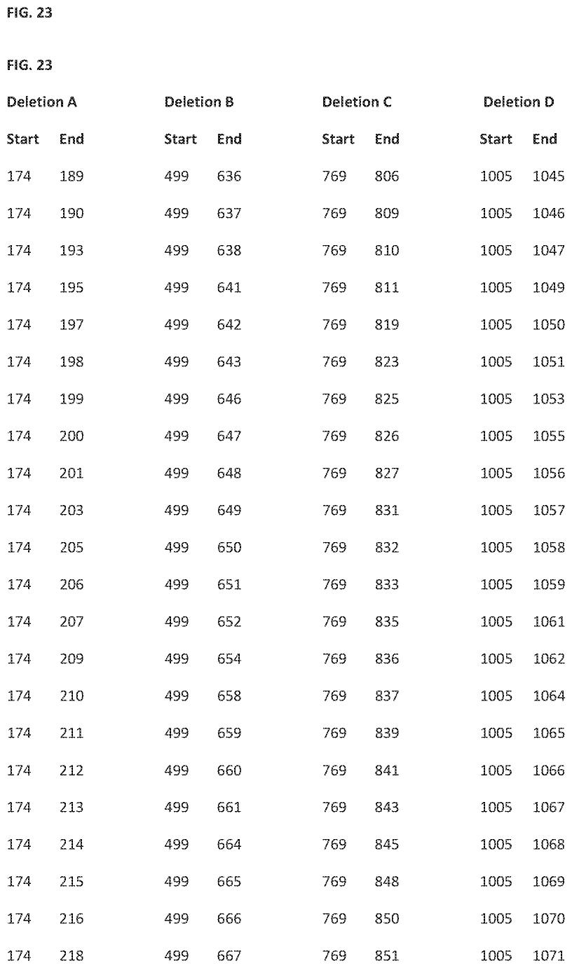

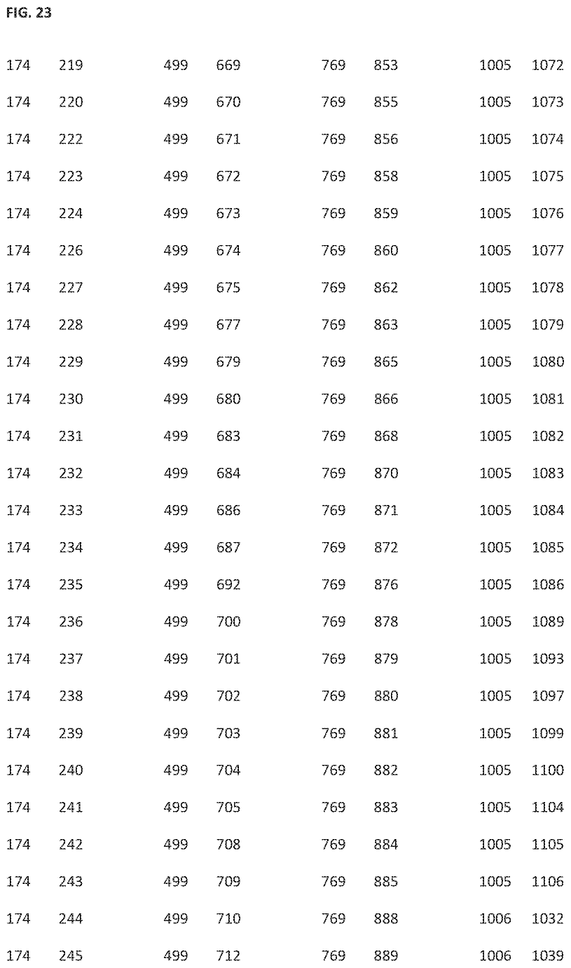

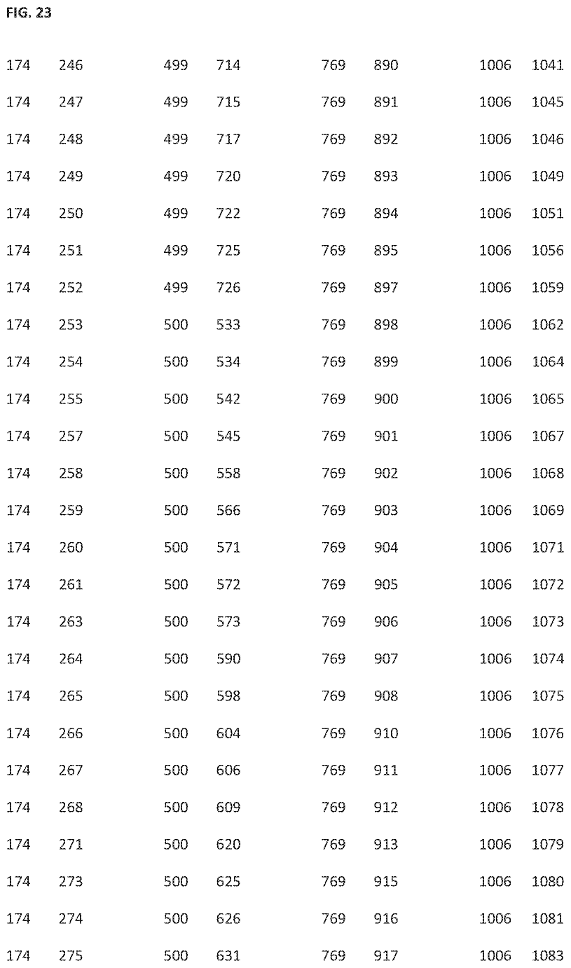

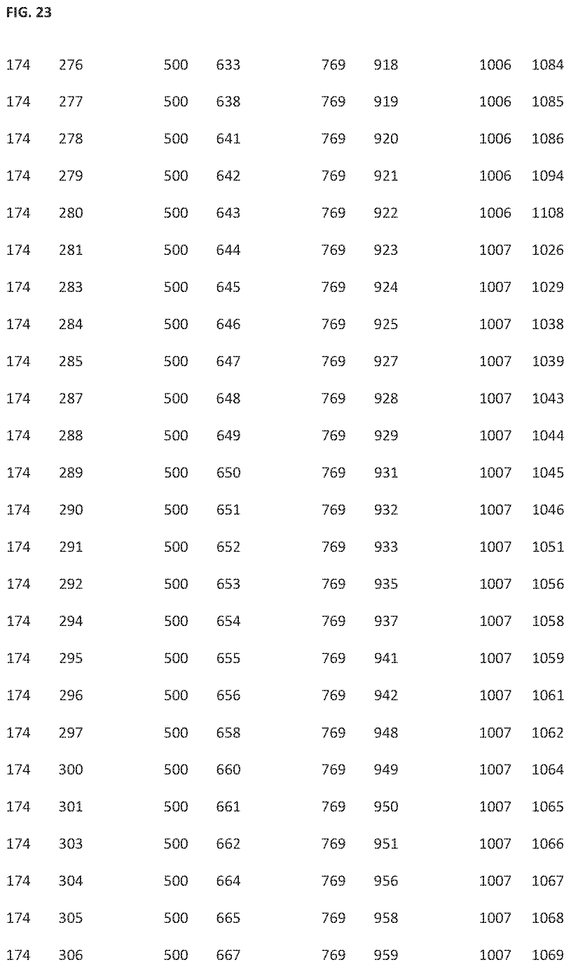

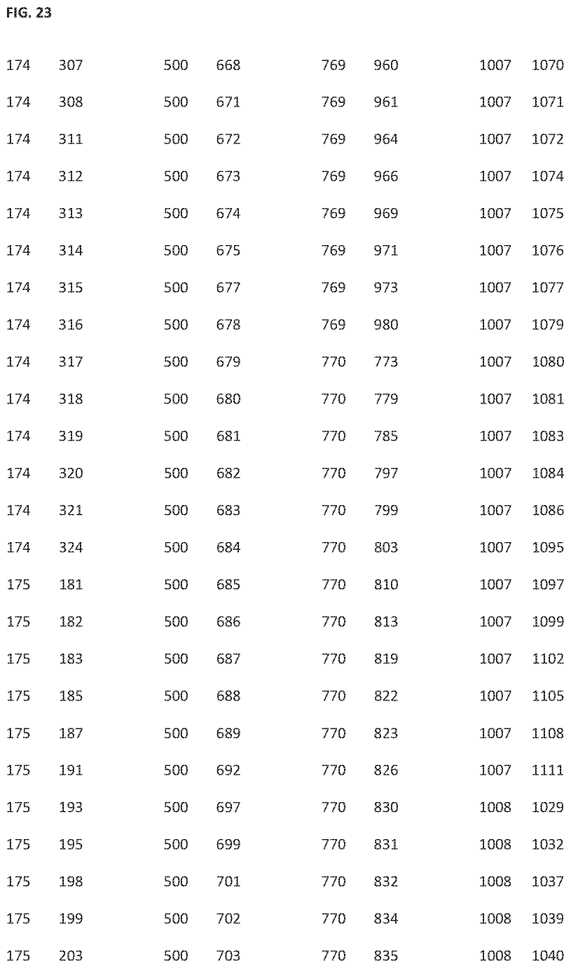

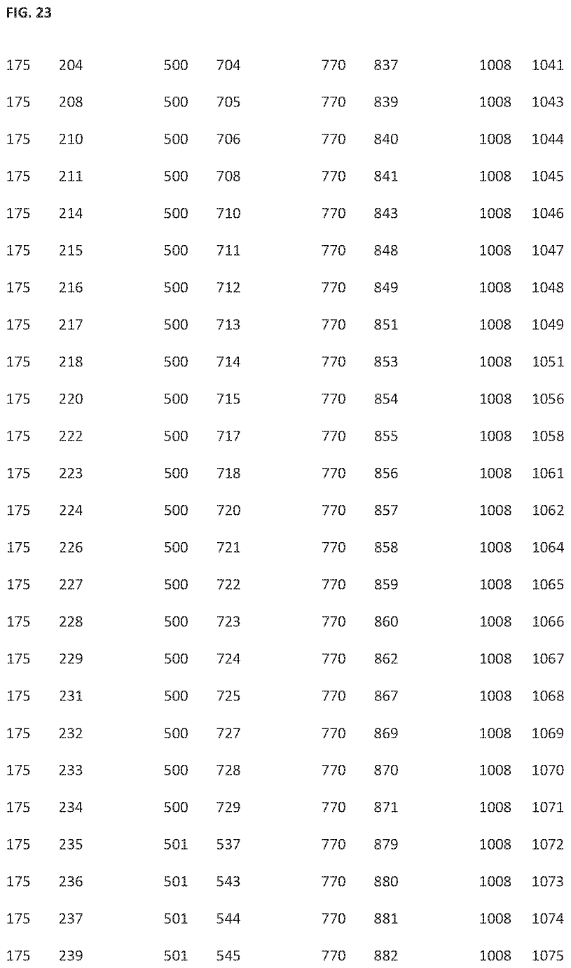

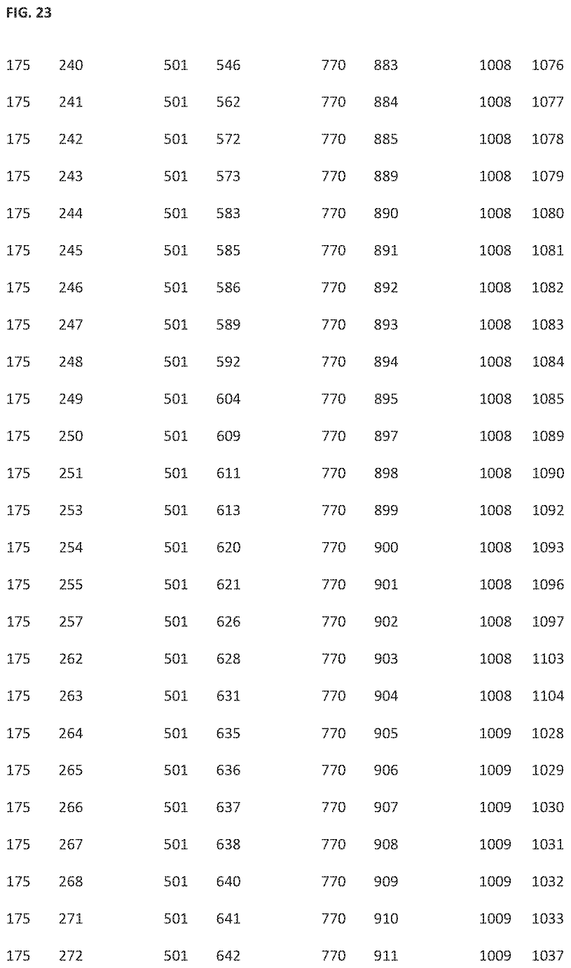

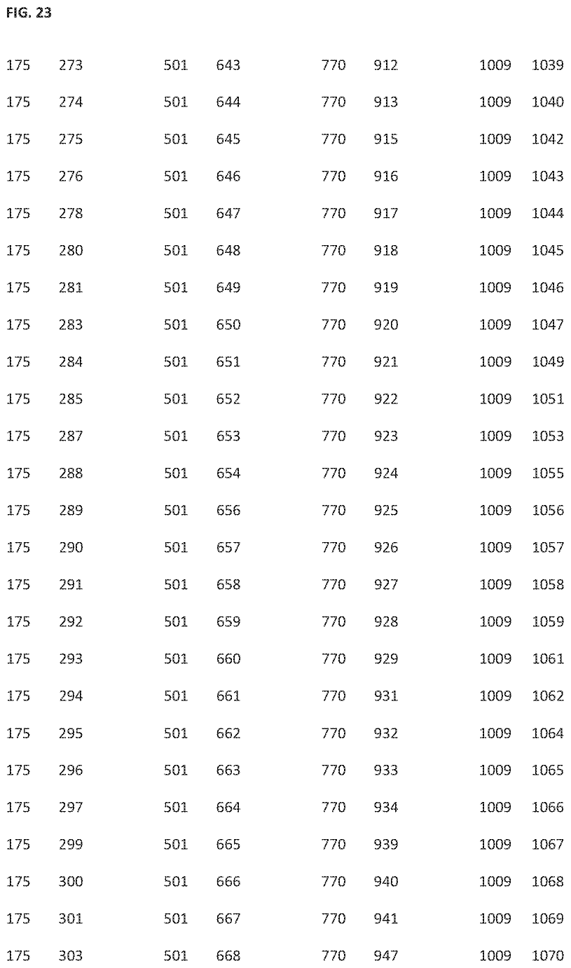

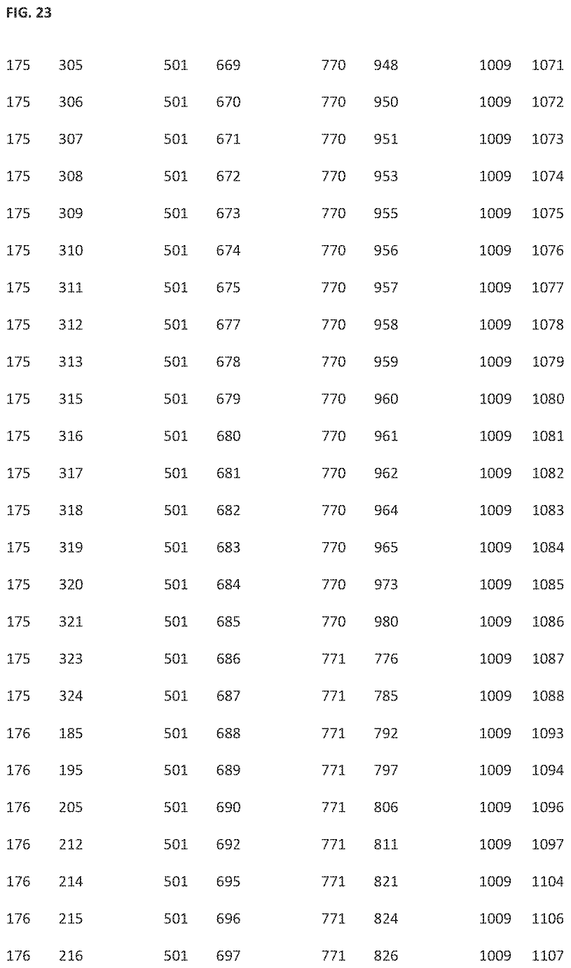

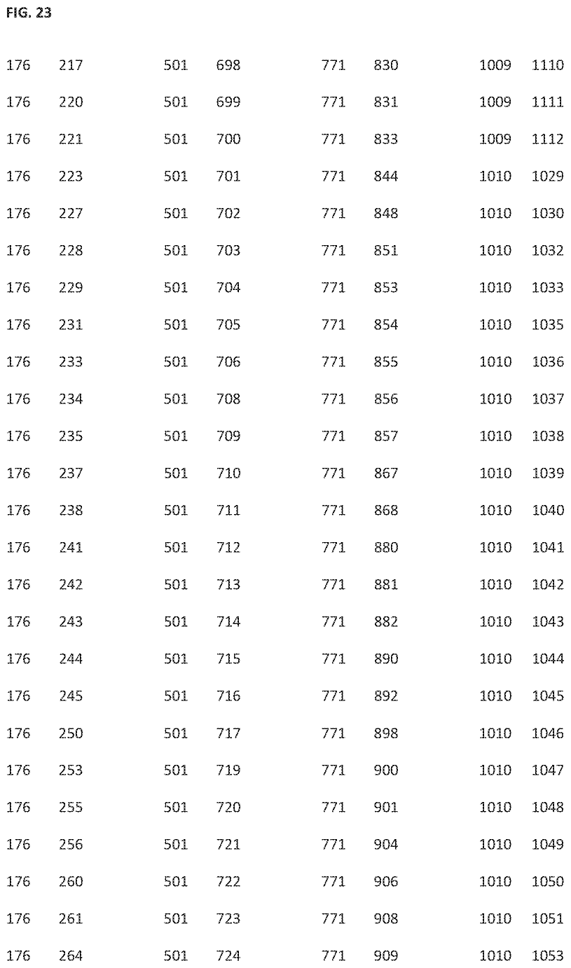

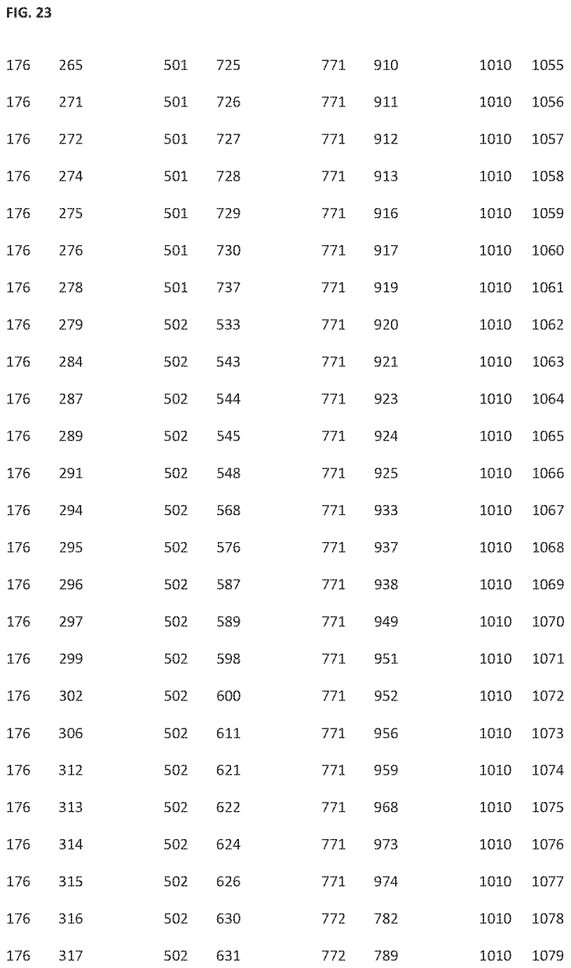

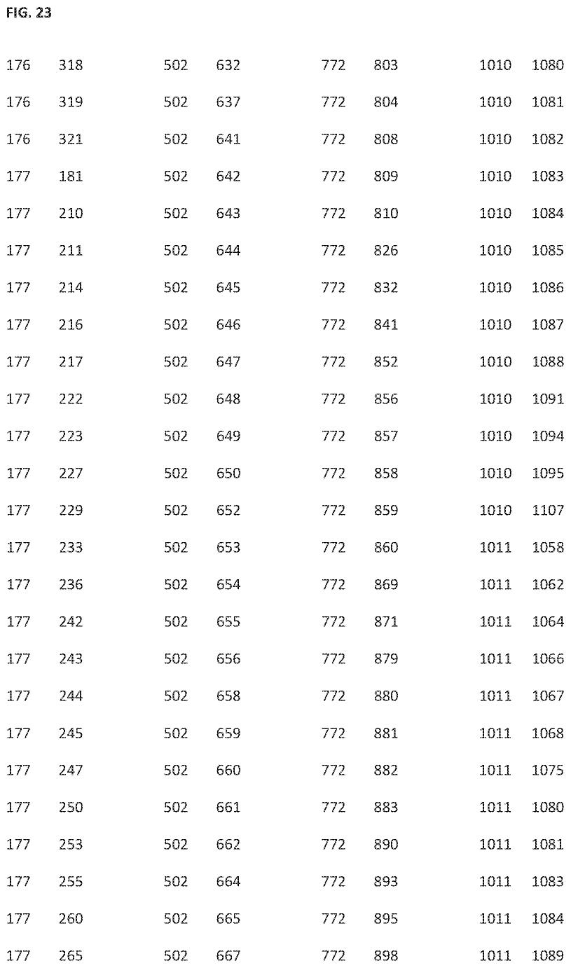

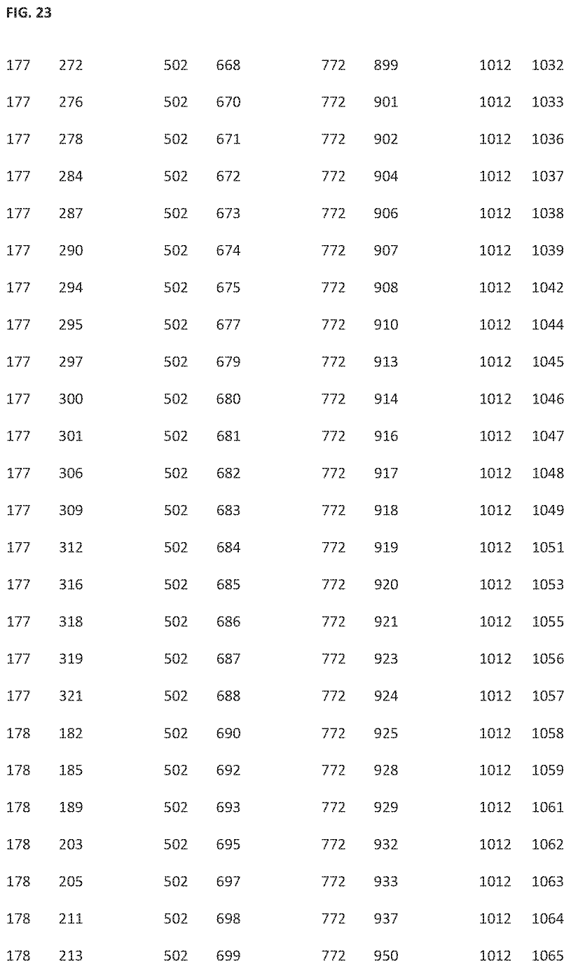

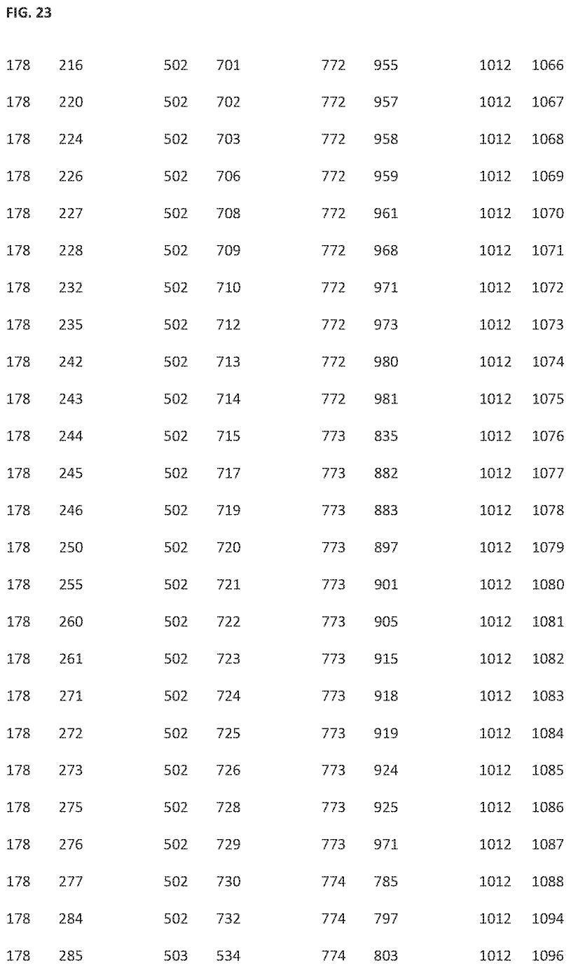

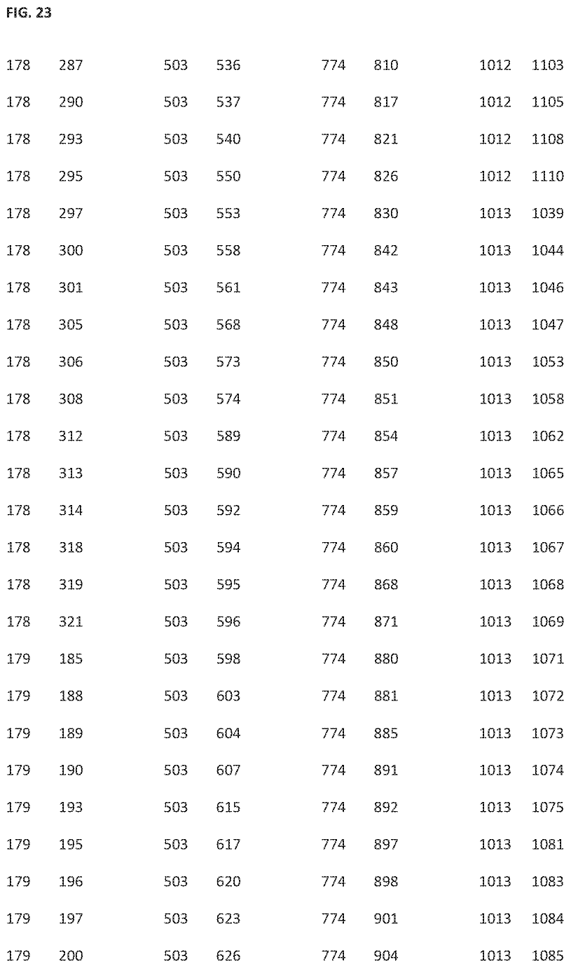

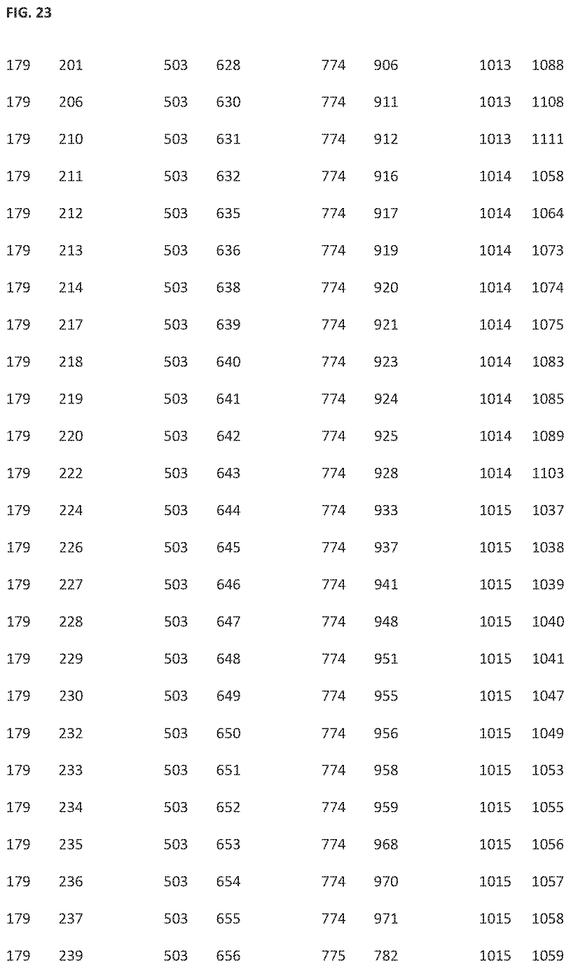

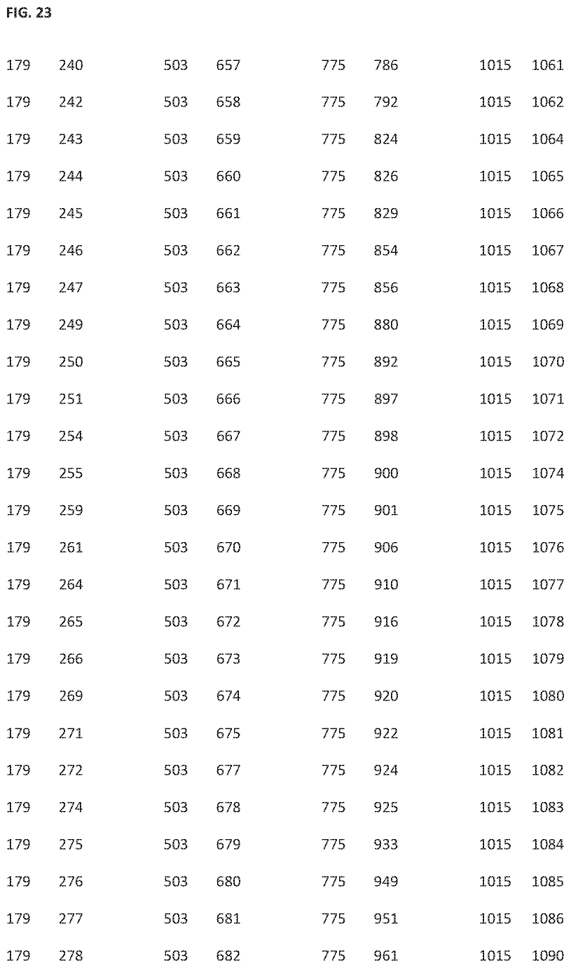

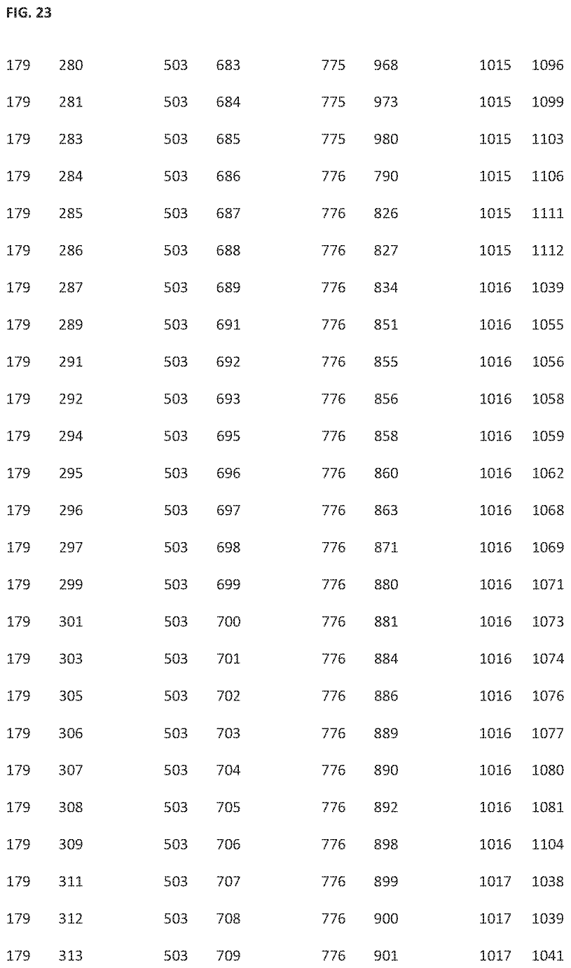

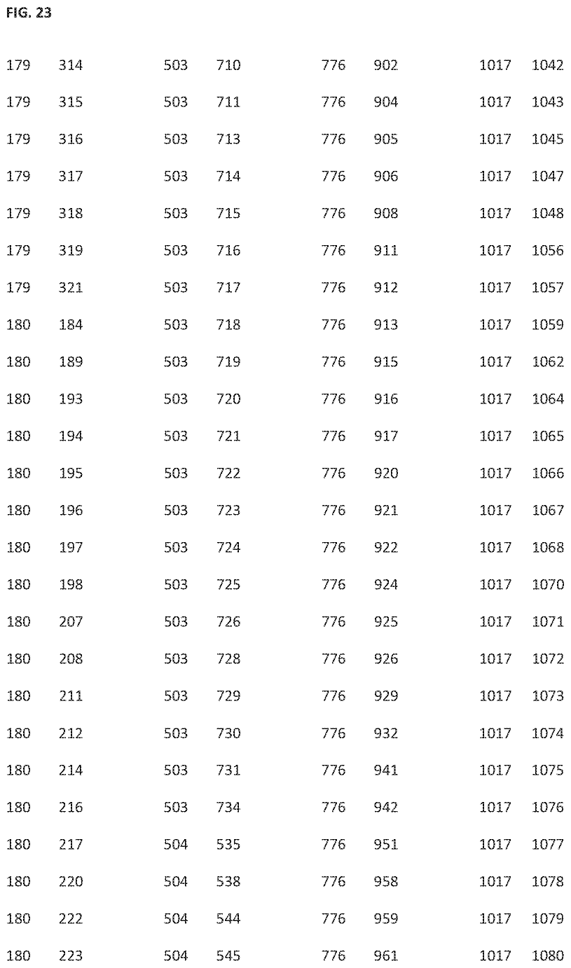

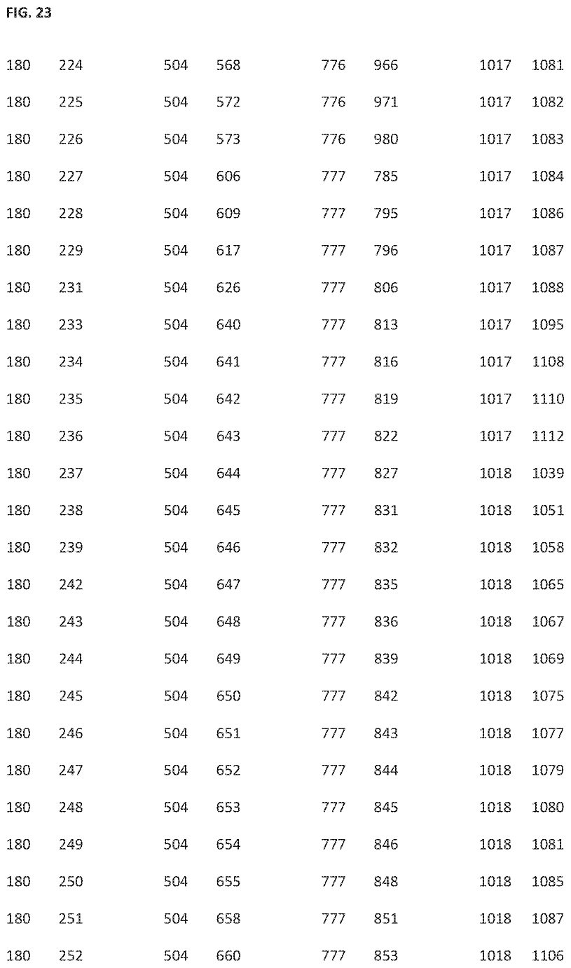

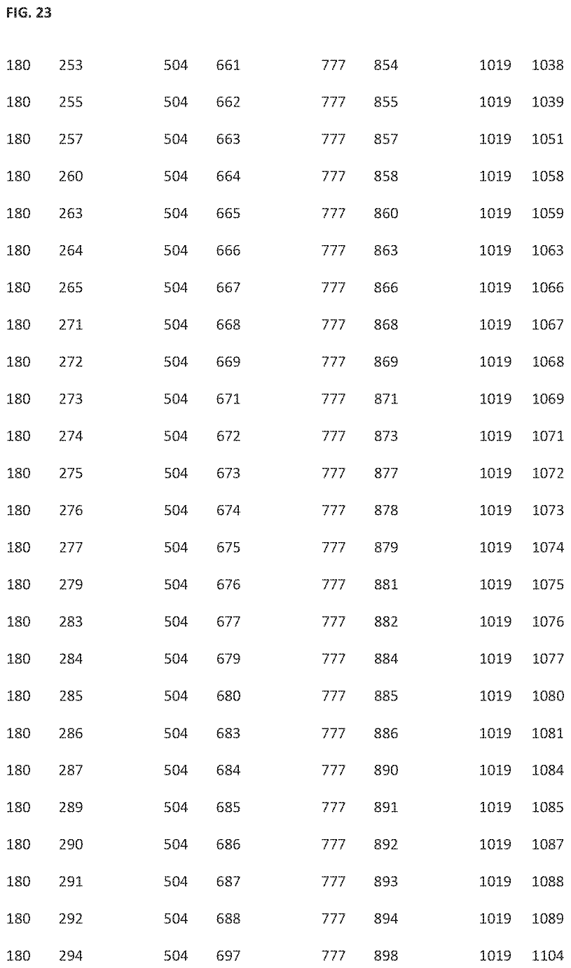

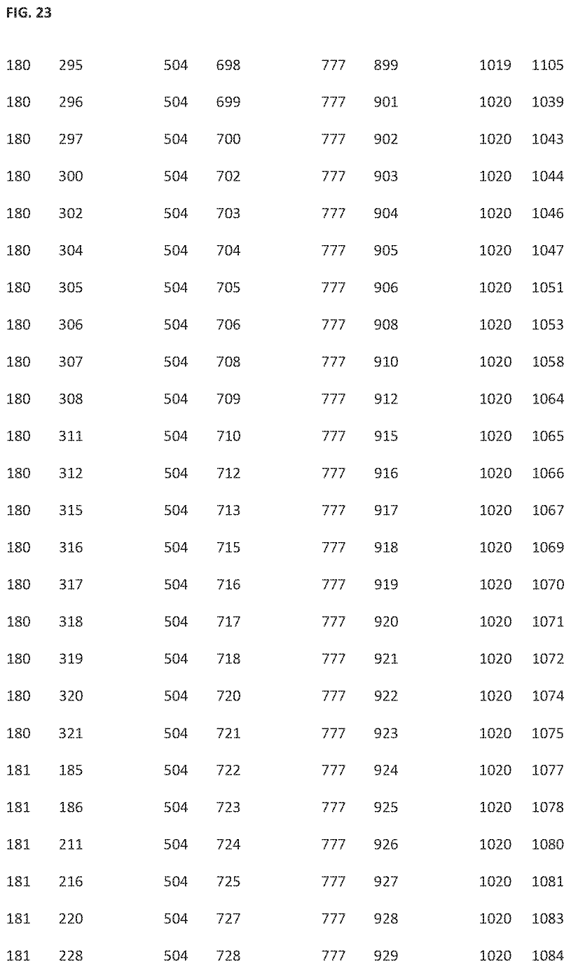

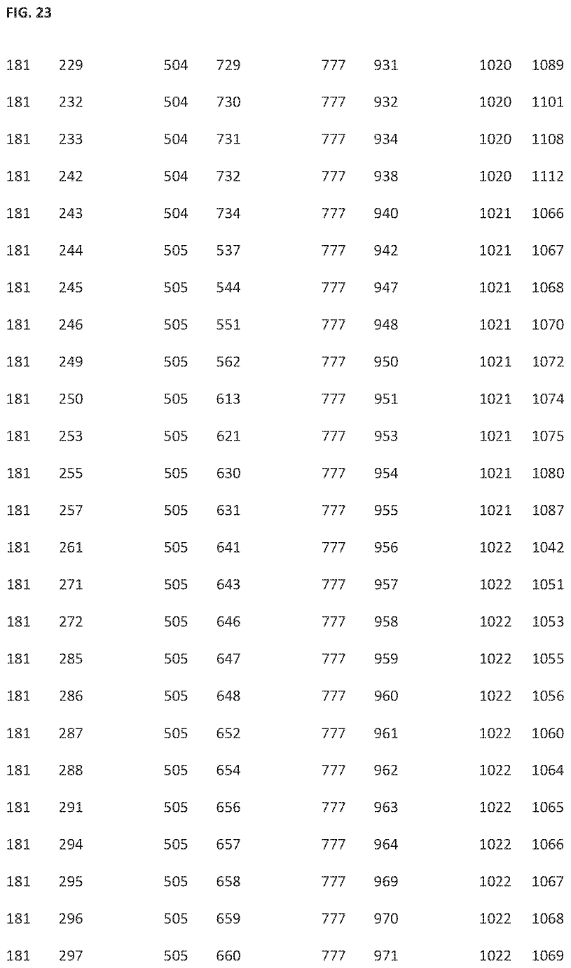

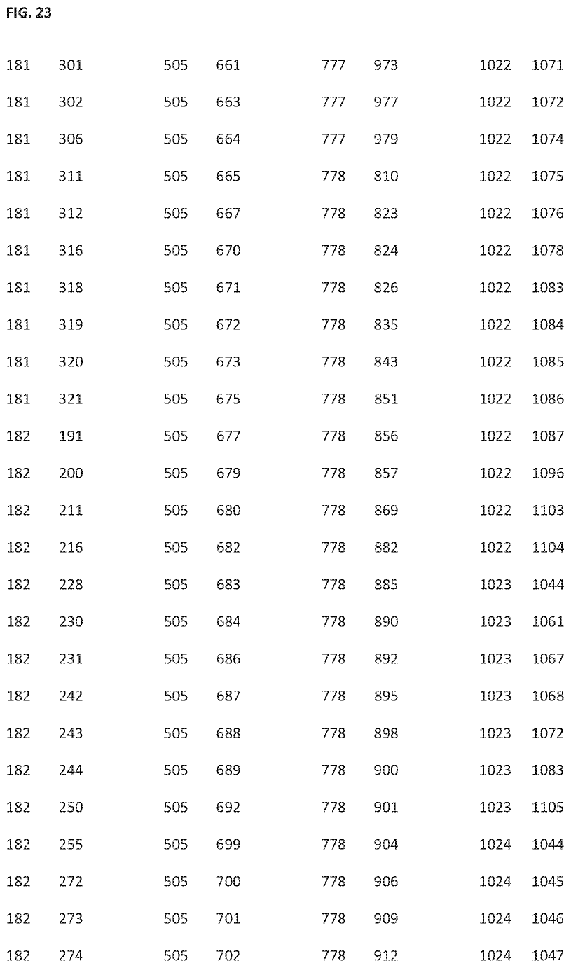

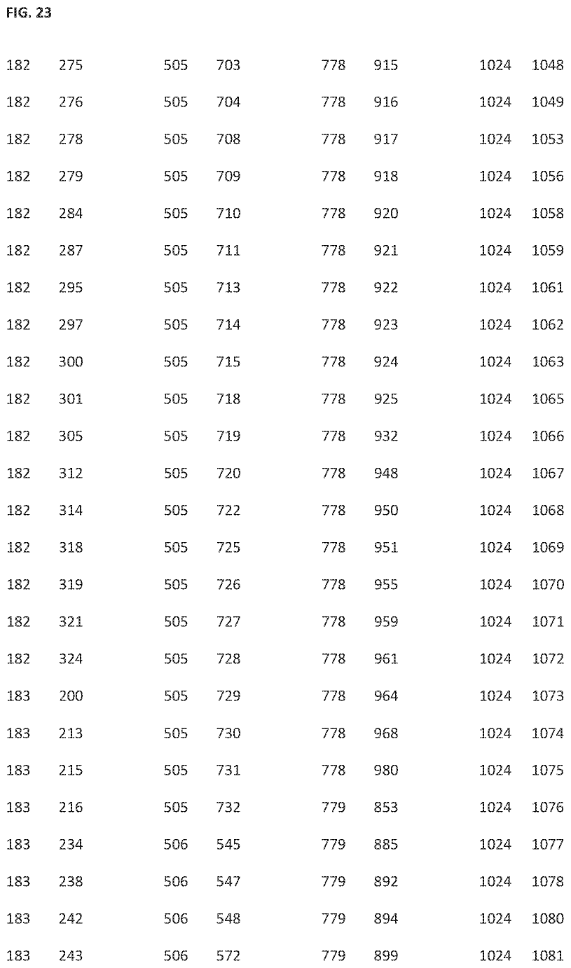

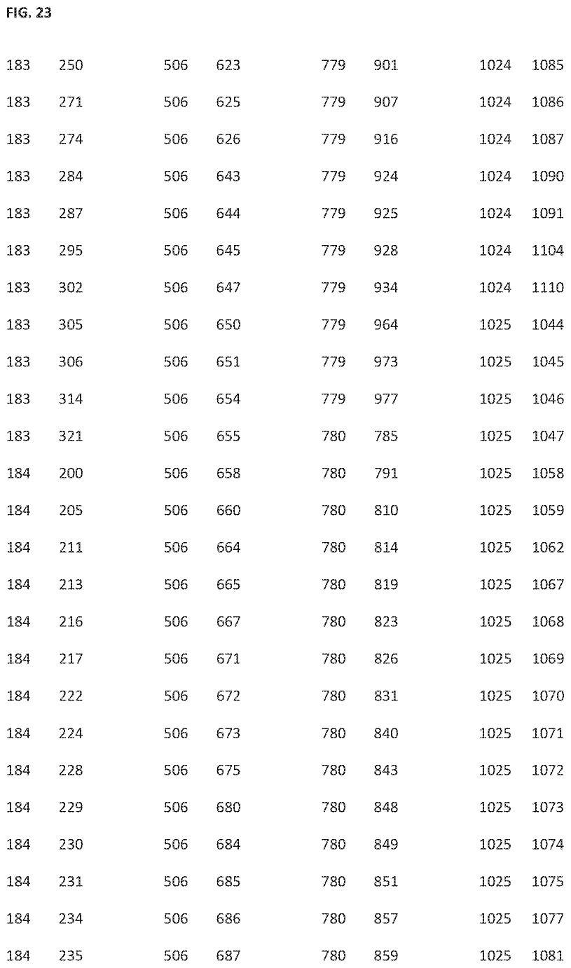

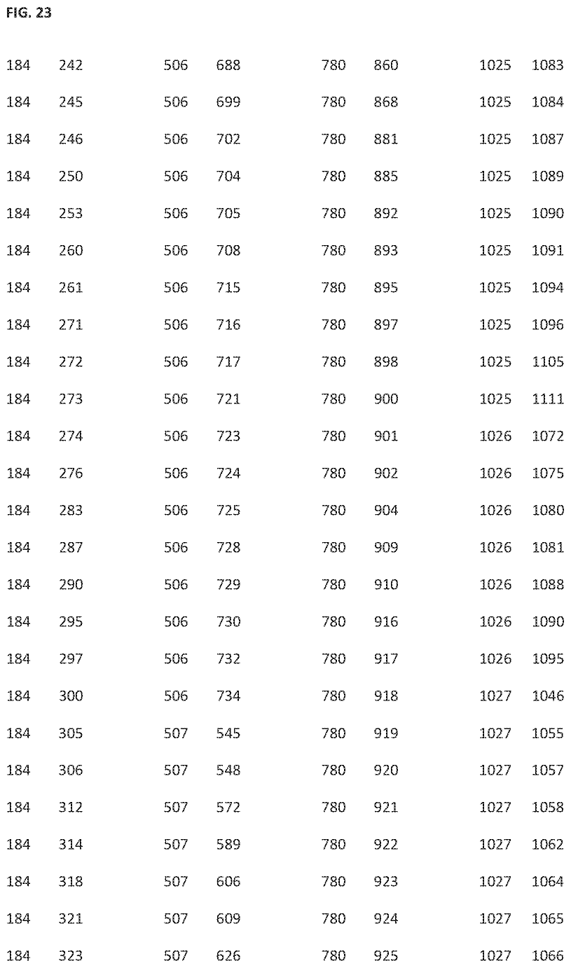

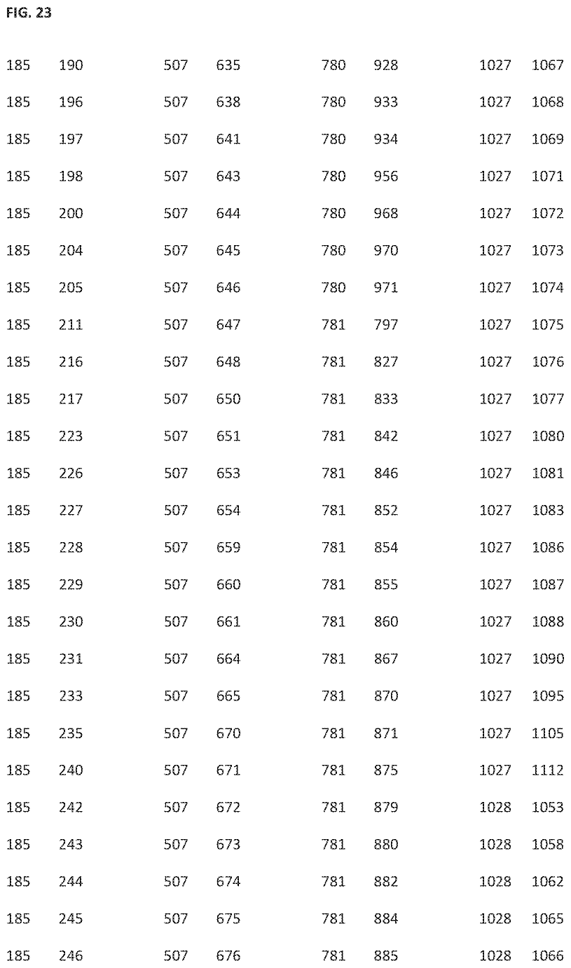

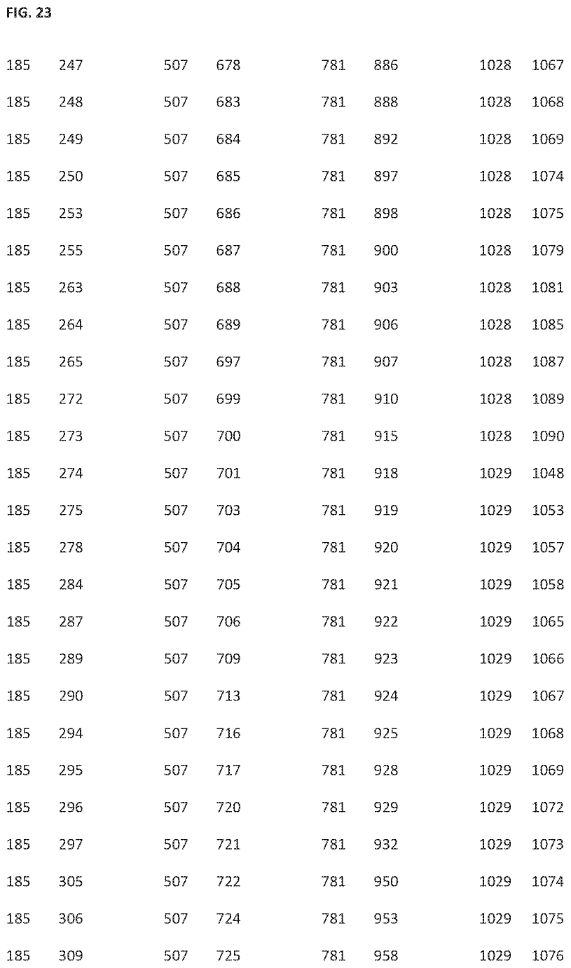

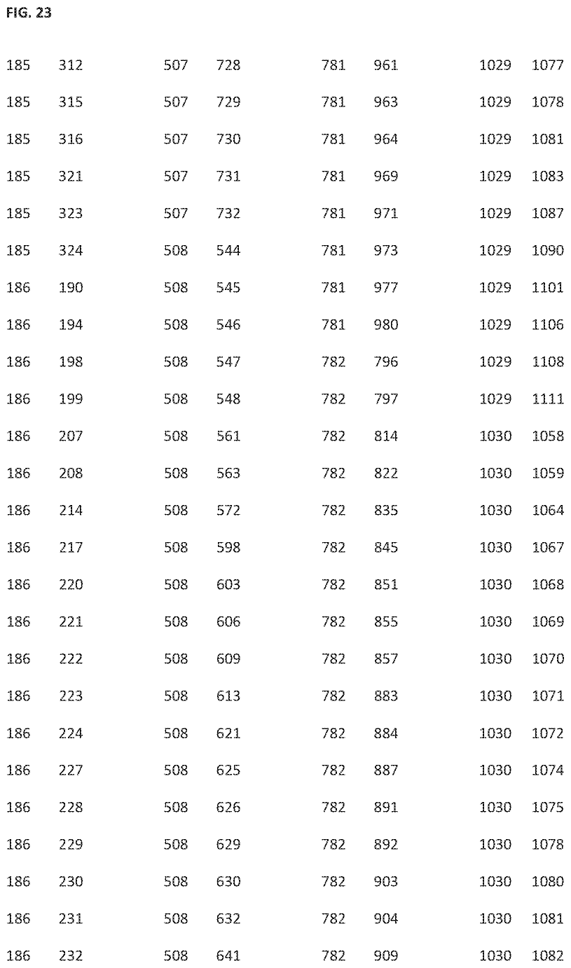

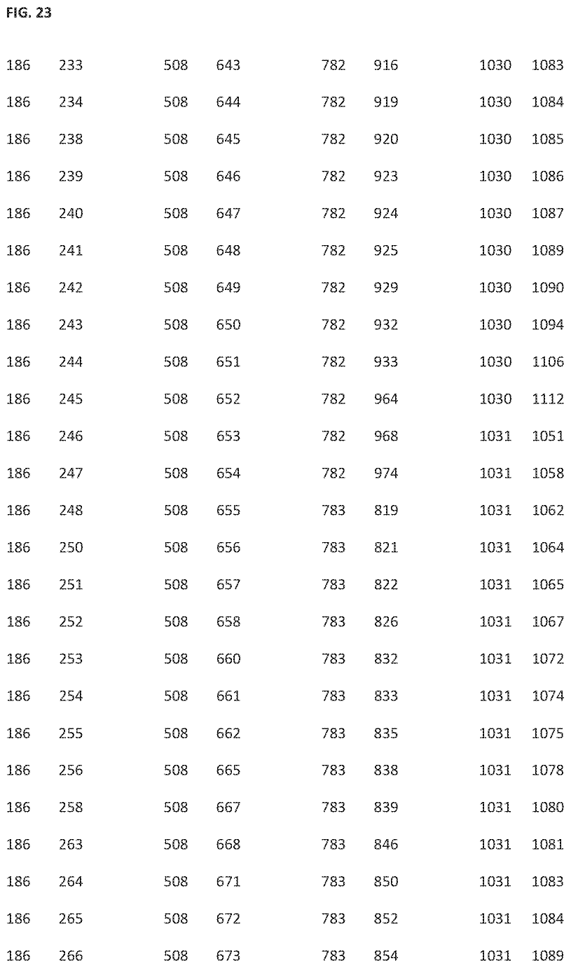

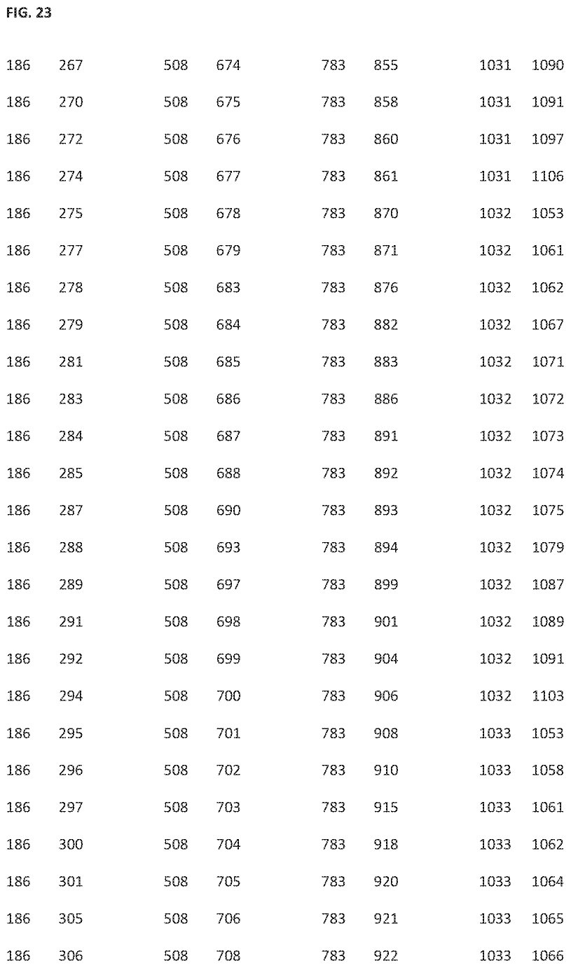

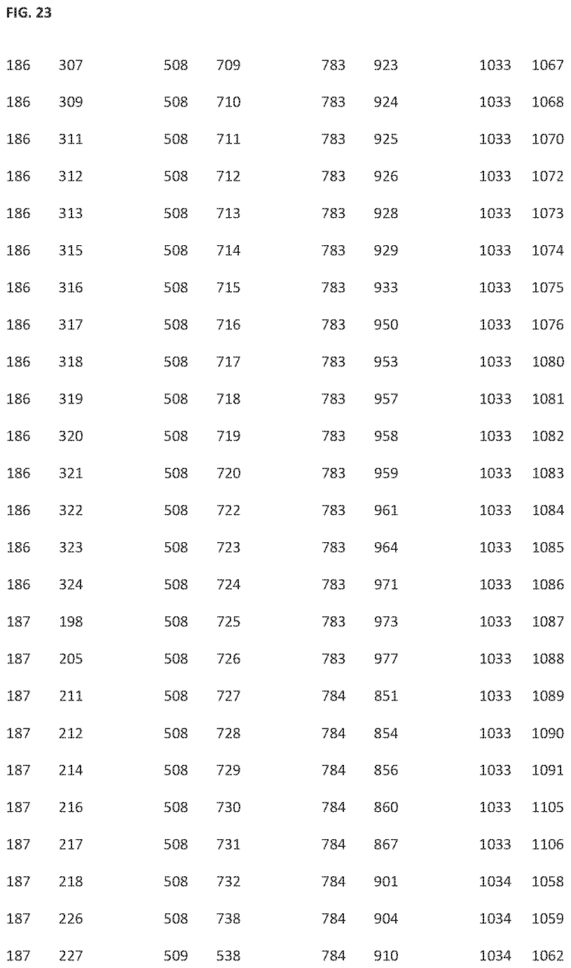

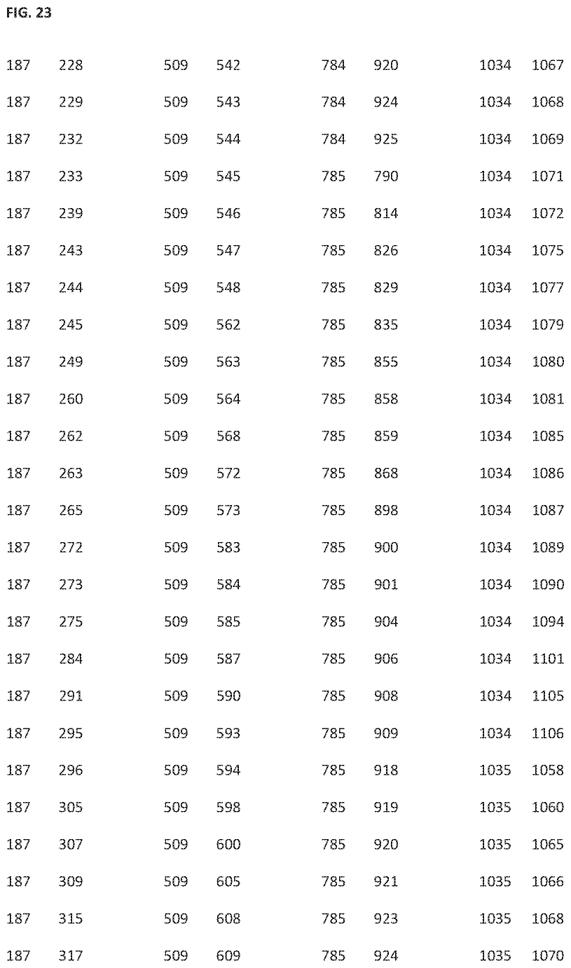

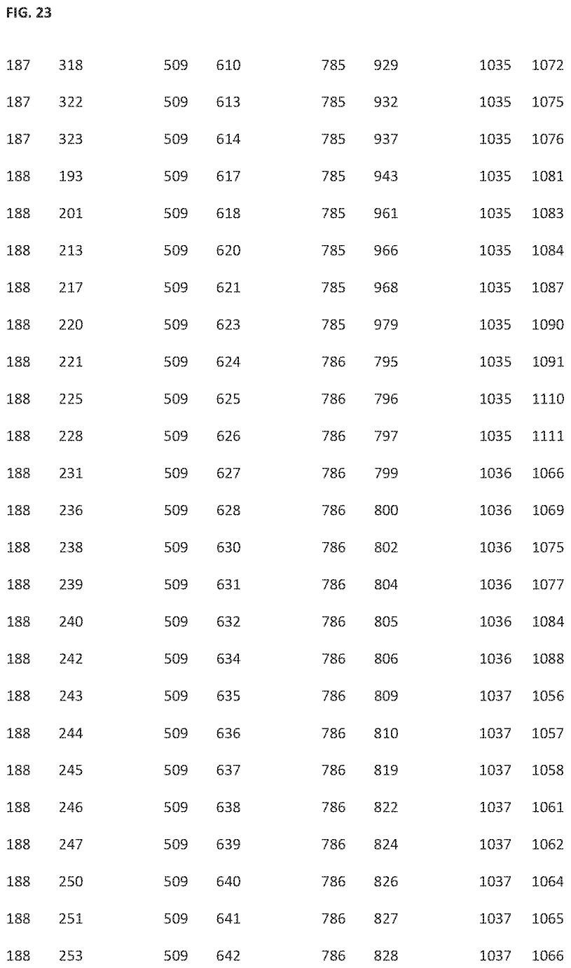

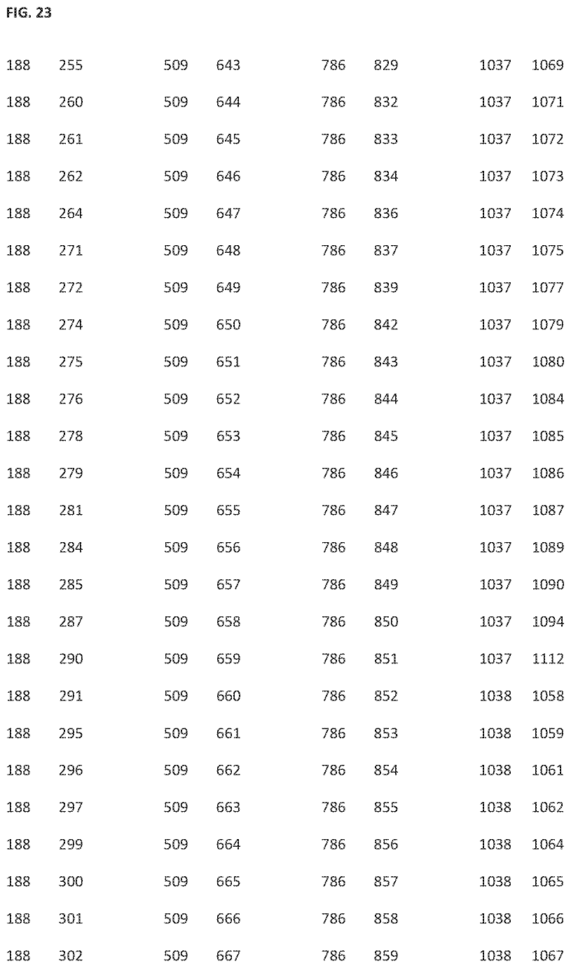

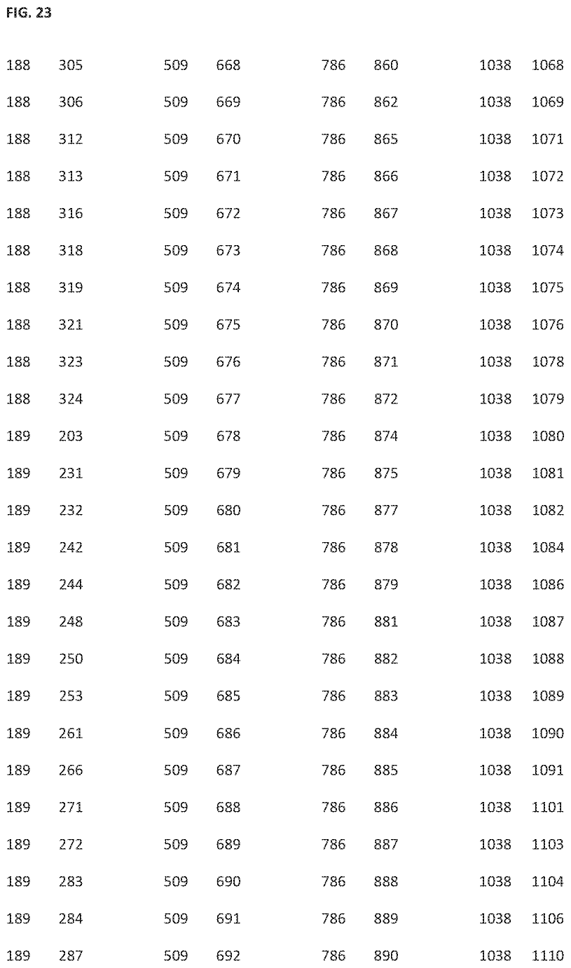

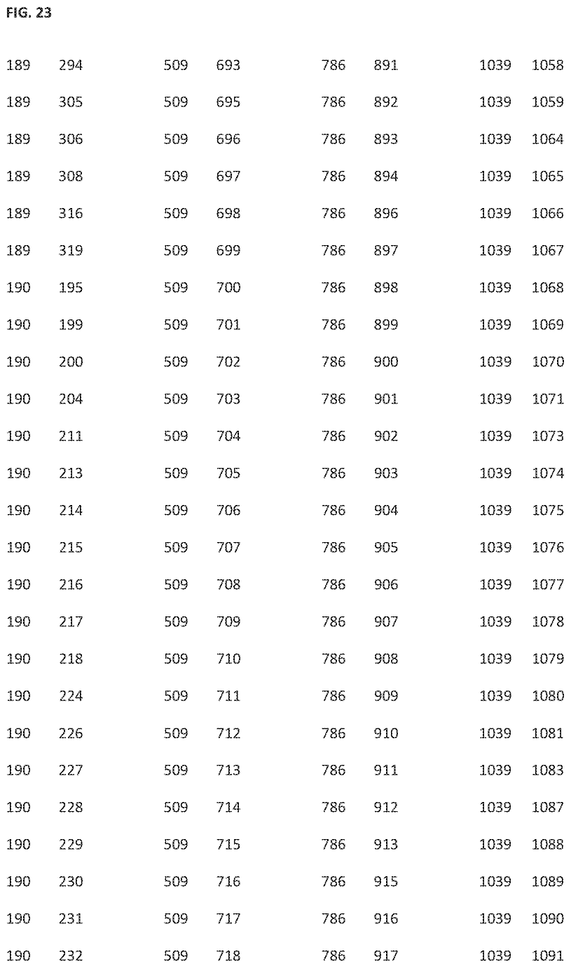

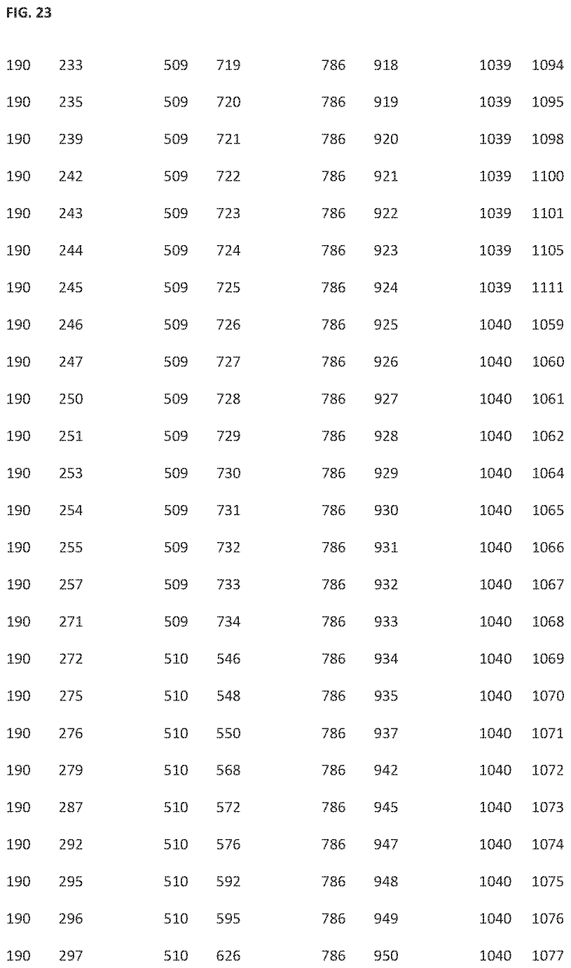

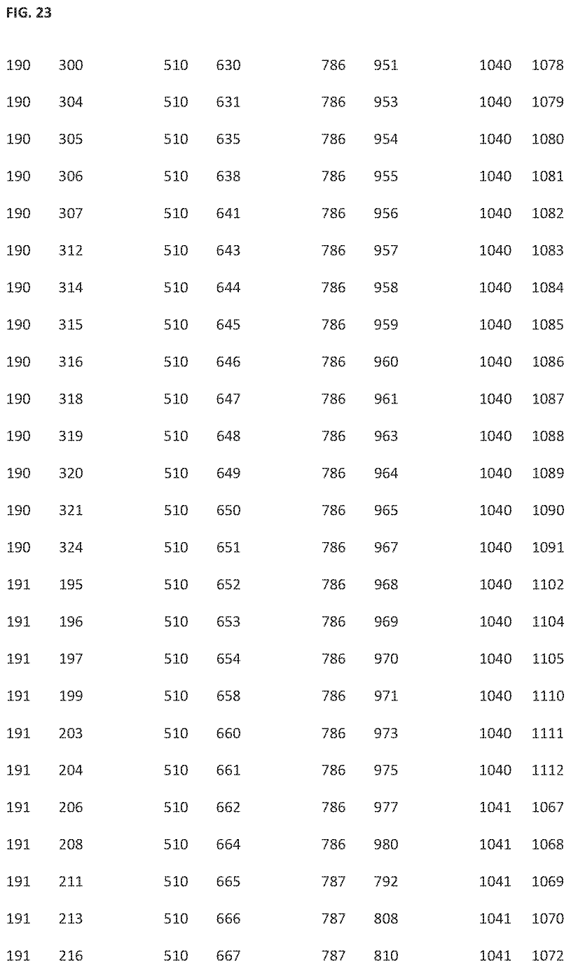

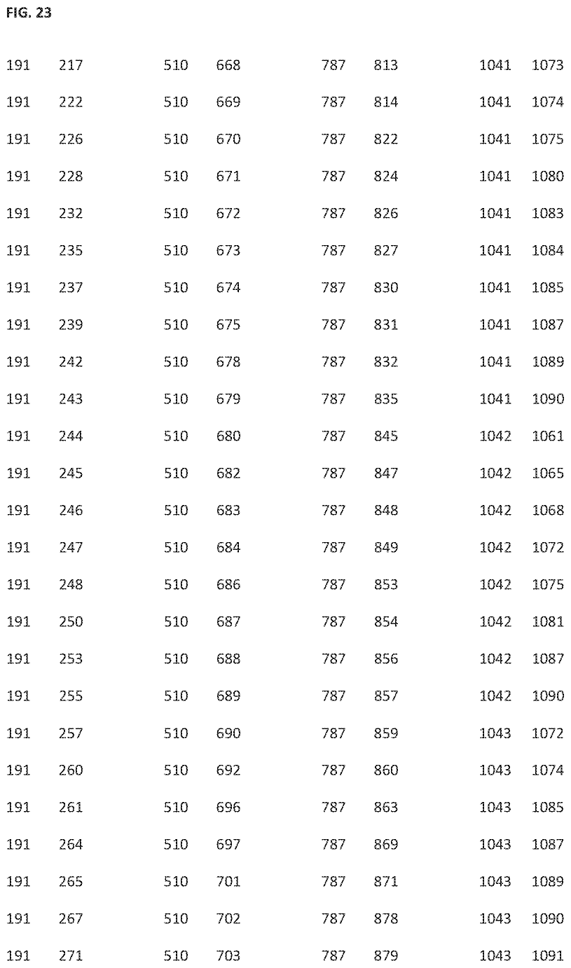

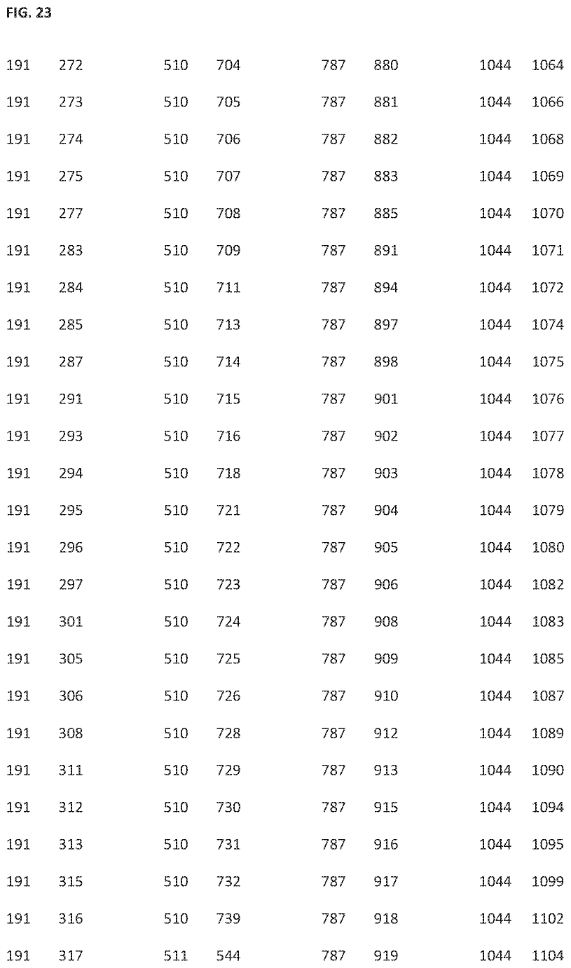

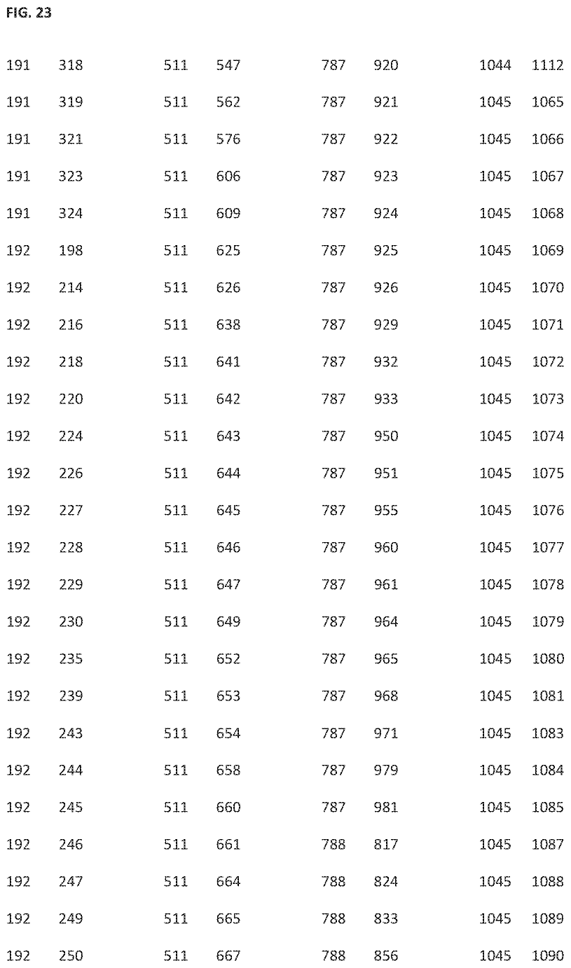

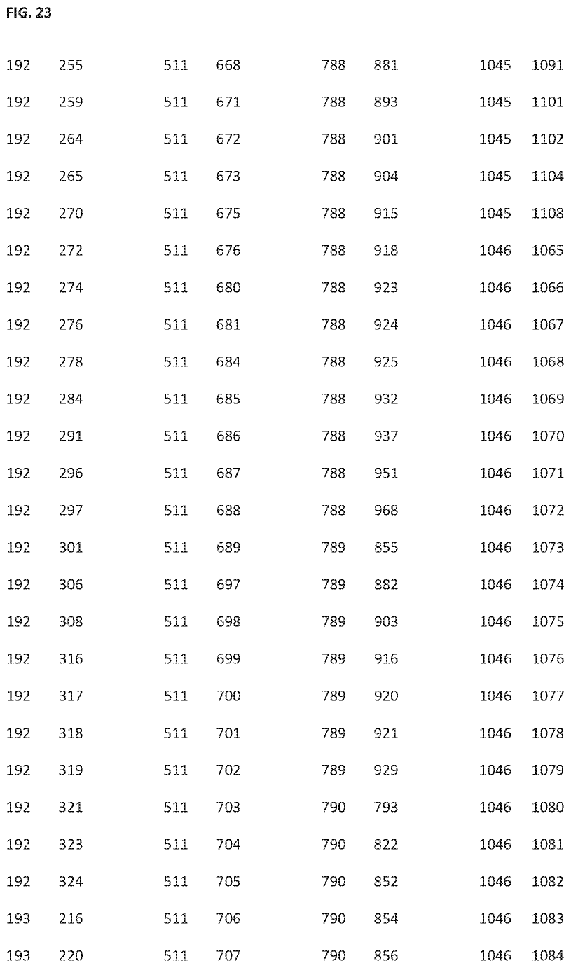

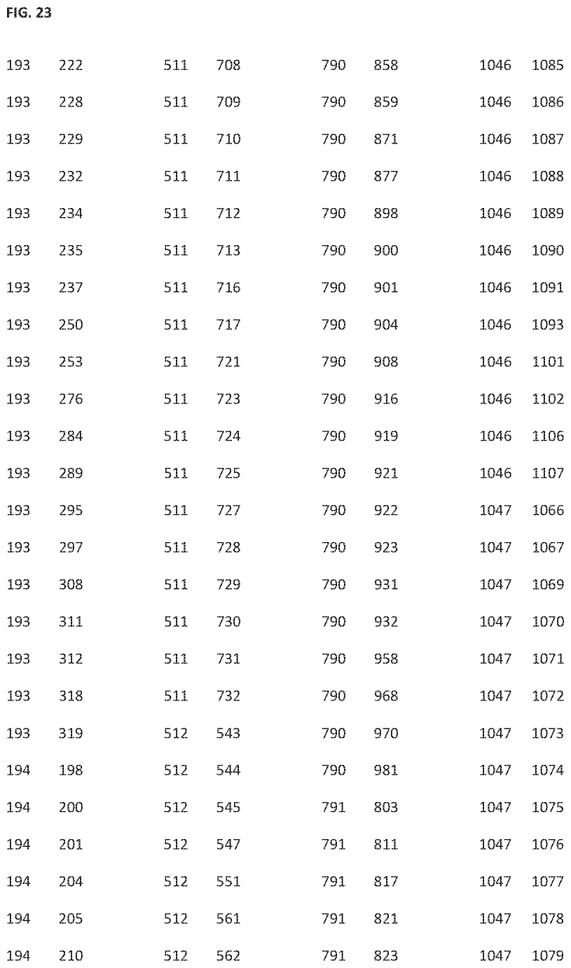

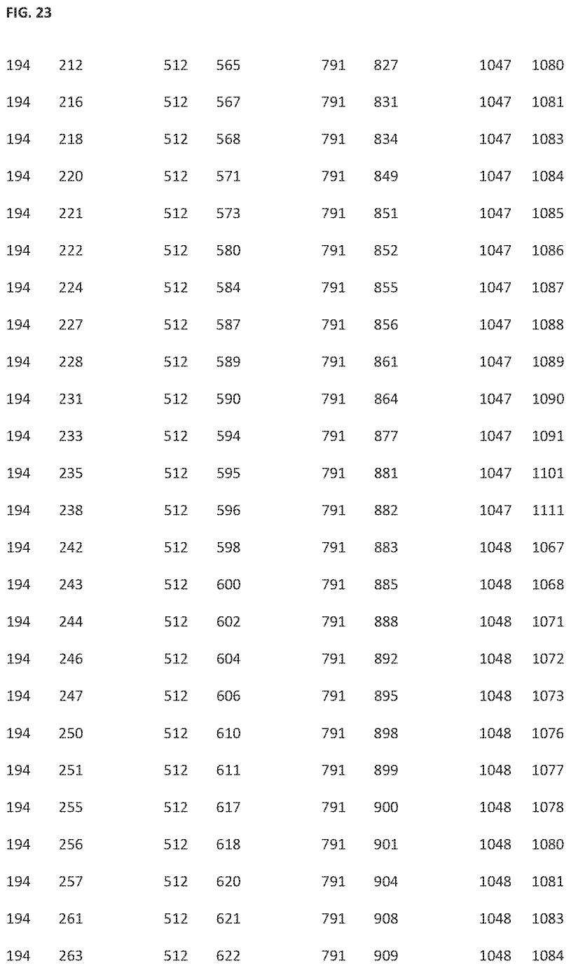

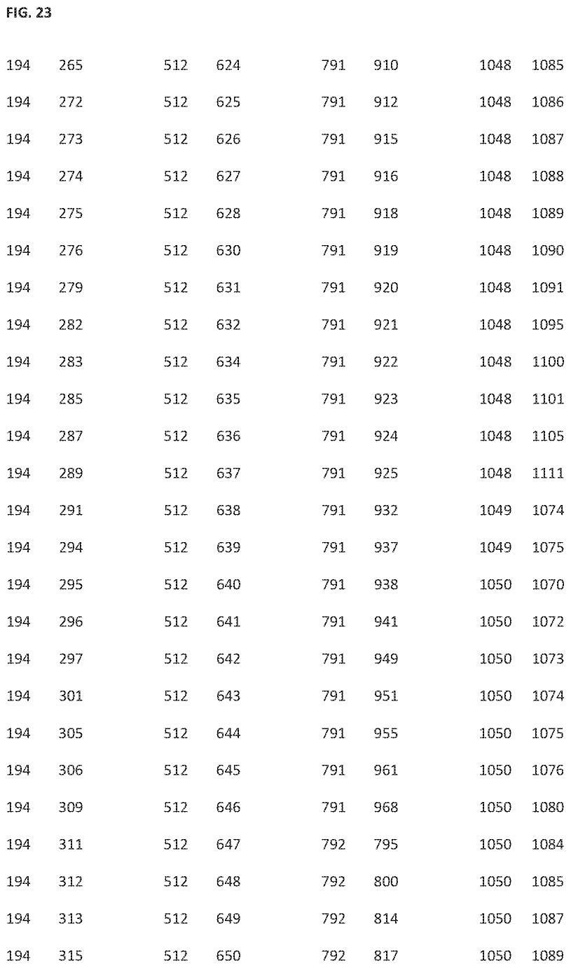

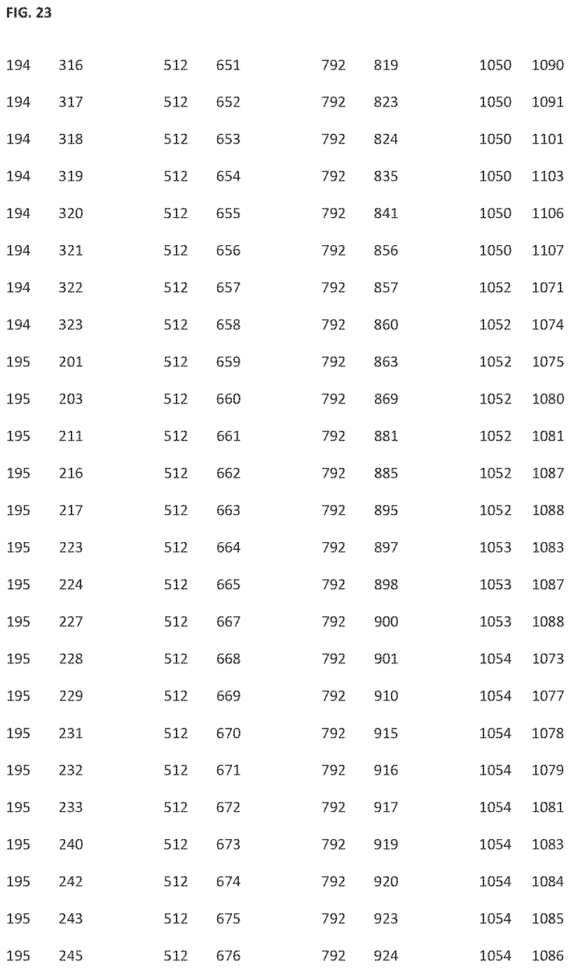

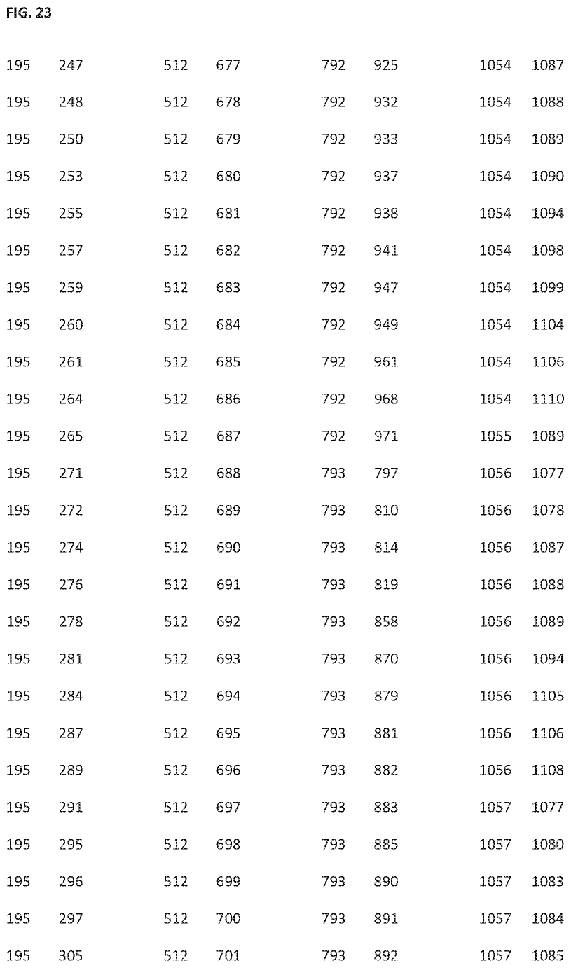

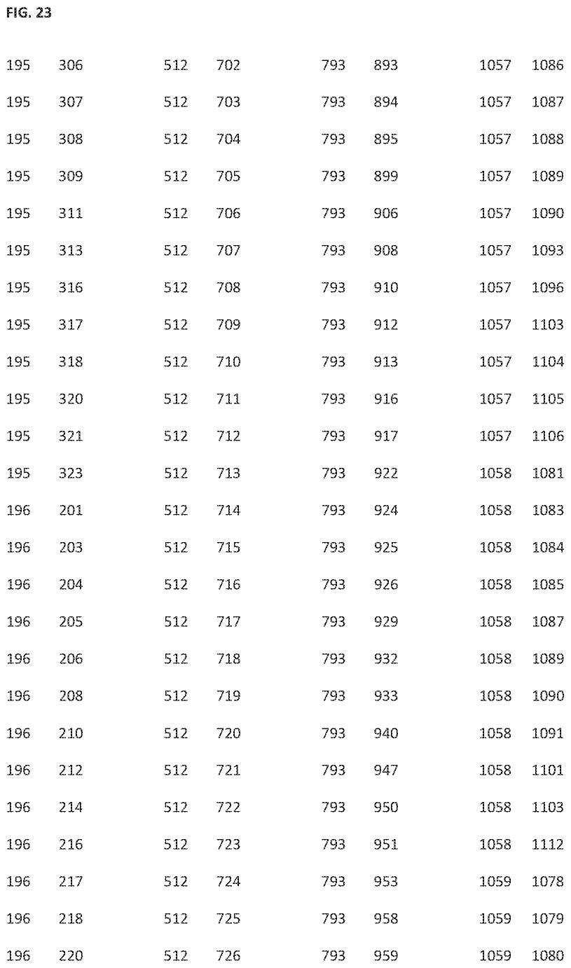

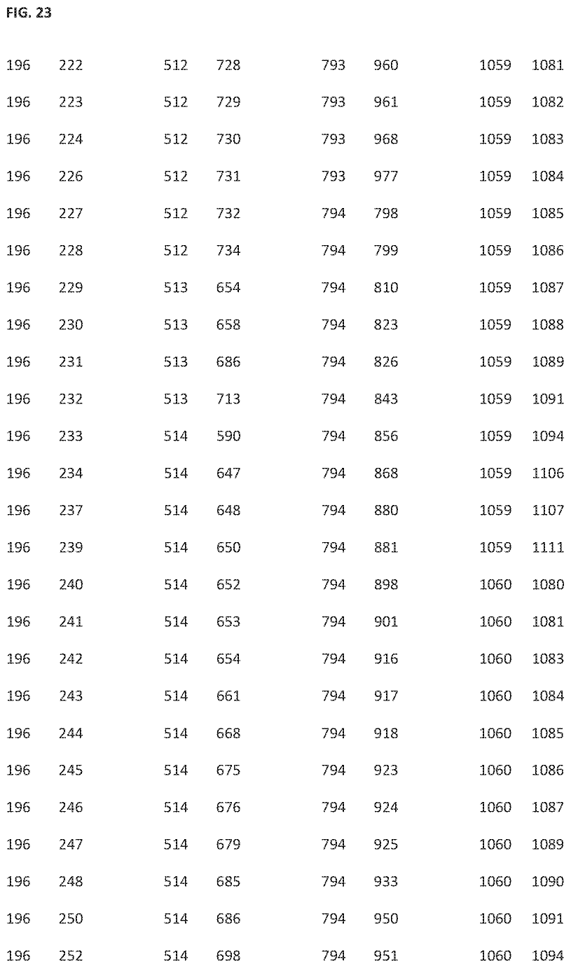

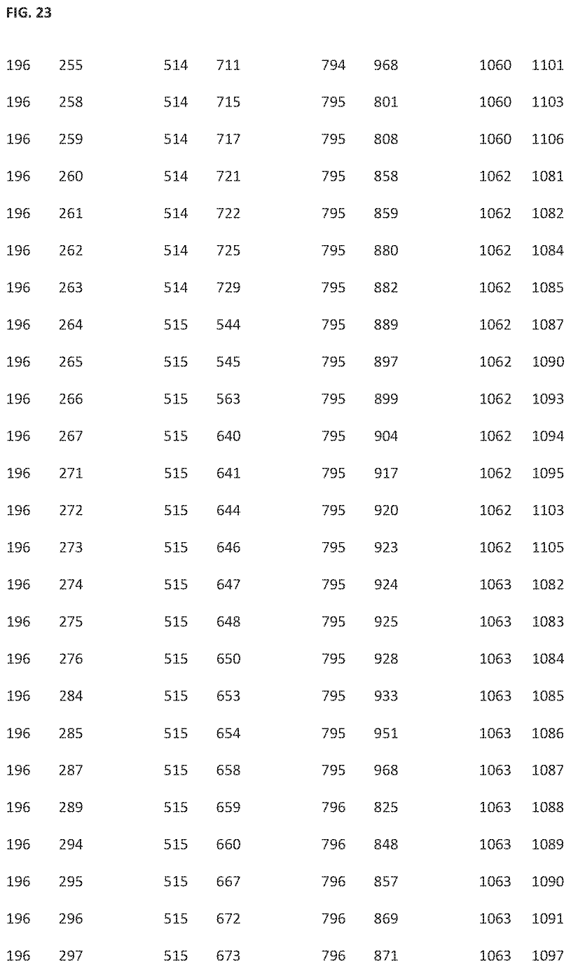

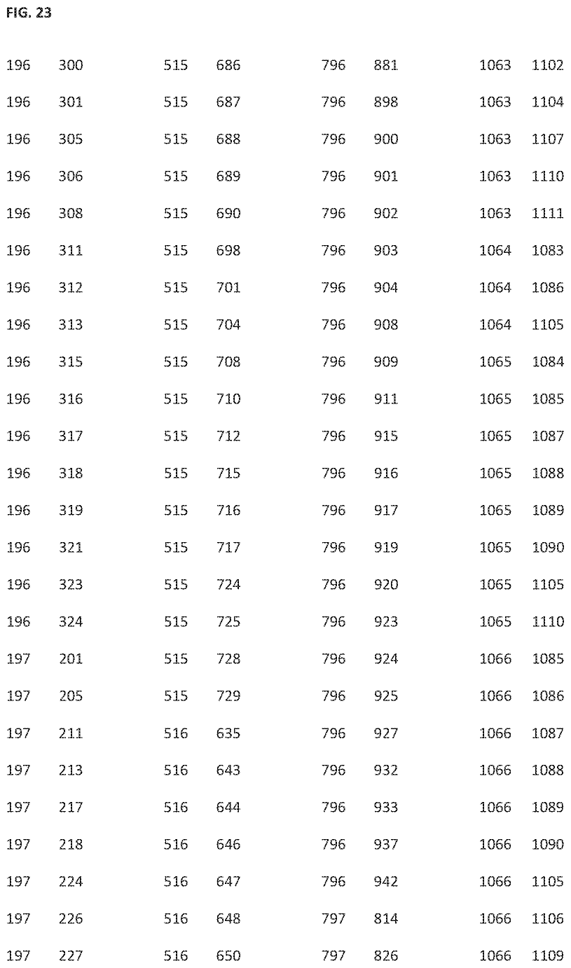

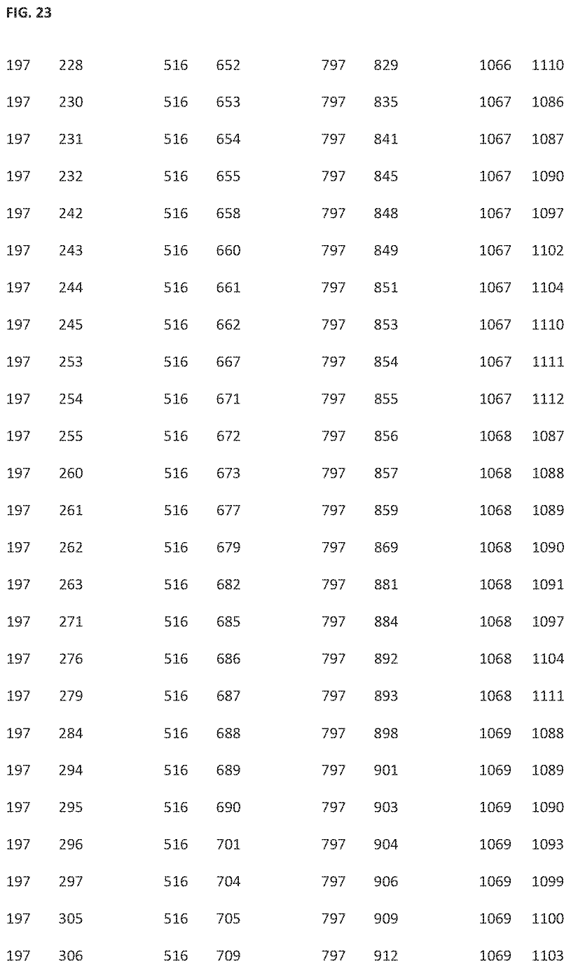

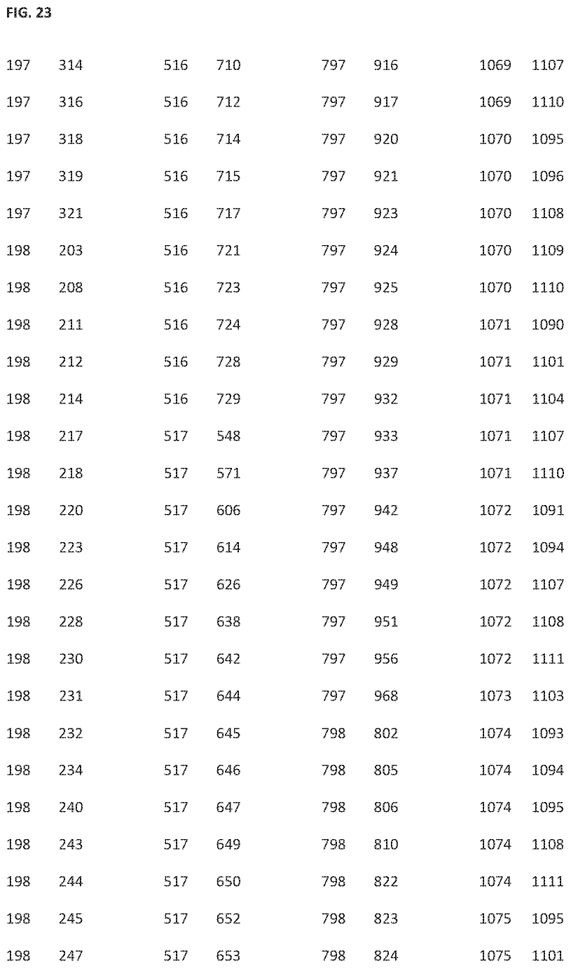

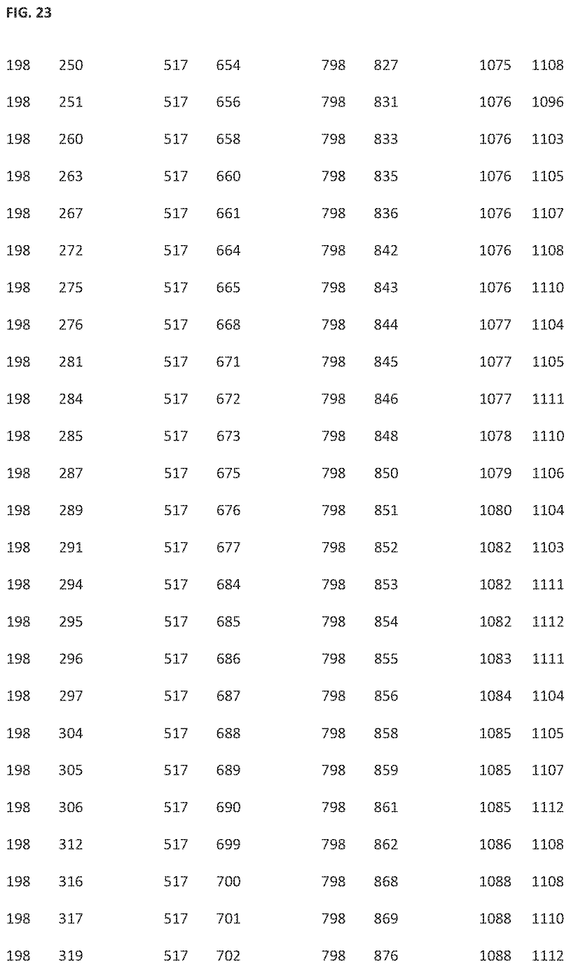

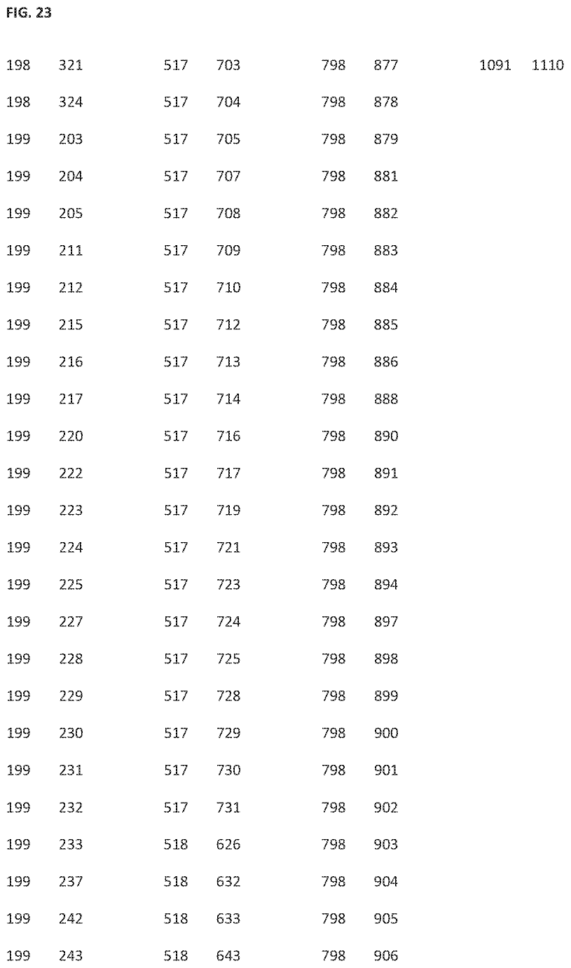

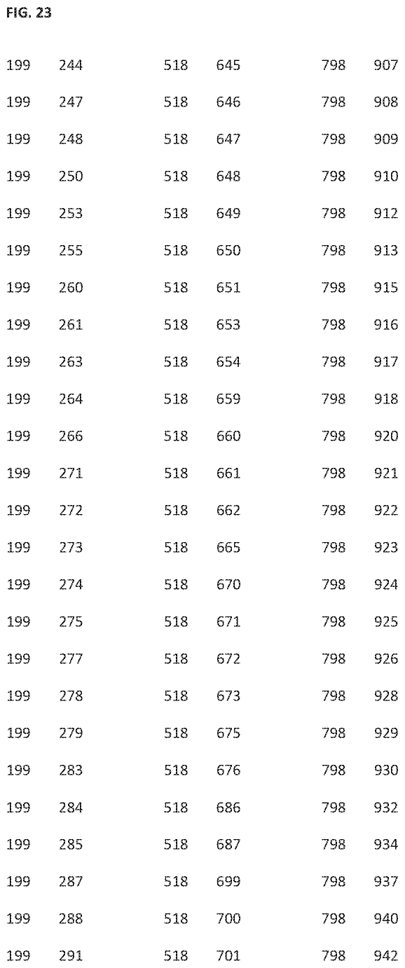

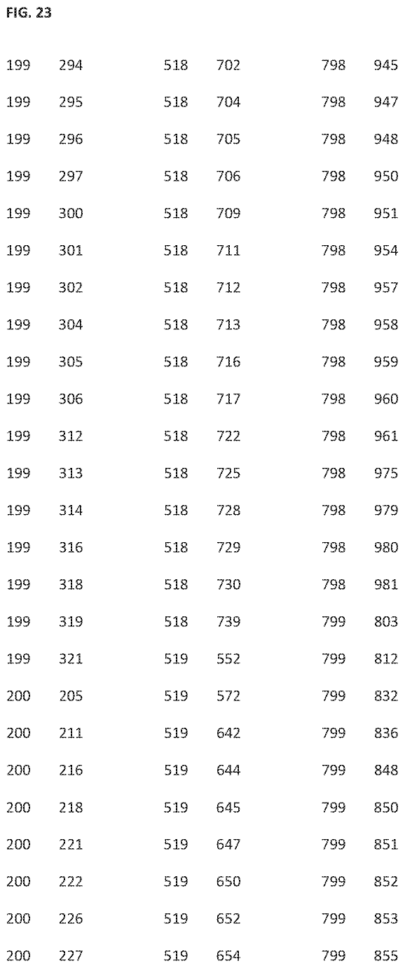

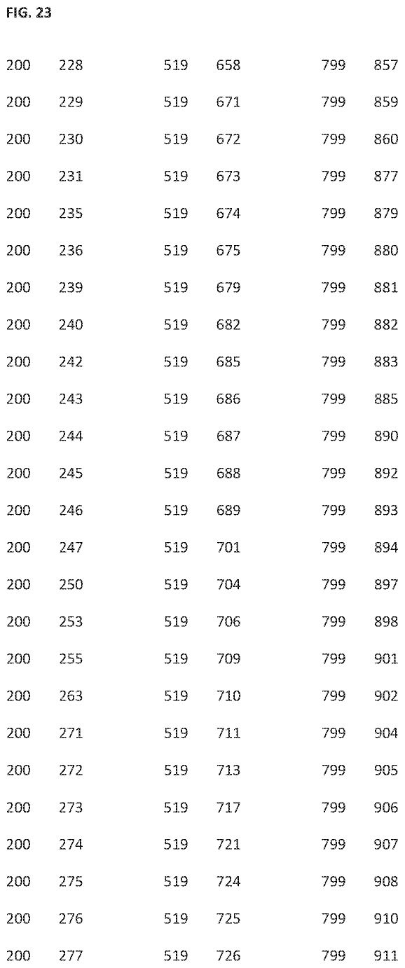

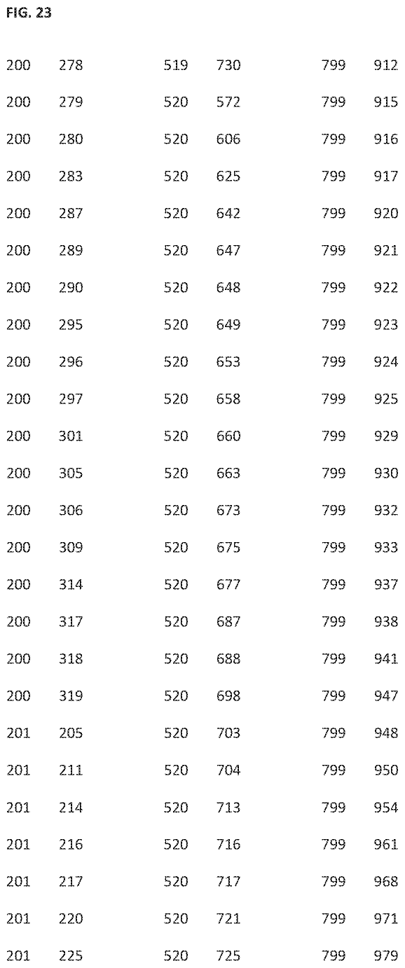

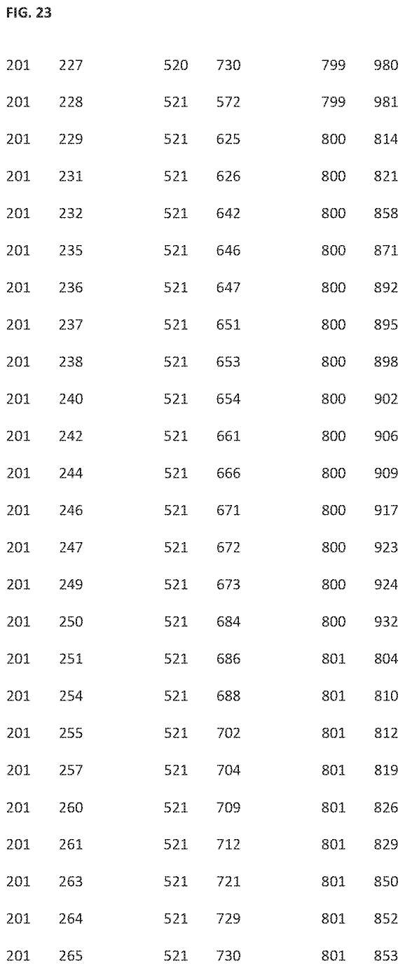

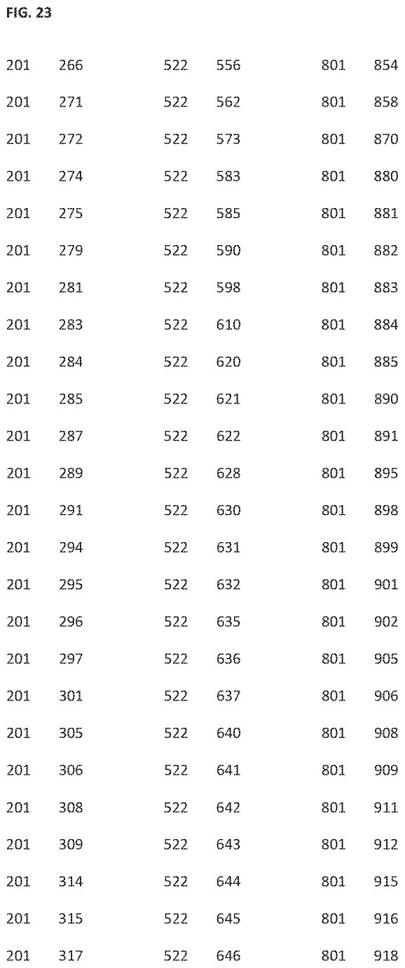

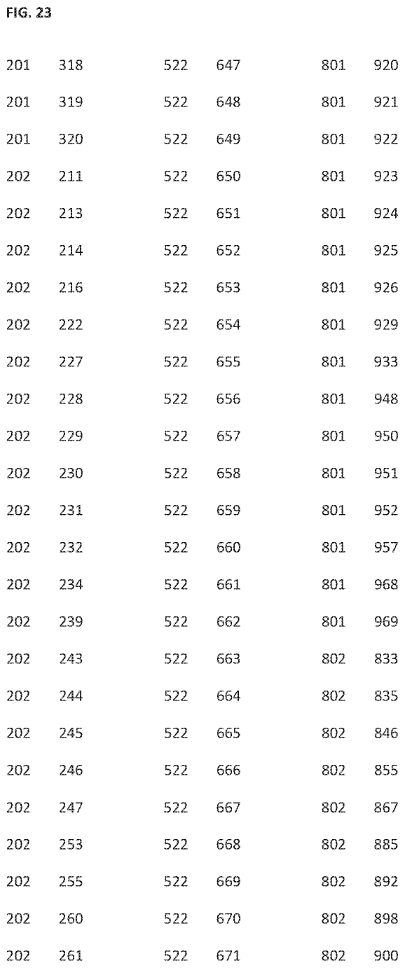

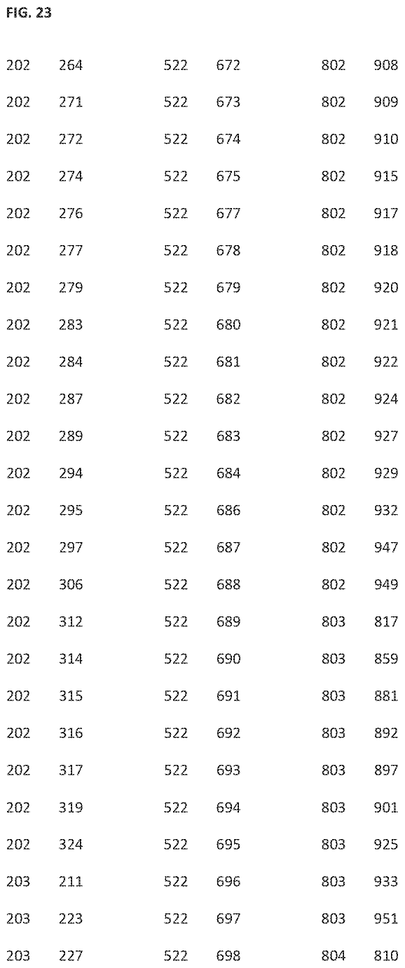

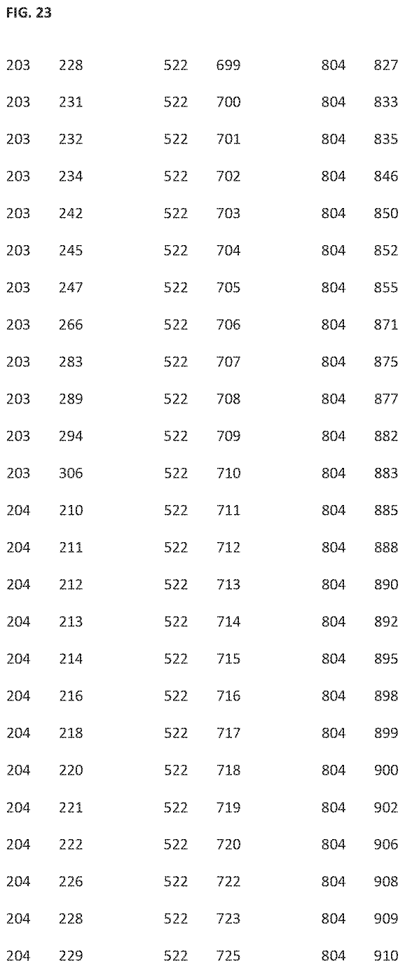

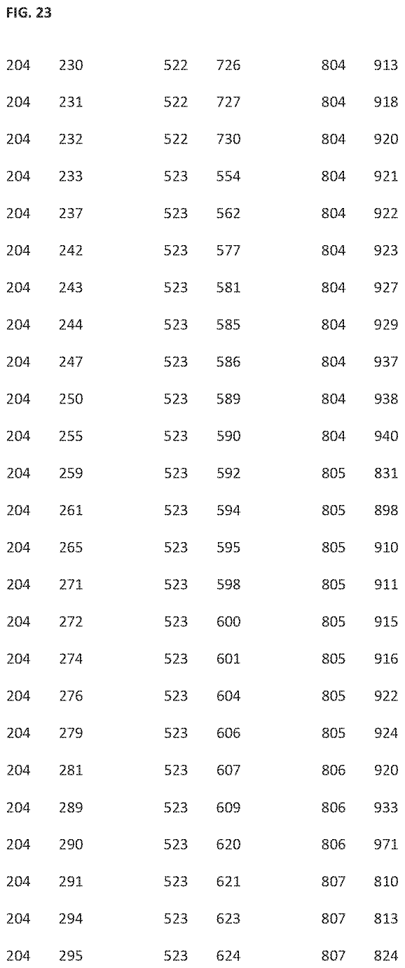

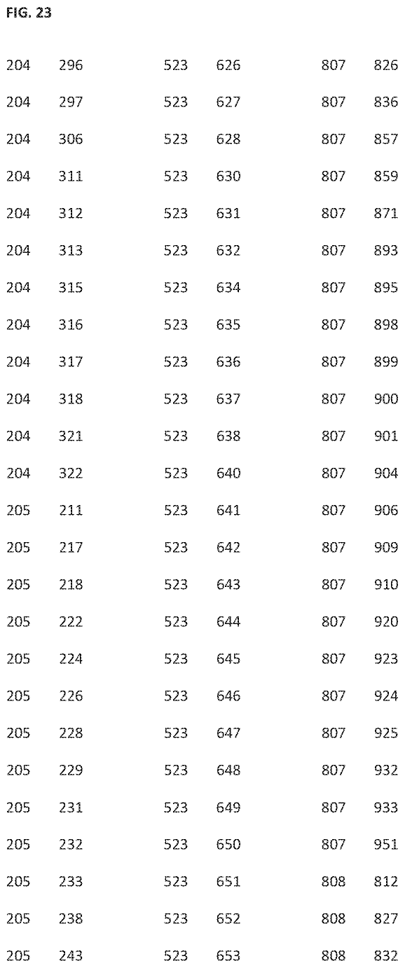

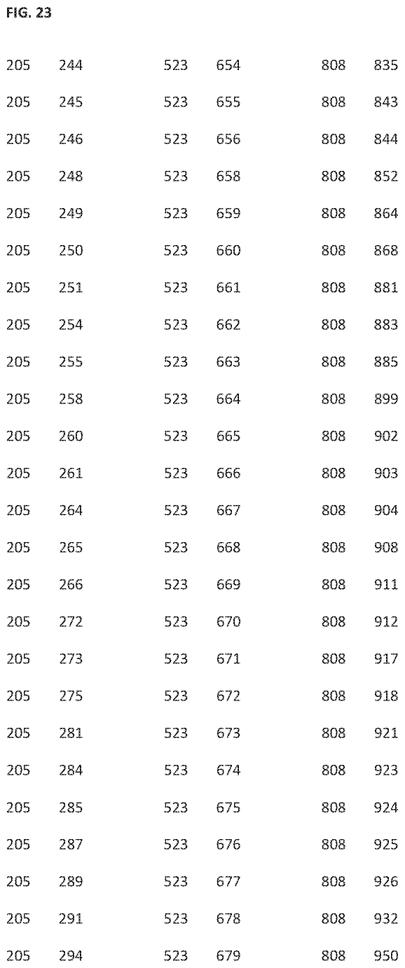

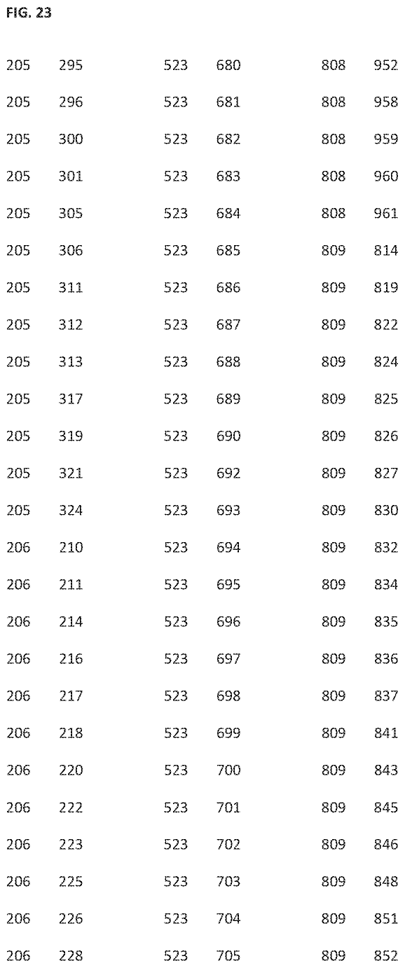

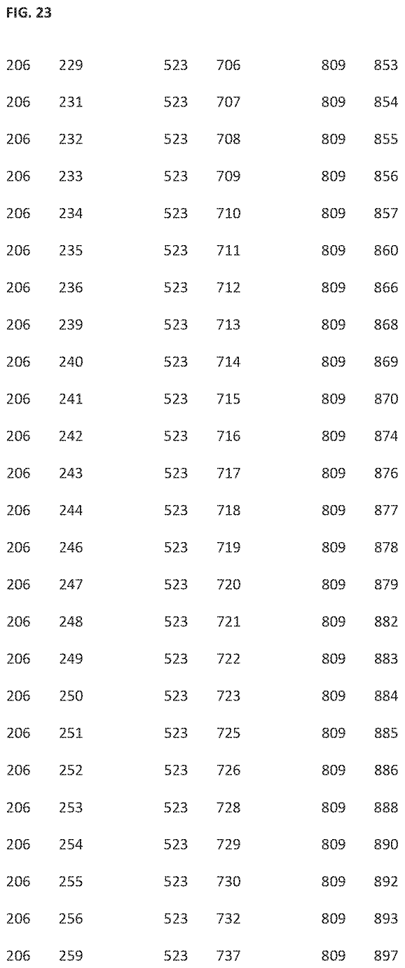

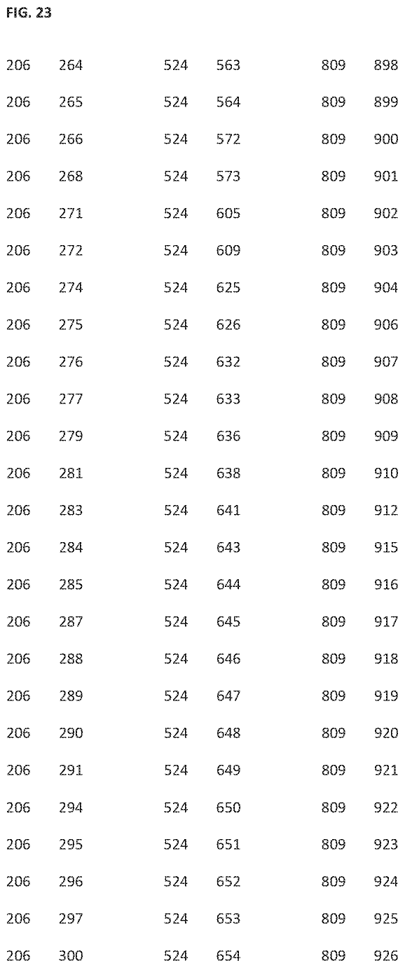

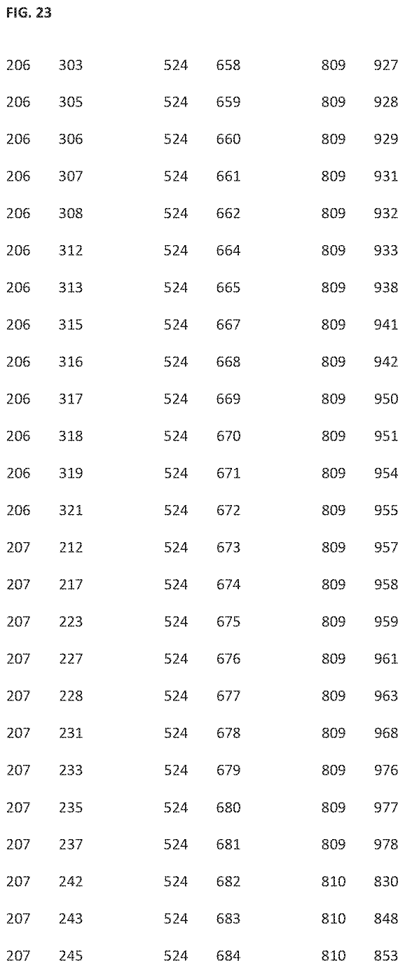

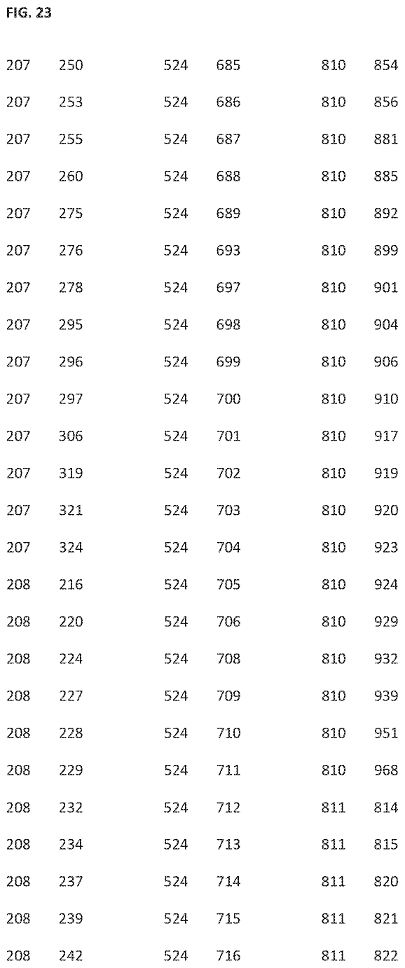

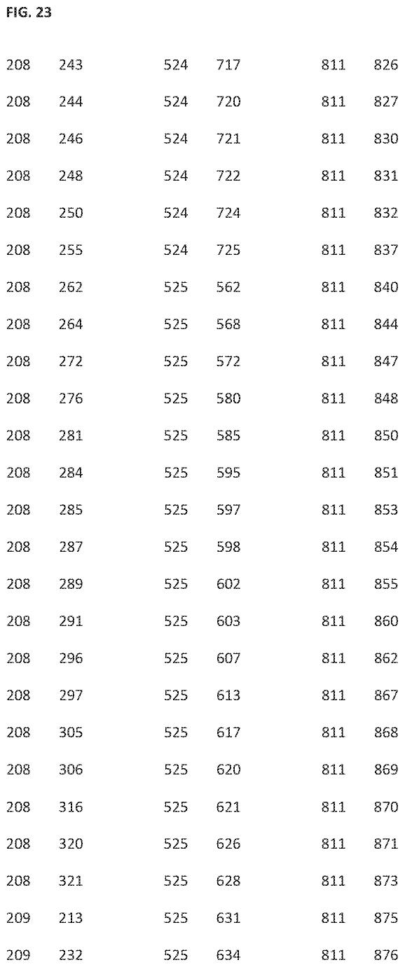

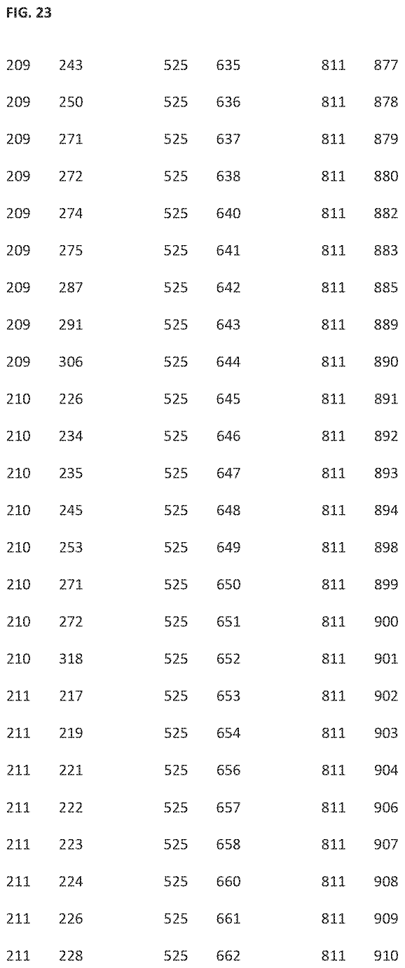

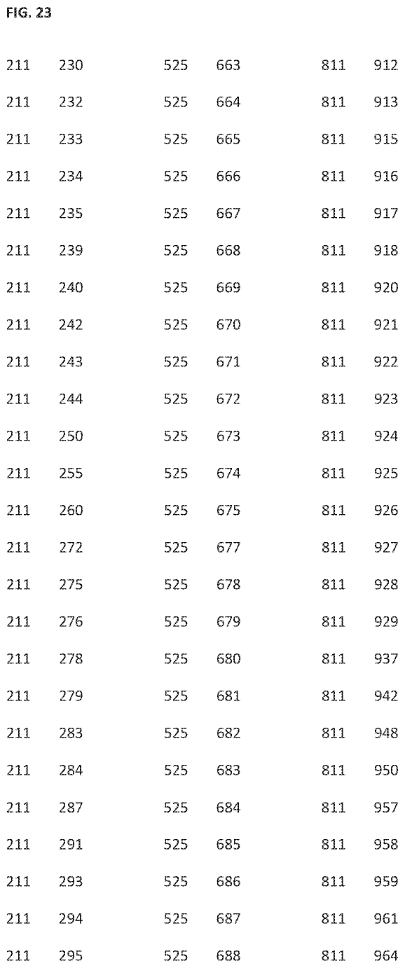

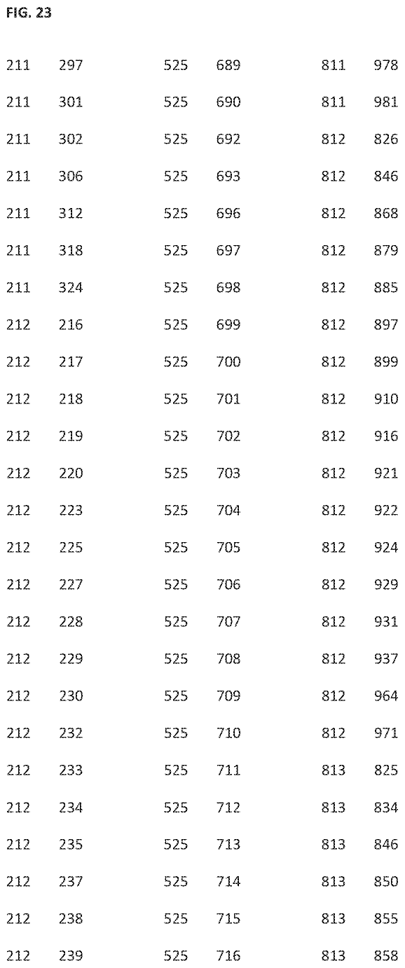

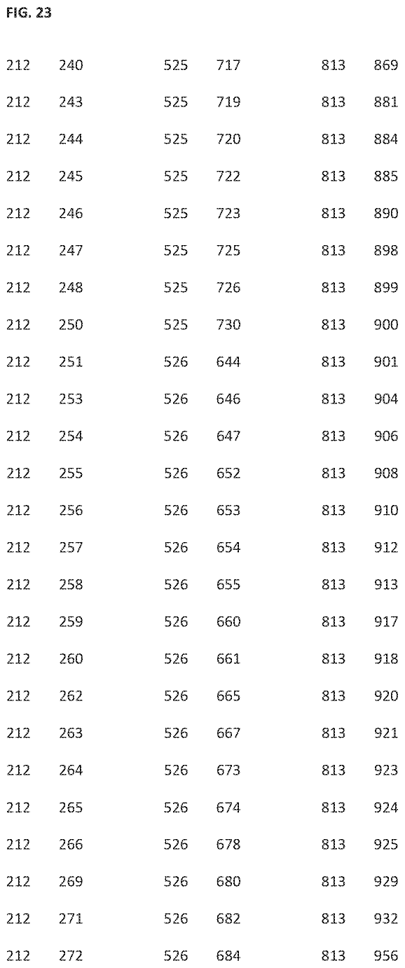

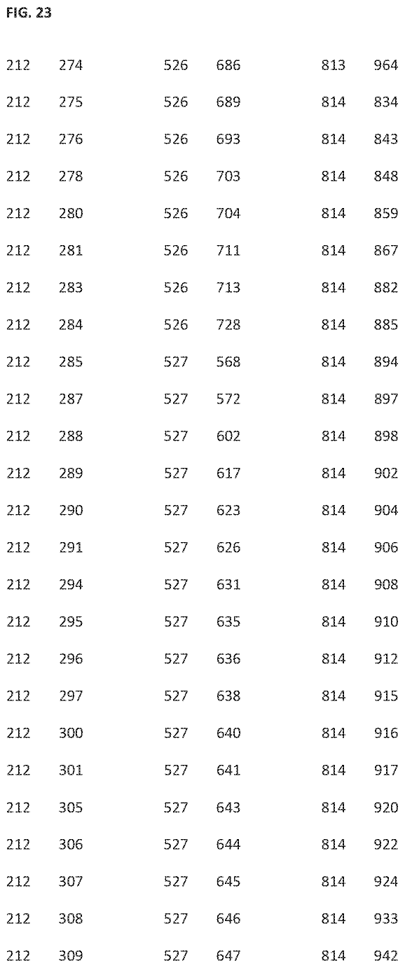

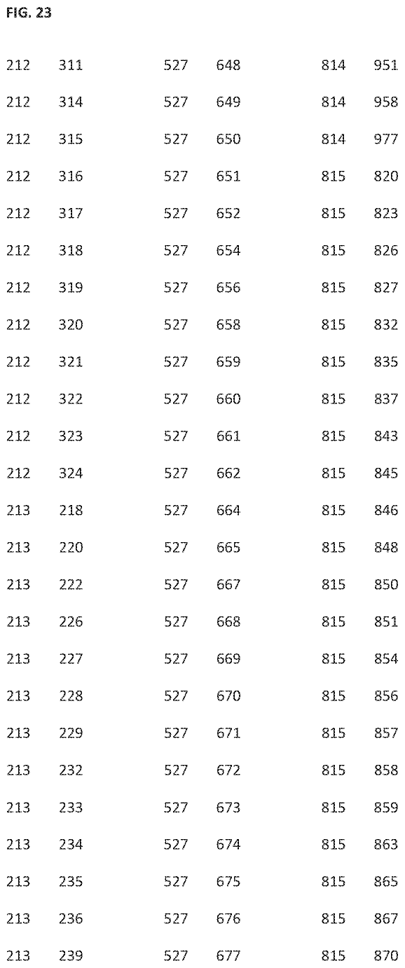

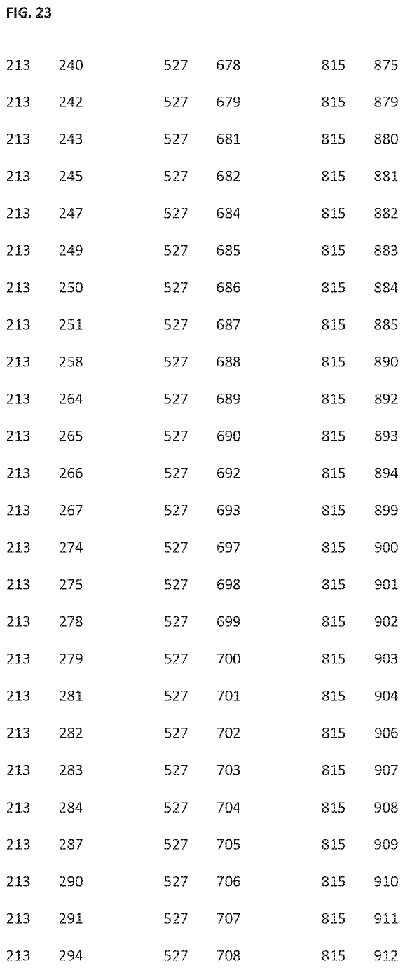

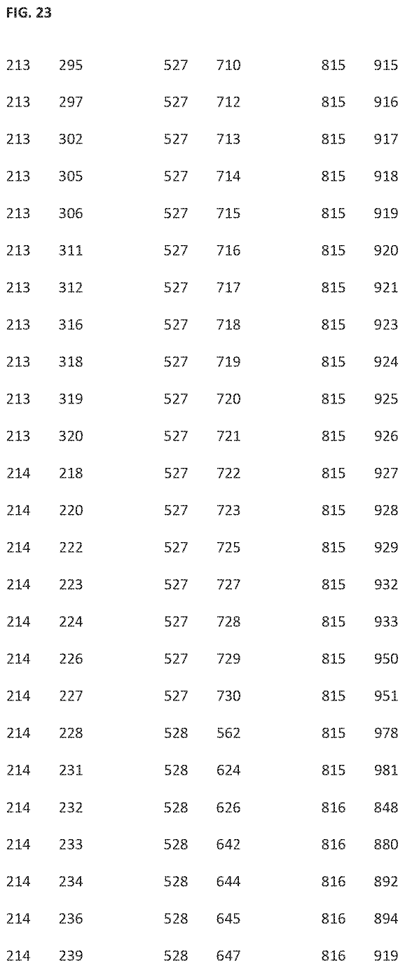

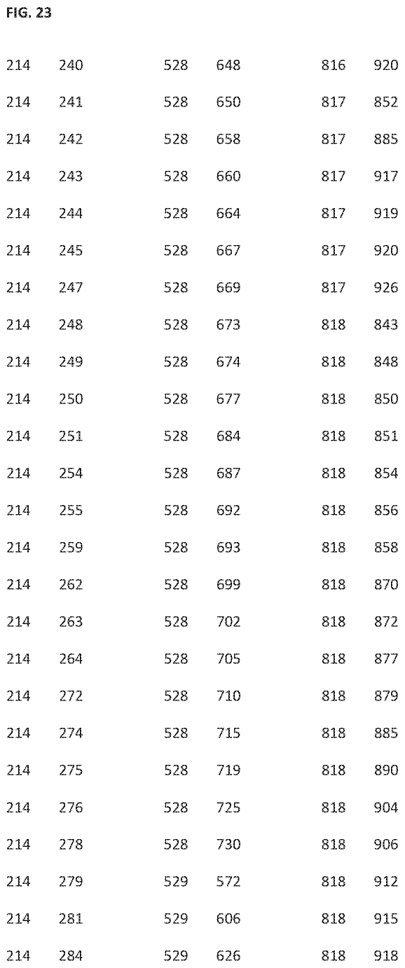

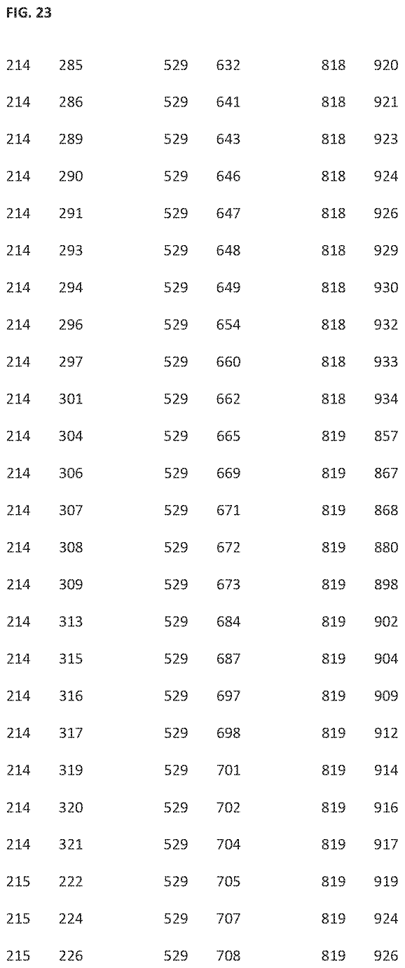

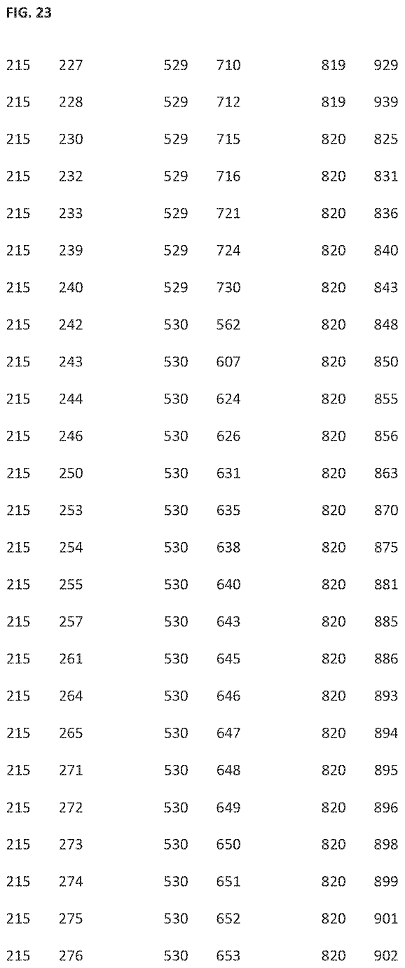

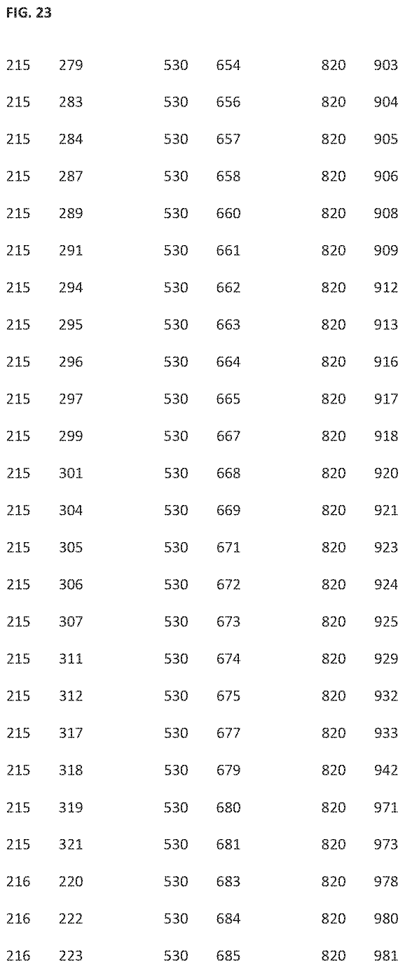

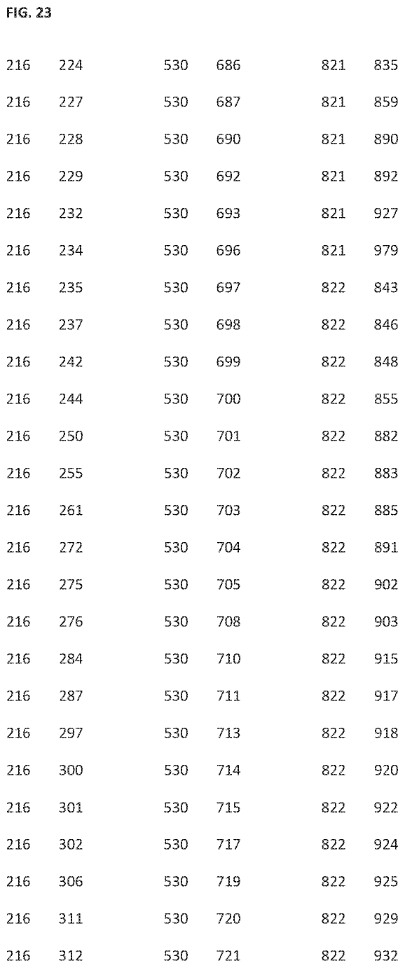

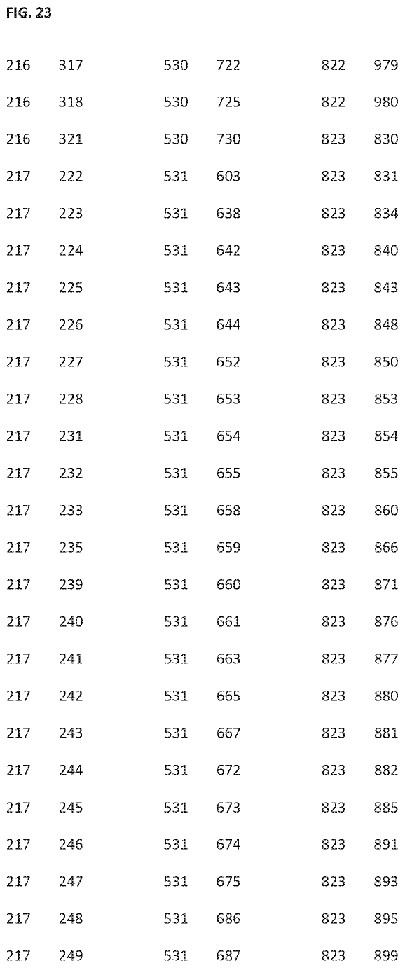

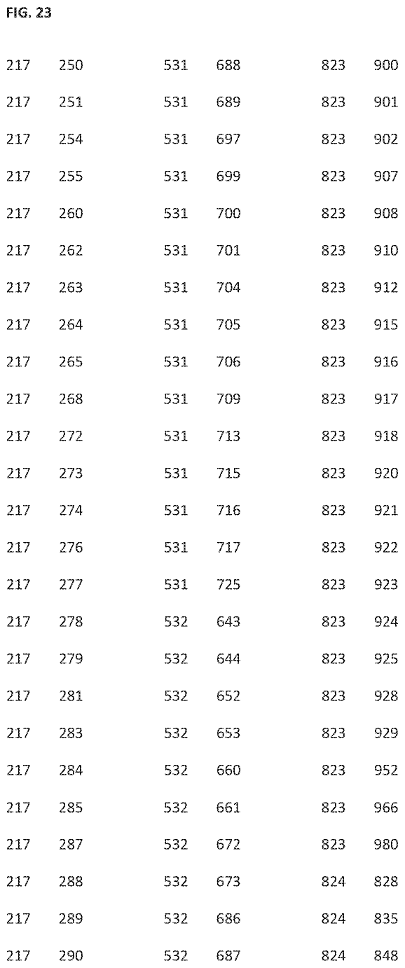

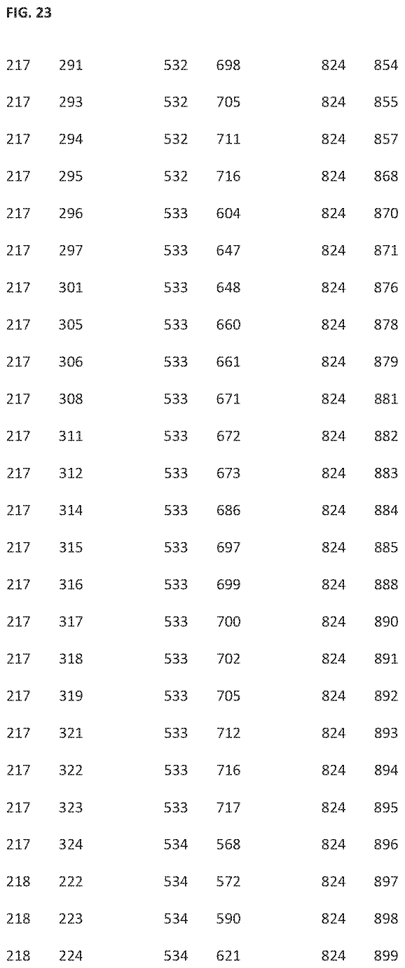

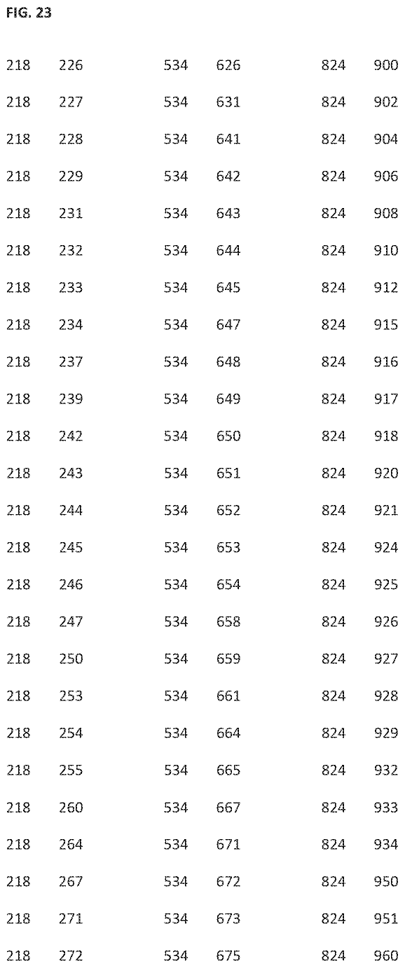

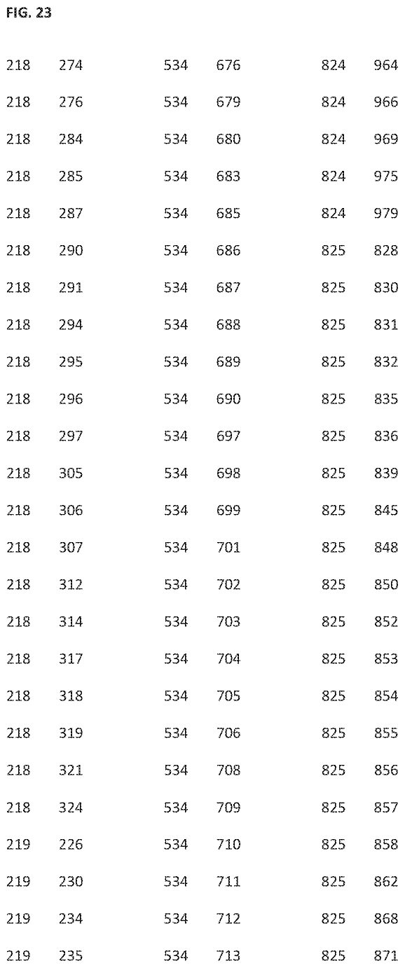

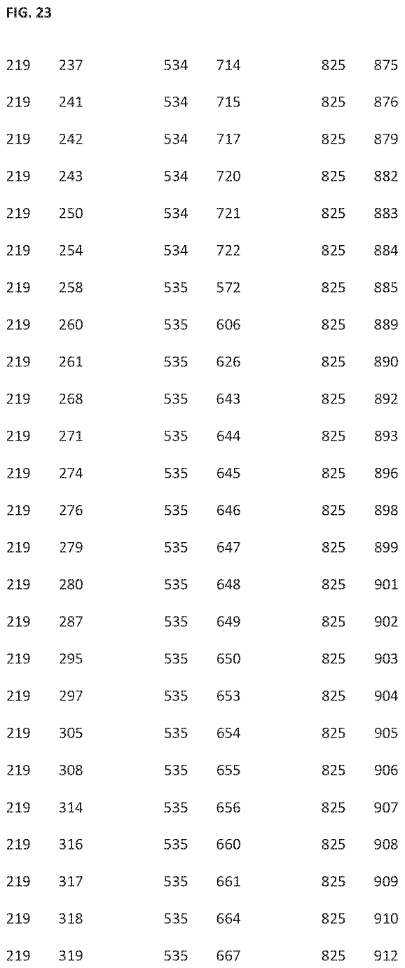

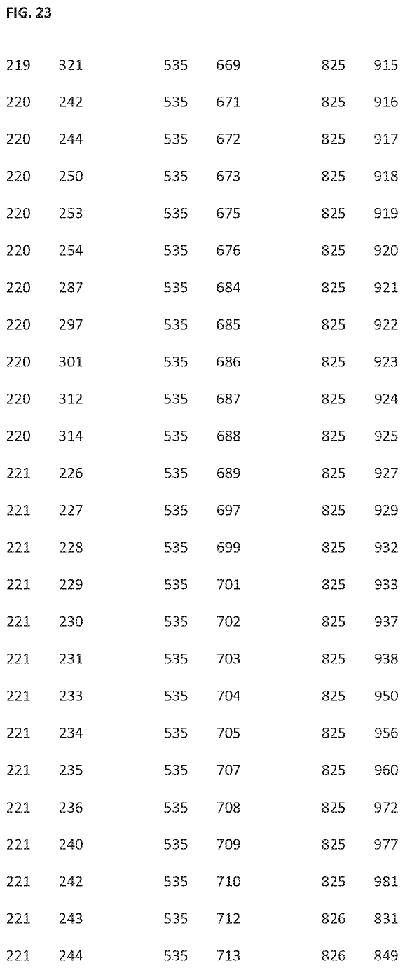

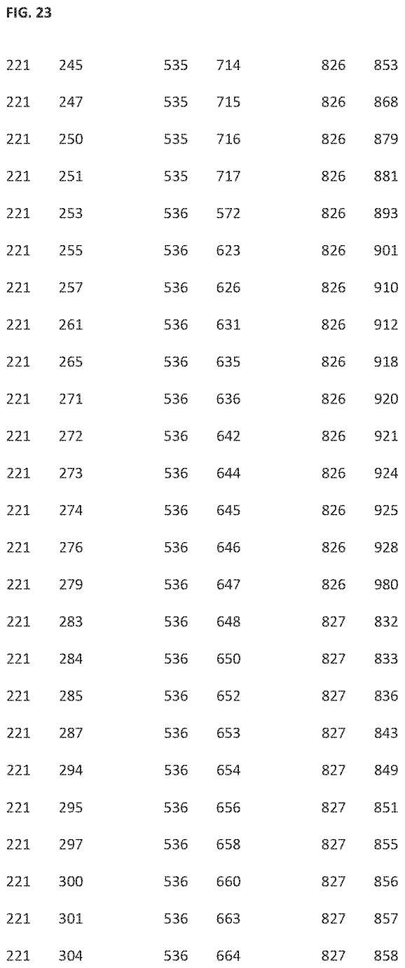

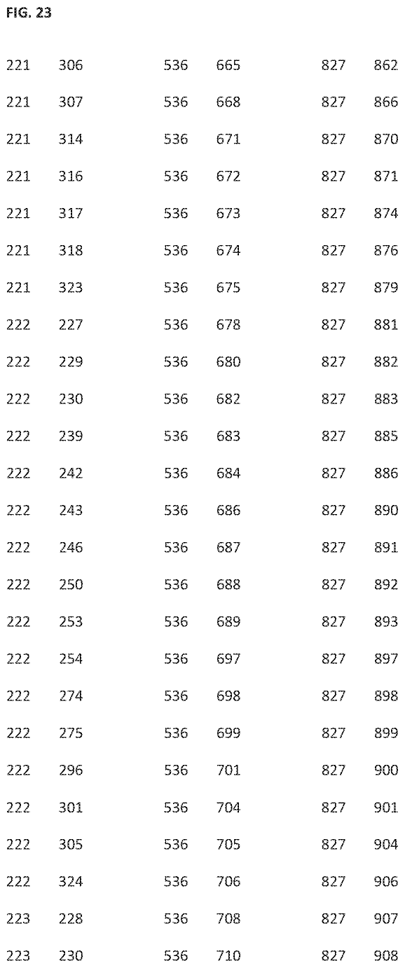

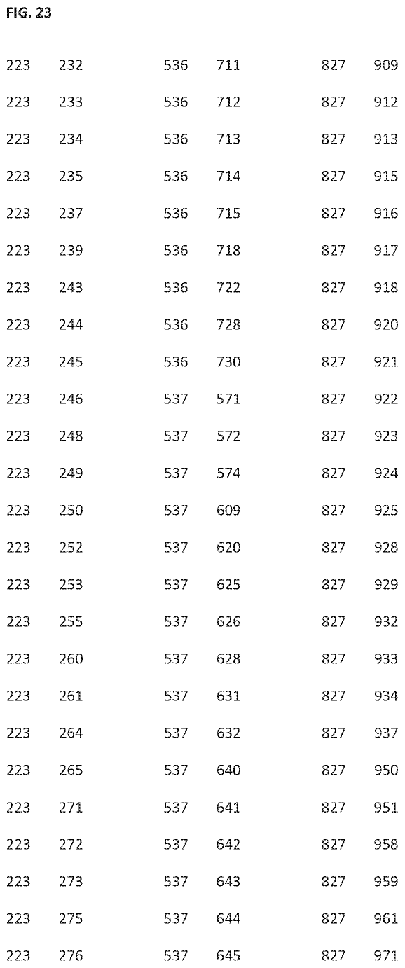

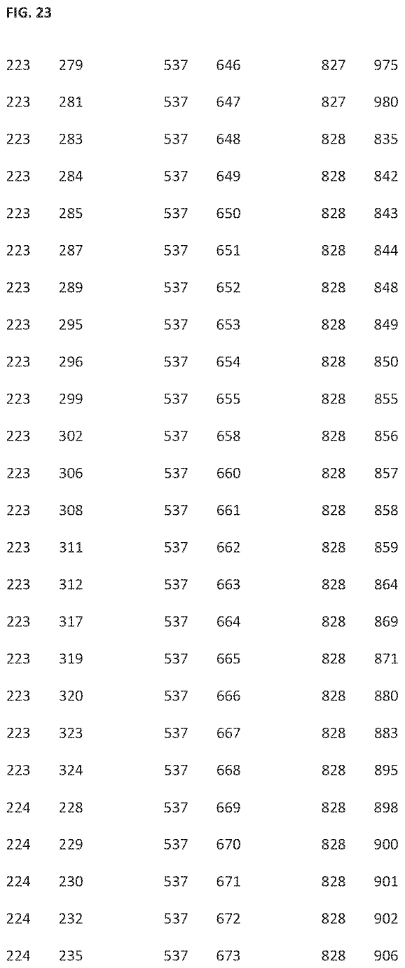

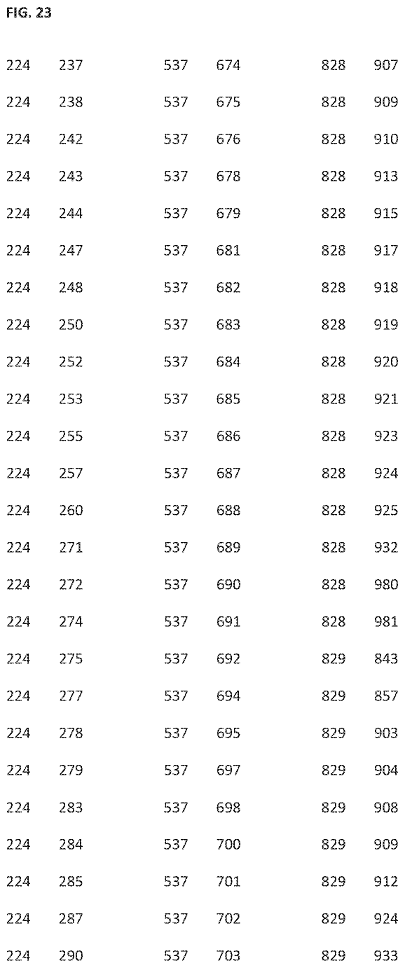

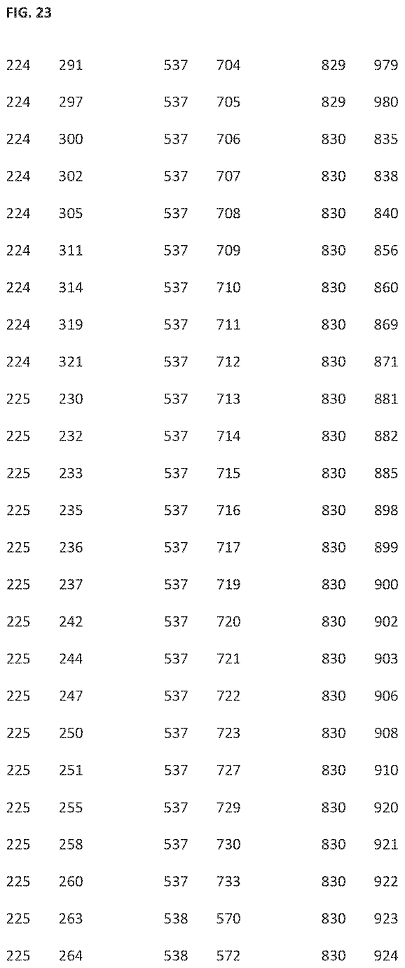

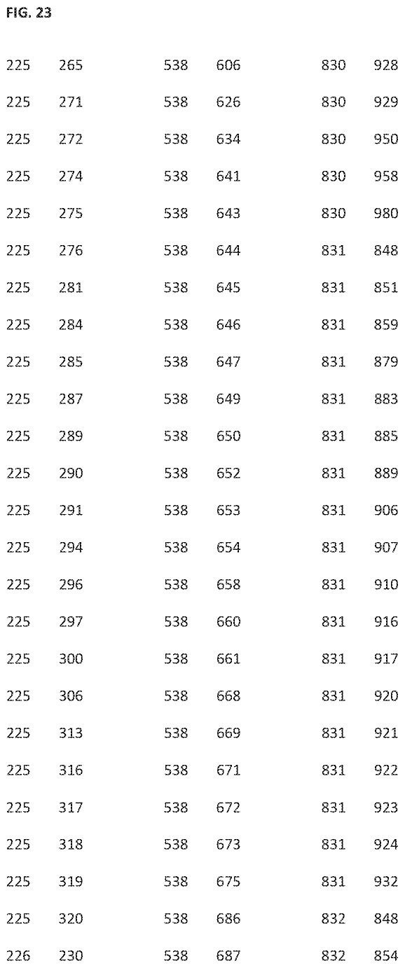

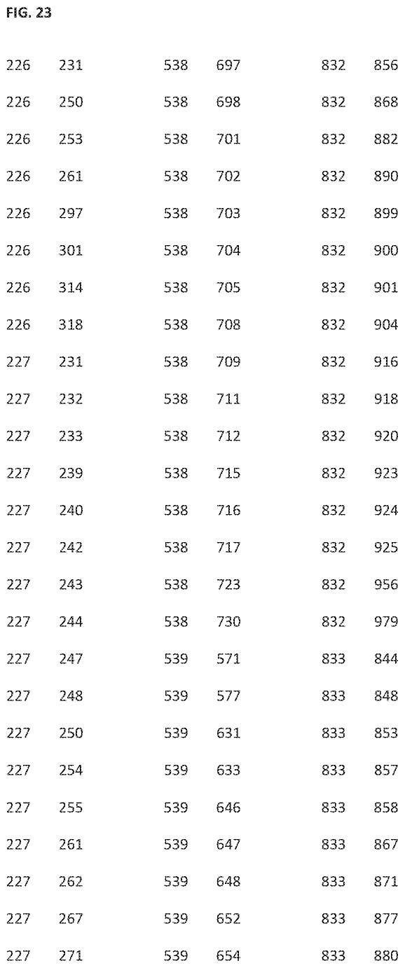

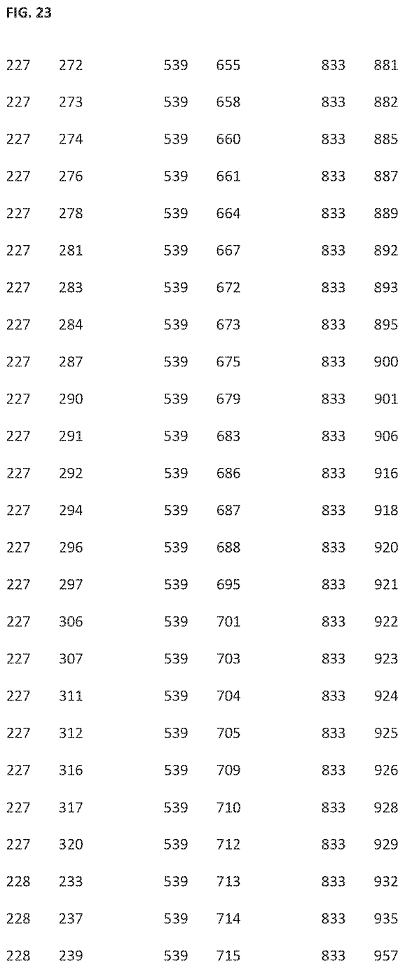

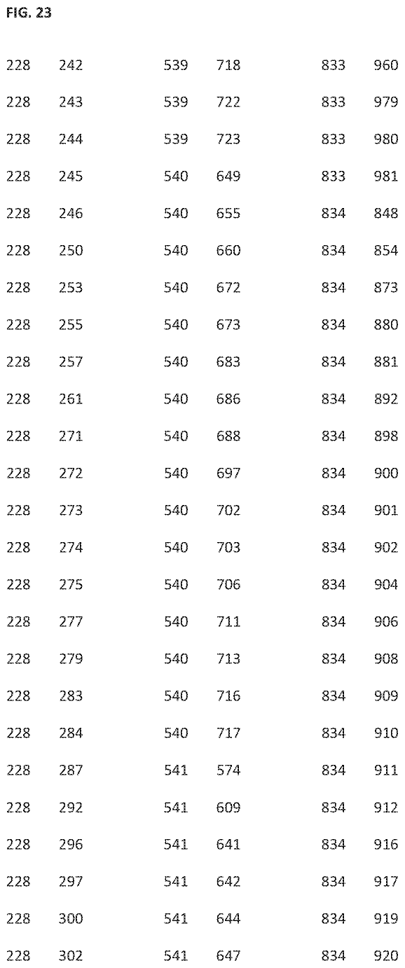

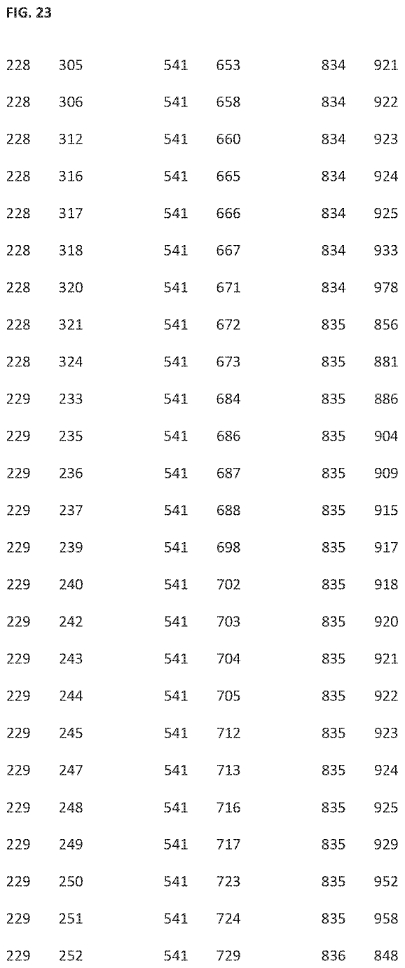

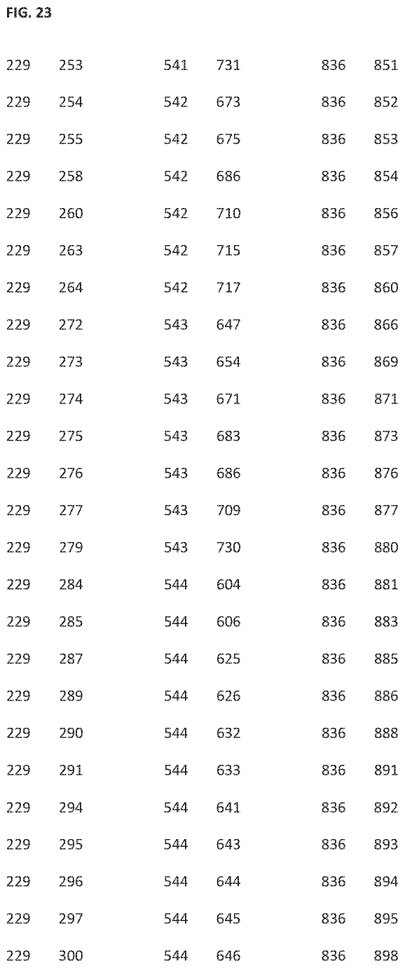

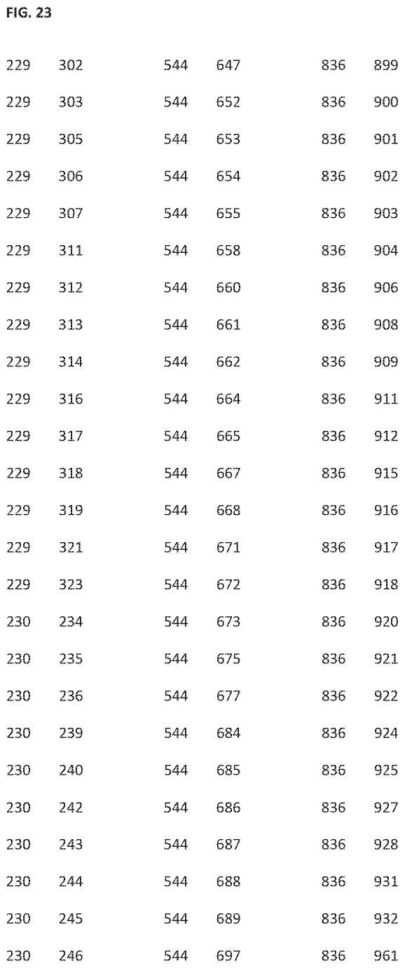

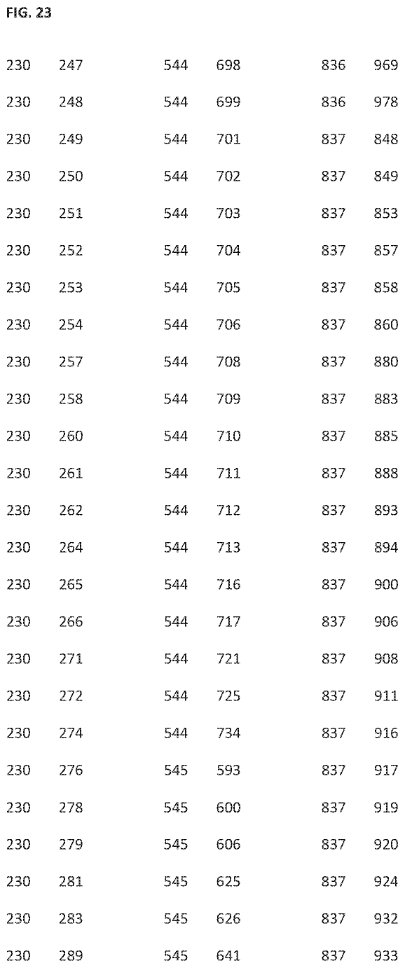

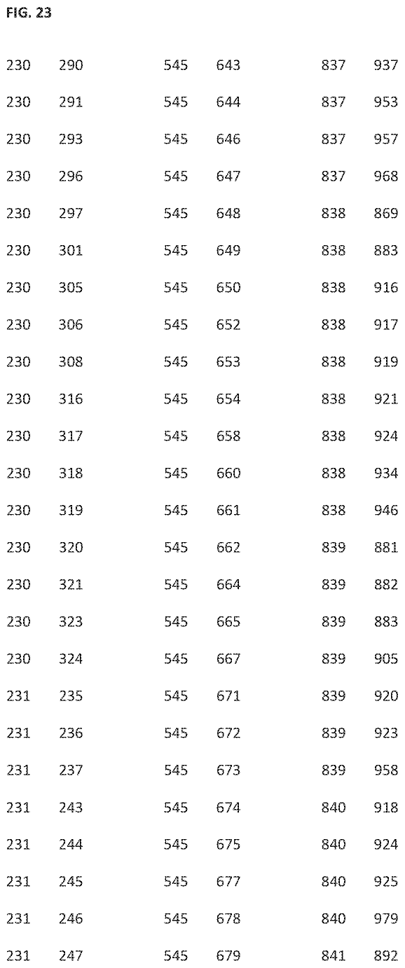

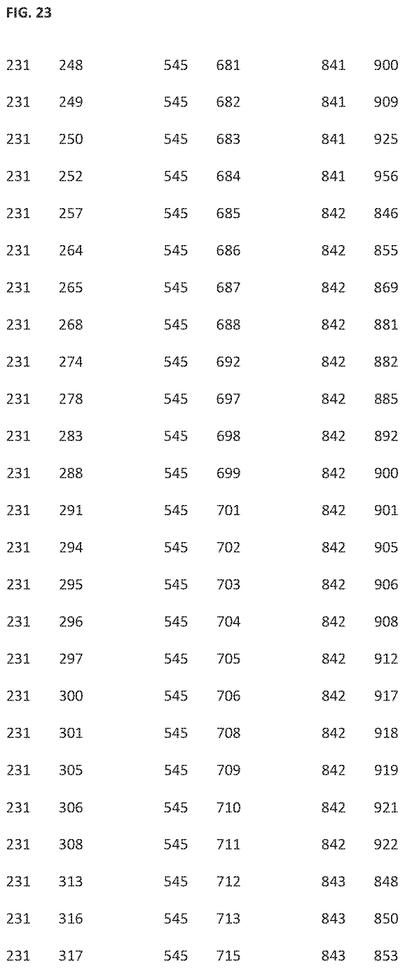

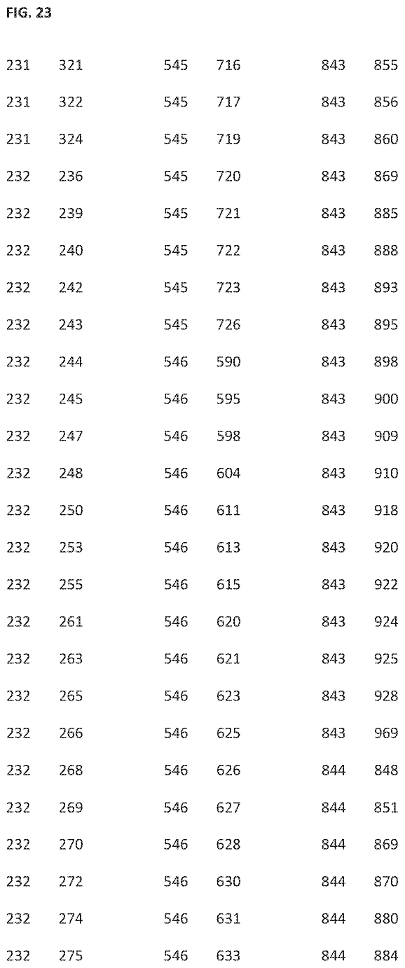

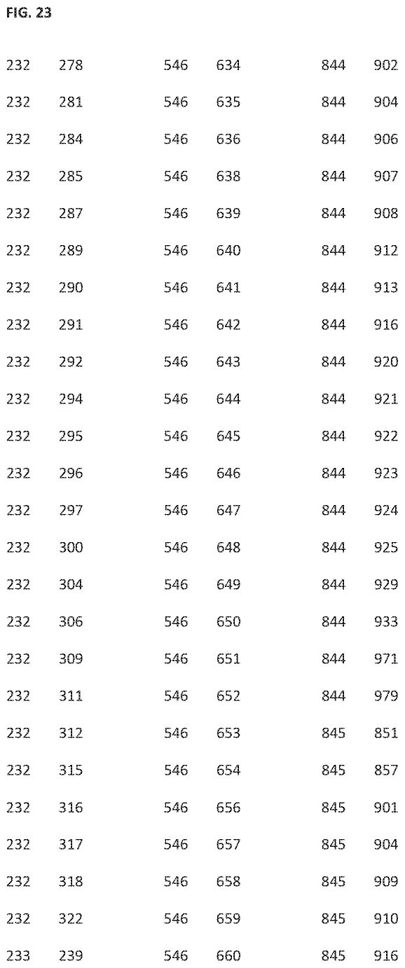

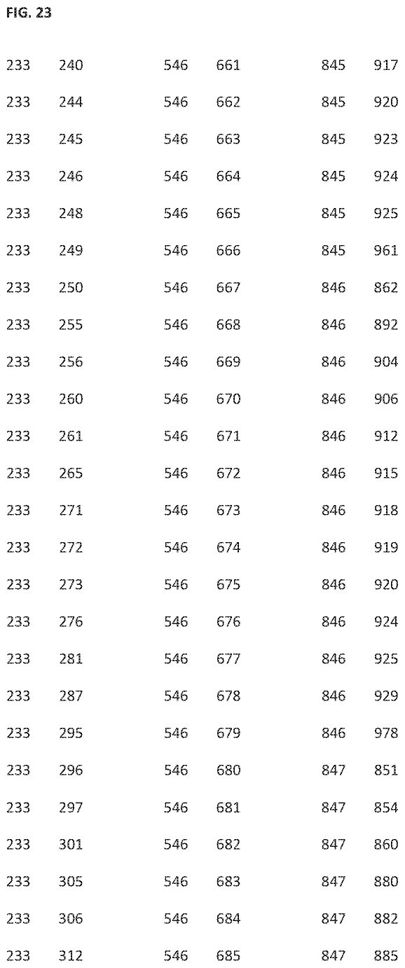

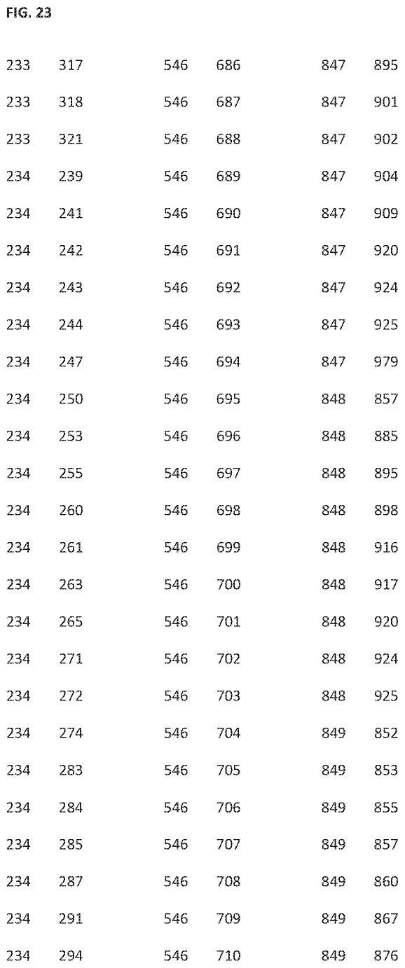

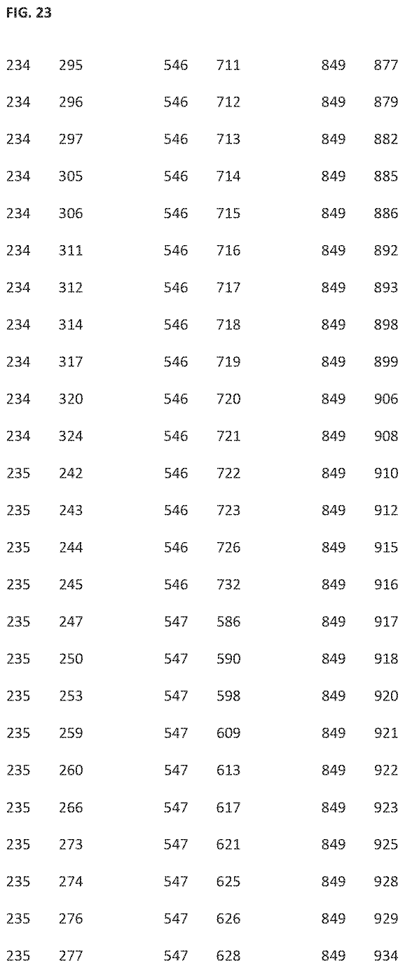

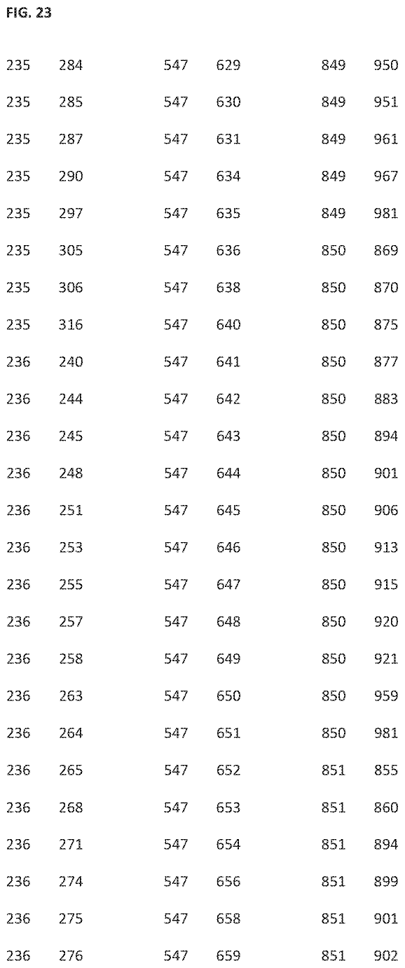

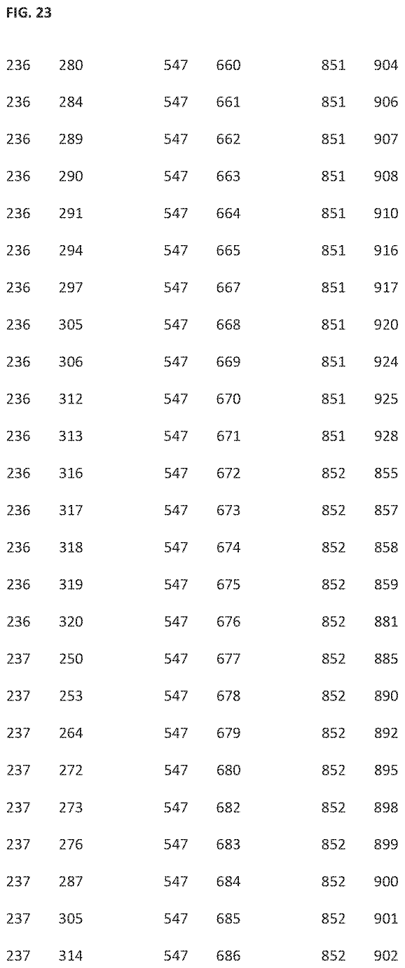

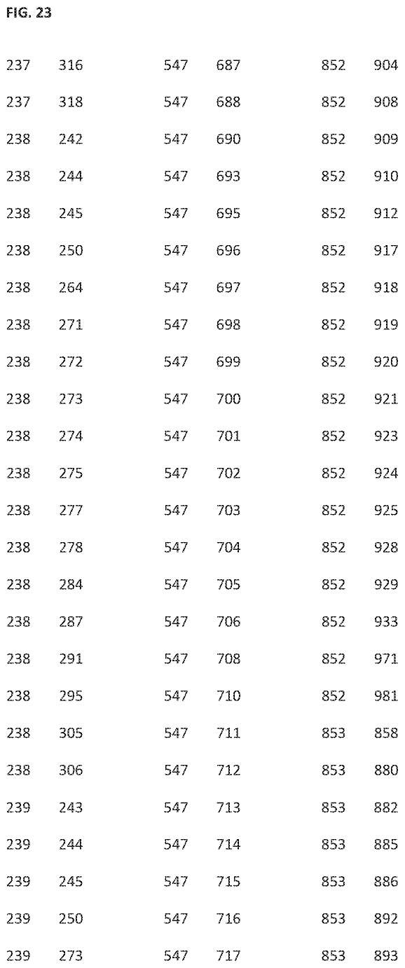

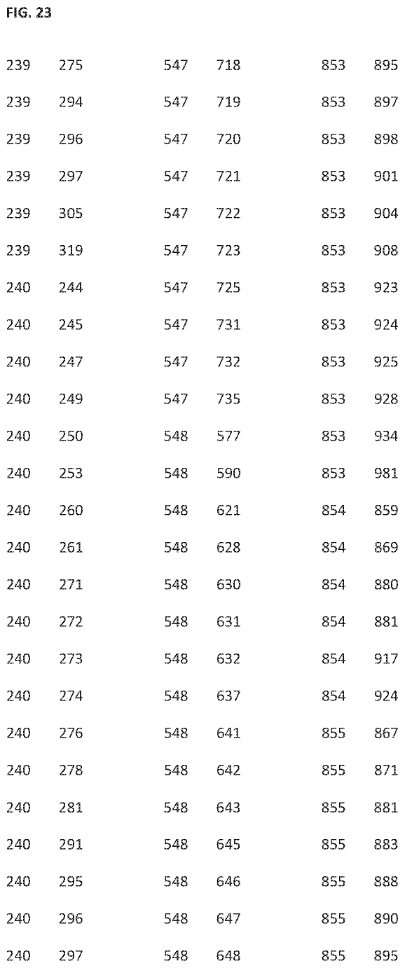

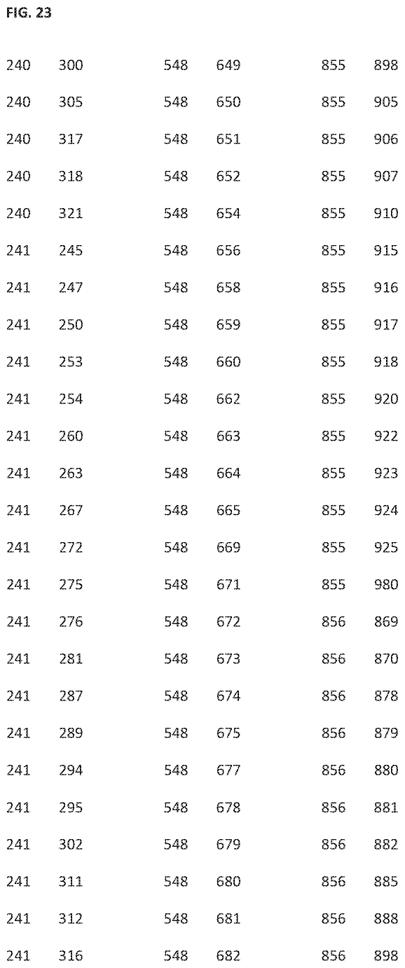

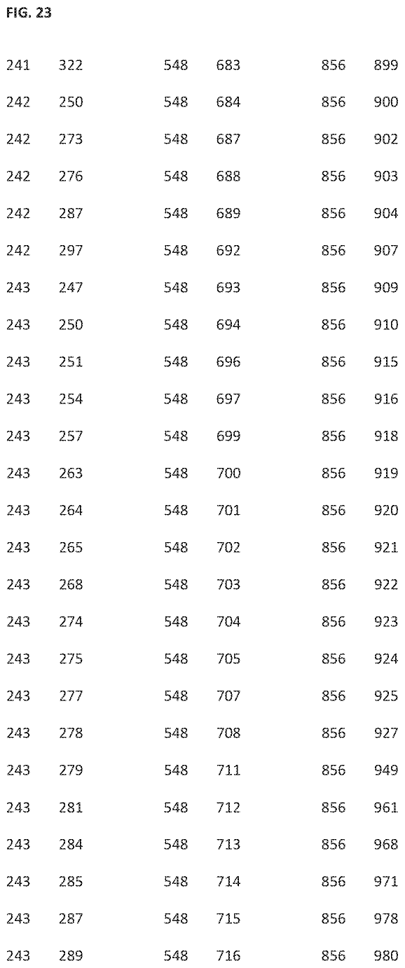

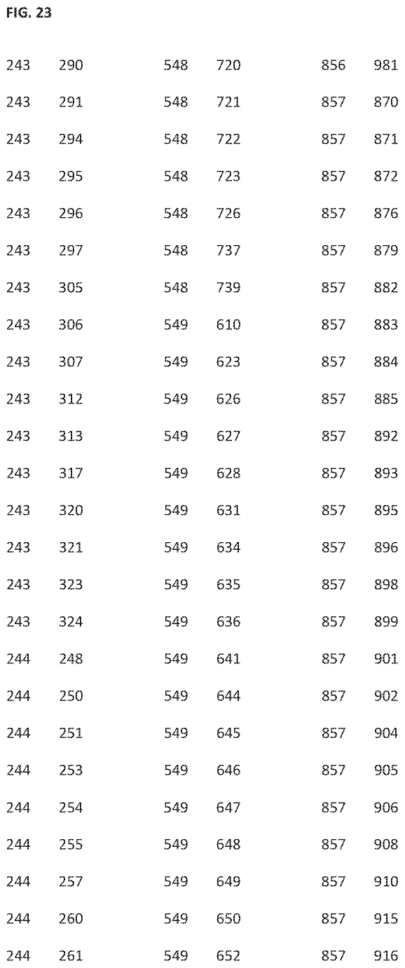

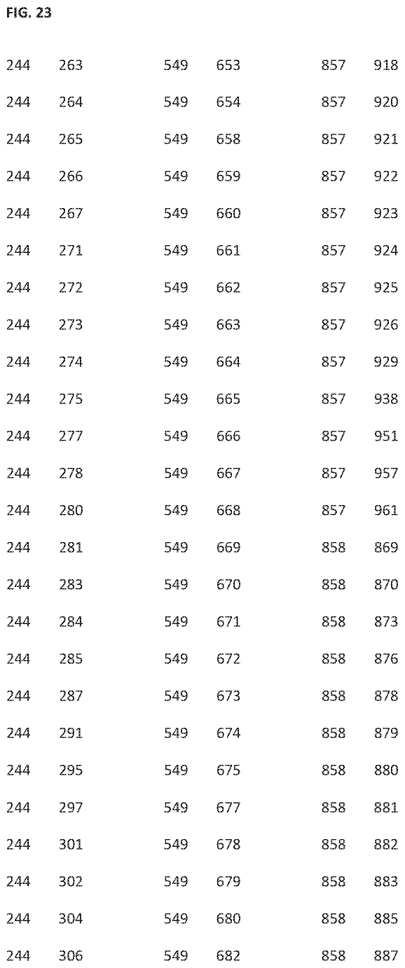

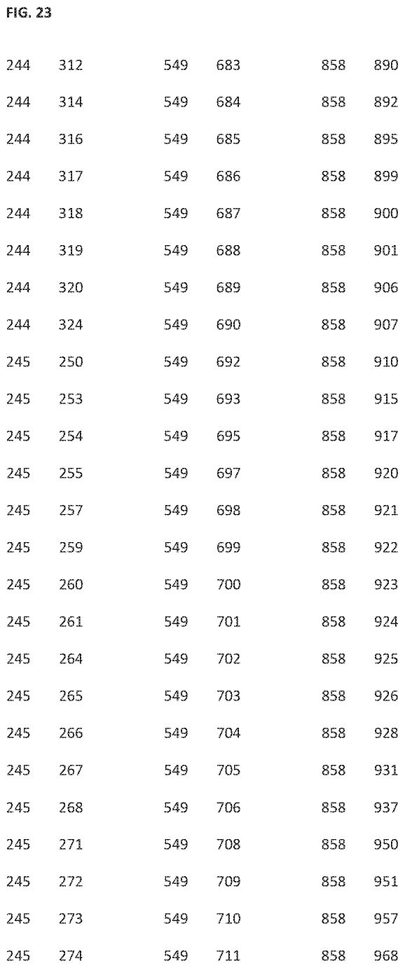

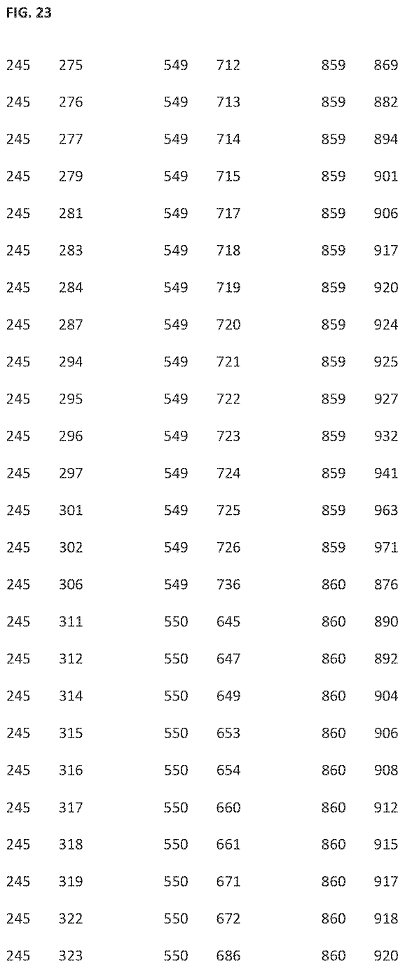

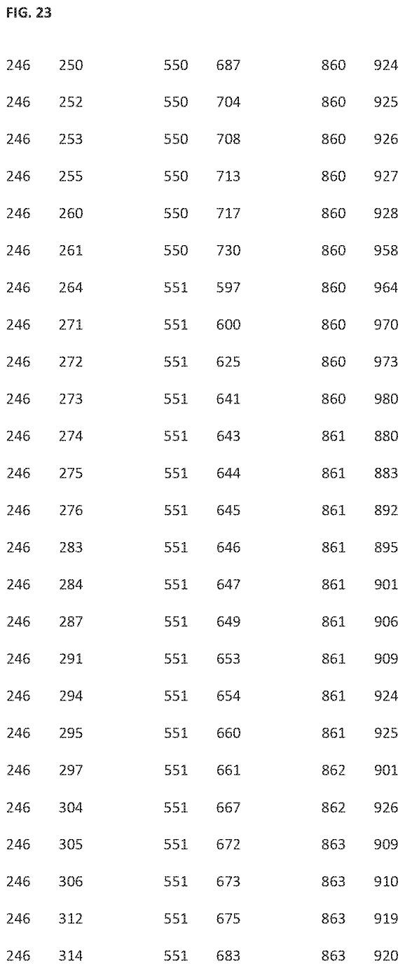

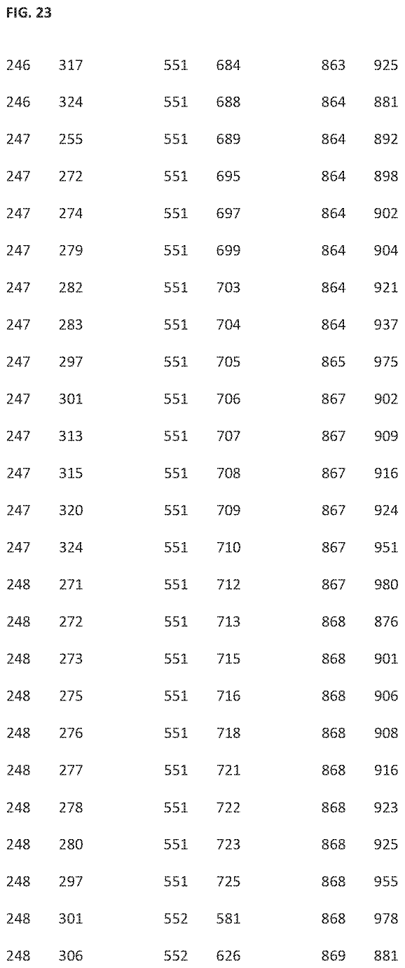

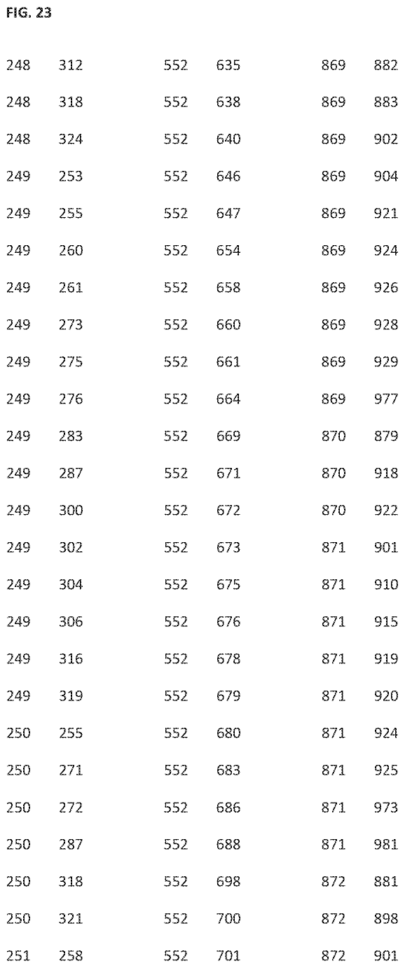

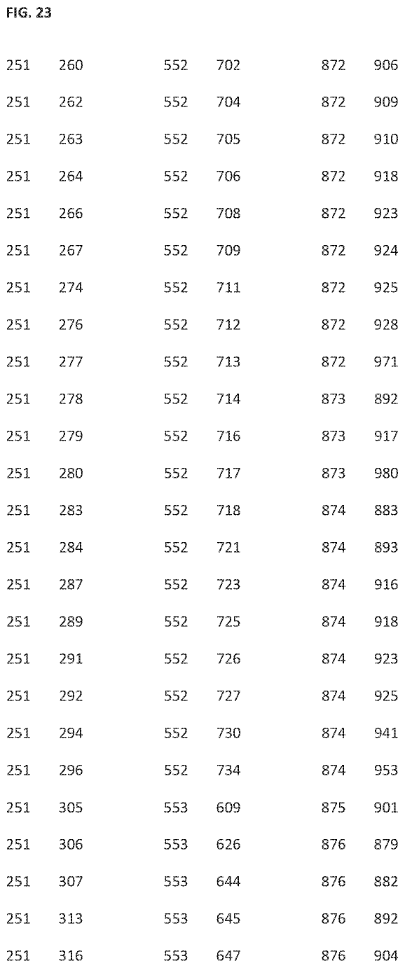

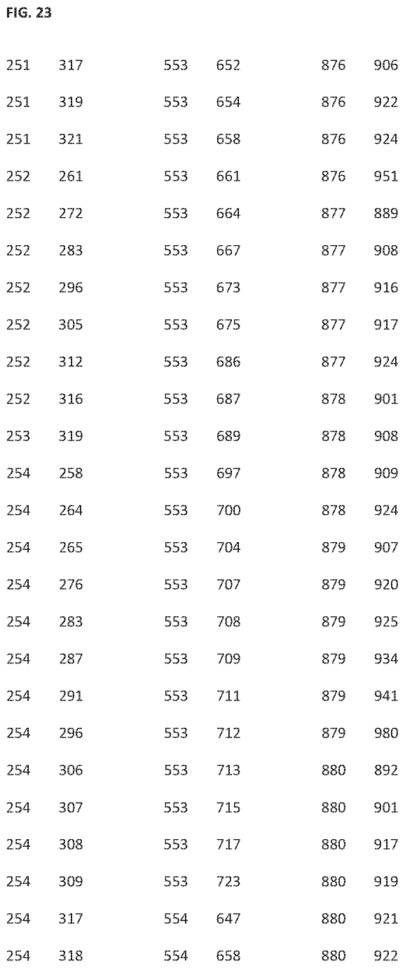

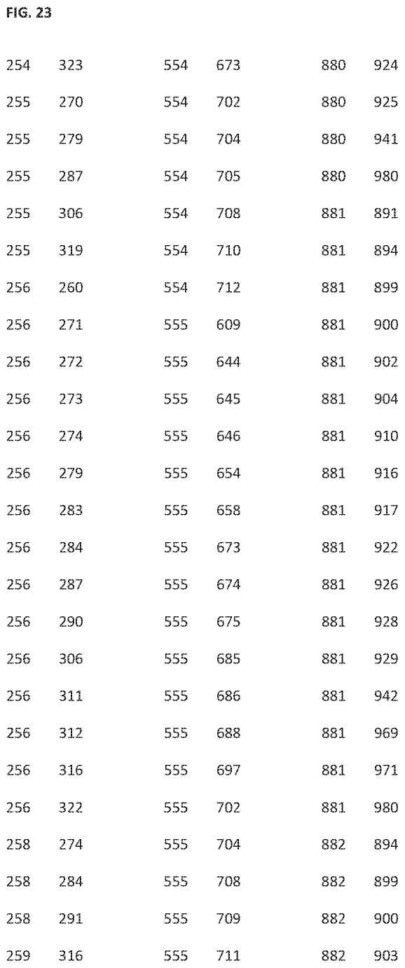

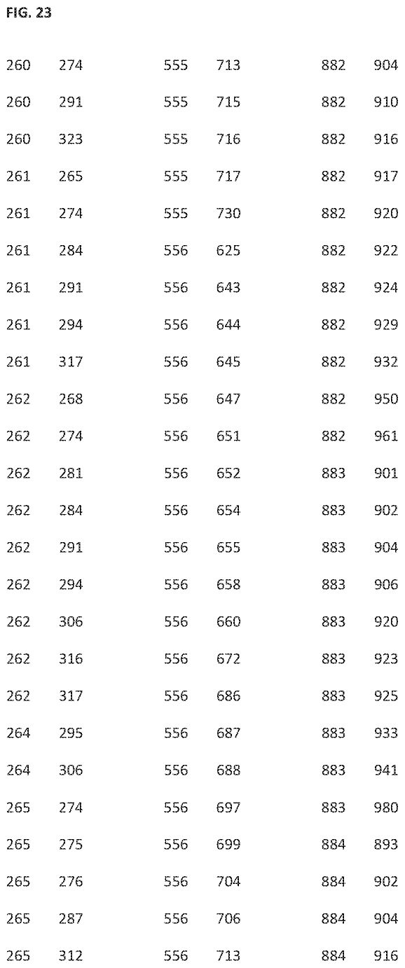

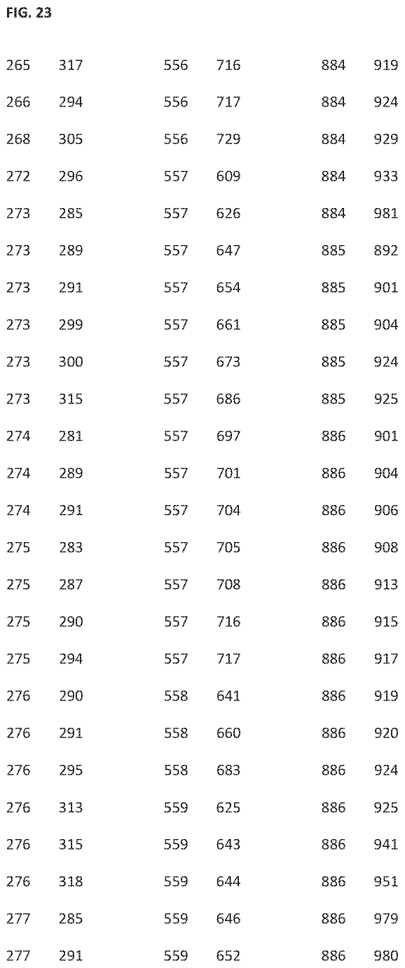

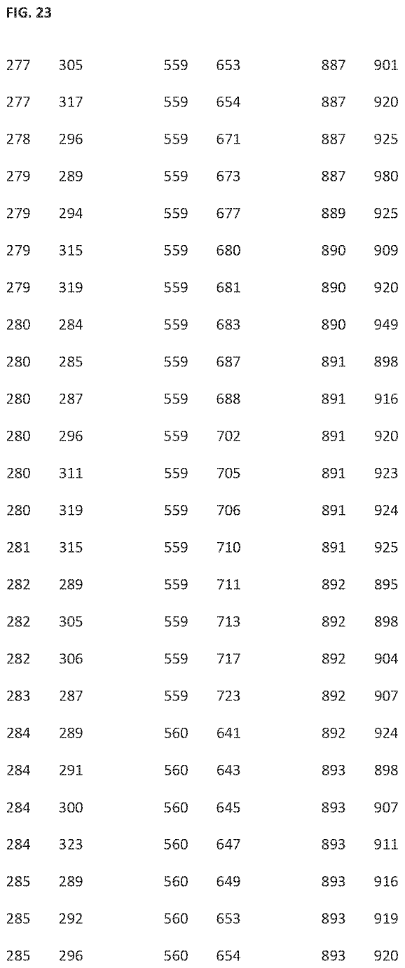

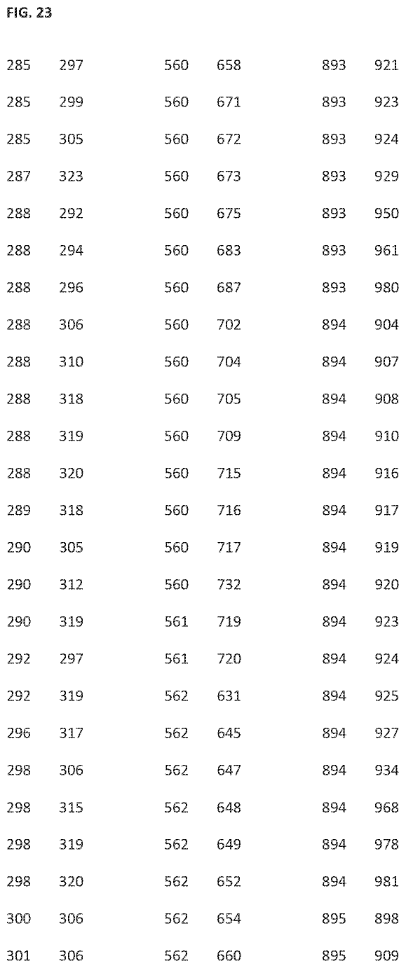

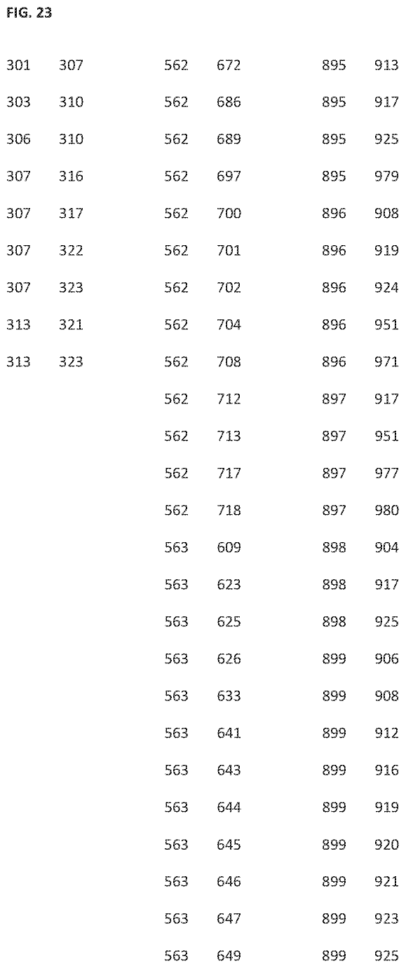

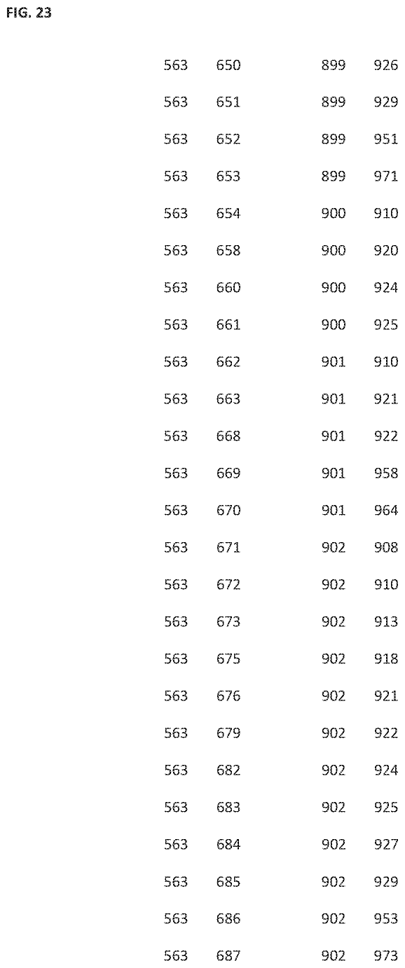

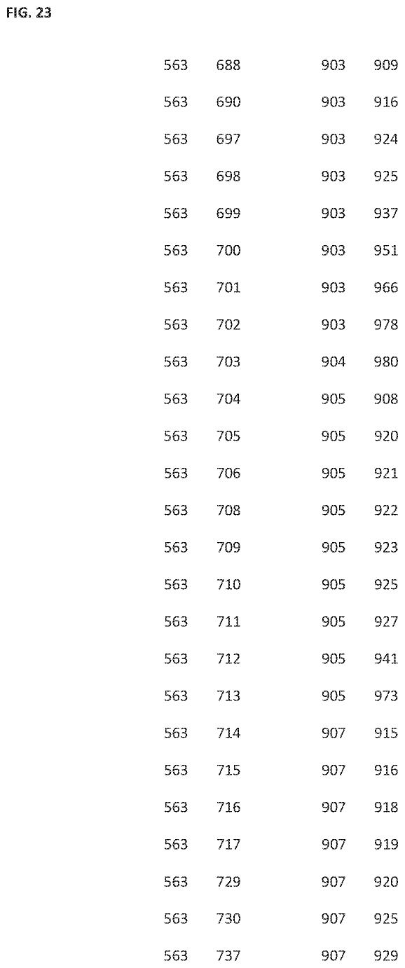

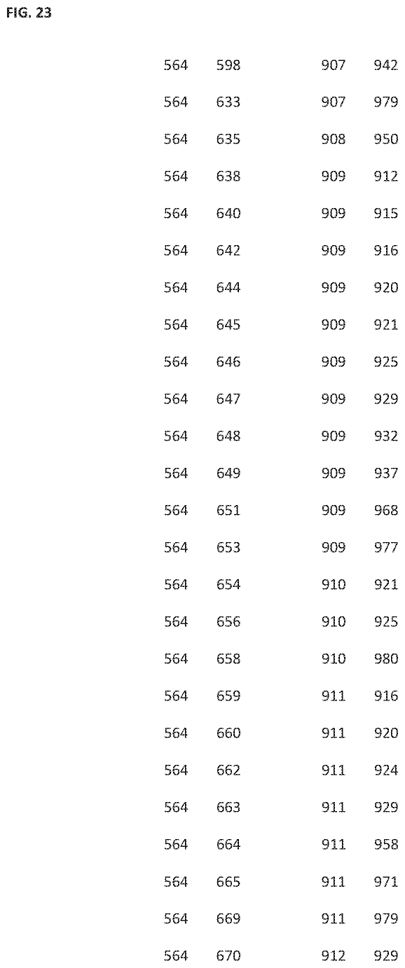

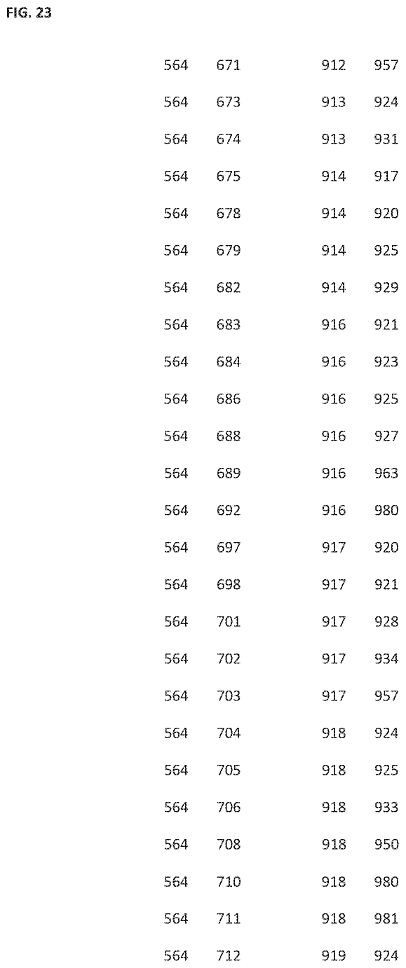

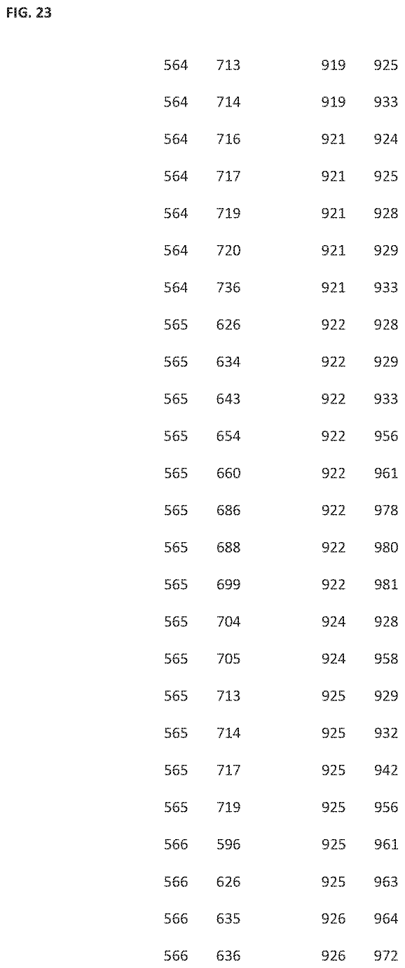

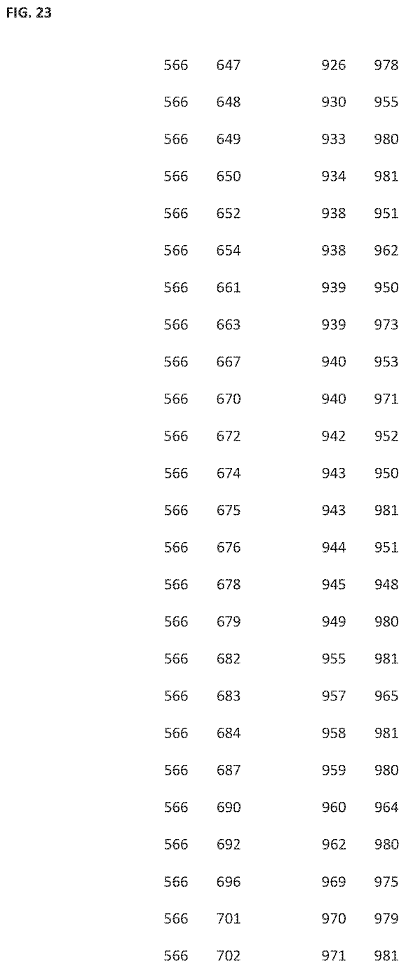

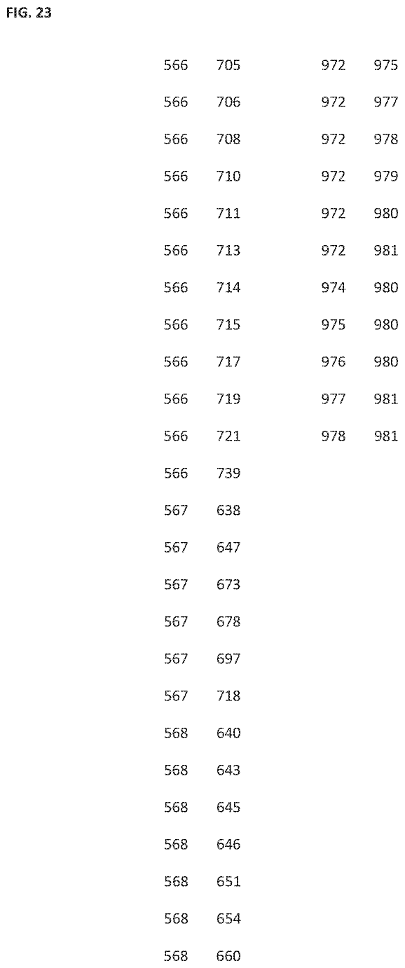

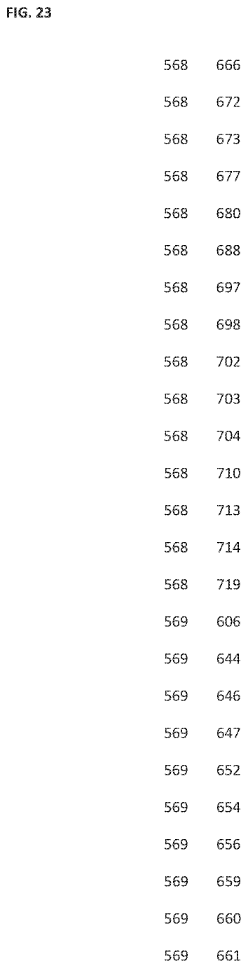

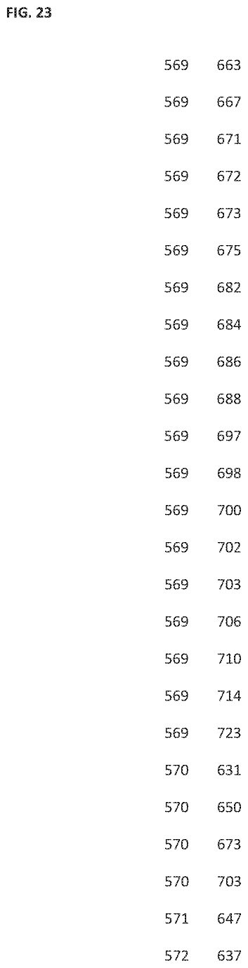

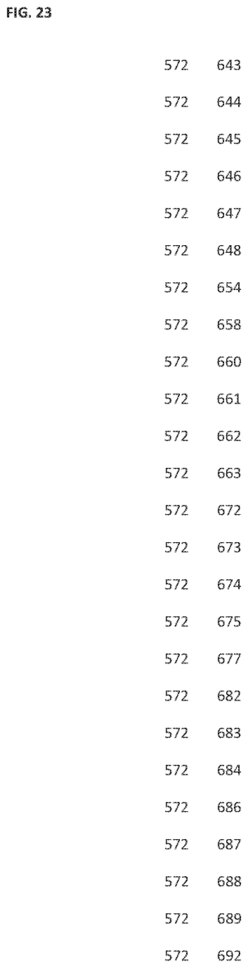

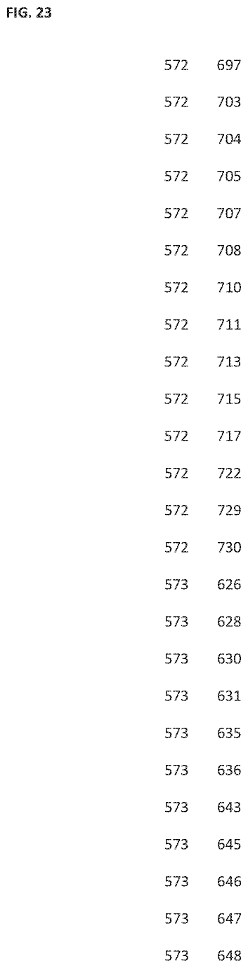

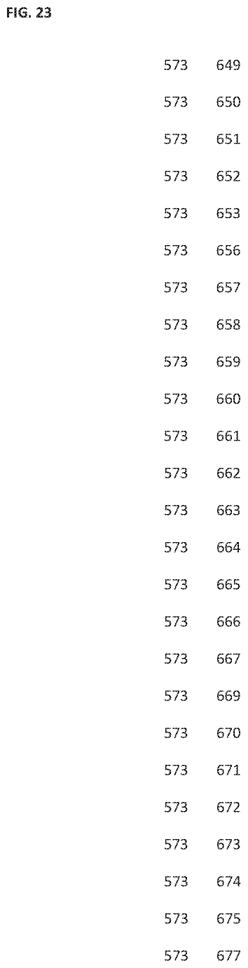

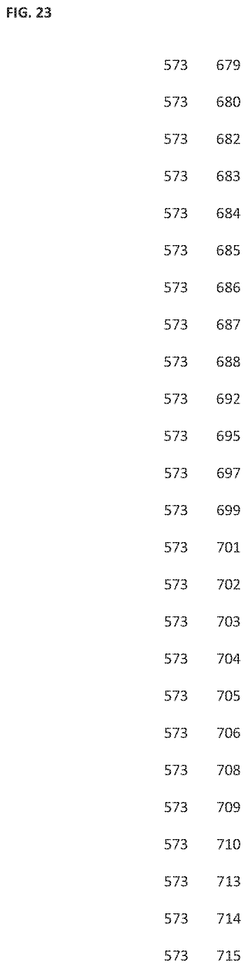

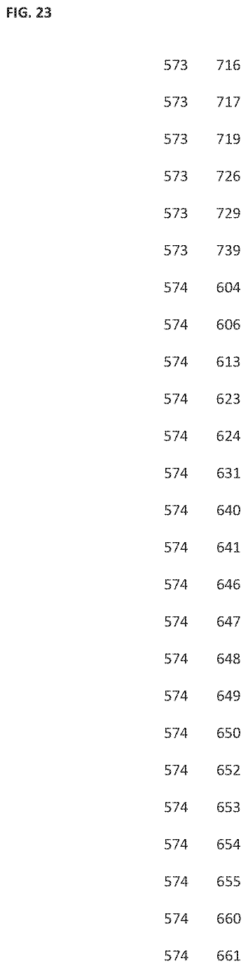

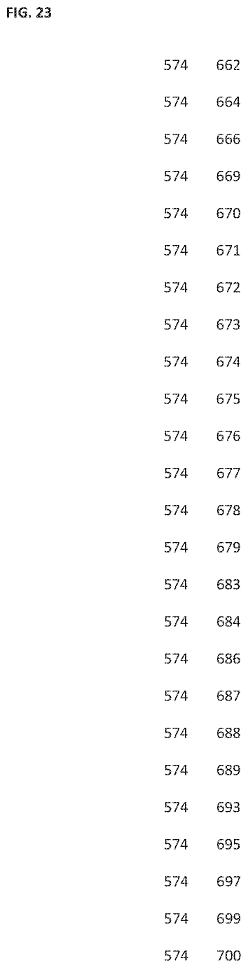

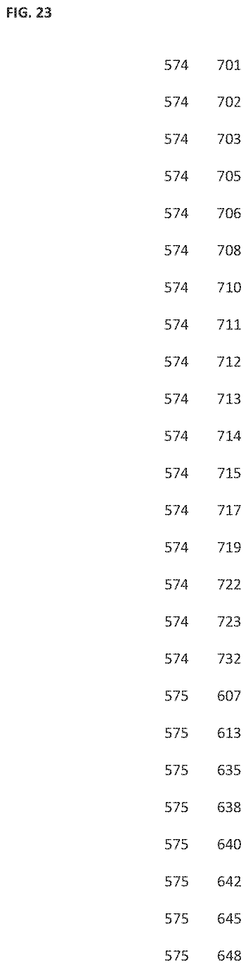

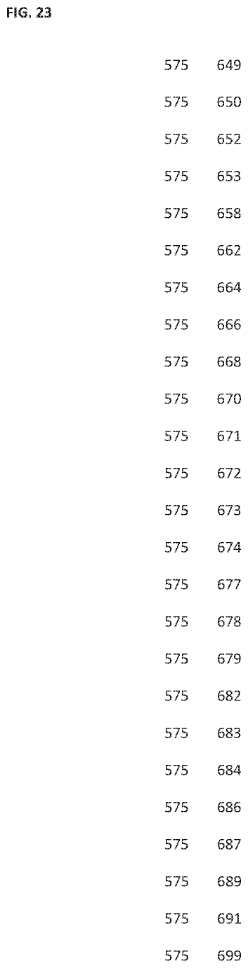

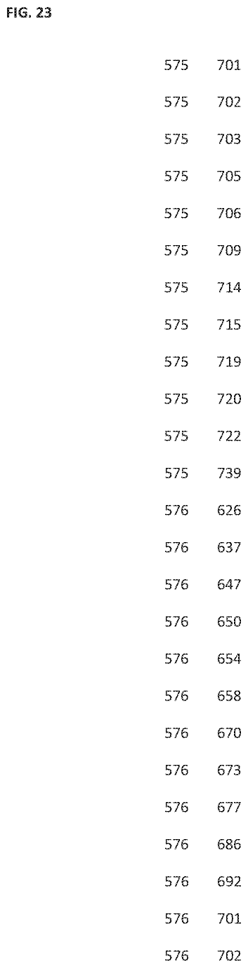

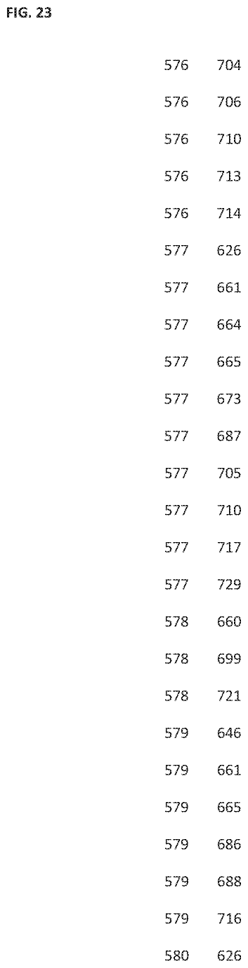

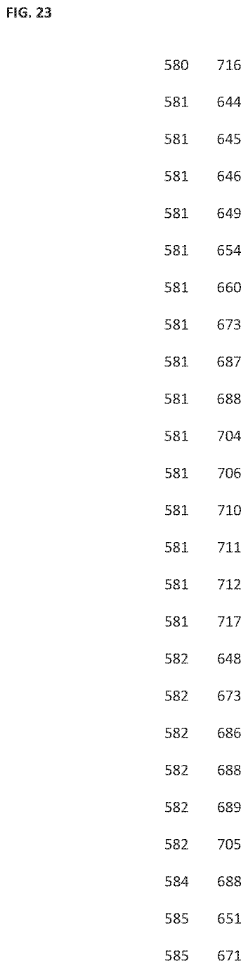

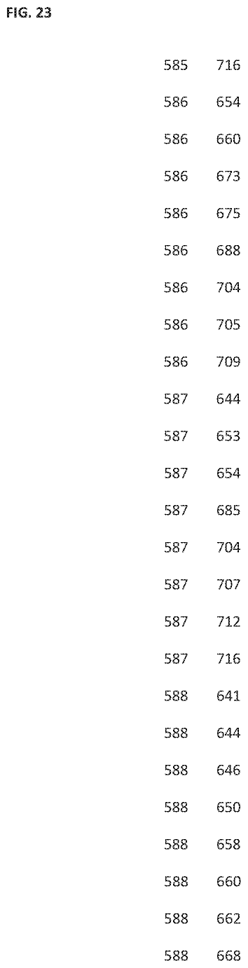

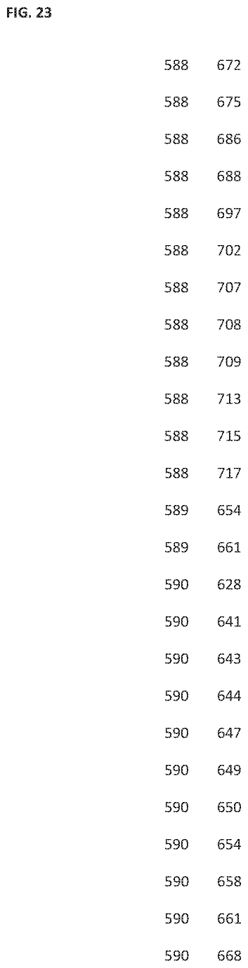

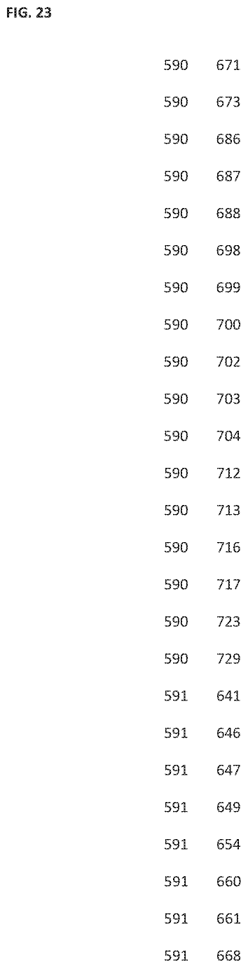

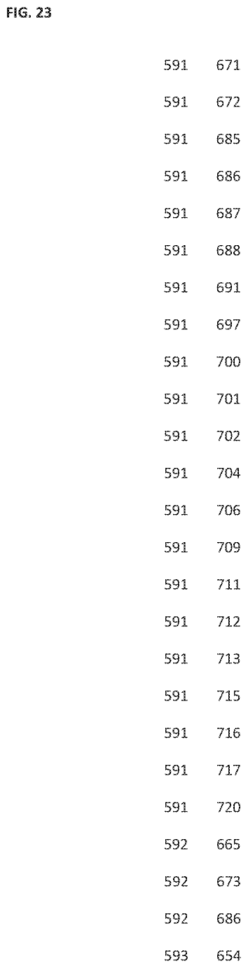

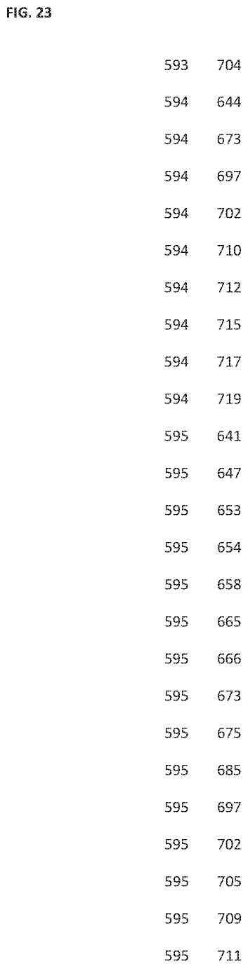

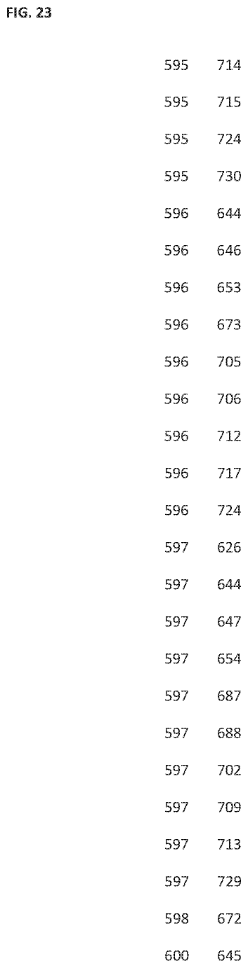

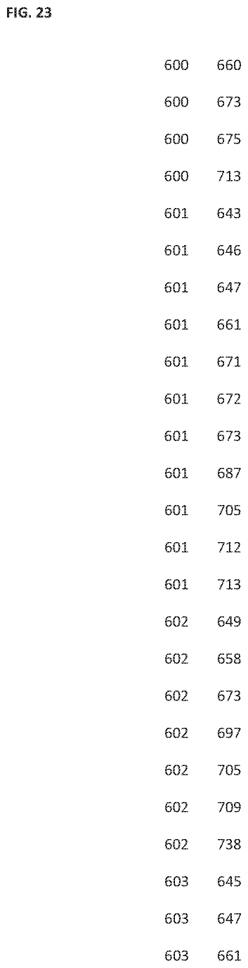

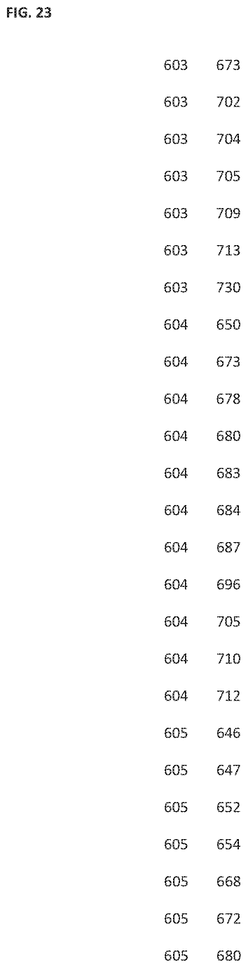

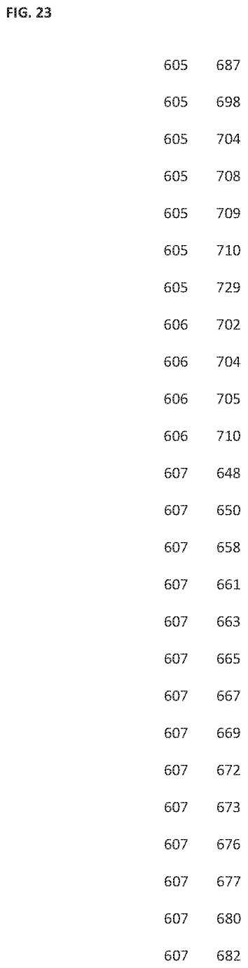

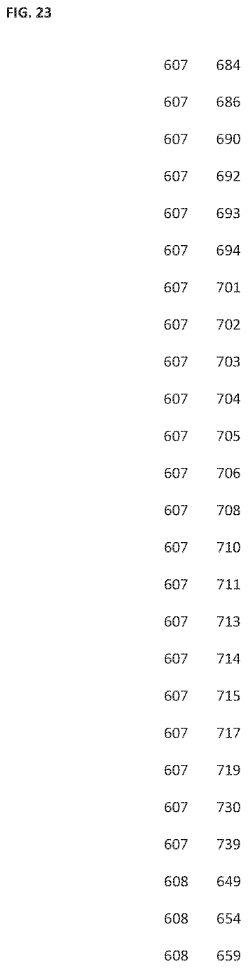

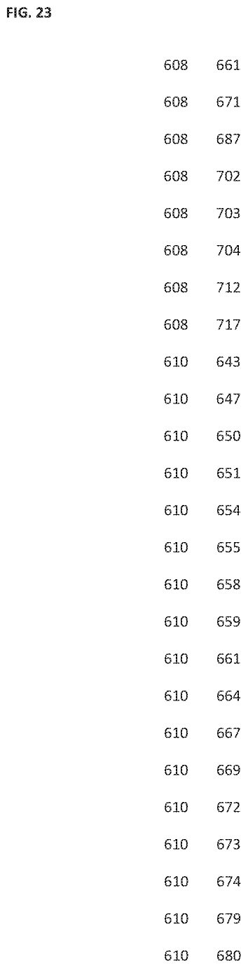

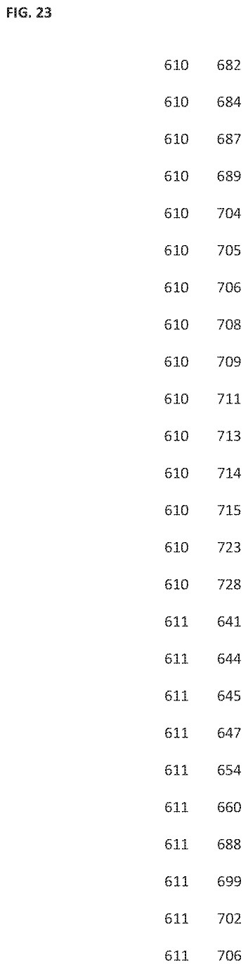

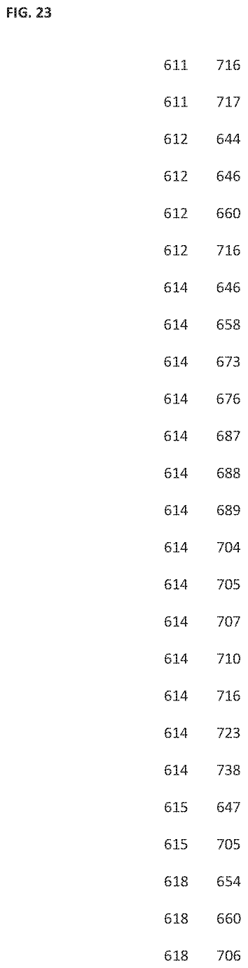

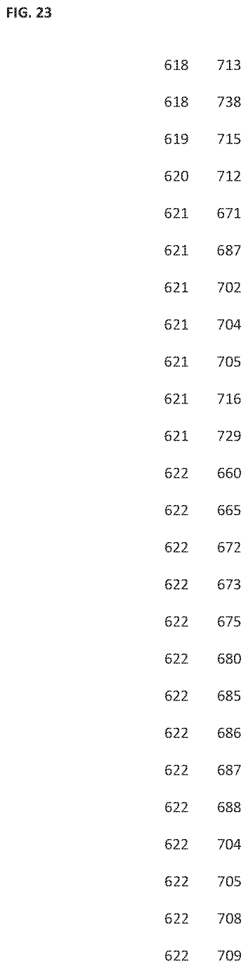

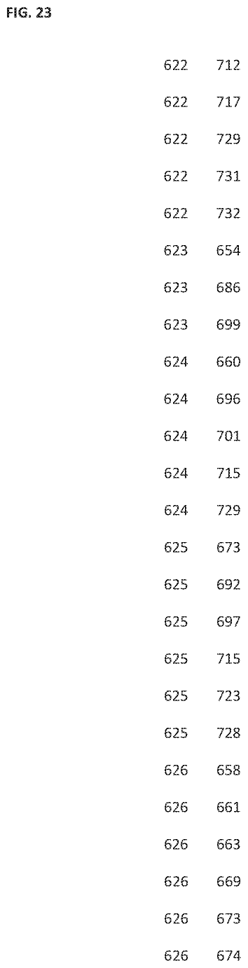

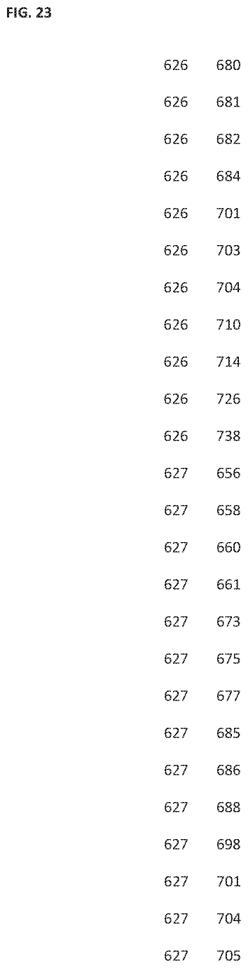

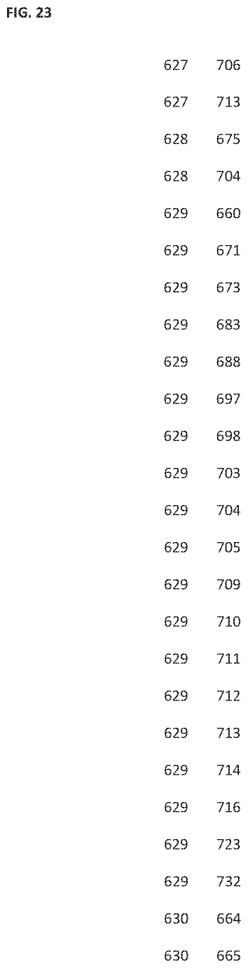

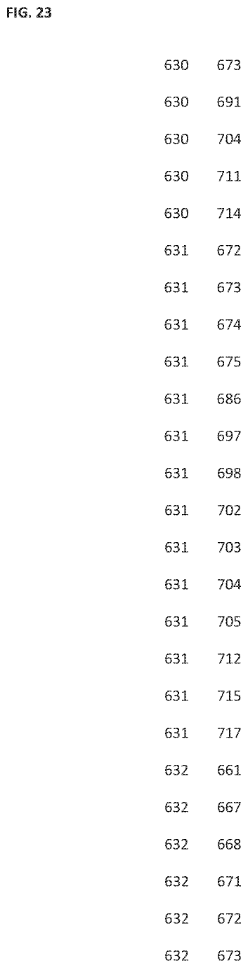

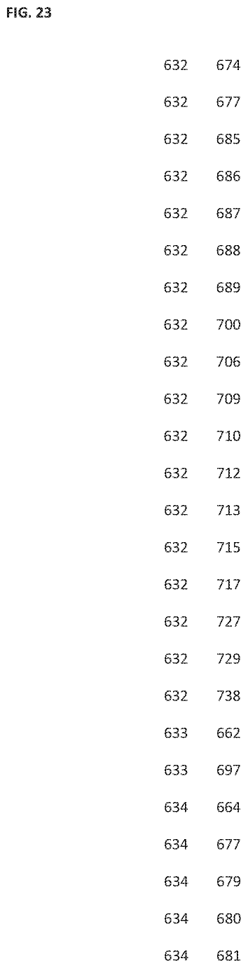

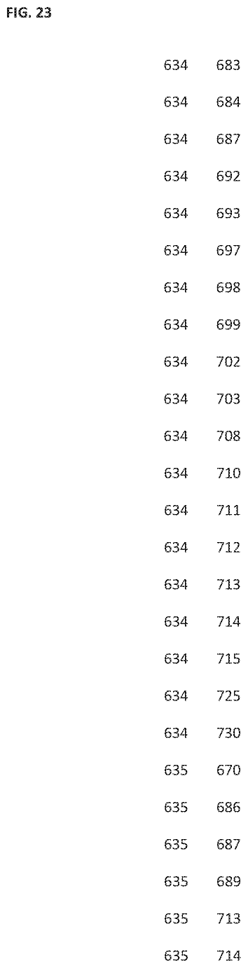

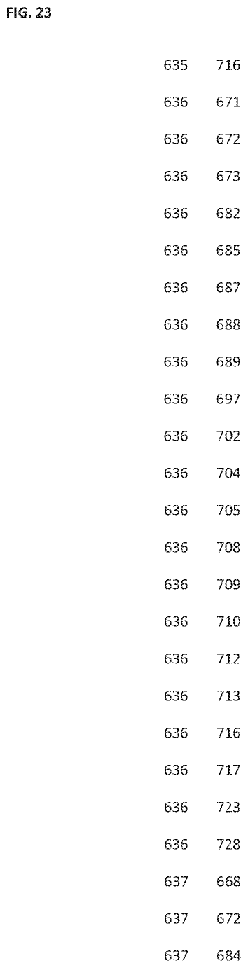

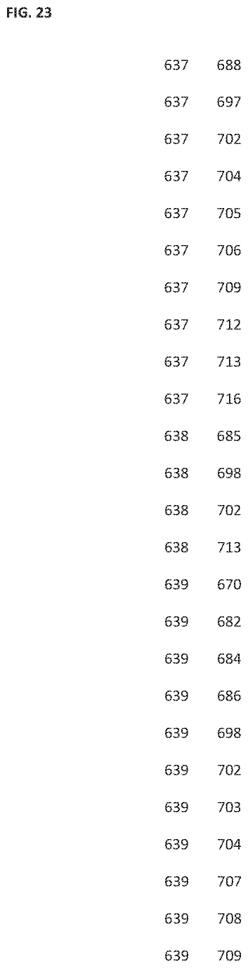

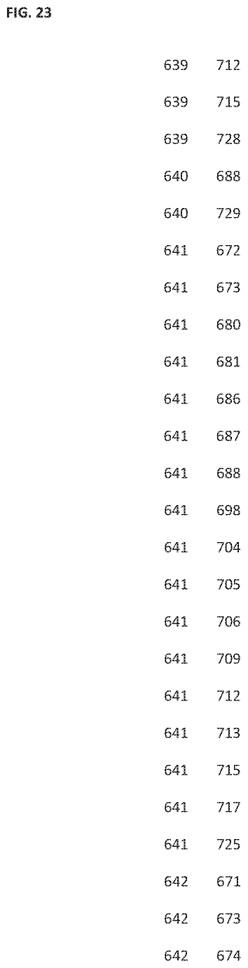

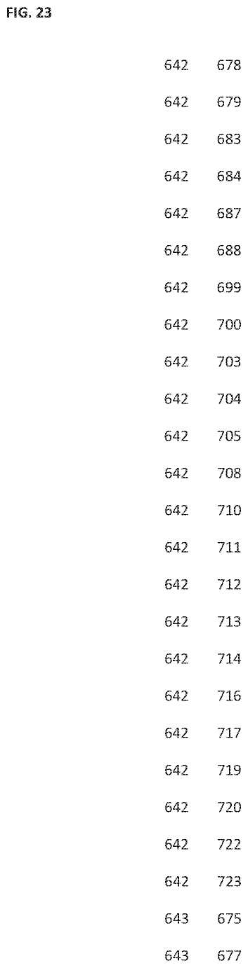

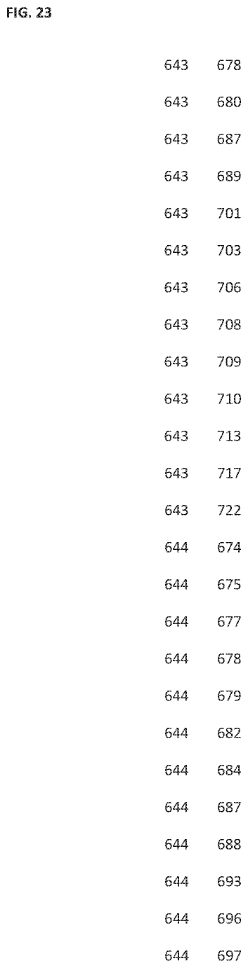

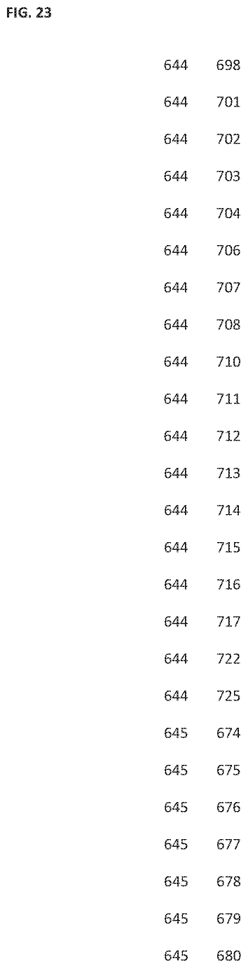

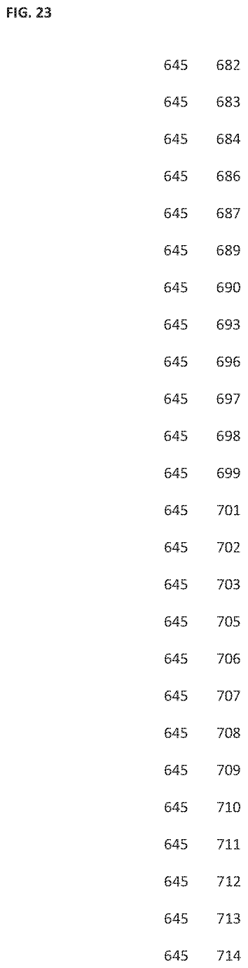

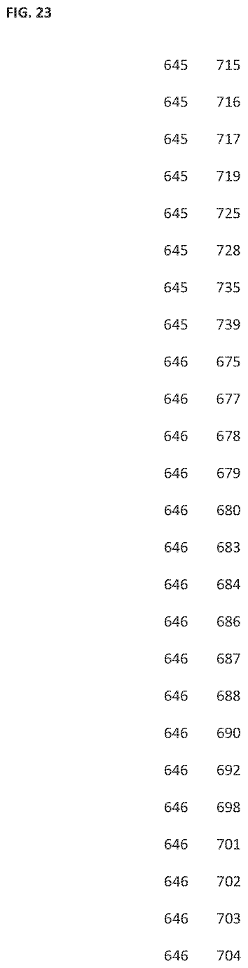

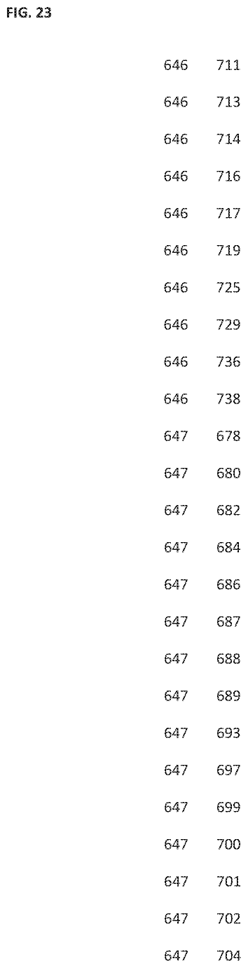

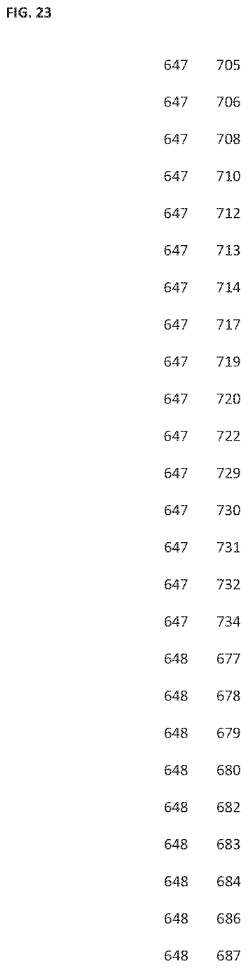

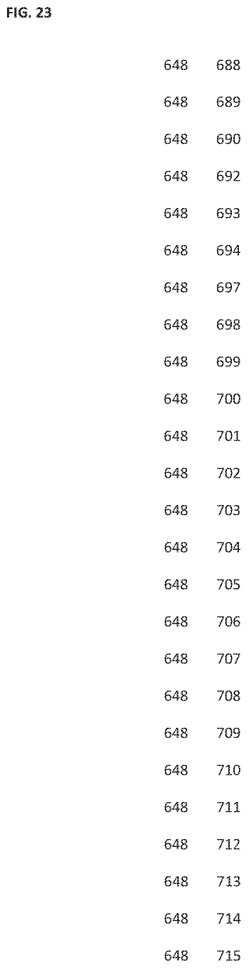

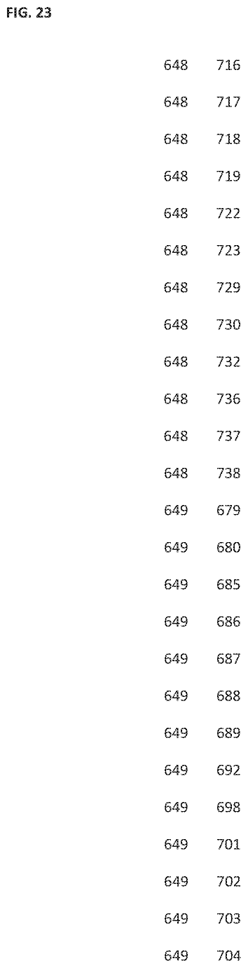

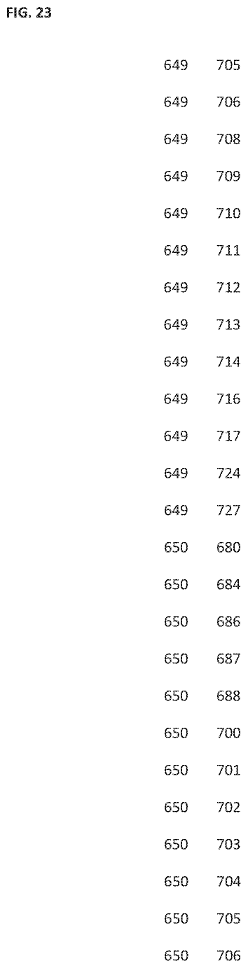

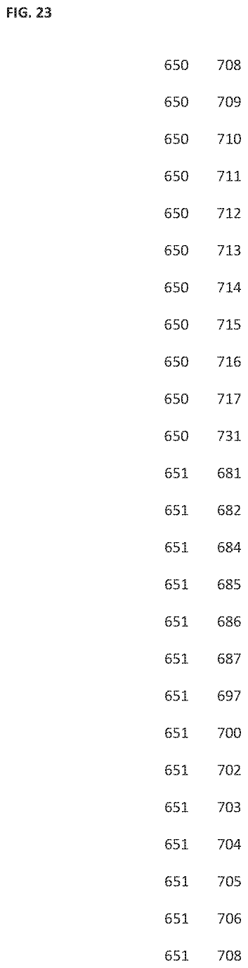

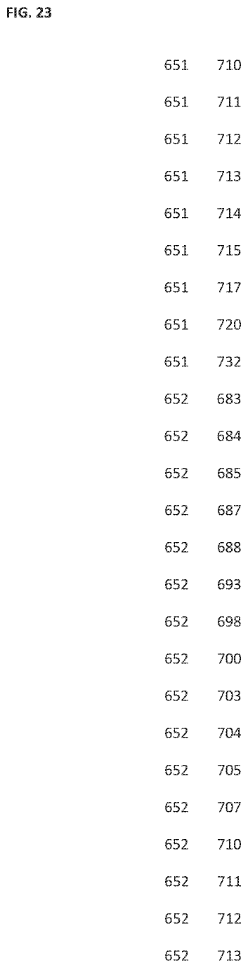

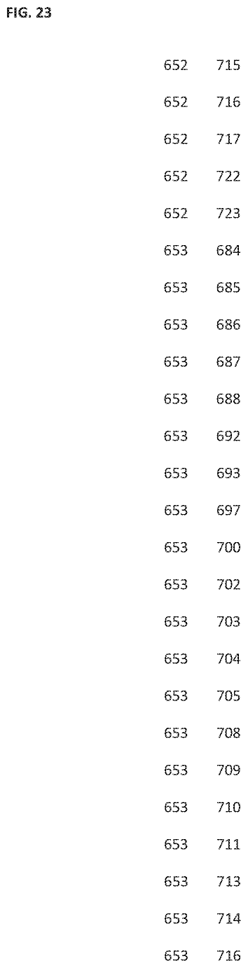

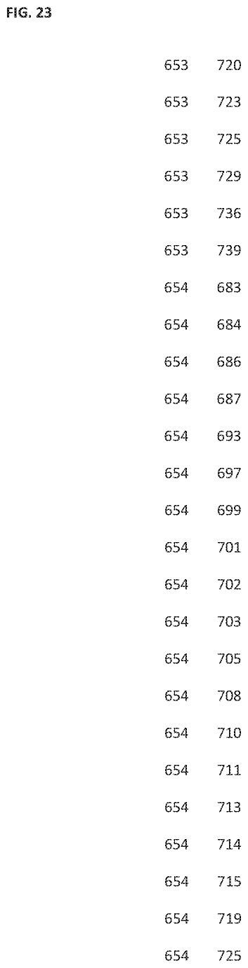

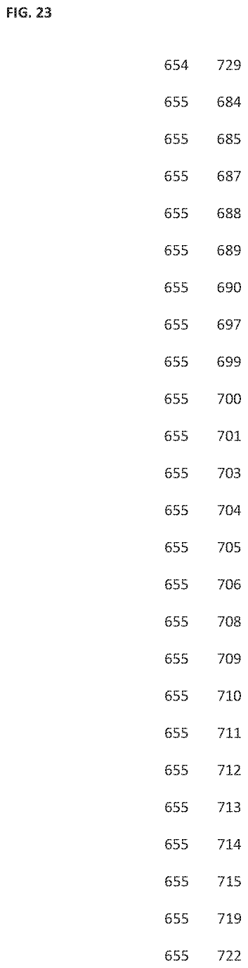

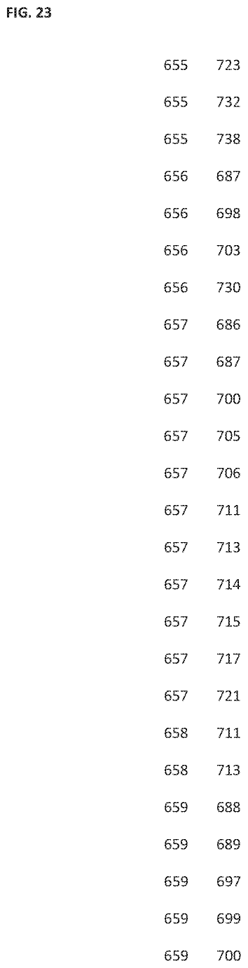

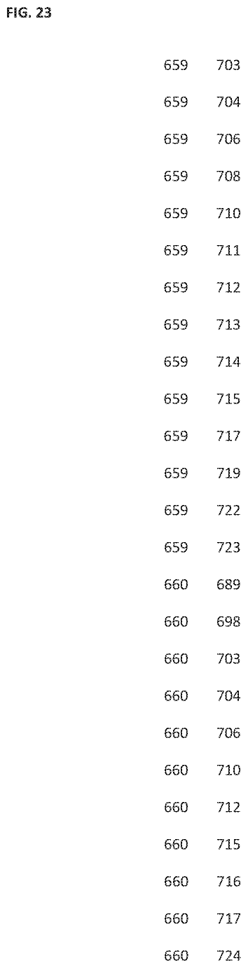

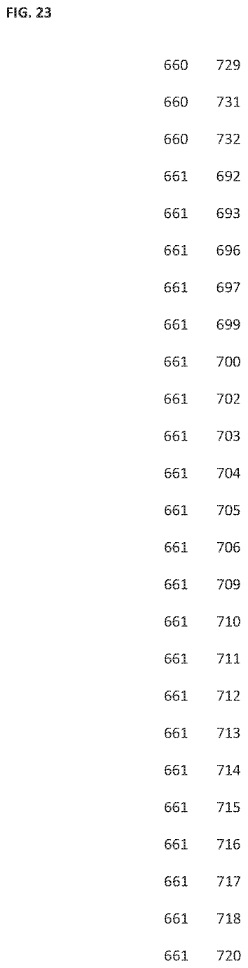

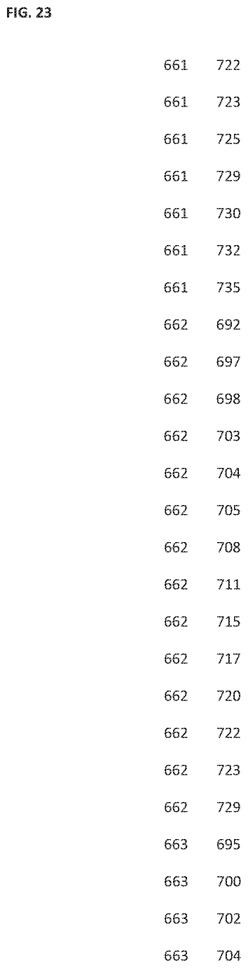

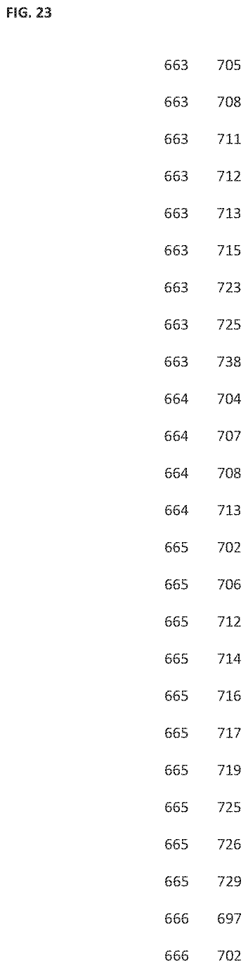

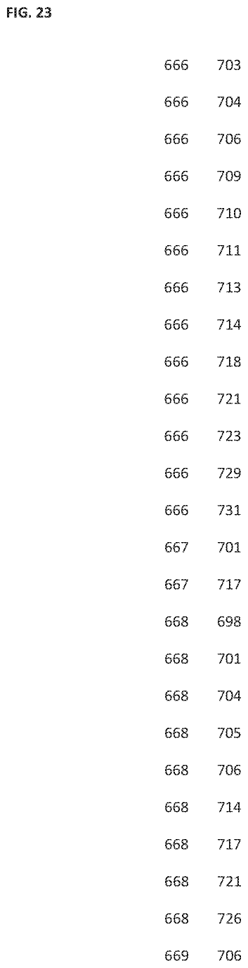

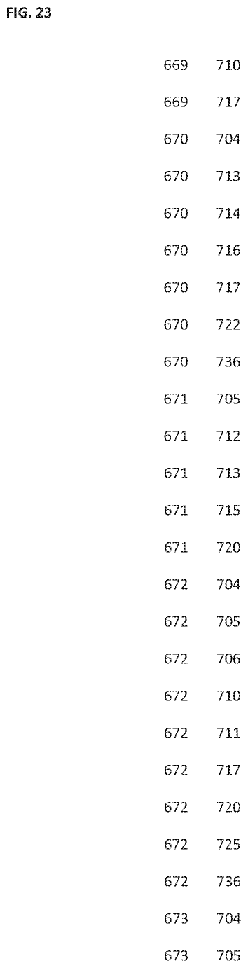

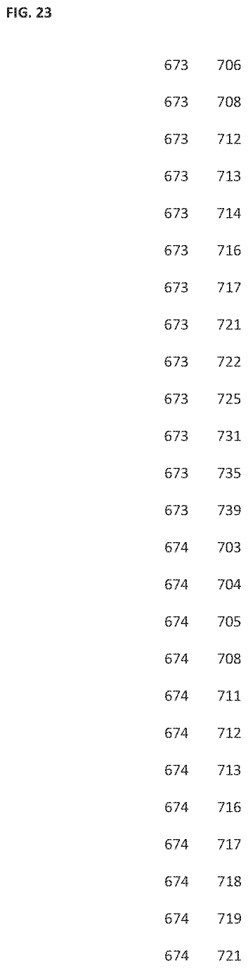

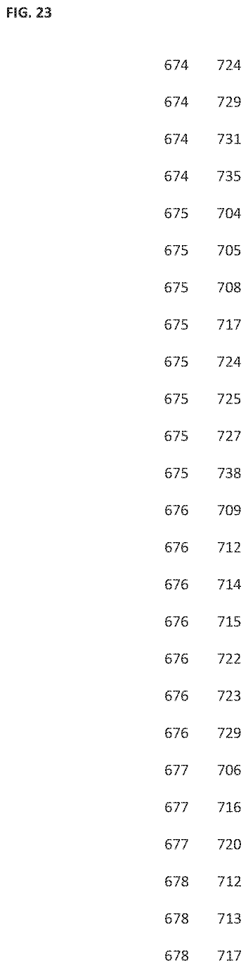

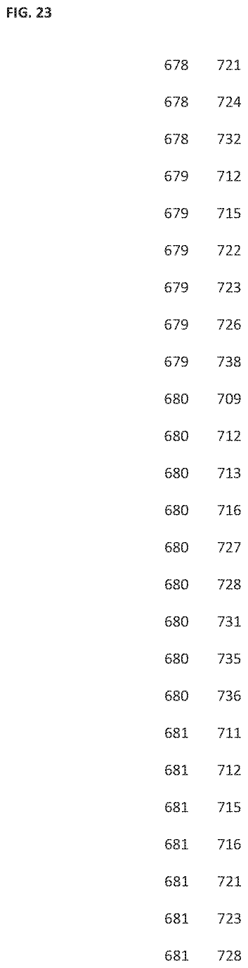

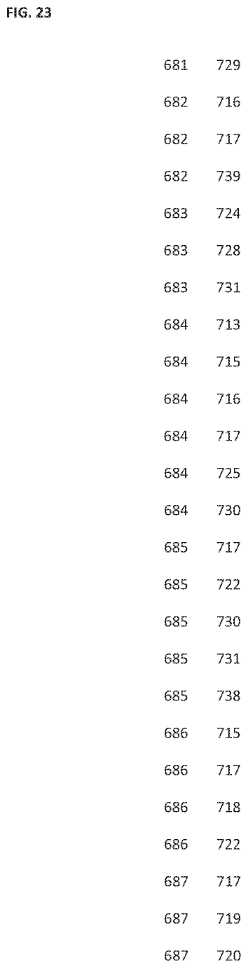

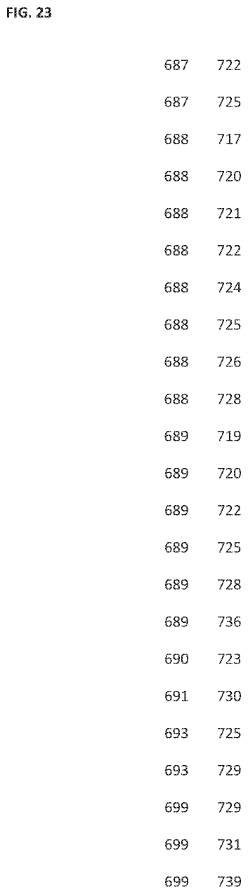

Non-limiting examples of RNA-guided effector polypeptides of the present disclosure that comprises one or more of “A,” “B,” “C,” and “D” deletions are depicted in FIG. 23. Examples of suitable combination of “A,” “B,” “C,” and “D” deletions are depicted in FIG. 23.

In some cases, an RNA-guided effector polypeptide of the present disclosure is a Cas9 protein that lacks all or a portion of one or more of following domains (in any combination): the Helical II domain, the Helical III domain, the HNH domain, and the RuvC III domain Any Cas9 protein can be used as a starting point for generation deletions, to generate an RNA-guided effector polypeptide of the present disclosure. Examples of suitable Cas9 proteins include, but are not limited to, those set forth in SEQ ID NOs: 5-816. In some cases, an RNA-guided effector polypeptide of the present disclosure comprises an “A” deletion based on the amino acid sequence of SEQ ID NO:5, or a corresponding deletion of a polypeptide as set forth in any one of SEQ ID NOs: 6-816. In some cases, an RNA-guided effector polypeptide of the present disclosure comprises an “A” deletion and a “B” deletion based on the amino acid sequence of SEQ ID NO:5, or a corresponding deletion of a polypeptide as set forth in any one of SEQ ID NOs: 6-816. In some cases, an RNA-guided effector polypeptide of the present disclosure comprises an “A” deletion and a “C” deletion based on the amino acid sequence of SEQ ID NO:5, or a corresponding deletion of a polypeptide as set forth in any one of SEQ ID NOs: 6-816. In some cases, an RNA-guided effector polypeptide of the present disclosure comprises an “A” deletion and a “D” deletion based on the amino acid sequence of SEQ ID NO:5, or a corresponding deletion of a polypeptide as set forth in any one of SEQ ID NOs: 6-816. In some cases, an RNA-guided effector polypeptide of the present disclosure comprises an “A” deletion, a “B” deletion, and a “C” deletion based on the amino acid sequence of SEQ ID NO:5, or a corresponding deletion of a polypeptide as set forth in any one of SEQ ID NOs: 6-816. In some cases, an RNA-guided effector polypeptide of the present disclosure comprises an “A” deletion a “B” deletion, a “C” deletion, and a “D” deletion based on the amino acid sequence of SEQ ID NO:5, or a corresponding deletion of a polypeptide as set forth in any one of SEQ ID NOs: 6-816. In some cases, an RNA-guided effector polypeptide of the present disclosure comprises a “B” deletion, a “C” deletion, and a “D” deletion based on the amino acid sequence of SEQ ID NO:5, or a corresponding deletion of a polypeptide as set forth in any one of SEQ ID NOs: 6-816. In some cases, an RNA-guided effector polypeptide of the present disclosure comprises an “A” deletion a “C” deletion and a “D” deletion based on the amino acid sequence of SEQ ID NO:5, or a corresponding deletion of a polypeptide as set forth in any one of SEQ ID NOs: 6-816. While the following description of various deletions refers to Spy Cas9, the description can be applied to a Cas9 comprising an amino acid sequence set forth in any one of SEQ ID NOs:5-816. The amino acid sequence of a “corresponding deletion of a polypeptide as set forth in any one of SEQ ID NOs:6-816” can be readily determined using a multiple sequence alignment of one or more of the amino acid sequences set forth in SEQ ID NOs:6-816 with the amino acid sequence set forth in SEQ ID NO:5 (Streptococcus pyogenes Cas9).

### “A” Deletion

In some cases, an RNA-guided effector polypeptide of the present disclosure comprises a deletion of from about 5 contiguous amino acids to about 158 contiguous amino acids (e.g., from about 5 to about 50, from about 50 to about 75, from about 75 to about 100, from about 100 to about 125, or from about 125 to about 158 contiguous amino acids) of a region corresponding to amino acids 166-324 of the Spy Cas9 amino acid sequence depicted in FIG. 1; and comprises one or more stretches of from 50 to 1000 contiguous amino acids (e.g., from 50 to 75, from 75 to 100, from 100 to 150, from 150 to 200, from 200 to 250, from 250 to 300, from 300 to 350, from 350 to 400, from 400 to 450, from 450 to 500, from 500 to 600, from 600 to 700, from 700 to 800, from 800 to 900, or from 900 to 1000, contiguous amino acids) having at least 85%, at least 90%, at least 95%, at least 98%, at least 99%, or 100%, amino acid sequence identity to corresponding stretches of from 50 to 1000 contiguous amino acids (e.g., from 50 to 75, from 75 to 100, from 100 to 150, from 150 to 200, from 200 to 250, from 250 to 300, from 300 to 350, from 350 to 400, from 400 to 450, from 450 to 500, from 500 to 600, from 600 to 700, from 700 to 800, from 800 to 900, or from 900 to 1000, contiguous amino acids) of the Spy Cas9 amino acid sequence depicted in FIG. 1. In some cases, an RNA-guided effector polypeptide of the present disclosure comprises only one deletion of from about 5 contiguous amino acids to about 158 contiguous amino acids (e.g., from about 5 to about 50, from about 50 to about 75, from about 75 to about 100, from about 100 to about 125, or from about 125 to about 158 contiguous amino acids) of a region corresponding to amino acids 166-324 of the Spy Cas9 amino acid sequence depicted in FIG. 1. In some cases, an RNA-guided effector polypeptide of the present disclosure comprising an “A” deletion has a length of from about 1210 amino acids to about 1363 amino acids, e.g., from about 1210 amino acids (aa) to about 1220 aa, from about 1220 aa to about 1230 aa, from about 1230 aa to about 1240 aa, from about 1240 aa to about 1250 aa, from about 1250 aa to about 1260 aa, from about 1260 aa to about 1270 aa, from about 1270 aa to about 1280 aa, from about 1280 aa to about 1290 aa, from about 1290 aa to about 1300 aa, from about 1300 aa to about 1310 aa, from about 1310 aa to about 1320 aa, from about 1320 aa to about 1330 aa, from about 1330 aa to about 1340 aa, from about 1340 aa to about 1350 aa, or from about 1350 aa to 1363 aa. In some cases, an RNA-guided effector polypeptide of the present disclosure comprising only an “A” deletion has a length of 1210 amino acids.

### “B” Deletion

In some cases, an RNA-guided effector polypeptide of the present disclosure comprises a deletion of from about 30 contiguous amino acids to about 255 contiguous amino acids (e.g., from about 30 to about 50, from about 50 to about 75, from about 75 to about 100, from about 100 to about 150, from about 150 to about 200, or from about 200 to about 255 contiguous amino acids) of a region corresponding to amino acids 484-739 of the Spy Cas9 amino acid sequence depicted in FIG. 1; and comprises one or more stretches of from 50 to 1000 contiguous amino acids (e.g., from 50 to 75, from 75 to 100, from 100 to 150, from 150 to 200, from 200 to 250, from 250 to 300, from 300 to 350, from 350 to 400, from 400 to 450, from 450 to 500, from 500 to 600, from 600 to 700, from 700 to 800, from 800 to 900, or from 900 to 1000, contiguous amino acids) having at least 85%, at least 90%, at least 95%, at least 98%, at least 99%, or 100%, amino acid sequence identity to corresponding stretches of from 50 to 1000 contiguous amino acids (e.g., from 50 to 75, from 75 to 100, from 100 to 150, from 150 to 200, from 200 to 250, from 250 to 300, from 300 to 350, from 350 to 400, from 400 to 450, from 450 to 500, from 500 to 600, from 600 to 700, from 700 to 800, from 800 to 900, or from 900 to 1000, contiguous amino acids) of the Spy Cas9 amino acid sequence depicted in FIG. 1. In some cases, an RNA-guided effector polypeptide of the present disclosure comprises only one deletion of from about 30 contiguous amino acids to about 255 contiguous amino acids (e.g., from about 30 to about 50, from about 50 to about 75, from about 75 to about 100, from about 100 to about 150, from about 150 to about 200, or from about 200 to about 255 contiguous amino acids) of a region corresponding to amino acids 484-739 of the Spy Cas9 amino acid sequence depicted in FIG. 1. In some cases, an RNA-guided effector polypeptide of the present disclosure comprising a “B” deletion has a length of from about 1113 amino acids to about 1338 amino acids, e.g., from about 1113 amino acids (aa) to about 1120 aa, from about 1120 aa to about 1130 aa, from about 1130 aa to about 1140 aa, from about 1140 aa to about 1150 aa, from about 1150 aa to about 1160 aa, from about 1160 aa to about 1170 aa, from about 1170 aa to about 1180 aa, from about 1180 aa to about 1190 aa, from about 1190 aa to about 1200 aa, from about 1200 aa to about 1225 aa, from about 1225 aa to about 1250 aa, from about 1250 aa to about 1275 aa, from about 1275 aa to about 1300 aa, from about 1300 aa to about 1325 aa, or from about 1325 aa to 1338 aa. In some cases, an RNA-guided effector polypeptide of the present disclosure comprising only a “B” deletion has a length of 1163 aa, 1164 aa, or 1165 aa.

### “C” Deletion

In some cases, an RNA-guided effector polypeptide of the present disclosure comprises a deletion of from about 4 contiguous amino acids to about 220 contiguous amino acids (e.g., from about 4 to about 25, from about 25 to about 50, from about 50 to about 75, from about 75 to about 100, from about 100 to about 150, from about 150 to about 200, or from about 200 to about 220, contiguous amino acids) of a region corresponding to amino acids 761-981 of the Spy Cas9 amino acid sequence depicted in FIG. 1; and comprises one or more stretches of from 50 to 1000 contiguous amino acids (e.g., from 50 to 75, from 75 to 100, from 100 to 150, from 150 to 200, from 200 to 250, from 250 to 300, from 300 to 350, from 350 to 400, from 400 to 450, from 450 to 500, from 500 to 600, from 600 to 700, from 700 to 800, from 800 to 900, or from 900 to 1000, contiguous amino acids) having at least 85%, at least 90%, at least 95%, at least 98%, at least 99%, or 100%, amino acid sequence identity to corresponding stretches of from 50 to 1000 contiguous amino acids (e.g., from 50 to 75, from 75 to 100, from 100 to 150, from 150 to 200, from 200 to 250, from 250 to 300, from 300 to 350, from 350 to 400, from 400 to 450, from 450 to 500, from 500 to 600, from 600 to 700, from 700 to 800, from 800 to 900, or from 900 to 1000, contiguous amino acids) of the Spy Cas9 amino acid sequence depicted in FIG. 1. In some cases, an RNA-guided effector polypeptide of the present disclosure comprises only one deletion of from about 4 contiguous amino acids to about 220 contiguous amino acids (e.g., from about 4 to about 25, from about 25 to about 50, from about 50 to about 75, from about 75 to about 100, from about 100 to about 150, from about 150 to about 200, or from about 200 to about 220, contiguous amino acids) of a region corresponding to amino acids 761-981 of the Spy Cas9 amino acid sequence depicted in FIG. 1. In some cases, an RNA-guided effector polypeptide of the present disclosure comprising a “C” deletion has a length of from about 1148 amino acids to about 1364 amino acids, e.g., from about 1148 amino acids (aa) to about 1175 aa, from about 1175 aa to about 1200 aa, from about 1200 aa to about 1225 aa, from about 1225 aa to about 1250 aa, from about 1250 aa to about 1275 aa, from about 1275 aa to about 1300 aa, from about 1300 aa to about 1325 aa, from about 1325 aa to 1350 aa, or from about 1350 aa to 1364 aa. In some cases, an RNA-guided effector polypeptide of the present disclosure comprising only a “C” deletion has a length of 1263 aa, 1264 aa, or 1265 aa.

### “D” Deletion

In some cases, an RNA-guided effector polypeptide of the present disclosure comprises a deletion of from about 20 contiguous amino acids to about 118 contiguous amino acids (e.g., from about 20 to about 40, from about 40 to about 60, from about 60 to about 80, from about 80 to about 100, or from about 100 to about 118 contiguous amino acids) of a region corresponding to amino acids 994-1112 of the Spy Cas9 amino acid sequence depicted in FIG. 1; and comprises one or more stretches of from 50 to 1000 contiguous amino acids (e.g., from 50 to 75, from 75 to 100, from 100 to 150, from 150 to 200, from 200 to 250, from 250 to 300, from 300 to 350, from 350 to 400, from 400 to 450, from 450 to 500, from 500 to 600, from 600 to 700, from 700 to 800, from 800 to 900, or from 900 to 1000, contiguous amino acids) having at least 85%, at least 90%, at least 95%, at least 98%, at least 99%, or 100%, amino acid sequence identity to corresponding stretches of from 50 to 1000 contiguous amino acids (e.g., from 50 to 75, from 75 to 100, from 100 to 150, from 150 to 200, from 200 to 250, from 250 to 300, from 300 to 350, from 350 to 400, from 400 to 450, from 450 to 500, from 500 to 600, from 600 to 700, from 700 to 800, from 800 to 900, or from 900 to 1000, contiguous amino acids) of the Spy Cas9 amino acid sequence depicted in FIG. 1. In some cases, an RNA-guided effector polypeptide of the present disclosure comprises only one deletion of from about 20 contiguous amino acids to about 118 contiguous amino acids (e.g., from about 20 to about 40, from about 40 to about 60, from about 60 to about 80, from about 80 to about 100, or from about 100 to about 118 contiguous amino acids) of a region corresponding to amino acids 994-1112 of the Spy Cas9 amino acid sequence depicted in FIG. 1. In some cases, an RNA-guided effector polypeptide of the present disclosure comprising a “D” deletion has a length of from about 1250 amino acids to about 1348 amino acids, e.g., from about 1250 amino acids (aa) to about 1275 aa, from about 1275 aa to about 1300 aa, from about 1300 aa to about 1325 aa, or from about 1325 aa to about 1348 aa. In some cases, an RNA-guided effector polypeptide of the present disclosure comprising only a “D” deletion has a length of 1297 aa, 1298 aa, or 1299 aa.

### “A+B” Deletion

In some cases, an RNA-guided effector polypeptide of the present disclosure comprises: i) a deletion of from about 5 contiguous amino acids to about 158 contiguous amino acids (e.g., from about 5 to about 50, from about 50 to about 75, from about 75 to about 100, from about 100 to about 125, or from about 125 to about 158 contiguous amino acids) of a region corresponding to amino acids 166-324 of the Spy Cas9 amino acid sequence depicted in FIG. 1; and ii) a deletion of from about 30 contiguous amino acids to about 255 contiguous amino acids (e.g., from about 30 to about 50, from about 50 to about 75, from about 75 to about 100, from about 100 to about 150, from about 150 to about 200, or from about 200 to about 255 contiguous amino acids) of a region corresponding to amino acids 484-739 of the Spy Cas9 amino acid sequence depicted in FIG. 1; and comprises one or more stretches of from 50 to 1000 contiguous amino acids (e.g., from 50 to 75, from 75 to 100, from 100 to 150, from 150 to 200, from 200 to 250, from 250 to 300, from 300 to 350, from 350 to 400, from 400 to 450, from 450 to 500, from 500 to 600, from 600 to 700, from 700 to 800, from 800 to 900, or from 900 to 1000, contiguous amino acids) having at least 85%, at least 90%, at least 95%, at least 98%, at least 99%, or 100%, amino acid sequence identity to corresponding stretches of from 50 to 1000 contiguous amino acids (e.g., from 50 to 75, from 75 to 100, from 100 to 150, from 150 to 200, from 200 to 250, from 250 to 300, from 300 to 350, from 350 to 400, from 400 to 450, from 450 to 500, from 500 to 600, from 600 to 700, from 700 to 800, from 800 to 900, or from 900 to 1000, contiguous amino acids) of the Spy Cas9 amino acid sequence depicted in FIG. 1. In some case, the RNA-guided effector polypeptide comprises no deletions other than the “A” and “B” deletions. In some cases, an RNA-guided effector polypeptide of the present disclosure comprising an “A” deletion and a “B” deletion has a length of from about 955 amino acids to about 1333 amino acids, e.g., from about 955 amino acids (aa) to about 975 aa, from about 975 aa to about 1000 aa, from about 1000 aa to about 1025 aa, from about 1025 aa to about 1050 aa, from about 1050 aa to about 1075 aa, from about 1075 aa to about 1100 aa, from about 1100 aa to about 1125 aa, from about 1125 aa to about 1150 aa, from about 1150 aa to about 1175 aa, from about 1175 aa to about 1200 aa, from about 1200 aa to about 1225 aa, from about 1225 aa to about 1250 aa, from about 1250 aa to about 1275 aa, from about 1275 aa to about 1300 aa, or from about 1300 aa to about 1333 aa. In some cases, an RNA-guided effector polypeptide of the present disclosure comprising only an “A” deletion and a “B” deletion has a length of from about 1045 aa to about 1050 aa. In some cases, an RNA-guided effector polypeptide of the present disclosure comprising only an “A” deletion and a “B” deletion has a length of 1046 aa, 1047 aa, or 1048 aa.

### “A+C” Deletion

In some cases, an RNA-guided effector polypeptide of the present disclosure comprises: i) a deletion of from about 5 contiguous amino acids to about 158 contiguous amino acids (e.g., from about 5 to about 50, from about 50 to about 75, from about 75 to about 100, from about 100 to about 125, or from about 125 to about 158 contiguous amino acids) of a region corresponding to amino acids 166-324 of the Spy Cas9 amino acid sequence depicted in FIG. 1; and ii) a deletion of from about 4 contiguous amino acids to about 220 contiguous amino acids (e.g., from about 4 to about 25, from about 25 to about 50, from about 50 to about 75, from about 75 to about 100, from about 100 to about 150, from about 150 to about 200, or from about 200 to about 220, contiguous amino acids) of a region corresponding to amino acids 761-981 of the Spy Cas9 amino acid sequence depicted in FIG. 1; and comprises one or more stretches of from 50 to 1000 contiguous amino acids (e.g., from 50 to 75, from 75 to 100, from 100 to 150, from 150 to 200, from 200 to 250, from 250 to 300, from 300 to 350, from 350 to 400, from 400 to 450, from 450 to 500, from 500 to 600, from 600 to 700, from 700 to 800, from 800 to 900, or from 900 to 1000, contiguous amino acids) having at least 85%, at least 90%, at least 95%, at least 98%, at least 99%, or 100%, amino acid sequence identity to corresponding stretches of from 50 to 1000 contiguous amino acids (e.g., from 50 to 75, from 75 to 100, from 100 to 150, from 150 to 200, from 200 to 250, from 250 to 300, from 300 to 350, from 350 to 400, from 400 to 450, from 450 to 500, from 500 to 600, from 600 to 700, from 700 to 800, from 800 to 900, or from 900 to 1000, contiguous amino acids) of the Spy Cas9 amino acid sequence depicted in FIG. 1. In some case, the RNA-guided effector polypeptide comprises no deletions other than the “A” and “C” deletions. In some cases, an RNA-guided effector polypeptide of the present disclosure comprising an “A” deletion and a “C” deletion has a length of from about 990 amino acids to about 1360 amino acids, e.g., from about 990 amino acids (aa) to about 1000 aa, from about 1000 aa to about 1025 aa, from about 1025 aa to about 1050 aa, from about 1050 aa to about 1075 aa, from about 1075 aa to about 1100 aa, from about 1100 aa to about 1125 aa, from about 1125 aa to about 1150 aa, from about 1150 aa to about 1175 aa, from about 1175 aa to about 1200 aa, from about 1200 aa to about 1225 aa, from about 1225 aa to about 1250 aa, from about 1250 aa to about 1275 aa, from about 1275 aa to about 1300 aa, from about 1300 aa to about 1325 aa, or from about 1325 aa to about 1360 aa. In some cases, an RNA-guided effector polypeptide of the present disclosure comprising only an “A” deletion and a “C” deletion has a length of from about 1140 aa to about 1150 aa. In some cases, an RNA-guided effector polypeptide of the present disclosure comprising an “A” deletion and a “C” deletion has a length of 1146 aa, 1147 aa, 1148 aa, 1149 aa, or 1150 aa.

### “A+D” Deletion

In some cases, an RNA-guided effector polypeptide of the present disclosure comprises: i) a deletion of from about 5 contiguous amino acids to about 158 contiguous amino acids (e.g., from about 5 to about 50, from about 50 to about 75, from about 75 to about 100, from about 100 to about 125, or from about 125 to about 158 contiguous amino acids) of a region corresponding to amino acids 166-324 of the Spy Cas9 amino acid sequence depicted in FIG. 1; and ii) a deletion of from about 20 contiguous amino acids to about 118 contiguous amino acids (e.g., from about 20 to about 40, from about 40 to about 60, from about 60 to about 80, from about 80 to about 100, or from about 100 to about 118 contiguous amino acids) of a region corresponding to amino acids 994-1112 of the Spy Cas9 amino acid sequence depicted in FIG. 1; and comprises one or more stretches of from 50 to 1000 contiguous amino acids (e.g., from 50 to 75, from 75 to 100, from 100 to 150, from 150 to 200, from 200 to 250, from 250 to 300, from 300 to 350, from 350 to 400, from 400 to 450, from 450 to 500, from 500 to 600, from 600 to 700, from 700 to 800, from 800 to 900, or from 900 to 1000, contiguous amino acids) having at least 85%, at least 90%, at least 95%, at least 98%, at least 99%, or 100%, amino acid sequence identity to corresponding stretches of from 50 to 1000 contiguous amino acids (e.g., from 50 to 75, from 75 to 100, from 100 to 150, from 150 to 200, from 200 to 250, from 250 to 300, from 300 to 350, from 350 to 400, from 400 to 450, from 450 to 500, from 500 to 600, from 600 to 700, from 700 to 800, from 800 to 900, or from 900 to 1000, contiguous amino acids) of the Spy Cas9 amino acid sequence depicted in FIG. 1. In some case, the RNA-guided effector polypeptide comprises no deletions other than the “A” and “D” deletions. In some cases, an RNA-guided effector polypeptide of the present disclosure comprising an “A” deletion and a “D” deletion has a length of from about 1090 amino acids to about 1343 amino acids, e.g., from about 1090 amino acids (aa) to about 1100 aa, from about 1100 aa to about 1125 aa, from about 1125 aa to about 1150 aa, from about 1150 aa to about 1175 aa, from about 1175 aa to about 1200 aa, from about 1200 aa to about 1225 aa, from about 1225 aa to about 1250 aa, from about 1250 aa to about 1275 aa, from about 1275 aa to about 1300 aa, from about 1300 aa to about 1325 aa, or from about 1325 aa to about 1343 aa. In some cases, an RNA-guided effector polypeptide of the present disclosure comprising an “A” deletion and a “D” deletion has a length of from about 1275 aa to about 1300 aa. In some cases, an RNA-guided effector polypeptide of the present disclosure comprising an “A” deletion and a “D” deletion has a length of 1280 aa, 1281 aa, 1282 aa, 1283 aa, or 1284 aa.

### “B”+“C” Deletions

In some cases, an RNA-guided effector polypeptide of the present disclosure comprises: i) a deletion of from about 30 contiguous amino acids to about 255 contiguous amino acids (e.g., from about 30 to about 50, from about 50 to about 75, from about 75 to about 100, from about 100 to about 150, from about 150 to about 200, or from about 200 to about 255 contiguous amino acids) of a region corresponding to amino acids 484-739 of the Spy Cas9 amino acid sequence depicted in FIG. 1; and ii) a deletion of from about 4 contiguous amino acids to about 220 contiguous amino acids (e.g., from about 4 to about 25, from about 25 to about 50, from about 50 to about 75, from about 75 to about 100, from about 100 to about 150, from about 150 to about 200, or from about 200 to about 220, contiguous amino acids) of a region corresponding to amino acids 761-981 of the Spy Cas9 amino acid sequence depicted in FIG. 1; and comprises one or more stretches of from 50 to 1000 contiguous amino acids (e.g., from 50 to 75, from 75 to 100, from 100 to 150, from 150 to 200, from 200 to 250, from 250 to 300, from 300 to 350, from 350 to 400, from 400 to 450, from 450 to 500, from 500 to 600, from 600 to 700, from 700 to 800, from 800 to 900, or from 900 to 1000, contiguous amino acids) having at least 85%, at least 90%, at least 95%, at least 98%, at least 99%, or 100%, amino acid sequence identity to corresponding stretches of from 50 to 1000 contiguous amino acids (e.g., from 50 to 75, from 75 to 100, from 100 to 150, from 150 to 200, from 200 to 250, from 250 to 300, from 300 to 350, from 350 to 400, from 400 to 450, from 450 to 500, from 500 to 600, from 600 to 700, from 700 to 800, from 800 to 900, or from 900 to 1000, contiguous amino acids) of the Spy Cas9 amino acid sequence depicted in FIG. 1. In some case, the RNA-guided effector polypeptide comprises no deletions other than the “B” and “C” deletions. In some cases, an RNA-guided effector polypeptide of the present disclosure comprising a “B” deletion and a “C” deletion has a length of from about 890 amino acids to about 1334 amino acids, e.g., from about 890 amino acids (aa) to about 900 aa, from about 900 aa to about 925 aa, from about 925 aa to about 950 aa, from about 950 aa to about 975 aa, from about 975 aa to about 1000 aa, from about 1000 aa to about 1025 aa, from about 1025 aa to about 1050 aa, from about 1050 aa to about 1075 aa, from about 1075 aa to about 1100 aa, from about 1100 aa to about 1125 aa, from about 1125 aa to about 1150 aa, from about 1150 aa to about 1175 aa, from about 1175 aa to about 1200 aa, from about 1200 aa to about 1225 aa, from about 1225 aa to about 1250 aa, from about 1250 aa to about 1275 aa, from about 1275 aa to about 1300 aa, from about 1300 aa to about 1325 aa, or from about 1325 aa to about 1334 aa. In some cases, an RNA-guided effector polypeptide of the present disclosure comprising a “B” deletion and a “C” deletion has a length of from about 1050 aa to about 1075 aa. In some cases, an RNA-guided effector polypeptide of the present disclosure comprising a “B” deletion and a “C” deletion has a length of 1058 aa, 1059 aa, 1060 aa, 1061 aa, or 1062 aa.

### “B”+“D” Deletion

In some cases, an RNA-guided effector polypeptide of the present disclosure comprises: i) a deletion of from about 30 contiguous amino acids to about 255 contiguous amino acids (e.g., from about 30 to about 50, from about 50 to about 75, from about 75 to about 100, from about 100 to about 150, from about 150 to about 200, or from about 200 to about 255 contiguous amino acids) of a region corresponding to amino acids 484-739 of the Spy Cas9 amino acid sequence depicted in FIG. 1; and ii) a deletion of from about 20 contiguous amino acids to about 118 contiguous amino acids (e.g., from about 20 to about 40, from about 40 to about 60, from about 60 to about 80, from about 80 to about 100, or from about 100 to about 118 contiguous amino acids) of a region corresponding to amino acids 994-1112 of the Spy Cas9 amino acid sequence depicted in FIG. 1; and comprises one or more stretches of from 50 to 1000 contiguous amino acids (e.g., from 50 to 75, from 75 to 100, from 100 to 150, from 150 to 200, from 200 to 250, from 250 to 300, from 300 to 350, from 350 to 400, from 400 to 450, from 450 to 500, from 500 to 600, from 600 to 700, from 700 to 800, from 800 to 900, or from 900 to 1000, contiguous amino acids) having at least 85%, at least 90%, at least 95%, at least 98%, at least 99%, or 100%, amino acid sequence identity to corresponding stretches of from 50 to 1000 contiguous amino acids (e.g., from 50 to 75, from 75 to 100, from 100 to 150, from 150 to 200, from 200 to 250, from 250 to 300, from 300 to 350, from 350 to 400, from 400 to 450, from 450 to 500, from 500 to 600, from 600 to 700, from 700 to 800, from 800 to 900, or from 900 to 1000, contiguous amino acids) of the Spy Cas9 amino acid sequence depicted in FIG. 1. In some case, the RNA-guided effector polypeptide comprises no deletions other than the “B” and “D” deletions. In some cases, an RNA-guided effector polypeptide of the present disclosure comprising a “B” deletion and a “D” deletion has a length of from about 995 amino acids to about 1318 amino acids, e.g., from about 995 amino acids (aa) to about 1000 aa, from about 1000 aa to about 1025 aa, from about 1025 aa to about 1050 aa, from about 1050 aa to about 1075 aa, from about 1075 aa to about 1100 aa, from about 1100 aa to about 1125 aa, from about 1125 aa to about 1150 aa, from about 1150 aa to about 1175 aa, from about 1175 aa to about 1200 aa, from about 1200 aa to about 1225 aa, from about 1225 aa to about 1250 aa, from about 1250 aa to about 1275 aa, from about 1275 aa to about 1300 aa, or from about 1300 aa to about 1318 aa. In some cases, an RNA-guided effector polypeptide of the present disclosure comprising a “B” deletion and a “D” deletion has a length of from about 1090 a to about 1100 aa. In some cases, an RNA-guided effector polypeptide of the present disclosure comprising a “B” deletion and a “D” deletion has a length of 1092 aa, 1093 aa, 1094 aa, or 1095 aa.

### “C”+“D” Deletions

In some cases, an RNA-guided effector polypeptide of the present disclosure comprises: i) a deletion of from about 4 contiguous amino acids to about 220 contiguous amino acids (e.g., from about 4 to about 25, from about 25 to about 50, from about 50 to about 75, from about 75 to about 100, from about 100 to about 150, from about 150 to about 200, or from about 200 to about 220, contiguous amino acids) of a region corresponding to amino acids 761-981 of the Spy Cas9 amino acid sequence depicted in FIG. 1; and ii) a deletion of from about 20 contiguous amino acids to about 118 contiguous amino acids (e.g., from about 20 to about 40, from about 40 to about 60, from about 60 to about 80, from about 80 to about 100, or from about 100 to about 118 contiguous amino acids) of a region corresponding to amino acids 994-1112 of the Spy Cas9 amino acid sequence depicted in FIG. 1; and comprises one or more stretches of from 50 to 1000 contiguous amino acids (e.g., from 50 to 75, from 75 to 100, from 100 to 150, from 150 to 200, from 200 to 250, from 250 to 300, from 300 to 350, from 350 to 400, from 400 to 450, from 450 to 500, from 500 to 600, from 600 to 700, from 700 to 800, from 800 to 900, or from 900 to 1000, contiguous amino acids) having at least 85%, at least 90%, at least 95%, at least 98%, at least 99%, or 100%, amino acid sequence identity to corresponding stretches of from 50 to 1000 contiguous amino acids (e.g., from 50 to 75, from 75 to 100, from 100 to 150, from 150 to 200, from 200 to 250, from 250 to 300, from 300 to 350, from 350 to 400, from 400 to 450, from 450 to 500, from 500 to 600, from 600 to 700, from 700 to 800, from 800 to 900, or from 900 to 1000, contiguous amino acids) of the Spy Cas9 amino acid sequence depicted in FIG. 1. In some case, the RNA-guided effector polypeptide comprises no deletions other than the “C” and “D” deletions. In some cases, an RNA-guided effector polypeptide of the present disclosure comprising a “C” deletion and a “D” deletion has a length of from about 1030 amino acids to about 1344 amino acids, e.g., from about 1030 amino acids (aa) to about 1050 aa, from about 1150 aa to about 1175 aa, from about 1175 aa to about 1200 aa, from about 1200 aa to about 1225 aa, from about 1225 aa to about 1250 aa, from about 1250 aa to about 1275 aa, from about 1275 aa to about 1300 aa, from about 1300 aa to about 1325 aa, or from about 1325 aa to about 1344 aa. In some cases, an RNA-guided effector polypeptide of the present disclosure comprising a “C” deletion and a “D” deletion has a length of from about 1190 to about 1200. In some cases, an RNA-guided effector polypeptide of the present disclosure comprising a “C” deletion and a “D” deletion has a length of 1192 aa, 1193 aa, 1194 aa, or 1195 aa.

### “A”+“B”+“C” Deletions

In some cases, an RNA-guided effector polypeptide of the present disclosure comprises: i) a deletion of from about 5 contiguous amino acids to about 158 contiguous amino acids (e.g., from about 5 to about 50, from about 50 to about 75, from about 75 to about 100, from about 100 to about 125, or from about 125 to about 158 contiguous amino acids) of a region corresponding to amino acids 166-324 of the Spy Cas9 amino acid sequence depicted in FIG. 1; ii) a deletion of from about 30 contiguous amino acids to about 255 contiguous amino acids (e.g., from about 30 to about 50, from about 50 to about 75, from about 75 to about 100, from about 100 to about 150, from about 150 to about 200, or from about 200 to about 255 contiguous amino acids) of a region corresponding to amino acids 484-739 of the Spy Cas9 amino acid sequence depicted in FIG. 1; and iii) a deletion of from about 4 contiguous amino acids to about 220 contiguous amino acids (e.g., from about 4 to about 25, from about 25 to about 50, from about 50 to about 75, from about 75 to about 100, from about 100 to about 150, from about 150 to about 200, or from about 200 to about 220, contiguous amino acids) of a region corresponding to amino acids 761-981 of the Spy Cas9 amino acid sequence depicted in FIG. 1; and comprises one or more stretches of from 50 to 1000 contiguous amino acids (e.g., from 50 to 75, from 75 to 100, from 100 to 150, from 150 to 200, from 200 to 250, from 250 to 300, from 300 to 350, from 350 to 400, from 400 to 450, from 450 to 500, from 500 to 600, from 600 to 700, from 700 to 800, from 800 to 900, or from 900 to 1000, contiguous amino acids) having at least 85%, at least 90%, at least 95%, at least 98%, at least 99%, or 100%, amino acid sequence identity to corresponding stretches of from 50 to 1000 contiguous amino acids (e.g., from 50 to 75, from 75 to 100, from 100 to 150, from 150 to 200, from 200 to 250, from 250 to 300, from 300 to 350, from 350 to 400, from 400 to 450, from 450 to 500, from 500 to 600, from 600 to 700, from 700 to 800, from 800 to 900, or from 900 to 1000, contiguous amino acids) of the Spy Cas9 amino acid sequence depicted in FIG. 1. In some case, the RNA-guided effector polypeptide comprises no deletions other than the “A,” “B,” and “C” deletions. In some cases, an RNA-guided effector polypeptide of the present disclosure comprising an “A” deletion, a “B” deletion, and a “C” deletion has a length of from about 735 amino acids to about 1329 amino acids, e.g., from about 735 amino acids (aa) to about 750 aa, from about 750 aa to about 775 aa, from about 775 aa to about 800 aa, from about 800 aa to about 825 aa, from about 825 aa to about 850 aa, from about 850 aa to about 875 aa, from about 875 aa to about 900 aa, from about 900 aa to about 925 aa, from about 925 aa to about 950 aa, from about 950 aa to about 975 aa, from about 975 aa to about 1000 aa, from about 1000 aa to about 1025 aa, from about 1025 aa to about 1050 aa, from about 1050 aa to about 1075 aa, from about 1075 aa to about 1100 aa, from about 1100 aa to about 1125 aa, from about 1125 aa to about 1150 aa, from about 1150 aa to about 1175 aa, from about 1175 aa to about 1200 aa, from about 1200 aa to about 1225 aa, from about 1225 aa to about 1250 aa, from about 1250 aa to about 1275 aa, from about 1275 aa to about 1300 aa, or from about 1300 aa to about 1329 aa. In some cases, an RNA-guided effector polypeptide of the present disclosure comprising an “A” deletion, a “B” deletion, and a “C” deletion has a length of from about 930 aa to about 970 aa. In some cases, an RNA-guided effector polypeptide of the present disclosure comprising an “A” deletion, a “B” deletion, and a “C” deletion has a length of from about 940 aa to about 950 aa.

### “A”+“B”+“D” Deletions

In some cases, an RNA-guided effector polypeptide of the present disclosure comprises: i) a deletion of from about 5 contiguous amino acids to about 158 contiguous amino acids (e.g., from about 5 to about 50, from about 50 to about 75, from about 75 to about 100, from about 100 to about 125, or from about 125 to about 158 contiguous amino acids) of a region corresponding to amino acids 166-324 of the Spy Cas9 amino acid sequence depicted in FIG. 1; ii) a deletion of from about 30 contiguous amino acids to about 255 contiguous amino acids (e.g., from about 30 to about 50, from about 50 to about 75, from about 75 to about 100, from about 100 to about 150, from about 150 to about 200, or from about 200 to about 255 contiguous amino acids) of a region corresponding to amino acids 484-739 of the Spy Cas9 amino acid sequence depicted in FIG. 1; and iii) a deletion of from about 20 contiguous amino acids to about 118 contiguous amino acids (e.g., from about 20 to about 40, from about 40 to about 60, from about 60 to about 80, from about 80 to about 100, or from about 100 to about 118 contiguous amino acids) of a region corresponding to amino acids 994-1112 of the Spy Cas9 amino acid sequence depicted in FIG. 1; and comprises one or more stretches of from 50 to 1000 contiguous amino acids (e.g., from 50 to 75, from 75 to 100, from 100 to 150, from 150 to 200, from 200 to 250, from 250 to 300, from 300 to 350, from 350 to 400, from 400 to 450, from 450 to 500, from 500 to 600, from 600 to 700, from 700 to 800, from 800 to 900, or from 900 to 1000, contiguous amino acids) having at least 85%, at least 90%, at least 95%, at least 98%, at least 99%, or 100%, amino acid sequence identity to corresponding stretches of from 50 to 1000 contiguous amino acids (e.g., from 50 to 75, from 75 to 100, from 100 to 150, from 150 to 200, from 200 to 250, from 250 to 300, from 300 to 350, from 350 to 400, from 400 to 450, from 450 to 500, from 500 to 600, from 600 to 700, from 700 to 800, from 800 to 900, or from 900 to 1000, contiguous amino acids) of the Spy Cas9 amino acid sequence depicted in FIG. 1. In some case, the RNA-guided effector polypeptide comprises no deletions other than the “A,” “B,” and “D” deletions. In some cases, an RNA-guided effector polypeptide of the present disclosure comprising an “A” deletion, a “B” deletion, and a “D” deletion has a length of from about 837 amino acids to about 1313 amino acids, e.g., from about 837 amino acids (aa) to about 850 aa, from about 850 aa to about 875 aa, from about 875 aa to about 900 aa, from about 900 aa to about 925 aa, from about 925 aa to about 950 aa, from about 950 aa to about 975 aa, from about 975 aa to about 1000 aa, from about 1000 aa to about 1025 aa, from about 1025 aa to about 1050 aa, from about 1050 aa to about 1075 aa, from about 1075 aa to about 1100 aa, from about 1100 aa to about 1125 aa, from about 1125 aa to about 1150 aa, from about 1150 aa to about 1175 aa, from about 1175 aa to about 1200 aa, from about 1200 aa to about 1225 aa, from about 1225 aa to about 1250 aa, from about 1250 aa to about 1275 aa, from about 1275 aa to about 1300 aa, or from about 1300 aa to about 1313 aa. In some cases, an RNA-guided effector polypeptide of the present disclosure comprising an “A” deletion, a “B” deletion, and a “D” deletion has a length of from about 970 aa to about 1000 aa, In some cases, an RNA-guided effector polypeptide of the present disclosure comprising an “A” deletion, a “B” deletion, and a “D” deletion has a length of from about 975 aa to about 980 aa.

### “A”+“C”+“D” Deletions

In some cases, an RNA-guided effector polypeptide of the present disclosure comprises: i) a deletion of from about 5 contiguous amino acids to about 158 contiguous amino acids (e.g., from about 5 to about 50, from about 50 to about 75, from about 75 to about 100, from about 100 to about 125, or from about 125 to about 158 contiguous amino acids) of a region corresponding to amino acids 166-324 of the Spy Cas9 amino acid sequence depicted in FIG. 1; ii) a deletion of from about 4 contiguous amino acids to about 220 contiguous amino acids (e.g., from about 4 to about 25, from about 25 to about 50, from about 50 to about 75, from about 75 to about 100, from about 100 to about 150, from about 150 to about 200, or from about 200 to about 220, contiguous amino acids) of a region corresponding to amino acids 761-981 of the Spy Cas9 amino acid sequence depicted in FIG. 1; and iii) a deletion of from about 20 contiguous amino acids to about 118 contiguous amino acids (e.g., from about 20 to about 40, from about 40 to about 60, from about 60 to about 80, from about 80 to about 100, or from about 100 to about 118 contiguous amino acids) of a region corresponding to amino acids 994-1112 of the Spy Cas9 amino acid sequence depicted in FIG. 1; and comprises one or more stretches of from 50 to 1000 contiguous amino acids (e.g., from 50 to 75, from 75 to 100, from 100 to 150, from 150 to 200, from 200 to 250, from 250 to 300, from 300 to 350, from 350 to 400, from 400 to 450, from 450 to 500, from 500 to 600, from 600 to 700, from 700 to 800, from 800 to 900, or from 900 to 1000, contiguous amino acids) having at least 85%, at least 90%, at least 95%, at least 98%, at least 99%, or 100%, amino acid sequence identity to corresponding stretches of from 50 to 1000 contiguous amino acids (e.g., from 50 to 75, from 75 to 100, from 100 to 150, from 150 to 200, from 200 to 250, from 250 to 300, from 300 to 350, from 350 to 400, from 400 to 450, from 450 to 500, from 500 to 600, from 600 to 700, from 700 to 800, from 800 to 900, or from 900 to 1000, contiguous amino acids) of the Spy Cas9 amino acid sequence depicted in FIG. 1. In some case, the RNA-guided effector polypeptide comprises no deletions other than the “A,” “C,” and “D” deletions. In some cases, an RNA-guided effector polypeptide of the present disclosure comprising an “A” deletion, a “C” deletion, and a “D” deletion has a length of from about 870 amino acids to about 1340 amino acids, e.g., from about 870 amino acids (aa) to about 900 aa, from about 900 aa to about 925 aa, from about 925 aa to about 950 aa, from about 950 aa to about 975 aa, from about 975 aa to about 1000 aa, from about 1000 aa to about 1025 aa, from about 1025 aa to about 1050 aa, from about 1050 aa to about 1075 aa, from about 1075 aa to about 1100 aa, from about 1100 aa to about 1125 aa, from about 1125 aa to about 1150 aa, from about 1150 aa to about 1175 aa, from about 1175 aa to about 1200 aa, from about 1200 aa to about 1225 aa, from about 1225 aa to about 1250 aa, from about 1250 aa to about 1275 aa, from about 1275 aa to about 1300 aa, from about 1300 aa to about 1325 aa, or from about 1325 aa to about 1340 aa. In some cases, an RNA-guided effector polypeptide of the present disclosure comprising an “A” deletion, a “C” deletion, and a “D” deletion has a length of from about 1070 aa to about 1100 aa. In some cases, an RNA-guided effector polypeptide of the present disclosure comprising an “A” deletion, a “C” deletion, and a “D” deletion has a length of from about 1075 aa to about 1080 aa.

### “B”+“C”+“D” Deletions

In some cases, an RNA-guided effector polypeptide of the present disclosure comprises: i) a deletion of from about 5 contiguous amino acids to about 158 contiguous amino acids (e.g., from about 5 to about 50, from about 50 to about 75, from about 75 to about 100, from about 100 to about 125, or from about 125 to about 158 contiguous amino acids) of a region corresponding to amino acids 166-324 of the Spy Cas9 amino acid sequence depicted in FIG. 1; ii) a deletion of from about 4 contiguous amino acids to about 220 contiguous amino acids (e.g., from about 4 to about 25, from about 25 to about 50, from about 50 to about 75, from about 75 to about 100, from about 100 to about 150, from about 150 to about 200, or from about 200 to about 220, contiguous amino acids) of a region corresponding to amino acids 761-981 of the Spy Cas9 amino acid sequence depicted in FIG. 1; and iii) a deletion of from about 20 contiguous amino acids to about 118 contiguous amino acids (e.g., from about 20 to about 40, from about 40 to about 60, from about 60 to about 80, from about 80 to about 100, or from about 100 to about 118 contiguous amino acids) of a region corresponding to amino acids 994-1112 of the Spy Cas9 amino acid sequence depicted in FIG. 1; and comprises one or more stretches of from 50 to 1000 contiguous amino acids (e.g., from 50 to 75, from 75 to 100, from 100 to 150, from 150 to 200, from 200 to 250, from 250 to 300, from 300 to 350, from 350 to 400, from 400 to 450, from 450 to 500, from 500 to 600, from 600 to 700, from 700 to 800, from 800 to 900, or from 900 to 1000, contiguous amino acids) having at least 85%, at least 90%, at least 95%, at least 98%, at least 99%, or 100%, amino acid sequence identity to corresponding stretches of from 50 to 1000 contiguous amino acids (e.g., from 50 to 75, from 75 to 100, from 100 to 150, from 150 to 200, from 200 to 250, from 250 to 300, from 300 to 350, from 350 to 400, from 400 to 450, from 450 to 500, from 500 to 600, from 600 to 700, from 700 to 800, from 800 to 900, or from 900 to 1000, contiguous amino acids) of the Spy Cas9 amino acid sequence depicted in FIG. 1. In some case, the RNA-guided effector polypeptide comprises no deletions other than the “B,” “C,” and “D” deletions. In some cases, an RNA-guided effector polypeptide of the present disclosure comprising a “B” deletion, a “C” deletion, and a “D” deletion has a length of from about 775 amino acids to about 1314 amino acids, e.g., from about 775 aa to about 800 aa, from about 800 aa to about 825 aa, from about 825 aa to about 850 aa, from about 850 aa to about 875 aa, from about 875 aa to about 900 aa, from about 900 aa to about 925 aa, from about 925 aa to about 950 aa, from about 950 aa to about 975 aa, from about 975 aa to about 1000 aa, from about 1000 aa to about 1025 aa, from about 1025 aa to about 1050 aa, from about 1050 aa to about 1075 aa, from about 1075 aa to about 1100 aa, from about 1100 aa to about 1125 aa, from about 1125 aa to about 1150 aa, from about 1150 aa to about 1175 aa, from about 1175 aa to about 1200 aa, from about 1200 aa to about 1225 aa, from about 1225 aa to about 1250 aa, from about 1250 aa to about 1275 aa, from about 1275 aa to about 1300 aa, or from about 1300 aa to about 1314 aa. In some cases, an RNA-guided effector polypeptide of the present disclosure comprising a “B” deletion, a “C” deletion, and a “D” deletion has a length of from about 980 aa to about 1000 aa. In some cases, an RNA-guided effector polypeptide of the present disclosure comprising a “B” deletion, a “C” deletion, and a “D” deletion has a length of from about 980 aa to about 990 aa.

### “A”+“B”+“C”+“D” Deletions

In some cases, an RNA-guided effector polypeptide of the present disclosure comprises: i) a deletion of from about 5 contiguous amino acids to about 158 contiguous amino acids (e.g., from about 5 to about 50, from about 50 to about 75, from about 75 to about 100, from about 100 to about 125, or from about 125 to about 158 contiguous amino acids) of a region corresponding to amino acids 166-324 of the Spy Cas9 amino acid sequence depicted in FIG. 1; ii) a deletion of from about 5 contiguous amino acids to about 158 contiguous amino acids (e.g., from about 5 to about 50, from about 50 to about 75, from about 75 to about 100, from about 100 to about 125, or from about 125 to about 158 contiguous amino acids) of a region corresponding to amino acids 166-324 of the Spy Cas9 amino acid sequence depicted in FIG. 1; iii) a deletion of from about 4 contiguous amino acids to about 220 contiguous amino acids (e.g., from about 4 to about 25, from about 25 to about 50, from about 50 to about 75, from about 75 to about 100, from about 100 to about 150, from about 150 to about 200, or from about 200 to about 220, contiguous amino acids) of a region corresponding to amino acids 761-981 of the Spy Cas9 amino acid sequence depicted in FIG. 1; and iv) a deletion of from about 20 contiguous amino acids to about 118 contiguous amino acids (e.g., from about 20 to about 40, from about 40 to about 60, from about 60 to about 80, from about 80 to about 100, or from about 100 to about 118 contiguous amino acids) of a region corresponding to amino acids 994-1112 of the Spy Cas9 amino acid sequence depicted in FIG. 1; and comprises one or more stretches of from 50 to 1000 contiguous amino acids (e.g., from 50 to 75, from 75 to 100, from 100 to 150, from 150 to 200, from 200 to 250, from 250 to 300, from 300 to 350, from 350 to 400, from 400 to 450, from 450 to 500, from 500 to 600, from 600 to 700, from 700 to 800, from 800 to 900, or from 900 to 1000, contiguous amino acids) having at least 85%, at least 90%, at least 95%, at least 98%, at least 99%, or 100%, amino acid sequence identity to corresponding stretches of from 50 to 1000 contiguous amino acids (e.g., from 50 to 75, from 75 to 100, from 100 to 150, from 150 to 200, from 200 to 250, from 250 to 300, from 300 to 350, from 350 to 400, from 400 to 450, from 450 to 500, from 500 to 600, from 600 to 700, from 700 to 800, from 800 to 900, or from 900 to 1000, contiguous amino acids) of the Spy Cas9 amino acid sequence depicted in FIG. 1.

In some cases, an RNA-guided effector polypeptide of the present disclosure comprising an “A” deletion, a “B” deletion, a “C” deletion, and a “D” deletion has a length of from about 615 amino acids to about 1310 amino acids, e.g., from about 615 amino acids (aa) to about 625 aa, from about 625 aa to about 650 aa, from about 650 aa to about 675 aa, from about 675 aa to about 700 aa, from about 700 aa to about 725 aa, from about 725 aa to about 750 aa, from about 750 aa to about 775 aa, from about 775 aa to about 800 aa, from about 800 aa to about 825 aa, from about 825 aa to about 850 aa, from about 850 aa to about 875 aa, from about 875 aa to about 900 aa, from about 900 aa to about 925 aa, from about 925 aa to about 950 aa, from about 950 aa to about 975 aa, from about 975 aa to about 1000 aa, from about 1000 aa to about 1025 aa, from about 1025 aa to about 1050 aa, from about 1050 aa to about 1075 aa, from about 1075 aa to about 1100 aa, from about 1100 aa to about 1125 aa, from about 1125 aa to about 1150 aa, from about 1150 aa to about 1175 aa, from about 1175 aa to about 1200 aa, from about 1200 aa to about 1225 aa, from about 1225 aa to about 1250 aa, from about 1250 aa to about 1275 aa, from about 1275 aa to about 1300 aa, or from about 1300 aa to about 1310 aa. In some cases, an RNA-guided effector polypeptide of the present disclosure comprising an “A” deletion, a “B” deletion, a “C” deletion, and a “D” deletion has a length of from about 870 aa to about 1000 aa. In some cases, an RNA-guided effector polypeptide of the present disclosure comprising an “A” deletion, a “B” deletion, a “C” deletion, and a “D” deletion has a length of from about 870 aa to about 900 aa. In some cases, an RNA-guided effector polypeptide of the present disclosure comprising an “A” deletion, a “B” deletion, a “C” deletion, and a “D” deletion has a length of from about 870 aa to about 875 aa.

### Examples of RNA-Guided Effector Polypeptides

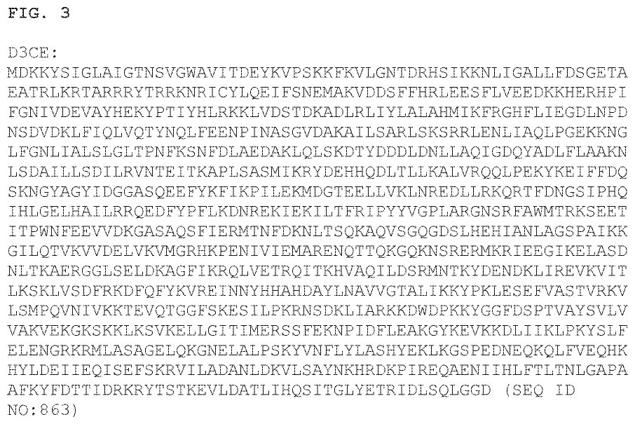

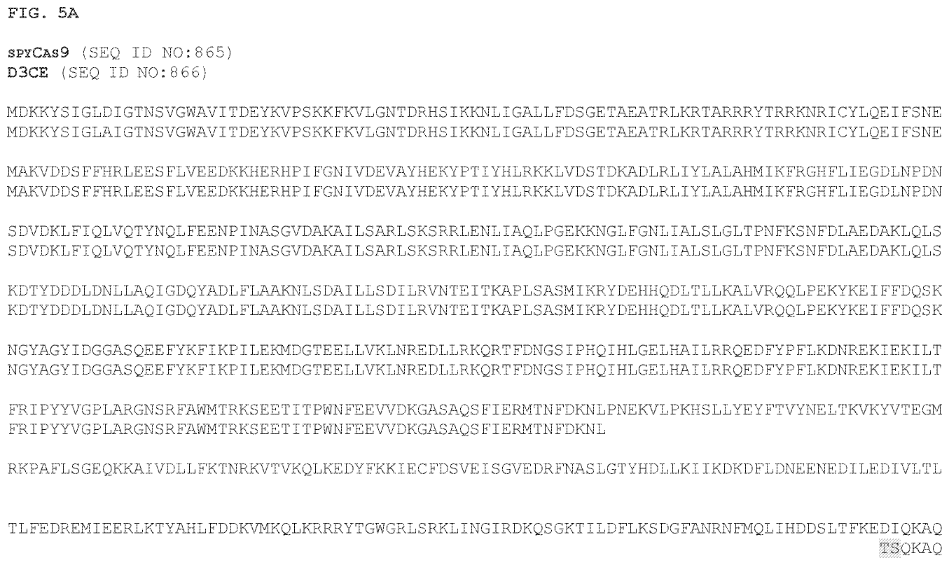

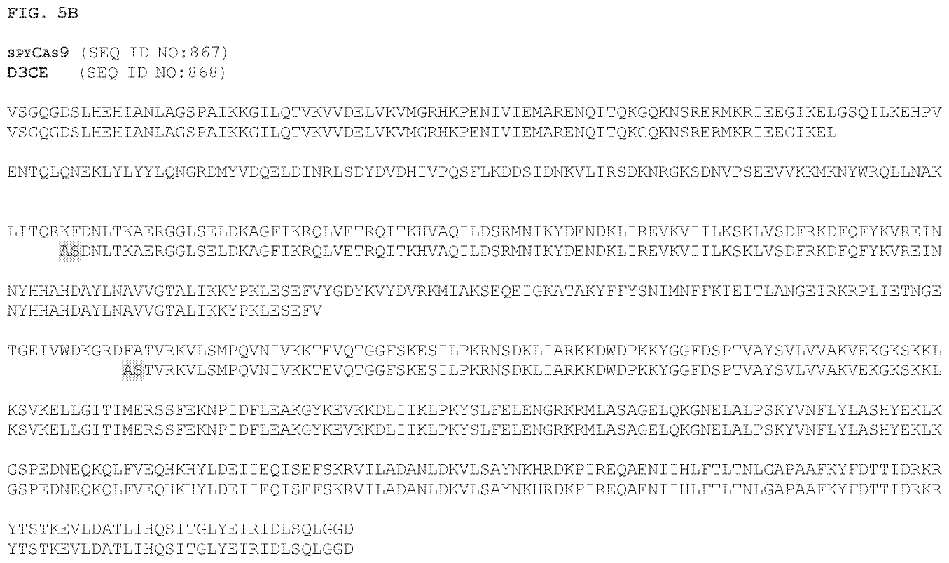

As an example, in some cases, an RNA-guided effector polypeptide of the present disclosure comprises an amino acid sequence having at least at least 85%, at least 90%, at least 95%, at least 98%, at least 99%, or 100%, amino acid sequence identity to the amino acid sequence of the D3CE polypeptide depicted in FIG. 3. The D3CE polypeptide depicted in FIG. 3 comprises a “B” deletion of amino acids 503-708, a “C” deletion of amino acids 792-897, and a “D” deletion of amino acids 1010-1081, compared to the amino acid numbering of the SpyCas9 amino acid sequence set forth in FIG. 1. The locations of the deletions, relative to the SpyCas9 amino acid sequence set forth in FIG. 1, are depicted in FIG. 5A-5B. D3CE has a length of 987 amino acids.

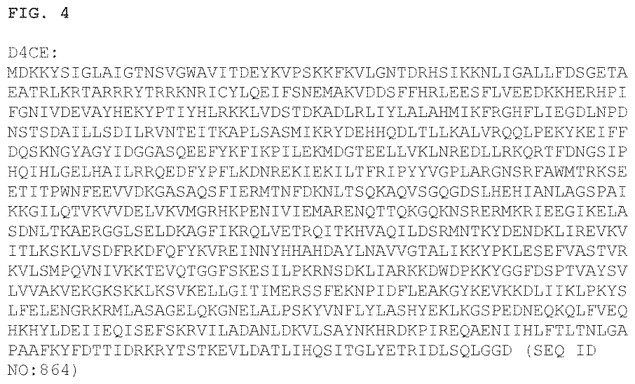

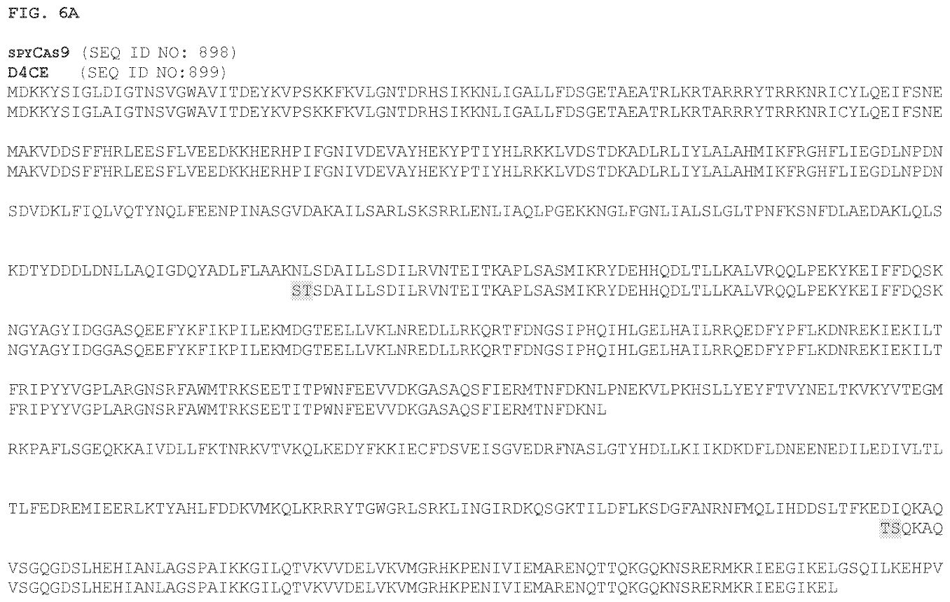

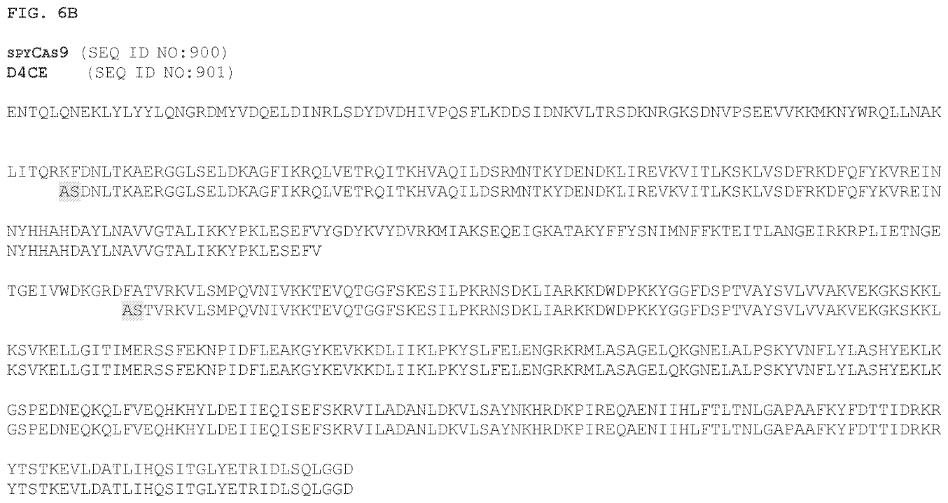

As another example, in some cases, an RNA-guided effector polypeptide of the present disclosure comprises an amino acid sequence having at least at least 85%, at least 90%, at least 95%, at least 98%, at least 99%, or 100%, amino acid sequence identity to the amino acid sequence of the D4CE polypeptide depicted in FIG. 4. The D4CE polypeptide depicted in FIG. 4 comprises an “A” deletion of amino acids 179-296, a “B” deletion of amino acids 503-708, a “C” deletion of amino acids 792-897, and a “D” deletion of amino acids 1010-1081, compared to the amino acid numbering of the SpyCas9 amino acid sequence set forth in FIG. 1. The locations of the deletions, relative to the SpyCas9 amino acid sequence set forth in FIG. 1, are depicted in FIG. 6A-6B. D4CE has a length of 874 amino acids.

As another example, in some cases, an RNA-guided effector polypeptide of the present disclosure comprises an amino acid sequence having at least at least 85%, at least 90%, at least 95%, at least 98%, at least 99%, or 100%, amino acid sequence identity to the amino acid sequence of the Variant 1 polypeptide depicted in FIG. 10B. As depicted in FIG. 10A, a Variant 1 polypeptide has an “A” deletion of amino acids 174-289 compared to the Spy Cas9 polypeptide depicted in FIG. 1; and has a length of about 1253 amino acids.

As another example, in some cases, an RNA-guided effector polypeptide of the present disclosure comprises an amino acid sequence having at least at least 85%, at least 90%, at least 95%, at least 98%, at least 99%, or 100%, amino acid sequence identity to the amino acid sequence of the Variant 2 polypeptide depicted in FIG. 11B. As depicted in FIG. 11A, a Variant 2 polypeptide has an “A” deletion of amino acids 174-306 compared to the Spy Cas9 polypeptide depicted in FIG. 1; and has a length of about 1235 amino acids.

As another example, in some cases, an RNA-guided effector polypeptide of the present disclosure comprises an amino acid sequence having at least at least 85%, at least 90%, at least 95%, at least 98%, at least 99%, or 100%, amino acid sequence identity to the amino acid sequence of the Variant 3 polypeptide depicted in FIG. 12B. As depicted in FIG. 12A, a Variant 3 polypeptide has an “A” deletion of amino acids 175-296 compared to the Spy Cas9 polypeptide depicted in FIG. 1; and has a length of about 1246 amino acids.

As another example, in some cases, an RNA-guided effector polypeptide of the present disclosure comprises an amino acid sequence having at least at least 85%, at least 90%, at least 95%, at least 98%, at least 99%, or 100%, amino acid sequence identity to the amino acid sequence of the Variant 4 polypeptide depicted in FIG. 13B. As depicted in FIG. 13A, a Variant 4 polypeptide has a “B” deletion of amino acids 522-702 compared to the Spy Cas9 polypeptide depicted in FIG. 1; and has a length of about 1187 amino acids.

As another example, in some cases, an RNA-guided effector polypeptide of the present disclosure comprises an amino acid sequence having at least at least 85%, at least 90%, at least 95%, at least 98%, at least 99%, or 100%, amino acid sequence identity to the amino acid sequence of the Variant 5 polypeptide depicted in FIG. 14B. As depicted in FIG. 14A, a Variant 5 polypeptide has a “B” deletion of amino acids 537-704 compared to the Spy Cas9 polypeptide depicted in FIG. 1; and has a length of about 1200 amino acids.

As another example, in some cases, an RNA-guided effector polypeptide of the present disclosure comprises an amino acid sequence having at least at least 85%, at least 90%, at least 95%, at least 98%, at least 99%, or 100%, amino acid sequence identity to the amino acid sequence of the Variant 6 polypeptide depicted in FIG. 15B. As depicted in FIG. 15A, a Variant 6 polypeptide has a “B” deletion of amino acids 523-664 compared to the Spy Cas9 polypeptide depicted in FIG. 1; and has a length of about 1226 amino acids.

As another example, in some cases, an RNA-guided effector polypeptide of the present disclosure comprises an amino acid sequence having at least at least 85%, at least 90%, at least 95%, at least 98%, at least 99%, or 100%, amino acid sequence identity to the amino acid sequence of the Variant 7 polypeptide depicted in FIG. 16B. As depicted in FIG. 16A, a Variant 7 polypeptide has a “C” deletion of amino acids 781-906 compared to the Spy Cas9 polypeptide depicted in FIG. 1; and has a length of about 1242 amino acids.

As another example, in some cases, an RNA-guided effector polypeptide of the present disclosure comprises an amino acid sequence having at least at least 85%, at least 90%, at least 95%, at least 98%, at least 99%, or 100%, amino acid sequence identity to the amino acid sequence of the Variant 8 polypeptide depicted in FIG. 17B. As depicted in FIG. 17A, a Variant 8 polypeptide has a “C” deletion of amino acids 820-932 compared to the Spy Cas9 polypeptide depicted in FIG. 1; and has a length of about 1255 amino acids.

As another example, in some cases, an RNA-guided effector polypeptide of the present disclosure comprises an amino acid sequence having at least at least 85%, at least 90%, at least 95%, at least 98%, at least 99%, or 100%, amino acid sequence identity to the amino acid sequence of the Variant 9 polypeptide depicted in FIG. 18B. As depicted in FIG. 18A, a Variant 9 polypeptide has a “C” deletion of amino acids 769-910 compared to the Spy Cas9 polypeptide depicted in FIG. 1; and has a length of about 1226 amino acids.

As another example, in some cases, an RNA-guided effector polypeptide of the present disclosure comprises an amino acid sequence having at least at least 85%, at least 90%, at least 95%, at least 98%, at least 99%, or 100%, amino acid sequence identity to the amino acid sequence of the Variant 10 polypeptide depicted in FIG. 19B. As depicted in FIG. 19A, a Variant 10 polypeptide has: i) a “B” deletion of amino acids 508-649; ii) a “C” deletion of amino acids 768-900; and iii) a “D” deletion of amino acids 1010-1081 compared to the Spy Cas9 polypeptide depicted in FIG. 1; and has a length of about 1021 amino acids.

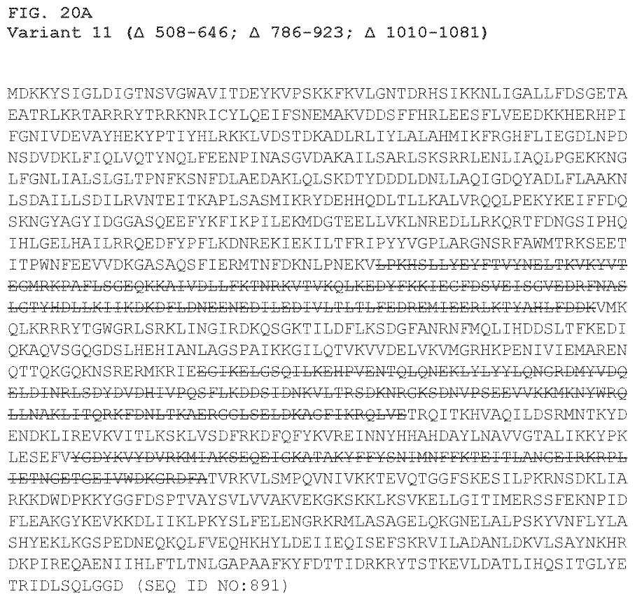

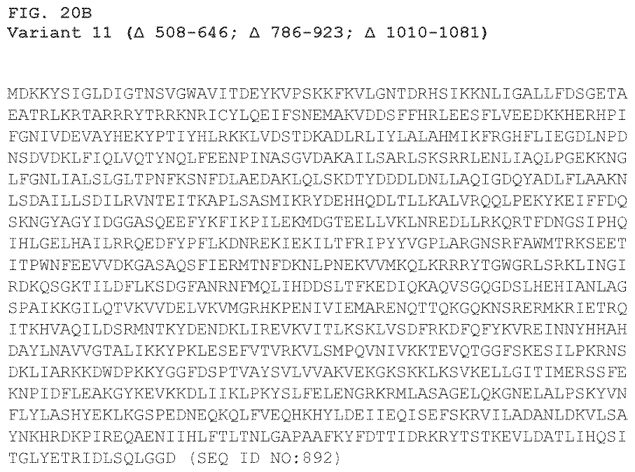

As another example, in some cases, an RNA-guided effector polypeptide of the present disclosure comprises an amino acid sequence having at least at least 85%, at least 90%, at least 95%, at least 98%, at least 99%, or 100%, amino acid sequence identity to the amino acid sequence of the Variant 11 polypeptide depicted in FIG. 20B. As depicted in FIG. 20A, a Variant 11 polypeptide has: i) a “B” deletion of amino acids 508-646; ii) a “C” deletion of amino acids 786-923; and iii) a “D” deletion of amino acids 1010-1081 compared to the Spy Cas9 polypeptide depicted in FIG. 1; and has a length of about 1019 amino acids.

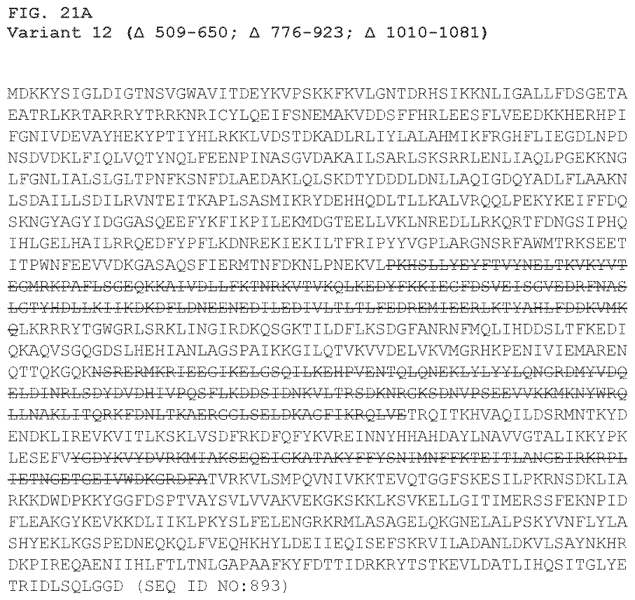

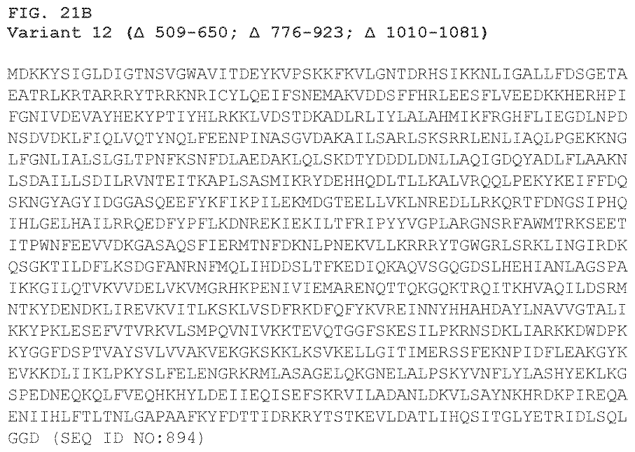

As another example, in some cases, an RNA-guided effector polypeptide of the present disclosure comprises an amino acid sequence having at least at least 85%, at least 90%, at least 95%, at least 98%, at least 99%, or 100%, amino acid sequence identity to the amino acid sequence of the Variant 12 polypeptide depicted in FIG. 21B. As depicted in FIG. 21A, a Variant 12 polypeptide has: i) a “B” deletion of amino acids 509-650; ii) a “C” deletion of amino acids 776-923; and iii) a “D” deletion of amino acids 1010-1081 compared to the Spy Cas9 polypeptide depicted in FIG. 1; and has a length of about 1006 amino acids.

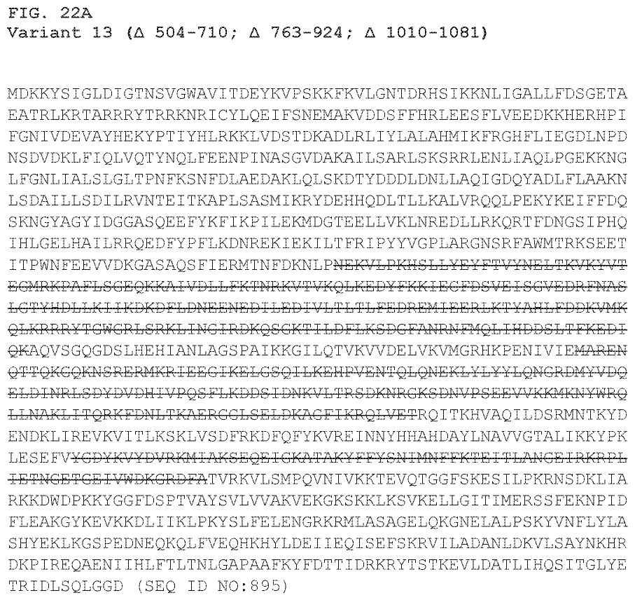

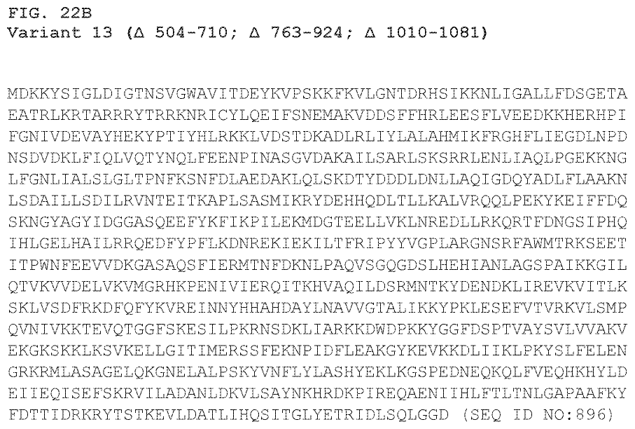

As another example, in some cases, an RNA-guided effector polypeptide of the present disclosure comprises an amino acid sequence having at least at least 85%, at least 90%, at least 95%, at least 98%, at least 99%, or 100%, amino acid sequence identity to the amino acid sequence of the Variant 13 polypeptide depicted in FIG. 22B. As depicted in FIG. 22A, a Variant 13 polypeptide has: i) a “B” deletion of amino acids 504-710; ii) a “C” deletion of amino acids 763-924; and iii) a “D” deletion of amino acids 1010-1081 compared to the Spy Cas9 polypeptide depicted in FIG. 1; and has a length of about 927 amino acids.

### Fusion Polypeptides Comprising an RNA-Guided Effector Polypeptide

The present disclosure provides a fusion polypeptide comprising: i) an RNA-guided effector polypeptide of the present disclosure; and ii) a heterologous fusion partner.

In some cases, the heterologous fusion partner is a polypeptide having a length of no more than 500 amino acids (aa), no more than 400 aa, no more than 300 aa, no more than 200 aa, or no more than 100 aa. In some cases, the heterologous fusion partner is a polypeptide having a length of from about 10 amino acids (aa) to about 25 aa, from about 25 aa to about 50 aa, from about 50 aa to about 100 aa, from about 100 aa to about 150 aa, from about 150 aa to about 200 aa, from about 200 aa to about 300 aa, from about 300 aa to about 400 aa, or from about 400 aa to about 500 aa.

The total length of the fusion polypeptide is in some cases from about 750 amino acids to about 1500 amino acids, e.g., from about 750 amino acids (aa) to about 775 aa, from about 775 aa to about 800 aa, from about 800 aa to about 825 aa, from about 825 aa to about 850 aa, from about 850 aa to about 875 aa, from about 875 aa to about 900 aa, from about 900 aa to about 925 aa, from about 925 aa to about 950 aa, from about 950 aa to about 975 aa, or from about 975 aa to about 1000 aa.

In other instances, the total length of the fusion polypeptide is from about 1000 amino acids to about 1500 amino acids, e.g., from about 1000 amino acids (aa) to about 1100 aa, from about 1100 aa to about 1200 aa, from about 1200 aa to about 1300 aa, from about 1300 aa to about 1400 aa, or from about 1400 aa to about 1500 aa. The total length of the fusion polypeptide is in some cases from about 1000 aa to about 1400 aa. The total length of the fusion polypeptide is in some cases from about 1000 aa to about 1300 aa. The total length of the fusion polypeptide is in some cases from about 1000 aa to about 1200 aa. The total length of the fusion polypeptide is in some cases from about 1000 aa to about 1100 aa.

Suitable heterologous fusion partners include, but are not limited to, enzymes that modify a nucleic acid; enzymes that modify a polypeptide (e.g., a polypeptide bounds to a target nucleic acid); non-enzymatic polypeptides that bind to a nucleic acid; and polypeptides that modify (e.g., control) transcription.

In some cases, the heterologous fusion partner exhibits nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity. In some cases, the heterologous fusion partner exhibits nuclease activity. In some cases, the heterologous fusion partner introduces a double strand break in a target DNA. In some cases, the heterologous fusion partner modifies a target polypeptide associated with a target DNA. In some cases, the heterologous fusion partner exhibits methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, or demyristoylation activity.

In some cases, the heterologous fusion partner is a nucleic acid-modifying enzyme. Examples of suitable nucleic acid-modifying enzymes include DNA editing enzymes; deaminases, such as activation induced deaminase and APOBEC proteins; nucleases; recombinases; glycosylases; methyltransferases; and the like. In other cases the heterologous fusion partner is a protein-modifying enzyme. For example, the heterologous fusion partner may be one that modifies a protein associated with a nucleic acid. Examples of suitable protein-modifying enzymes include histone-modifying enzymes, acetylases, kinases, methyltransferases, ubiquitin ligases, SUMO ligases, demethylases, deacetylases, phosphatases, and the like.

In some cases the fusion partner can modulate transcription (e.g., inhibit transcription, increase transcription) of a target DNA. For example, in some cases the fusion partner is a protein (or a domain from a protein) that inhibits transcription (e.g., a transcriptional repressor, a protein that functions via recruitment of transcription inhibitor proteins, modification of target DNA such as methylation, recruitment of a DNA modifier, modulation of histones associated with target DNA, recruitment of a histone modifier such as those that modify acetylation and/or methylation of histones, and the like). In some cases the fusion partner is a protein (or a domain from a protein) that increases transcription (e.g., a transcription activator, a protein that acts via recruitment of transcription activator proteins, modification of target DNA such as demethylation, recruitment of a DNA modifier, modulation of histones associated with target DNA, recruitment of a histone modifier such as those that modify acetylation and/or methylation of histones, and the like).

Examples of proteins (or fragments thereof) that can be used in increase transcription include but are not limited to: transcriptional activators such as VP16, VP64, VP48, VP160, p65 subdomain (e.g., from NFkB), and activation domain of EDLL and/or TAL activation domain (e.g., for activity in plants); histone lysine methyltransferases such as SET1A, SET1B, MLL1 to 5, ASH1, SYMD2, NSD1, and the like; histone lysine demethylases such as JHDM2a/b, UTX, JMJD3, and the like; histone acetyltransferases such as GCN5, PCAF, CBP, p300, TAF1, TIP60/PLIP, MOZ/MYST3, MORF/MYST4, SRC1, ACTR, P160, CLOCK, and the like; and DNA demethylases such as Ten-Eleven Translocation (TET) dioxygenase 1 (TET1CD), TET1, DME, DML1, DML2, ROS1, and the like.

Examples of proteins (or fragments thereof) that can be used in decrease transcription include but are not limited to: transcriptional repressors such as the Krüppel associated box (KRAB or SKD); KOX1 repression domain; the Mad mSIN3 interaction domain (SID); the ERF repressor domain (ERD), the SRDX repression domain (e.g., for repression in plants), and the like; histone lysine methyltransferases such as Pr-SET7/8, SUV4-20H1, RIZ1, and the like; histone lysine demethylases such as JMJD2A/JHDM3A, JMJD2B, JMJD2C/GASC1, JMJD2D, JARID1A/RBP2, JARID1B/PLU-1, JARID1C/SMCX, JARID1D/SMCY, and the like; histone lysine deacetylases such as HDAC1, HDAC2, HDAC3, HDAC8, HDAC4, HDAC5, HDAC7, HDAC9, SIRT1, SIRT2, HDAC11, and the like; DNA methylases such as HhaI DNA m5c-methyltransferase (M.HhaI), DNA methyltransferase 1 (DNMT1), DNA methyltransferase 3a (DNMT3a), DNA methyltransferase 3b (DNMT3b), METI, DRM3 (plants), ZMET2, CMT1, CMT2 (plants), and the like; and periphery recruitment elements such as Lamin A, Lamin B, and the like.

In some cases, the heterologous fusion partner is a nuclease that provides for cleavage of a double-stranded DNA. In some cases, the heterologous fusion partner is a nickase. In some cases, a fusion polypeptide of the present disclosure includes a heterologous polypeptide that has enzymatic activity that modifies a target nucleic acid (e.g., nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity).

In some cases the fusion partner has enzymatic activity that modifies a protein associated with the target nucleic acid (e.g., ssRNA, dsRNA, ssDNA, dsDNA) (e.g., a histone, an RNA binding protein, a DNA binding protein, and the like). Examples of enzymatic activity (that modifies a protein associated with a target nucleic acid) that can be provided by the fusion partner include but are not limited to: methyltransferase activity such as that provided by a histone methyltransferase (HMT) (e.g., suppressor of variegation 3-9 homolog 1 (SUV39H1, also known as KMT1A), euchromatic histone lysine methyltransferase 2 (G9A, also known as KMT1C and EHMT2), SUV39H2, ESET/SETDB1, and the like, SET1A, SET1B, MLL1 to 5, ASH1, SYMD2, NSD1, DOT1L, Pr-SET7/8, SUV4-20H1, EZH2, RIZ1), demethylase activity such as that provided by a histone demethylase (e.g., Lysine Demethylase 1A (KDM1A also known as LSD1), JHDM2a/b, JMJD2A/JHDM3A, JMJD2B, JMJD2C/GASC1, JMJD2D, JARID1A/RBP2, JARID1B/PLU-1, JARID1C/SMCX, JARID1D/SMCY, UTX, JMJD3, and the like), acetyltransferase activity such as that provided by a histone acetylase transferase (e.g., catalytic core/fragment of the human acetyltransferase p300, GCN5, PCAF, CBP, TAF1, TIP60/PLIP, MOZ/MYST3, MORF/MYST4, HBO1/MYST2, HMOF/MYST1, SRC1, ACTR, P160, CLOCK, and the like), deacetylase activity such as that provided by a histone deacetylase (e.g., HDAC1, HDAC2, HDAC3, HDAC8, HDAC4, HDAC5, HDAC7, HDAC9, SIRT1, SIRT2, HDAC11, and the like), kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, and demyristoylation activity.

In some cases the fusion partner has enzymatic activity that modifies the target nucleic acid (e.g., ssRNA, dsRNA, ssDNA, dsDNA). Examples of enzymatic activity that can be provided by the fusion partner include but are not limited to: nuclease activity such as that provided by a restriction enzyme (e.g., FokI nuclease), methyltransferase activity such as that provided by a methyltransferase (e.g., HhaI DNA m5c-methyltransferase (M.HhaI), DNA methyltransferase 1 (DNMT1), DNA methyltransferase 3a (DNMT3a), DNA methyltransferase 3b (DNMT3b), METI, DRM3 (plants), ZMET2, CMT1, CMT2 (plants), and the like); demethylase activity such as that provided by a demethylase (e.g., Ten-Eleven Translocation (TET) dioxygenase 1 (TET1CD), TET1, DME, DML1, DML2, ROS1, and the like), DNA repair activity, DNA damage activity, deamination activity such as that provided by a deaminase (e.g., a cytosine deaminase enzyme such as rat APOBEC1), dismutase activity, alkylation activity, depurination activity, oxidation activity, pyrimidine dimer forming activity, integrase activity such as that provided by an integrase and/or resolvase (e.g., Gin invertase such as the hyperactive mutant of the Gin invertase, GinH106Y; human immunodeficiency virus type 1 integrase (IN); Tn3 resolvase; and the like), transposase activity, recombinase activity such as that provided by a recombinase (e.g., catalytic domain of Gin recombinase), polymerase activity, ligase activity, helicase activity, photolyase activity, and glycosylase activity).

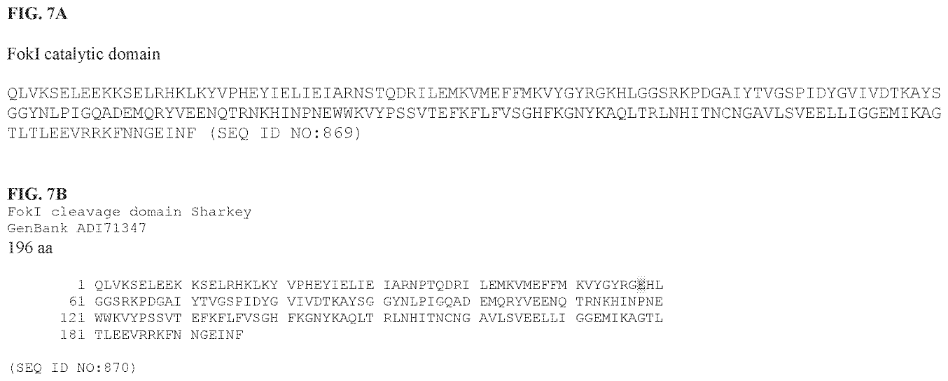

As one non-limiting example, in some cases, the heterologous fusion partner: i) is a FokI nuclease comprising an amino acid sequence having at least at least 85%, at least 90%, at least 95%, at least 98%, at least 99%, or 100%, amino acid sequence identity to the FokI polypeptide depicted in FIG. 7A or FIG. 7B; and ii) has a length of from about 195 amino acids to about 200 amino acids.

In some cases, the heterologous fusion partner is a deaminase. Suitable deaminases include a cytidine deaminase and an adenosine deaminase.

In some cases, a suitable deaminase provides for generation of a G→T substitution. In some cases, a suitable deaminase provides for generation of a G→C substitution. In some cases, a suitable deaminase provides for generation of a C→A substitution. In some cases, a suitable deaminase provides for generation of a C→G substitution. In some cases, a suitable deaminase provides for generation of an A→T substitution. In some cases, a suitable deaminase provides for generation of an A→G substitution. In some cases, a suitable deaminase provides for generation of an A→C substitution. In some cases, a suitable deaminase provides for generation of a T→A substitution. In some cases, a suitable deaminase provides for generation of a T→G substitution. In some cases, a suitable deaminase provides for generation of a T→C substitution. In some cases, a suitable deaminase provides for generation of a G→A substitution. In some cases, a suitable deaminase provides for generation of a C→T substitution.

Adenosine deaminases suitable as heterologous fusion partners include any enzyme that is capable of deaminating adenosine in DNA. In some cases, the deaminase is a TadA deaminase.

In some cases, a suitable adenosine deaminase comprises an amino acid sequence having at least 80%, at least 85%, at least 90%, at least 95%, at least 98%, at least 99%, or 100%, amino acid sequence identity to the following amino acid sequence:

(SEQ ID NO: 1)
MSEVEFSHEYWMRHALTLAKRAWDEREVPVGAVLVHNNRVIGEGWNRPIGR
HDPTAHAEIMALRQGGLVMQNYRLIDATLYVTLEPCVMCAGAMIHSRIGRV
VFGARDAKTGAAGSLMDVLHHPGMNHRVEITEGILADECAALLSDFFRMRR
QEIKAQKKAQSSTD

In some cases, a suitable adenosine deaminase comprises an amino acid sequence having at least 80%, at least 85%, at least 90%, at least 95%, at least 98%, at least 99%, or 100%, amino acid sequence identity to the following amino acid sequence:

(SEQ ID NO: 2)
MRRAFITGVFFLSEVEFSHEYWMRHALTLAKRAWDEREVPVGAVLVHNNRV
IGEGWNRPIGRHDPTAHAEIMALRQGGLVMQNYRLIDATLYVTLEPCVMCA
GAMIHSRIGRVVFGARDAKTGAAGSLMDVLHHPGMNHRVEITEGILADECA
ALLSDFFRMRRQEIKAQKKAQSSTD.

In some cases, a suitable adenosine deaminase comprises an amino acid sequence having at least 80%, at least 85%, at least 90%, at least 95%, at least 98%, at least 99%, or 100%, amino acid sequence identity to the following Staphylococcus aureus TadA amino acid sequence:

(SEQ ID NO: 3)
MGSHMTNDIYFMTLAIEEAKKAAQLGEVPIGAIITKDDEVIARAHNLRETL
QQPTAHAEHIAIERAAKVLGSWRLEGCTLYVTLEPCVMCAGTIVMSRIPRV
VYGADDPKGGCSGSLMNLLQQSNFNHRAIVDKGVLKEACSTLLTTFFKNLR
ANKKSTN:

In some cases, a suitable adenosine deaminase comprises an amino acid sequence having at least 80%, at least 85%, at least 90%, at least 95%, at least 98%, at least 99%, or 100%, amino acid sequence identity to the following Bacillus subtilis TadA amino acid sequence:

(SEQ ID NO: 4)
MTQDELYMKEAIKEAKKAEEKGEVPIGAVLVINGEIIARAHNLRETEQRSI
AHAEMLVIDEACKALGTWRLEGATLYVTLEPCPMCAGAVVLSRVEKVVFGA
FDPKGGCSGTLMNLLQEERFNHQAEVVSGVLEEECGGMLSAFFRELRKKKK
AARKNLSE

In some cases, a suitable adenosine deaminase comprises an amino acid sequence having at least 80%, at least 85%, at least 90%, at least 95%, at least 98%, at least 99%, or 100%, amino acid sequence identity to the following Salmonella typhimurium TadA:

(SEQ ID NO: 817)
MPPAFITGVTSLSDVELDHEYWMRHALTLAKRAWDEREVPVGAVLVHNHRV
IGEGWNRPIGRHDPTAHAEIMALRQGGLVLQNYRLLDTTLYVTLEPCVMCA
GAMVHSRIGRVVFGARDAKTGAAGSLIDVLHHPGMNHRVEIIEGVLRDECA
TLLSDFFRMRRQEIKALKKADRAEGAGPAV.

In some cases, a suitable adenosine deaminase comprises an amino acid sequence having at least 80%, at least 85%, at least 90%, at least 95%, at least 98%, at least 99%, or 100%, amino acid sequence identity to the following Shewanella putrefaciens TadA amino acid sequence:

(SEQ ID NO: 818)
MDEYWMQVAMQMAEKAEAAGEVPVGAVLVKDGQQIATGYNLSISQHDPTAH
AEILCLRSAGKKLENYRLLDATLYITLEPCAMCAGAMVHSRIARVVYGARD
EKTGAAGTVVNLLQHPAFNHQVEVTSGVLAEACSAQLSRFFKRRRDEKKAL
KLAQRAQQGIE

In some cases, a suitable adenosine deaminase comprises an amino acid sequence having at least 80%, at least 85%, at least 90%, at least 95%, at least 98%, at least 99%, or 100%, amino acid sequence identity to the following Haemophilus influenzae F3031 TadA amino acid sequence:

(SEQ ID NO: 819)
MDAAKVRSEFDEKMMRYALELADKAEALGEIPVGAVLVDDARNIIGEGWNL
SIVQSDPTAHAEIIALRNGAKNIQNYRLLNSTLYVTLEPCTMCAGAILHSR
IKRLVFGASDYKTGAIGSRFHFFDDYKMNHTLEITSGVLAEECSQKLSTFF
QKRREEKKIEKALLKSLSDK

In some cases, a suitable adenosine deaminase comprises an amino acid sequence having at least 80%, at least 85%, at least 90%, at least 95%, at least 98%, at least 99%, or 100%, amino acid sequence identity to the following Caulobacter crescentus TadA amino acid sequence:

(SEQ ID NO: 820)
MRTDESEDQDHRMMRLALDAARAAAEAGETPVGAVILDPSTGEVIATAGNG
PIAAHDPTAHAEIAAMRAAAAKLGNYRLTDLTLVVTLEPCAMCAGAISHAR
IGRVVFGADDPKGGAVVHGPKFFAQPTCHWRPEVTGGVLADESADLLRGFF
RARRKAKI

In some cases, a suitable adenosine deaminase comprises an amino acid sequence having at least 80%, at least 85%, at least 90%, at least 95%, at least 98%, at least 99%, or 100%, amino acid sequence identity to the following Geobacter sulfurreducens TadA amino acid sequence:

(SEQ ID NO: 821)
MSSLKKTPIRDDAYWMGKAIREAAKAAARDEVPIGAVIVRDGAVIGRGHNL
REGSNDPSAHAEMIAIRQAARRSANWRLTGATLYVTLEPCLMCMGAIILAR
LERVVFGCYDPKGGAAGSLYDLSADPRLNHQVRLSPGVCQEECGTMLSDFF
RDLRRRKKAKATPALFIDERKVPPEP

Cytidine deaminases suitable as heterologous fusion partners include any enzyme that is capable of deaminating cytidine in DNA.

In some cases, the cytidine deaminase is a deaminase from the apolipoprotein B mRNA-editing complex (APOBEC) family of deaminases. In some cases, the APOBEC family deaminase is selected from the group consisting of APOBEC1 deaminase, APOBEC2 deaminase, APOBEC3A deaminase, APOBEC3B deaminase, APOBEC3C deaminase, APOBEC3D deaminase, APOBEC3F deaminase, APOBEC3G deaminase, and APOBEC3H deaminase. In some cases, the cytidine deaminase is an activation induced deaminase (AID).

In some cases, a suitable cytidine deaminase comprises an amino acid sequence having at least 80%, at least 85%, at least 90%, at least 95%, at least 98%, at least 99%, or 100%, amino acid sequence identity to the following amino acid sequence:

(SEQ ID NO: 822)
MDSLLMNRRKFLYQFKNVRWAKGRRETYLCYVVKRRDSATSFSLDFGYLRN
KNGCHVELLFLRYISDWDLDPGRCYRVTWFTSWSPCYDCARHVADFLRGNP
NLSLRIFTARLYFCEDRKAEPEGLRRLHRAGVQIAIMTFKDYFYCWNTFVE
NHERTFKAWEGLHENSVRLSRQLRRILLPLYEVDDLRDAFRTLGL

In some cases, a suitable cytidine deaminase is an AID and comprises an amino acid sequence having at least 80%, at least 85%, at least 90%, at least 95%, at least 98%, at least 99%, or 100%, amino acid sequence identity to the following amino acid sequence: MDSLLMNRRK

(SEQ ID NO: 823)
FLYQFKNVRW AKGRRETYLC YVVKRRDSAT SFSLDFGYLR
NKNGCHVELL FLRYISDWDL DPGRCYRVTW FTSWSPCYDC
ARHVADFLRG NPNLSLRIFT ARLYFCEDRK AEPEGLRRLH
RAGVQIAIMT FKENHERTFK AWEGLHENSV RLSRQLRRIL
LPLYEVDDLR DAFRTLGL.

In some cases, a suitable cytidine deaminase is an AID and comprises an amino acid sequence having at least 80%, at least 85%, at least 90%, at least 95%, at least 98%, at least 99%, or 100%, amino acid sequence identity to the following amino acid sequence: MDSLLMNRRK

(SEQ ID NO: 824)
FLYQFKNVRW AKGRRETYLC YVVKRRDSAT SFSLDFGYLR
NKNGCHVELL FLRYISDWDL DPGRCYRVTW FTSWSPCYDC
ARHVADFLRG NPNLSLRIFT ARLYFCEDRK AEPEGLRRLH
RAGVQIAIMT FKDYFYCWNT FVENHERTFK AWEGLHENSV
RLSRQLRRIL LPLYEVDDLR DAFRTLGL.

### Additional Polypeptides

In some cases, a fusion polypeptide of the present disclosure comprises one or more additional polypeptides, where suitable additional polypeptides include, e.g., a nuclear localization signal (NLS), a cell-penetrating peptide (CPP), an endosomolytic peptide, and the like. In some cases, a heterologous polypeptide (a fusion partner) provides for subcellular localization, i.e., the heterologous polypeptide contains a subcellular localization sequence (e.g., a nuclear localization signal (NLS) for targeting to the nucleus, a sequence to keep the fusion protein out of the nucleus, e.g., a nuclear export sequence (NES), a sequence to keep the fusion protein retained in the cytoplasm, a mitochondrial localization signal for targeting to the mitochondria, a chloroplast localization signal for targeting to a chloroplast, an ER retention signal, and the like). In some cases, a fusion polypeptide of the present disclosure does not include a NLS so that the protein is not targeted to the nucleus. In some cases, the heterologous polypeptide can provide a tag (i.e., the heterologous polypeptide is a detectable label) for ease of tracking and/or purification (e.g., a fluorescent protein, e.g., green fluorescent protein (GFP), yellow fluorescent protein (YFP), red fluorescent protein (RFP), cyan fluorescent protein (CFP), mCherry, tdTomato, and the like; a histidine tag, e.g., a 6×His tag; a hemagglutinin (HA) tag; a FLAG tag; a Myc tag; and the like).

In some cases, a fusion polypeptide of the present disclosure includes (is fused to) a nuclear localization signal (NLS) (e.g., in some cases 2 or more, 3 or more, 4 or more, or 5 or more NLSs). Thus, in some cases, a fusion polypeptide of the present disclosure includes one or more NLSs (e.g., 2 or more, 3 or more, 4 or more, or 5 or more NLSs). In some cases, one or more NLSs (2 or more, 3 or more, 4 or more, or 5 or more NLSs) are positioned at or near (e.g., within 50 amino acids of) the N-terminus and/or the C-terminus. In some cases, one or more NLSs (2 or more, 3 or more, 4 or more, or 5 or more NLSs) are positioned at or near (e.g., within 50 amino acids of) the N-terminus. In some cases, one or more NLSs (2 or more, 3 or more, 4 or more, or 5 or more NLSs) are positioned at or near (e.g., within 50 amino acids of) the C-terminus. In some cases, one or more NLSs (3 or more, 4 or more, or 5 or more NLSs) are positioned at or near (e.g., within 50 amino acids of) both the N-terminus and the C-terminus. In some cases, an NLS is positioned at the N-terminus and an NLS is positioned at the C-terminus.

Non-limiting examples of NLSs include an NLS sequence derived from: the NLS of the SV40 virus large T-antigen, having the amino acid sequence PKKKRKV (SEQ ID NO: 825); the NLS from nucleoplasmin (e.g., the nucleoplasmin bipartite NLS with the sequence KRPAATKKAGQAKKKK (SEQ ID NO: 826)); the c-myc NLS having the amino acid sequence PAAKRVKLD (SEQ ID NO: 827) or RQRRNELKRSP (SEQ ID NO:828); the hRNPA1 M9 NLS having the sequence NQSSNFGPMKGGNFGGRSSGPYGGGGQYFAKPRNQGGY (SEQ ID NO: 829); the sequence RMRIZFKNKGKDTAELRRRRVEVSVELRKAKKDEQILKRRNV (SEQ ID NO: 830) of the IBB domain from importin-alpha; the sequences VSRKRPRP (SEQ ID NO: 831) and PPKKARED (SEQ ID NO: 832) of the myoma T protein; the sequence PQPKKKPL (SEQ ID NO: 833) of human p53; the sequence SALIKKKKKMAP (SEQ ID NO: 834) of mouse c-abl IV; the sequences DRLRR (SEQ ID NO: 835) and PKQKKRK (SEQ ID NO: 836) of the influenza virus NS1; the sequence RKLKKKIKKL (SEQ ID NO: 837) of the Hepatitis virus delta antigen; the sequence REKKKFLKRR (SEQ ID NO: 838) of the mouse Mx1 protein; the sequence KRKGDEVDGVDEVAKKKSKK (SEQ ID NO: 839) of the human poly(ADP-ribose) polymerase; and the sequence RKCLQAGMNLEARKTKK (SEQ ID NO: 840) of the steroid hormone receptors (human) glucocorticoid. In general, NLS (or multiple NLSs) are of sufficient strength to drive accumulation of the protein in a detectable amount in the nucleus of a eukaryotic cell. Detection of accumulation in the nucleus may be performed by any suitable technique.

In some cases, a fusion polypeptide of the present disclosure comprises: a) an RNA-guided effector polypeptide of the present disclosure; and b) a chloroplast transit peptide. Thus, for example, an RNA-guided effector polypeptide/guide RNA complex can be targeted to the chloroplast. In some cases, this targeting may be achieved by the presence of an N-terminal extension, called a chloroplast transit peptide (CTP) or plastid transit peptide. Chromosomal transgenes from bacterial sources must have a sequence encoding a CTP sequence fused to a sequence encoding an expressed polypeptide if the expressed polypeptide is to be compartmentalized in the plant plastid (e.g. chloroplast). Accordingly, localization of an exogenous polypeptide to a chloroplast is often 1 accomplished by means of operably linking a polynucleotide sequence encoding a CTP sequence to the 5′ region of a polynucleotide encoding the exogenous polypeptide. The CTP is removed in a processing step during translocation into the plastid. Processing efficiency may, however, be affected by the amino acid sequence of the CTP and nearby sequences at the N-terminus of the peptide. Other options for targeting to the chloroplast which have been described are the maize cab-m7 signal sequence (U.S. Pat. No. 7,022,896, WO 97/41228) a pea glutathione reductase signal sequence (WO 97/41228) and the CTP described in US2009029861.

In some cases, a fusion polypeptide of the present disclosure can comprise: a) an RNA-guided effector polypeptide of the present disclosure; and b) an endosomal escape peptide. In some cases, an endosomal escape polypeptide comprises the amino acid sequence GLFXALLXLLXSLWXLLLXA (SEQ ID NO:841), wherein each X is independently selected from lysine, histidine, and arginine. In some cases, an endosomal escape polypeptide comprises the amino acid sequence GLFHALLHLLHSLWHLLLHA (SEQ ID NO:842).

In some instances, an RNA-guided effector polypeptide of the present disclosure is fused to a fusion partner via a linker polypeptide (e.g., one or more linker polypeptides). The linker polypeptide may have any of a variety of amino acid sequences. Proteins can be joined by a spacer peptide, generally of a flexible nature, although other chemical linkages are not excluded. Suitable linkers include polypeptides of between 4 amino acids and 40 amino acids in length, or between 4 amino acids and 25 amino acids in length. These linkers can be produced by using synthetic, linker-encoding oligonucleotides to couple the proteins, or can be encoded by a nucleic acid sequence encoding the fusion protein. Peptide linkers with a degree of flexibility can be used. The linking peptides may have virtually any amino acid sequence, bearing in mind that the preferred linkers will have a sequence that results in a generally flexible peptide. The use of small amino acids, such as glycine and alanine, are of use in creating a flexible peptide. The creation of such sequences is routine to those of skill in the art. A variety of different linkers are commercially available and are considered suitable for use.

Examples of linker polypeptides include glycine polymers, glycine-serine polymers (including, for example, GSGGS (SEQ ID NO: 843), GGSGGS (SEQ ID NO: 844), and GGGS (SEQ ID NO: 845), glycine-alanine polymers, alanine-serine polymers. Exemplary linkers can comprise amino acid sequences including, but not limited to, GGSG (SEQ ID NO: 846), GGSGG (SEQ ID NO: 847), GSGSG (SEQ ID NO: 848), GSGGG (SEQ ID NO: 849), GGGSG (SEQ ID NO: 850), GSSSG (SEQ ID NO: 851), GGGGS (SEQ ID NO:852) and the like. The ordinarily skilled artisan will recognize that design of a peptide conjugated to any desired element can include linkers that are all or partially flexible, such that the linker can include a flexible linker as well as one or more portions that confer less flexible structure.

### Nucleic Acids and Recombinant Expression Vectors

The present disclosure provides a nucleic acid comprising a nucleotide sequence encoding an RNA-guided effector polypeptide of the present disclosure. The present disclosure provides a nucleic acid comprising a nucleotide sequence encoding a fusion polypeptide of the present disclosure.

The present disclosure provides one or more nucleic acids comprising one or more of: a donor polynucleotide sequence, a nucleotide sequence encoding an RNA-guided effector polypeptide of the present disclosure, a guide RNA, and a nucleotide sequence encoding a guide RNA (which can include two separate nucleotide sequences in the case of dual guide RNA format or which can include a singe nucleotide sequence in the case of single guide RNA format).

The present disclosure provides a recombinant expression vector that comprises a nucleotide sequence encoding an RNA-guided effector polypeptide of the present disclosure. The present disclosure provides a recombinant expression vector that comprises a nucleotide sequence encoding a fusion polypeptide of the present disclosure. The present disclosure provides a recombinant expression vector that comprises: a) a nucleotide sequence encoding an RNA-guided effector polypeptide of the present disclosure; and b) a nucleotide sequence encoding a guide RNA(s). The present disclosure provides a recombinant expression vector that comprises: a) a nucleotide sequence encoding an RNA-guided effector polypeptide of the present disclosure; and b) a nucleotide sequence encoding a guide RNA(s). In some cases, the nucleotide sequence encoding the RNA-guided effector protein and/or the nucleotide sequence encoding the guide RNA is operably linked to a promoter that is operable in a cell type of choice (e.g., a prokaryotic cell, a eukaryotic cell, a plant cell, an animal cell, a mammalian cell, a primate cell, a rodent cell, a human cell, etc.).

In some cases, a nucleotide sequence encoding an RNA-guided effector polypeptide of the present disclosure is codon optimized. This type of optimization can entail a mutation of an RNA-guided effector polypeptide-encoding nucleotide sequence to mimic the codon preferences of the intended host organism or cell while encoding the same protein. Thus, the codons can be changed, but the encoded protein remains unchanged. For example, if the intended target cell was a human cell, a human codon-optimized RNA-guided effector polypeptide-encoding nucleotide sequence could be used. As another non-limiting example, if the intended host cell were a mouse cell, then a mouse codon-optimized RNA-guided effector polypeptide-encoding nucleotide sequence could be generated. As another non-limiting example, if the intended host cell were a plant cell, then a plant codon-optimized RNA-guided effector polypeptide-encoding nucleotide sequence could be generated. As another non-limiting example, if the intended host cell were an insect cell, then an insect codon-optimized RNA-guided effector polypeptide-encoding nucleotide sequence could be generated.

The present disclosure provides one or more recombinant expression vectors that include (in different recombinant expression vectors in some cases, and in the same recombinant expression vector in some cases): (i) a nucleotide sequence of a donor template nucleic acid (where the donor template comprises a nucleotide sequence having homology to a target sequence of a target nucleic acid (e.g., a target genome)); (ii) a nucleotide sequence that encodes a guide RNA that hybridizes to a target sequence of the target locus of the targeted genome (e.g., a single or dual guide RNA) (e.g., operably linked to a promoter that is operable in a target cell such as a eukaryotic cell); and (iii) a nucleotide sequence encoding RNA-guided effector polypeptide of the present disclosure, or a fusion polypeptide of the present disclosure (e.g., operably linked to a promoter that is operable in a target cell such as a eukaryotic cell). The present disclosure provides one or more recombinant expression vectors that include (in different recombinant expression vectors in some cases, and in the same recombinant expression vector in some cases): (i) a nucleotide sequence of a donor template nucleic acid (where the donor template comprises a nucleotide sequence having homology to a target sequence of a target nucleic acid (e.g., a target genome)); and (ii) a nucleotide sequence that encodes a guide RNA that hybridizes to a target sequence of the target locus of the targeted genome (e.g., a single or dual guide RNA) (e.g., operably linked to a promoter that is operable in a target cell such as a eukaryotic cell). The present disclosure provides one or more recombinant expression vectors that include (in different recombinant expression vectors in some cases, and in the same recombinant expression vector in some cases): (i) a nucleotide sequence that encodes a guide RNA that hybridizes to a target sequence of the target locus of the targeted genome (e.g., a single or dual guide RNA) (e.g., operably linked to a promoter that is operable in a target cell such as a eukaryotic cell); and (ii) a nucleotide sequence encoding an RNA-guided effector polypeptide of the present disclosure or a fusion polypeptide of the present disclosure (e.g., operably linked to a promoter that is operable in a target cell such as a eukaryotic cell).

Suitable expression vectors include viral expression vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., Gene Ther 5:1088 1097, 1999; WO 94/12649, WO 93/03769; WO 93/19191; WO 94/28938; WO 95/11984 and WO 95/00655); adeno-associated virus (AAV) (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like. In some cases, a recombinant expression vector of the present disclosure is a recombinant adeno-associated virus (AAV) vector. In some cases, a recombinant expression vector of the present disclosure is a recombinant lentivirus vector. In some cases, a recombinant expression vector of the present disclosure is a recombinant retroviral vector.

Depending on the host/vector system utilized, any of a number of suitable transcription and translation control elements, including constitutive and inducible promoters, transcription enhancer elements, transcription terminators, etc. may be used in the expression vector.

In some embodiments, a nucleotide sequence encoding a guide RNA is operably linked to a control element, e.g., a transcriptional control element, such as a promoter. In some embodiments, a nucleotide sequence encoding an RNA-guided effector polypeptide of the present disclosure or a fusion polypeptide of the present disclosure is operably linked to a control element, e.g., a transcriptional control element, such as a promoter.

The transcriptional control element can be a promoter. In some cases, the promoter is a constitutively active promoter. In some cases, the promoter is a regulatable promoter. In some cases, the promoter is an inducible promoter. In some cases, the promoter is a tissue-specific promoter. In some cases, the promoter is a cell type-specific promoter. In some cases, the transcriptional control element (e.g., the promoter) is functional in a targeted cell type or targeted cell population. For example, in some cases, the transcriptional control element can be functional in eukaryotic cells, e.g., hematopoietic stem cells (e.g., mobilized peripheral blood (mPB) CD34(+) cell, bone marrow (BM) CD34(+) cell, etc.).

Non-limiting examples of eukaryotic promoters (promoters functional in a eukaryotic cell) include EF1α, those from cytomegalovirus (CMV) immediate early, herpes simplex virus (HSV) thymidine kinase, early and late SV40, long terminal repeats (LTRs) from retrovirus, and mouse metallothionein-I. Selection of the appropriate vector and promoter is well within the level of ordinary skill in the art. Non-limiting examples of tissue specific promoters include Syn1 (neurons); GFAP (astrocytes); B29 (B cells); hAAT (liver); albumin (liver); MCR (muscle); SPB (lung), and the like. The expression vector may also contain a ribosome binding site for translation initiation and a transcription terminator. The expression vector may also include appropriate sequences for amplifying expression. The expression vector may also include nucleotide sequences encoding protein tags (e.g., 6×His tag, hemagglutinin tag, fluorescent protein, etc.) that can be fused to the RNA-guided effector polypeptide of the present disclosure.

In some cases, a nucleotide sequence encoding a guide RNA and/or an RNA-guided effector polypeptide of the present disclosure or a fusion polypeptide of the present disclosure is operably linked to an inducible promoter. In some cases, a nucleotide sequence encoding a guide RNA and/or an RNA-guided effector polypeptide of the present disclosure or a fusion polypeptide of the present disclosure is operably linked to a constitutive promoter.

A promoter can be a constitutively active promoter (i.e., a promoter that is constitutively in an active/“ON” state), it may be an inducible promoter (i.e., a promoter whose state, active/“ON” or inactive/“OFF”, is controlled by an external stimulus, e.g., the presence of a particular temperature, compound, or protein), it may be a spatially restricted promoter (i.e., transcriptional control element, enhancer, etc.)(e.g., tissue specific promoter, cell type specific promoter, etc.), and it may be a temporally restricted promoter (i.e., the promoter is in the “ON” state or “OFF” state during specific stages of embryonic development or during specific stages of a biological process, e.g., hair follicle cycle in mice).

Suitable promoters can be derived from viruses and can therefore be referred to as viral promoters, or they can be derived from any organism, including prokaryotic or eukaryotic organisms. Suitable promoters can be used to drive expression by any RNA polymerase (e.g., pol I, pol II, pol III). Exemplary promoters include, but are not limited to the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6) (Miyagishi et al., Nature Biotechnology 20, 497-500 (2002)), an enhanced U6 promoter (e.g., Xia et al., Nucleic Acids Res. 2003 Sep. 1; 31(17)), a human H1 promoter (H1), and the like.

In some cases, a nucleotide sequence encoding a guide RNA is operably linked to (under the control of) a promoter operable in a eukaryotic cell (e.g., a U6 promoter, an enhanced U6 promoter, an H1 promoter, and the like). As would be understood by one of ordinary skill in the art, when expressing an RNA (e.g., a guide RNA) from a nucleic acid (e.g., an expression vector) using a U6 promoter (e.g., in a eukaryotic cell), or another PolIII promoter, the RNA may need to be mutated if there are several Ts in a row (coding for Us in the RNA). This is because a string of Ts (e.g., 5 Ts) in DNA can act as a terminator for polymerase III (PolIII). Thus, in order to ensure transcription of a guide RNA (e.g., the activator portion and/or targeter portion, in dual guide or single guide format) in a eukaryotic cell it may sometimes be necessary to modify the sequence encoding the guide RNA to eliminate runs of Ts. In some cases, a nucleotide sequence encoding an RNA-guided effector polypeptide of the present disclosure or a fusion polypeptide of the present disclosure is operably linked to a promoter operable in a eukaryotic cell (e.g., a CMV promoter, an EF1α promoter, an estrogen receptor-regulated promoter, and the like).

Examples of inducible promoters include, but are not limited to T7 RNA polymerase promoter, T3 RNA polymerase promoter, Isopropyl-beta-D-thiogalactopyranoside (IPTG)-regulated promoter, lactose induced promoter, heat shock promoter, Tetracycline-regulated promoter, Steroid-regulated promoter, Metal-regulated promoter, estrogen receptor-regulated promoter, etc. Inducible promoters can therefore be regulated by molecules including, but not limited to, doxycycline; estrogen and/or an estrogen analog; IPTG; etc.

Inducible promoters suitable for use include any inducible promoter described herein or known to one of ordinary skill in the art. Examples of inducible promoters include, without limitation, chemically/biochemically-regulated and physically-regulated promoters such as alcohol-regulated promoters, rapamycin-regulated promoters, tetracycline-regulated promoters (e.g., anhydrotetracycline (aTc)-responsive promoters and other tetracycline-responsive promoter systems, which include a tetracycline repressor protein (tetR), a tetracycline operator sequence (tetO) and a tetracycline transactivator fusion protein (tTA)), steroid-regulated promoters (e.g., promoters based on the rat glucocorticoid receptor, human estrogen receptor, moth ecdysone receptors, and promoters from the steroid/retinoid/thyroid receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), and light-regulated promoters (e.g., light responsive promoters from plant cells).

In some cases, the promoter is a spatially restricted promoter (i.e., cell type specific promoter, tissue specific promoter, etc.) such that in a multi-cellular organism, the promoter is active (i.e., “ON”) in a subset of specific cells. Spatially restricted promoters may also be referred to as enhancers, transcriptional control elements, control sequences, etc. Any convenient spatially restricted promoter may be used as long as the promoter is functional in the targeted host cell (e.g., eukaryotic cell; prokaryotic cell).

In some cases, the promoter is a reversible promoter. Suitable reversible promoters, including reversible inducible promoters are known in the art. Such reversible promoters may be isolated and derived from many organisms, e.g., eukaryotes and prokaryotes. Modification of reversible promoters derived from a first organism for use in a second organism, e.g., a first prokaryote and a second a eukaryote, a first eukaryote and a second a prokaryote, etc., is well known in the art. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (AlcR), etc.), tetracycline regulated promoters, (e.g., promoter systems including TetActivators, TetON, TetOFF, etc.), steroid regulated promoters (e.g., rat glucocorticoid receptor promoter systems, human estrogen receptor promoter systems, retinoid promoter systems, thyroid promoter systems, ecdysone promoter systems, mifepristone promoter systems, etc.), metal regulated promoters (e.g., metallothionein promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soybean heat shock promoter, etc.), light regulated promoters, synthetic inducible promoters, and the like.

Methods of introducing a nucleic acid (e.g., a nucleic acid comprising a donor polynucleotide sequence, one or more nucleic acids encoding an RNA-guided effector polypeptide of the present disclosure or a fusion polypeptide of the present disclosure and/or a guide RNA, and the like) into a host cell are known in the art, and any convenient method can be used to introduce a nucleic acid (e.g., an expression construct) into a cell. Suitable methods include e.g., viral infection, transfection, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct microinjection, nanoparticle-mediated nucleic acid delivery, and the like.

Introducing the recombinant expression vector into cells can occur in any culture media and under any culture conditions that promote the survival of the cells. Introducing the recombinant expression vector into a target cell can be carried out in vivo or ex vivo. Introducing the recombinant expression vector into a target cell can be carried out in vitro.

In some embodiments, an RNA-guided effector polypeptide of the present disclosure or a fusion polypeptide of the present disclosure can be provided as RNA. The RNA can be provided by direct chemical synthesis or may be transcribed in vitro from a DNA (e.g., encoding the RNA-guided effector polypeptide of the present disclosure or the fusion polypeptide of the present disclosure). Once synthesized, the RNA may be introduced into a cell by any of the well-known techniques for introducing nucleic acids into cells (e.g., microinjection, electroporation, transfection, etc.).

Nucleic acids may be provided to the cells using well-developed transfection techniques; see, e.g. Angel and Yanik (2010) PLoS ONE 5(7): e11756, and the commercially available TransMessenger® reagents from Qiagen, Stemfect™ RNA Transfection Kit from Stemgent, and TransIT®-mRNA Transfection Kit from Minis Bio LLC. See also Beumer et al. (2008) PNAS 105(50):19821-19826.

Vectors may be provided directly to a target host cell. In other words, the cells are contacted with vectors comprising the subject nucleic acids (e.g., recombinant expression vectors having the donor template sequence and encoding the guide RNA; recombinant expression vectors encoding an RNA-guided effector polypeptide of the present disclosure or a fusion polypeptide of the present disclosure; etc.) such that the vectors are taken up by the cells. Methods for contacting cells with nucleic acid vectors that are plasmids, include electroporation, calcium chloride transfection, microinjection, and lipofection are well known in the art. For viral vector delivery, cells can be contacted with viral particles comprising the subject viral expression vectors.

Retroviruses, for example, lentiviruses, are suitable for use in methods of the present disclosure. Commonly used retroviral vectors are “defective”, i.e. unable to produce viral proteins required for productive infection. Rather, replication of the vector requires growth in a packaging cell line. To generate viral particles comprising nucleic acids of interest, the retroviral nucleic acids comprising the nucleic acid are packaged into viral capsids by a packaging cell line. Different packaging cell lines provide a different envelope protein (ecotropic, amphotropic or xenotropic) to be incorporated into the capsid, this envelope protein determining the specificity of the viral particle for the cells (ecotropic for murine and rat; amphotropic for most mammalian cell types including human, dog and mouse; and xenotropic for most mammalian cell types except murine cells). The appropriate packaging cell line may be used to ensure that the cells are targeted by the packaged viral particles. Methods of introducing subject vector expression vectors into packaging cell lines and of collecting the viral particles that are generated by the packaging lines are well known in the art. Nucleic acids can also introduced by direct micro-injection (e.g., injection of RNA).

Vectors used for providing the nucleic acids encoding a guide RNA and/or an RNA-guided effector polypeptide of the present disclosure or a fusion polypeptide of the present disclosure to a target host cell can include suitable promoters for driving the expression, that is, transcriptional activation, of the nucleic acid of interest. In other words, in some cases, the nucleic acid of interest will be operably linked to a promoter. This may include ubiquitously acting promoters, for example, the CMV-β-actin promoter, or inducible promoters, such as promoters that are active in particular cell populations or that respond to the presence of drugs such as tetracycline. By transcriptional activation, it is intended that transcription will be increased above basal levels in the target cell by 10 fold, by 100 fold, more usually by 1000 fold. In addition, vectors used for providing a nucleic acid encoding a guide RNA and/or an RNA-guided effector polypeptide of the present disclosure or a fusion polypeptide of the present disclosure to a cell may include nucleic acid sequences that encode for selectable markers in the target cells, so as to identify cells that have taken up the guide RNA and/or the RNA-guided effector polypeptide of the present disclosure or the fusion polypeptide of the present disclosure.

A nucleic acid comprising a nucleotide sequence encoding an RNA-guided effector polypeptide of the present disclosure, or a fusion polypeptide of the present disclosure, is in some cases an RNA. Thus, an RNA-guided effector polypeptide of the present disclosure or a fusion polypeptide of the present disclosure can be introduced into cells as RNA. Methods of introducing RNA into cells are known in the art and may include, for example, direct injection, transfection, incorporation of RNA into lipid nanoparticles, or any other method used for the introduction of DNA. An RNA-guided effector polypeptide of the present disclosure or a fusion polypeptide of the present disclosure protein may instead be provided to cells as a polypeptide. Such a polypeptide may optionally be fused to a polypeptide domain that increases solubility of the product. The domain may be linked to the polypeptide through a defined protease cleavage site, e.g. a TEV sequence, which is cleaved by TEV protease. The linker may also include one or more flexible sequences, e.g. from 1 to 10 glycine residues. In some embodiments, the cleavage of the fusion protein is performed in a buffer that maintains solubility of the product, e.g. in the presence of from 0.5 to 2 M urea, in the presence of polypeptides and/or polynucleotides that increase solubility, and the like. Domains of interest include endosomolytic domains, e.g. influenza HA domain; and other polypeptides that aid in production, e.g. IF2 domain, GST domain, GRPE domain, and the like. The polypeptide may be formulated for improved stability. For example, the peptides may be PEGylated, where the polyethyleneoxy group provides for enhanced lifetime in the blood stream.

Additionally or alternatively, an RNA-guided effector polypeptide of the present disclosure or a fusion polypeptide of the present disclosure may be fused to a polypeptide permeant domain to promote uptake by the cell. A number of permeant domains are known in the art and may be used in the non-integrating polypeptides of the present disclosure, including peptides, peptidomimetics, and non-peptide carriers. For example, a permeant peptide may be derived from the third alpha helix of Drosophila melanogaster transcription factor Antennapaedia, referred to as penetratin, which comprises the amino acid sequence RQIKIWFQNRRMKWKK (SEQ ID NO: 853). As another example, the permeant peptide comprises the HIV-1 tat basic region amino acid sequence, which may include, for example, amino acids 49-57 of naturally-occurring tat protein. Other permeant domains include polyarginine motifs, for example, the region of amino acids 34-56 of HIV-1 rev protein, nona-arginine, octa-arginine, and the like. (See, for example, Futaki et al. (2003) Curr Protein Pept Sci. 2003 April; 4(2): 87-9 and 446; and Wender et al. (2000) Proc. Natl. Acad. Sci. U.S.A 2000 Nov. 21; 97(24):13003-8; published U.S. Patent applications 20030220334; 20030083256; 20030032593; and 20030022831, herein specifically incorporated by reference for the teachings of translocation peptides and peptoids). The nona-arginine (R9) sequence is one of the more efficient PTDs that have been characterized (Wender et al. 2000; Uemura et al. 2002). The site at which the fusion is made may be selected in order to optimize the biological activity, secretion or binding characteristics of the polypeptide. The optimal site will be determined by routine experimentation.

An RNA-guided effector polypeptide of the present disclosure or a fusion polypeptide of the present disclosure may be produced in vitro or by eukaryotic cells or by prokaryotic cells, and it may be further processed by unfolding, e.g. heat denaturation, dithiothreitol reduction, etc. and may be further refolded, using methods known in the art.

Modifications of interest that do not alter primary sequence include chemical derivatization of polypeptides, e.g., acylation, acetylation, carboxylation, amidation, etc. Also included are modifications of glycosylation, e.g. those made by modifying the glycosylation patterns of a polypeptide during its synthesis and processing or in further processing steps; e.g. by exposing the polypeptide to enzymes which affect glycosylation, such as mammalian glycosylating or deglycosylating enzymes. Also embraced are sequences that have phosphorylated amino acid residues, e.g. phosphotyrosine, phosphoserine, or phosphothreonine.

Also suitable for inclusion in embodiments of the present disclosure are nucleic acids (e.g., encoding a guide RNA, encoding an RNA-guided effector polypeptide of the present disclosure or a fusion polypeptide of the present disclosure) that have been modified using ordinary molecular biological techniques and synthetic chemistry so as to improve their resistance to proteolytic degradation, to change the target sequence specificity, to optimize solubility properties, to alter protein activity (e.g., transcription modulatory activity, enzymatic activity, etc.) or to render them more suitable. Analogs of such polypeptides include those containing residues other than naturally occurring L-amino acids, e.g. D-amino acids or non-naturally occurring synthetic amino acids. D-amino acids may be substituted for some or all of the amino acid residues.

An RNA-guided effector polypeptide of the present disclosure or a fusion polypeptide of the present disclosure may be prepared by in vitro synthesis, using conventional methods as known in the art. Various commercial synthetic apparatuses are available, for example, automated synthesizers by Applied Biosystems, Inc., Beckman, etc. By using synthesizers, naturally occurring amino acids may be substituted with unnatural amino acids. The particular sequence and the manner of preparation will be determined by convenience, economics, purity required, and the like.

If desired, various groups may be introduced into the peptide during synthesis or during expression, which allow for linking to other molecules or to a surface. Thus cysteines can be used to make thioethers, histidines for linking to a metal ion complex, carboxyl groups for forming amides or esters, amino groups for forming amides, and the like.

An RNA-guided effector polypeptide of the present disclosure or a fusion polypeptide of the present disclosure may also be isolated and purified in accordance with conventional methods of recombinant synthesis. A lysate may be prepared of the expression host and the lysate purified using high performance liquid chromatography (HPLC), exclusion chromatography, gel electrophoresis, affinity chromatography, or other purification technique. For the most part, the compositions which are used will comprise 20% or more by weight of the desired product, e.g. 75% or more by weight, 95% or more by weight, or 99.5% or more by weight, in relation to contaminants related to the method of preparation of the product and its purification. Usually, the percentages will be based upon total protein. Thus, in some cases, an RNA-guided effector polypeptide of the present disclosure or a fusion polypeptide of the present disclosure is at least 80% pure, at least 85% pure, at least 90% pure, at least 95% pure, at least 98% pure, or at least 99% pure (e.g., free of contaminants, proteins other than the RNA-guided effector polypeptide of the present disclosure or the fusion polypeptide of the present disclosure or other macromolecules, etc.).

To induce cleavage or any desired modification to a target nucleic acid (e.g., genomic DNA), or any desired modification to a polypeptide associated with target nucleic acid, a guide RNA and/or an RNA-guided effector polypeptide of the present disclosure or a fusion polypeptide of the present disclosure and/or the donor template sequence, whether they be introduced as nucleic acids or polypeptides, are provided to the cells for about 30 minutes to about 24 hours, e.g., 1 hour, 1.5 hours, 2 hours, 2.5 hours, 3 hours, 3.5 hours 4 hours, 5 hours, 6 hours, 7 hours, 8 hours, 12 hours, 16 hours, 18 hours, 20 hours, or any other period from about 30 minutes to about 24 hours, which may be repeated with a frequency of about every day to about every 4 days, e.g., every 1.5 days, every 2 days, every 3 days, or any other frequency from about every day to about every four days. The agent(s) may be provided to the subject cells one or more times, e.g. one time, twice, three times, or more than three times, and the cells allowed to incubate with the agent(s) for some amount of time following each contacting event e.g. 16-24 hours, after which time the media is replaced with fresh media and the cells are cultured further.

In cases in which two or more different targeting complexes are provided to the cell (e.g., two different guide RNAs that are complementary to different sequences within the same or different target nucleic acid), the complexes may be provided simultaneously (e.g. as two polypeptides and/or nucleic acids), or delivered simultaneously. Alternatively, they may be provided consecutively, e.g. the targeting complex being provided first, followed by the second targeting complex, etc. or vice versa.

To improve the delivery of a DNA vector into a target cell, the DNA can be protected from damage and its entry into the cell facilitated, for example, by using lipoplexes and polyplexes. Thus, in some cases, a nucleic acid of the present disclosure (e.g., a recombinant expression vector of the present disclosure) can be covered with lipids in an organized structure like a micelle or a liposome. When the organized structure is complexed with DNA it is called a lipoplex. There are three types of lipids, anionic (negatively-charged), neutral, or cationic (positively-charged). Lipoplexes that utilize cationic lipids have proven utility for gene transfer. Cationic lipids, due to their positive charge, naturally complex with the negatively charged DNA. Also as a result of their charge, they interact with the cell membrane. Endocytosis of the lipoplex then occurs, and the DNA is released into the cytoplasm. The cationic lipids also protect against degradation of the DNA by the cell.

Complexes of polymers with DNA are called polyplexes. Most polyplexes consist of cationic polymers and their production is regulated by ionic interactions. One large difference between the methods of action of polyplexes and lipoplexes is that polyplexes cannot release their DNA load into the cytoplasm, so to this end, co-transfection with endosome-lytic agents (to lyse the endosome that is made during endocytosis) such as inactivated adenovirus must occur. However, this is not always the case; polymers such as polyethylenimine have their own method of endosome disruption as does chitosan and trimethylchitosan.

Dendrimers, a highly branched macromolecule with a spherical shape, may be also be used to genetically modify stem cells. The surface of the dendrimer particle may be functionalized to alter its properties. In particular, it is possible to construct a cationic dendrimer (i.e., one with a positive surface charge). When in the presence of genetic material such as a DNA plasmid, charge complementarity leads to a temporary association of the nucleic acid with the cationic dendrimer. On reaching its destination, the dendrimer-nucleic acid complex can be taken up into a cell by endocytosis.

In some cases, a nucleic acid of the disclosure (e.g., an expression vector) includes an insertion site for a guide sequence of interest. For example, a nucleic acid can include an insertion site for a guide sequence of interest, where the insertion site is immediately adjacent to a nucleotide sequence encoding the portion of a guide RNA that does not change when the guide sequence is changed to hybridized to a desired target sequence (e.g., sequences that contribute to the effector polypeptide binding aspect of the guide RNA, e.g., the sequences that contribute to the dsRNA duplex(es) of the guide RNA—this portion of the guide RNA can also be referred to as the ‘scaffold’ or ‘constant region’ of the guide RNA). Thus, in some cases, a subject nucleic acid (e.g., an expression vector) includes a nucleotide sequence encoding a guide RNA, except that the portion encoding the guide sequence portion of the guide RNA is an insertion sequence (an insertion site). An insertion site is any nucleotide sequence used for the insertion of a desired sequence. “Insertion sites” for use with various technologies are known to those of ordinary skill in the art and any convenient insertion site can be used. An insertion site can be for any method for manipulating nucleic acid sequences. For example, in some cases the insertion site is a multiple cloning site (MCS) (e.g., a site including one or more restriction enzyme recognition sequences), a site for ligation independent cloning, a site for recombination based cloning (e.g., recombination based on att sites), a nucleotide sequence recognized by a CRISPR/Cas (e.g. Cas9) based technology, a Cre site, a FLP site, and the like.

An insertion site can be any desirable length, and can depend on the type of insertion site (e.g., can depend on whether (and how many) the site includes one or more restriction enzyme recognition sequences, whether the site includes a target site for a CRISPR/Cas protein, etc.). In some cases, an insertion site of a subject nucleic acid is 3 or more nucleotides (nt) in length (e.g., 5 or more, 8 or more, 10 or more, 15 or more, 17 or more, 18 or more, 19 or more, 20 or more or 25 or more, or 30 or more nt in length). In some cases, the length of an insertion site of a subject nucleic acid has a length in a range of from 2 to 50 nucleotides (nt) (e.g., from 2 to 40 nt, from 2 to 30 nt, from 2 to 25 nt, from 2 to 20 nt, from 5 to 50 nt, from 5 to 40 nt, from 5 to 30 nt, from 5 to 25 nt, from 5 to 20 nt, from 10 to 50 nt, from 10 to 40 nt, from 10 to 30 nt, from 10 to 25 nt, from 10 to 20 nt, from 17 to 50 nt, from 17 to 40 nt, from 17 to 30 nt, from 17 to 25 nt). In some cases, the length of an insertion site of a subject nucleic acid has a length in a range of from 5 to 40 nt.

In some cases, a nucleic acid of the present disclosure (e.g., an expression vector) comprises: i) a nucleotide sequence encoding an RNA-guided effector polypeptide of the present disclosure; and ii) an insertion site for a nucleic acid comprising a nucleotide sequence encoding a heterologous fusion partner of interest. The insertion site can be located relative to the nucleotide sequence encoding the RNA-guided effector polypeptide, such that, after insertion of a nucleic acid comprising a nucleotide sequence encoding a heterologous fusion partner of interest, the resulting fusion polypeptide would comprise, in order from N-terminus to C-terminus: i) the RNA-guided effector polypeptide; and ii) the fusion partner. The insertion site can be located relative to the nucleotide sequence encoding the RNA-guided effector polypeptide, such that, after insertion of a nucleic acid comprising a nucleotide sequence encoding a heterologous fusion partner of interest, the resulting fusion polypeptide would comprise, in order from N-terminus to C-terminus: i) the fusion partner; and ii) the RNA-guided effector polypeptide. An insertion site is any nucleotide sequence used for the insertion of a desired sequence. “Insertion sites” for use with various technologies are known to those of ordinary skill in the art and any convenient insertion site can be used. An insertion site can be for any method for manipulating nucleic acid sequences. For example, in some cases the insertion site is a multiple cloning site (MCS) (e.g., a site including one or more restriction enzyme recognition sequences), a site for ligation independent cloning, a site for recombination based cloning (e.g., recombination based on att sites), a nucleotide sequence recognized by a CRISPR/Cas (e.g. Cas9) based technology, a Cre site, a FLP site, and the like.

### Guide RNA

A nucleic acid that binds to a Cas9 protein and targets the complex to a specific location within a target nucleic acid is referred to herein as a “Cas9 guide RNA” or, simply “a guide RNA.” A Cas9 guide RNA binds to an RNA-guided effector polypeptide of the present disclosure, forming an RNA-guided effector polypeptide/guide RNA complex, and targets the complex to a specific location (nucleotide sequence) within a target nucleic acid. A Cas9 guide RNA binds to a fusion polypeptide of the present disclosure, forming a fusion polypeptide/guide RNA complex, and targets the complex to a specific location (nucleotide sequence) within a target nucleic acid. A guide RNA that binds to a Cas9 polypeptide (e.g., a Spy Cas9 polypeptide as depicted in FIG. 1) also binds to an RNA-guided effector polypeptide of the present disclosure, and to a fusion polypeptide of the present disclosure.

A Cas9 guide RNA (can be said to include two segments, a first segment (referred to herein as a “targeting segment”); and a second segment (referred to herein as a “protein-binding segment”). By “segment” it is meant a segment/section/region of a molecule, e.g., a contiguous stretch of nucleotides in a nucleic acid molecule. A segment can also mean a region/section of a complex such that a segment may comprise regions of more than one molecule.

The first segment (targeting segment) of a Cas9 guide RNA includes a nucleotide sequence (a guide sequence) that is complementary to (and therefore hybridizes with) a specific sequence (a target site) within a target nucleic acid (e.g., a target ssRNA, a target ssDNA, the complementary strand of a double stranded target DNA, etc.). The protein-binding segment (or “protein-binding sequence”) interacts with (binds to) a Cas9 polypeptide, an RNA-guided effector polypeptide of the present disclosure, or a fusion polypeptide of the present disclosure. The protein-binding segment of a subject Cas9 guide RNA includes two complementary stretches of nucleotides that hybridize to one another to form a double stranded RNA duplex (dsRNA duplex). Site-specific binding and/or cleavage of a target nucleic acid (e.g., genomic DNA) can occur at locations (e.g., target sequence of a target locus) determined by base-pairing complementarity between the Cas9 guide RNA (the guide sequence of the Cas9 guide RNA) and the target nucleic acid.

A Cas9 guide RNA and a Cas9 protein (or an RNA-guided effector polypeptide of the present disclosure, or a fusion polypeptide of the present disclosure) form a complex (e.g., bind via non-covalent interactions). The Cas9 guide RNA provides target specificity to the complex by including a targeting segment, which includes a guide sequence (a nucleotide sequence that is complementary to a sequence of a target nucleic acid). The Cas9 protein (or an RNA-guided effector polypeptide of the present disclosure, or a fusion polypeptide of the present disclosure) of the complex provides the site-specific activity (e.g., binding activity provided by an RNA-guided effector polypeptide; binding activity provided by the RNA-guided effector portion of a fusion polypeptide of the present disclosure; or an activity provided by the fusion partner of a fusion polypeptide of the present disclosure). In other words, the Cas9 protein (or an RNA-guided effector polypeptide of the present disclosure, or a fusion polypeptide of the present disclosure) is guided to a target nucleic acid sequence (e.g. a target sequence in a chromosomal nucleic acid, e.g., a chromosome; a target sequence in an extrachromosomal nucleic acid, e.g. an episomal nucleic acid, a minicircle, an ssRNA, an ssDNA, etc.; a target sequence in a mitochondrial nucleic acid; a target sequence in a chloroplast nucleic acid; a target sequence in a plasmid; a target sequence in a viral nucleic acid; etc.) by virtue of its association with the Cas9 guide RNA.

The “guide sequence” also referred to as the “targeting sequence” of a guide RNA can be modified so that the guide RNA can target a Cas9 protein (or an RNA-guided effector polypeptide of the present disclosure, or a fusion polypeptide of the present disclosure) to any desired sequence of any desired target nucleic acid, with the exception that the protospacer adjacent motif (PAM) sequence can be taken into account. Thus, for example, a guide RNA can have a targeting segment with a sequence (a guide sequence) that has complementarity with (e.g., can hybridize to) a sequence in a nucleic acid in a eukaryotic cell, e.g., a viral nucleic acid, a eukaryotic nucleic acid (e.g., a eukaryotic chromosome, chromosomal sequence, a eukaryotic RNA, etc.), and the like.

In some cases, a Cas9 guide RNA includes two separate nucleic acid molecules: an “activator” and a “targeter” and is referred to herein as a “dual Cas9 guide RNA”, a “double-molecule Cas9 guide RNA”, or a “two-molecule Cas9 guide RNA” a “dual guide RNA”, or a “dgRNA.” In some cases, the activator and targeter are covalently linked to one another (e.g., via intervening nucleotides) and the guide RNA is referred to as a “single guide RNA”, a “Cas9 single guide RNA”, a “single-molecule Cas9 guide RNA,” or a “one-molecule Cas9 guide RNA”, or simply “sgRNA.”

A guide RNA comprises a crRNA-like (“CRISPR RNA”/“targeter”/“crRNA”/“crRNA repeat”) molecule and a corresponding tracrRNA-like (“trans-acting CRISPR RNA”/“activator”/“tracrRNA”) molecule. A crRNA-like molecule (targeter) comprises both the targeting segment (single stranded) of the guide RNA and a stretch (“duplex-forming segment”) of nucleotides that forms one half of the dsRNA duplex of the protein-binding segment of the guide RNA. A corresponding tracrRNA-like molecule (activator/tracrRNA) comprises a stretch of nucleotides (duplex-forming segment) that forms the other half of the dsRNA duplex of the protein-binding segment of the guide nucleic acid. In other words, a stretch of nucleotides of a crRNA-like molecule are complementary to and hybridize with a stretch of nucleotides of a tracrRNA-like molecule to form the dsRNA duplex of the protein-binding domain of the Cas9 guide RNA. As such, each targeter molecule can be said to have a corresponding activator molecule (which has a region that hybridizes with the targeter). The targeter molecule additionally provides the targeting segment. Thus, a targeter and an activator molecule (as a corresponding pair) hybridize to form a Cas9 guide RNA. The exact sequence of a given crRNA or tracrRNA molecule is characteristic of the species in which the RNA molecules are found. A subject dual Cas9 guide RNA can include any corresponding activator and targeter pair.

The term “activator” or “activator RNA” is used herein to mean a tracrRNA-like molecule (tracrRNA: “trans-acting CRISPR RNA”) of a Cas9 dual guide RNA (and therefore of a Cas9 single guide RNA when the “activator” and the “targeter” are linked together by, e.g., intervening nucleotides). Thus, for example, a Cas9 guide RNA (dgRNA or sgRNA) comprises an activator sequence (e.g., a tracrRNA sequence). A tracr molecule (a tracrRNA) is a naturally existing molecule that hybridizes with a CRISPR RNA molecule (a crRNA) to form a Cas9 dual guide RNA. The term “activator” is used herein to encompass naturally existing tracrRNAs, but also to encompass tracrRNAs with modifications (e.g., truncations, sequence variations, base modifications, backbone modifications, linkage modifications, etc.) where the activator retains at least one function of a tracrRNA (e.g., contributes to the dsRNA duplex to which Cas9 protein (or an RNA-guided effector polypeptide of the present disclosure, or a fusion polypeptide of the present disclosure) binds). In some cases the activator provides one or more stem loops that can interact with Cas9 protein (or an RNA-guided effector polypeptide of the present disclosure, or a fusion polypeptide of the present disclosure). An activator can be referred to as having a tracr sequence (tracrRNA sequence) and in some cases is a tracrRNA, but the term “activator” is not limited to naturally existing tracrRNAs.

The term “targeter” or “targeter RNA” is used herein to refer to a crRNA-like molecule (crRNA: “CRISPR RNA”) of a Cas9 dual guide RNA (and therefore of a Cas9 single guide RNA when the “activator” and the “targeter” are linked together, e.g., by intervening nucleotides). Thus, for example, a Cas9 guide RNA (dgRNA or sgRNA) comprises a targeting segment (which includes nucleotides that hybridize with (are complementary to) a target nucleic acid, and a duplex-forming segment (e.g., a duplex forming segment of a crRNA, which can also be referred to as a crRNA repeat). Because the sequence of a targeting segment (the segment that hybridizes with a target sequence of a target nucleic acid) of a targeter is modified by a user to hybridize with a desired target nucleic acid, the sequence of a targeter will often be a non-naturally occurring sequence. However, the duplex-forming segment of a targeter (described in more detail below), which hybridizes with the duplex-forming segment of an activator, can include a naturally existing sequence (e.g., can include the sequence of a duplex-forming segment of a naturally existing crRNA, which can also be referred to as a crRNA repeat). Thus, the term targeter is used herein to distinguish from naturally occurring crRNAs, despite the fact that part of a targeter (e.g., the duplex-forming segment) often includes a naturally occurring sequence from a crRNA. However, the term “targeter” encompasses naturally occurring crRNAs.

A Cas9 guide RNA can also be said to include 3 parts: (i) a targeting sequence (a nucleotide sequence that hybridizes with a sequence of the target nucleic acid); (ii) an activator sequence (as described above)(in some cases, referred to as a tracr sequence); and (iii) a sequence that hybridizes to at least a portion of the activator sequence to form a double stranded duplex. A targeter has (i) and (iii); while an activator has (ii).

A Cas9 guide RNA (e.g. a dual guide RNA or a single guide RNA) can be comprised of any corresponding activator and targeter pair. In some cases, the duplex forming segments can be swapped between the activator and the targeter. In other words, in some cases, the targeter includes a sequence of nucleotides from a duplex forming segment of a tracrRNA (which sequence would normally be part of an activator) while the activator includes a sequence of nucleotides from a duplex forming segment of a crRNA (which sequence would normally be part of a targeter).

As noted above, a targeter comprises both the targeting segment (single stranded) of the Cas9 guide RNA and a stretch (“duplex-forming segment”) of nucleotides that forms one half of the dsRNA duplex of the protein-binding segment of the Cas9 guide RNA. A corresponding tracrRNA-like molecule (activator) comprises a stretch of nucleotides (a duplex-forming segment) that forms the other half of the dsRNA duplex of the protein-binding segment of the Cas9 guide RNA. In other words, a stretch of nucleotides of the targeter is complementary to and hybridizes with a stretch of nucleotides of the activator to form the dsRNA duplex of the protein-binding segment of a Cas9 guide RNA. As such, each targeter can be said to have a corresponding activator (which has a region that hybridizes with the targeter). The targeter molecule additionally provides the targeting segment. Thus, a targeter and an activator (as a corresponding pair) hybridize to form a Cas9 guide RNA. The particular sequence of a given naturally existing crRNA or tracrRNA molecule is characteristic of the species in which the RNA molecules are found. Examples of suitable activator and targeter are well known in the art.

### Targeting Segment of a Cas9 Guide RNA

The first segment of a subject guide nucleic acid includes a guide sequence (i.e., a targeting sequence)(a nucleotide sequence that is complementary to a sequence (a target site) in a target nucleic acid). In other words, the targeting segment of a subject guide nucleic acid can interact with a target nucleic acid (e.g., double stranded DNA (dsDNA)) in a sequence-specific manner via hybridization (i.e., base pairing). As such, the nucleotide sequence of the targeting segment may vary (depending on the target) and can determine the location within the target nucleic acid that the Cas9 guide RNA and the target nucleic acid will interact. The targeting segment of a Cas9 guide RNA can be modified (e.g., by genetic engineering)/designed to hybridize to any desired sequence (target site) within a target nucleic acid (e.g., a eukaryotic target nucleic acid such as genomic DNA).

The targeting segment can have a length of 7 or more nucleotides (nt) (e.g., 8 or more, 9 or more, 10 or more, 12 or more, 15 or more, 20 or more, 25 or more, 30 or more, or 40 or more nucleotides). In some cases, the targeting segment can have a length of from 7 to 100 nucleotides (nt) (e.g., from 7 to 80 nt, from 7 to 60 nt, from 7 to 40 nt, from 7 to 30 nt, from 7 to 25 nt, from 7 to 22 nt, from 7 to 20 nt, from 7 to 18 nt, from 8 to 80 nt, from 8 to 60 nt, from 8 to 40 nt, from 8 to 30 nt, from 8 to 25 nt, from 8 to 22 nt, from 8 to 20 nt, from 8 to 18 nt, from 10 to 100 nt, from 10 to 80 nt, from 10 to 60 nt, from 10 to 40 nt, from 10 to 30 nt, from 10 to 25 nt, from 10 to 22 nt, from 10 to 20 nt, from 10 to 18 nt, from 12 to 100 nt, from 12 to 80 nt, from 12 to 60 nt, from 12 to 40 nt, from 12 to 30 nt, from 12 to 25 nt, from 12 to 22 nt, from 12 to 20 nt, from 12 to 18 nt, from 14 to 100 nt, from 14 to 80 nt, from 14 to 60 nt, from 14 to 40 nt, from 14 to 30 nt, from 14 to 25 nt, from 14 to 22 nt, from 14 to 20 nt, from 14 to 18 nt, from 16 to 100 nt, from 16 to 80 nt, from 16 to 60 nt, from 16 to 40 nt, from 16 to 30 nt, from 16 to 25 nt, from 16 to 22 nt, from 16 to 20 nt, from 16 to 18 nt, from 18 to 100 nt, from 18 to 80 nt, from 18 to 60 nt, from 18 to 40 nt, from 18 to 30 nt, from 18 to 25 nt, from 18 to 22 nt, or from 18 to 20 nt).

The nucleotide sequence (the targeting sequence) of the targeting segment that is complementary to a nucleotide sequence (target site) of the target nucleic acid can have a length of 10 nt or more. For example, the targeting sequence of the targeting segment that is complementary to a target site of the target nucleic acid can have a length of 12 nt or more, 15 nt or more, 18 nt or more, 19 nt or more, or 20 nt or more. In some cases, the nucleotide sequence (the targeting sequence) of the targeting segment that is complementary to a nucleotide sequence (target site) of the target nucleic acid has a length of 12 nt or more. In some cases, the nucleotide sequence (the targeting sequence) of the targeting segment that is complementary to a nucleotide sequence (target site) of the target nucleic acid has a length of 18 nt or more.

For example, the targeting sequence of the targeting segment that is complementary to a target sequence of the target nucleic acid can have a length of from 10 to 100 nucleotides (nt) (e.g., from 10 to 90 nt, from 10 to 75 nt, from 10 to 60 nt, from 10 to 50 nt, from 10 to 35 nt, from 10 to 30 nt, from 10 to 25 nt, from 10 to 22 nt, from 10 to 20 nt, from 12 to 100 nt, from 12 to 90 nt, from 12 to 75 nt, from 12 to 60 nt, from 12 to 50 nt, from 12 to 35 nt, from 12 to 30 nt, from 12 to 25 nt, from 12 to 22 nt, from 12 to 20 nt, from 15 to 100 nt, from 15 to 90 nt, from 15 to 75 nt, from 15 to 60 nt, from 15 to 50 nt, from 15 to 35 nt, from 15 to 30 nt, from 15 to 25 nt, from 15 to 22 nt, from 15 to 20 nt, from 17 to 100 nt, from 17 to 90 nt, from 17 to 75 nt, from 17 to 60 nt, from 17 to 50 nt, from 17 to 35 nt, from 17 to 30 nt, from 17 to 25 nt, from 17 to 22 nt, from 17 to 20 nt, from 18 to 100 nt, from 18 to 90 nt, from 18 to 75 nt, from 18 to 60 nt, from 18 to 50 nt, from 18 to 35 nt, from 18 to 30 nt, from 18 to 25 nt, from 18 to 22 nt, or from 18 to 20 nt). In some cases, the targeting sequence of the targeting segment that is complementary to a target sequence of the target nucleic acid has a length of from 15 nt to 30 nt. In some cases, the targeting sequence of the targeting segment that is complementary to a target sequence of the target nucleic acid has a length of from 15 nt to 25 nt. In some cases, the targeting sequence of the targeting segment that is complementary to a target sequence of the target nucleic acid has a length of from 18 nt to 30 nt. In some cases, the targeting sequence of the targeting segment that is complementary to a target sequence of the target nucleic acid has a length of from 18 nt to 25 nt. In some cases, the targeting sequence of the targeting segment that is complementary to a target sequence of the target nucleic acid has a length of from 18 nt to 22 nt. In some cases, the targeting sequence of the targeting segment that is complementary to a target site of the target nucleic acid is 20 nucleotides in length. In some cases, the targeting sequence of the targeting segment that is complementary to a target site of the target nucleic acid is 19 nucleotides in length.

The percent complementarity between the targeting sequence (guide sequence) of the targeting segment and the target site of the target nucleic acid can be 60% or more (e.g., 65% or more, 70% or more, 75% or more, 80% or more, 85% or more, 90% or more, 95% or more, 97% or more, 98% or more, 99% or more, or 100%). In some cases, the percent complementarity between the targeting sequence of the targeting segment and the target site of the target nucleic acid is 100% over the seven contiguous 5′-most nucleotides of the target site of the target nucleic acid. In some cases, the percent complementarity between the targeting sequence of the targeting segment and the target site of the target nucleic acid is 60% or more over about 20 contiguous nucleotides. In some cases, the percent complementarity between the targeting sequence of the targeting segment and the target site of the target nucleic acid is 100% over the fourteen contiguous 5′-most nucleotides of the target site of the target nucleic acid and as low as 0% or more over the remainder. In such a case, the targeting sequence can be considered to be 14 nucleotides in length. In some cases, the percent complementarity between the targeting sequence of the targeting segment and the target site of the target nucleic acid is 100% over the seven contiguous 5′-most nucleotides of the target site of the target nucleic acid and as low as 0% or more over the remainder. In such a case, the targeting sequence can be considered to be 20 nucleotides in length.

In some cases, the percent complementarity between the targeting sequence of the targeting segment and the target site of the target nucleic acid is 100% over the 7 contiguous 5′-most nucleotides of the target site of the target nucleic acid (which can be complementary to the 3′-most nucleotides of the targeting sequence of the Cas9 guide RNA). In some cases, the percent complementarity between the targeting sequence of the targeting segment and the target site of the target nucleic acid is 100% over the 8 contiguous 5′-most nucleotides of the target site of the target nucleic acid (which can be complementary to the 3′-most nucleotides of the targeting sequence of the Cas9 guide RNA). In some cases, the percent complementarity between the targeting sequence of the targeting segment and the target site of the target nucleic acid is 100% over the 9 contiguous 5′-most nucleotides of the target site of the target nucleic acid (which can be complementary to the 3′-most nucleotides of the targeting sequence of the Cas9 guide RNA). In some cases, the percent complementarity between the targeting sequence of the targeting segment and the target site of the target nucleic acid is 100% over the 10 contiguous 5′-most nucleotides of the target site of the target nucleic acid (which can be complementary to the 3′-most nucleotides of the targeting sequence of the Cas9 guide RNA). In some cases, the percent complementarity between the targeting sequence of the targeting segment and the target site of the target nucleic acid is 100% over the 17 contiguous 5′-most nucleotides of the target site of the target nucleic acid (which can be complementary to the 3′-most nucleotides of the targeting sequence of the Cas9 guide RNA). In some cases, the percent complementarity between the targeting sequence of the targeting segment and the target site of the target nucleic acid is 100% over the 18 contiguous 5′-most nucleotides of the target site of the target nucleic acid (which can be complementary to the 3′-most nucleotides of the targeting sequence of the Cas9 guide RNA). In some cases, the percent complementarity between the targeting sequence of the targeting segment and the target site of the target nucleic acid is 60% or more (e.g., e.g., 65% or more, 70% or more, 75% or more, 80% or more, 85% or more, 90% or more, 95% or more, 97% or more, 98% or more, 99% or more, or 100%) over about 20 contiguous nucleotides.

In some cases, the percent complementarity between the targeting sequence of the targeting segment and the target site of the target nucleic acid is 100% over the 7 contiguous 5′-most nucleotides of the target site of the target nucleic acid and as low as 0% or more over the remainder. In such a case, the targeting sequence can be considered to be 7 nucleotides in length. In some cases, the percent complementarity between the targeting sequence of the targeting segment and the target site of the target nucleic acid is 100% over the 8 contiguous 5′-most nucleotides of the target site of the target nucleic acid and as low as 0% or more over the remainder. In such a case, the targeting sequence can be considered to be 8 nucleotides in length. In some cases, the percent complementarity between the targeting sequence of the targeting segment and the target site of the target nucleic acid is 100% over the 9 contiguous 5′-most nucleotides of the target site of the target nucleic acid and as low as 0% or more over the remainder. In such a case, the targeting sequence can be considered to be 9 nucleotides in length. In some cases, the percent complementarity between the targeting sequence of the targeting segment and the target site of the target nucleic acid is 100% over the 10 contiguous 5′-most nucleotides of the target site of the target nucleic acid and as low as 0% or more over the remainder. In such a case, the targeting sequence can be considered to be 10 nucleotides in length. In some cases, the percent complementarity between the targeting sequence of the targeting segment and the target site of the target nucleic acid is 100% over the 11 contiguous 5′-most nucleotides of the target site of the target nucleic acid and as low as 0% or more over the remainder. In such a case, the targeting sequence can be considered to be 11 nucleotides in length. In some cases, the percent complementarity between the targeting sequence of the targeting segment and the target site of the target nucleic acid is 100% over the 12 contiguous 5′-most nucleotides of the target site of the target nucleic acid and as low as 0% or more over the remainder. In such a case, the targeting sequence can be considered to be 12 nucleotides in length. In some cases, the percent complementarity between the targeting sequence of the targeting segment and the target site of the target nucleic acid is 100% over the 13 contiguous 5′-most nucleotides of the target site of the target nucleic acid and as low as 0% or more over the remainder. In such a case, the targeting sequence can be considered to be 13 nucleotides in length. In some cases, the percent complementarity between the targeting sequence of the targeting segment and the target site of the target nucleic acid is 100% over the 14 contiguous 5′-most nucleotides of the target site of the target nucleic acid and as low as 0% or more over the remainder. In such a case, the targeting sequence can be considered to be 14 nucleotides in length. In some cases, the percent complementarity between the targeting sequence of the targeting segment and the target site of the target nucleic acid is 100% over the 17 contiguous 5′-most nucleotides of the target site of the target nucleic acid and as low as 0% or more over the remainder. In such a case, the targeting sequence can be considered to be 17 nucleotides in length. In some cases, the percent complementarity between the targeting sequence of the targeting segment and the target site of the target nucleic acid is 100% over the 18 contiguous 5′-most nucleotides of the target site of the target nucleic acid and as low as 0% or more over the remainder. In such a case, the targeting sequence can be considered to be 18 nucleotides in length.

Examples of various Cas9 proteins and Cas9 guide RNAs (as well as information regarding requirements related to protospacer adjacent motif (PAM) sequences present in targeted nucleic acids) can be found in the art, for example, see Jinek et al., Science. 2012 Aug. 17; 337(6096):816-21; Chylinski et al., RNA Biol. 2013 May; 10(5):726-37; Ma et al., Biomed Res Int. 2013; 2013:270805; Hou et al., Proc Natl Acad Sci USA. 2013 Sep. 24; 110(39):15644-9; Jinek et al., Elife. 2013; 2:e00471; Pattanayak et al., Nat Biotechnol. 2013 September; 31(9):839-43; Qi et al., Cell. 2013 Feb. 28; 152(5):1173-83; Wang et al., Cell. 2013 May 9; 153(4):910-8; Auer et al., Genome Res. 2013 Oct. 31; Chen et al., Nucleic Acids Res. 2013 Nov. 1; 41(20):e19; Cheng et al., Cell Res. 2013 October; 23(10):1163-71; Cho et al., Genetics. 2013 November; 195(3):1177-80; DiCarlo et al., Nucleic Acids Res. 2013 April; 41(7):4336-43; Dickinson et al., Nat Methods. 2013 October; 10(10):1028-34; Ebina et al., Sci Rep. 2013; 3:2510; Fujii et al., Nucleic Acids Res. 2013 Nov. 1; 41(20):e187; Hu et al., Cell Res. 2013 November; 23(11):1322-5; Jiang et al., Nucleic Acids Res. 2013 Nov. 1; 41(20):e188; Larson et al., Nat Protoc. 2013 November; 8(11):2180-96; Mali et al., Nat Methods. 2013 October; 10(10):957-63; Nakayama et al., Genesis. 2013 December; 51(12):835-43; Ran et al., Nat Protoc. 2013 November; 8(11):2281-308; Ran et al., Cell. 2013 Sep. 12; 154(6):1380-9; Upadhyay et al., G3 (Bethesda). 2013 Dec. 9; 3(12):2233-8; Walsh et al., Proc Natl Acad Sci USA. 2013 Sep. 24; 110(39):15514-5; Xie et al., Mol Plant. 2013 Oct. 9; Yang et al., Cell. 2013 Sep. 12; 154(6):1370-9; Briner et al., Mol Cell. 2014 Oct. 23; 56(2):333-9; and U.S. patents and patent applications: U.S. Pat. Nos. 8,906,616; 8,895,308; 8,889,418; 8,889,356; 8,871,445; 8,865,406; 8,795,965; 8,771,945; 8,697,359; 20140068797; 20140170753; 20140179006; 20140179770; 20140186843; 20140186919; 20140186958; 20140189896; 20140227787; 20140234972; 20140242664; 20140242699; 20140242700; 20140242702; 20140248702; 20140256046; 20140273037; 20140273226; 20140273230; 20140273231; 20140273232; 20140273233; 20140273234; 20140273235; 20140287938; 20140295556; 20140295557; 20140298547; 20140304853; 20140309487; 20140310828; 20140310830; 20140315985; 20140335063; 20140335620; 20140342456; 20140342457; 20140342458; 20140349400; 20140349405; 20140356867; 20140356956; 20140356958; 20140356959; 20140357523; 20140357530; 20140364333; and 20140377868; all of which are hereby incorporated by reference in their entirety.

### Nucleic Acid Modifications

In some embodiments, a nucleic acid of the present disclosure (e.g., a guide RNA; e.g., a guide RNA present in a system of the present disclosure, a composition of the present disclosure, or a kit of the present disclosure) has one or more modifications, e.g., a base modification, a backbone modification, etc., to provide the nucleic acid with a new or enhanced feature (e.g., improved stability). A nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to the 2′, the 3′, or the 5′ hydroxyl moiety of the sugar. In forming oligonucleotides, the phosphate groups covalently link adjacent nucleosides to one another to form a linear polymeric compound. In turn, the respective ends of this linear polymeric compound can be further joined to form a circular compound; however, linear compounds are suitable. In addition, linear compounds may have internal nucleotide base complementarity and may therefore fold in a manner as to produce a fully or partially double-stranded compound. Within oligonucleotides, the phosphate groups are commonly referred to as forming the internucleoside backbone of the oligonucleotide. The normal linkage or backbone of RNA and DNA is a 3′ to 5′ phosphodiester linkage.

Suitable nucleic acid modifications include, but are not limited to: 2′Omethyl modified nucleotides, 2′ Fluoro modified nucleotides, locked nucleic acid (LNA) modified nucleotides, peptide nucleic acid (PNA) modified nucleotides, nucleotides with phosphorothioate linkages, and a 5′ cap (e.g., a 7-methylguanylate cap (m7G)). Additional details and additional modifications are described below.

A 2′-O-Methyl modified nucleotide (also referred to as 2′-O-Methyl RNA) is a naturally occurring modification of RNA found in tRNA and other small RNAs that arises as a post-transcriptional modification. Oligonucleotides can be directly synthesized that contain 2′-O-Methyl RNA. This modification increases Tm of RNA:RNA duplexes but results in only small changes in RNA:DNA stability. It is stabile with respect to attack by single-stranded ribonucleases and is typically 5 to 10-fold less susceptible to DNases than DNA. It is commonly used in antisense oligos as a means to increase stability and binding affinity to the target message.

2′ Fluoro modified nucleotides (e.g., 2′ Fluoro bases) have a fluorine modified ribose which increases binding affinity (Tm) and also confers some relative nuclease resistance when compared to native RNA. These modifications are commonly employed in ribozymes and siRNAs to improve stability in serum or other biological fluids.

LNA bases have a modification to the ribose backbone that locks the base in the C3′-endo position, which favors RNA A-type helix duplex geometry. This modification significantly increases Tm and is also very nuclease resistant. Multiple LNA insertions can be placed in an oligo at any position except the 3′-end. Applications have been described ranging from antisense oligos to hybridization probes to SNP detection and allele specific PCR. Due to the large increase in Tm conferred by LNAs, they also can cause an increase in primer dimer formation as well as self-hairpin formation. In some cases, the number of LNAs incorporated into a single oligo is 10 bases or less.

The phosphorothioate (PS) bond (i.e., a phosphorothioate linkage) substitutes a sulfur atom for a non-bridging oxygen in the phosphate backbone of a nucleic acid (e.g., an oligo). This modification renders the internucleotide linkage resistant to nuclease degradation. Phosphorothioate bonds can be introduced between the last 3-5 nucleotides at the 5′- or 3′-end of the oligo to inhibit exonuclease degradation. Including phosphorothioate bonds within the oligo (e.g., throughout the entire oligo) can help reduce attack by endonucleases as well.

In some embodiments, a guide RNA has one or more nucleotides that are 2′-O-Methyl modified nucleotides. In some embodiments, a guide RNA has one or more 2′ Fluoro modified nucleotides. In some embodiments, a guide RNA has one or more LNA bases. In some embodiments, a guide RNA has one or more nucleotides that are linked by a phosphorothioate bond (i.e., a guide RNA has one or more phosphorothioate linkages). In some embodiments, a guide RNA has a 5′ cap (e.g., a 7-methylguanylate cap (m7G)). In some embodiments, a guide RNA has a combination of modified nucleotides. For example, a guide RNA can have a 5′ cap (e.g., a 7-methylguanylate cap (m7G)) in addition to having one or more nucleotides with other modifications (e.g., a 2′-O-Methyl nucleotide and/or a 2′ Fluoro modified nucleotide and/or a LNA base and/or a phosphorothioate linkage).

### Modified Backbones and Modified Internucleoside Linkages

Examples of a guide RNAs containing modifications include a guide RNAs containing modified backbones or non-natural internucleoside linkages. Guide RNAs having modified backbones include those that retain a phosphorus atom in the backbone and those that do not have a phosphorus atom in the backbone.

Suitable modified oligonucleotide backbones containing a phosphorus atom therein include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, phosphorodiamidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Suitable oligonucleotides having inverted polarity comprise a single 3′ to 3′ linkage at the 3′-most internucleotide linkage i.e. a single inverted nucleoside residue which may be a basic (the nucleobase is missing or has a hydroxyl group in place thereof). Various salts (such as, for example, potassium or sodium), mixed salts and free acid forms are also included.

In some embodiments, a subject nucleic acid (e.g., a guide RNA) comprises one or more phosphorothioate and/or heteroatom internucleoside linkages, in particular —CH₂—NH—O—CH₂—, —CH₂—N(CH₃)—O—CH₂— (known as a methylene (methylimino) or MMI backbone), —CH₂—O—N(CH₃)—CH₂—, —CH₂—N(CH₃)—N(CH₃)—CH₂— and —O—N(CH₃)—CH₂—CH₂— (wherein the native phosphodiester internucleotide linkage is represented as —O—P(═O)(OH)—O—CH₂—). MMI type internucleoside linkages are disclosed in the above referenced U.S. Pat. No. 5,489,677, the disclosure of which is incorporated herein by reference in its entirety. Suitable amide internucleoside linkages are disclosed in U.S. Pat. No. 5,602,240, the disclosure of which is incorporated herein by reference in its entirety.

Also suitable are nucleic acids (e.g., a guide RNA) having morpholino backbone structures as described in, e.g., U.S. Pat. No. 5,034,506. For example, in some embodiments, a subject nucleic acid comprises a 6-membered morpholino ring in place of a ribose ring. In some of these embodiments, a phosphorodiamidate or other non-phosphodiester internucleoside linkage replaces a phosphodiester linkage.

Suitable modified polynucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

### Mimetics

A subject nucleic acid (e.g., a guide RNA) can be a nucleic acid mimetic. The term “mimetic” as it is applied to polynucleotides is intended to include polynucleotides wherein only the furanose ring or both the furanose ring and the internucleotide linkage are replaced with non-furanose groups, replacement of only the furanose ring is also referred to in the art as being a sugar surrogate. The heterocyclic base moiety or a modified heterocyclic base moiety is maintained for hybridization with an appropriate target nucleic acid. One such nucleic acid, a polynucleotide mimetic that has been shown to have excellent hybridization properties, is referred to as a peptide nucleic acid (PNA). In PNA, the sugar-backbone of a polynucleotide is replaced with an amide containing backbone, in particular an aminoethylglycine backbone. The nucleotides are retained and are bound directly or indirectly to aza nitrogen atoms of the amide portion of the backbone.

One polynucleotide mimetic that has been reported to have excellent hybridization properties is a peptide nucleic acid (PNA). The backbone in PNA compounds is two or more linked aminoethylglycine units which gives PNA an amide containing backbone. The heterocyclic base moieties are bound directly or indirectly to aza nitrogen atoms of the amide portion of the backbone. Representative U.S. patents that describe the preparation of PNA compounds include, but are not limited to: U.S. Pat. Nos. 5,539,082; 5,714,331; and 5,719,262, the disclosures of which are incorporated herein by reference in their entirety.

Another class of polynucleotide mimetic that has been studied is based on linked morpholino units (morpholino nucleic acid) having heterocyclic bases attached to the morpholino ring. A number of linking groups have been reported that link the morpholino monomeric units in a morpholino nucleic acid. One class of linking groups has been selected to give a non-ionic oligomeric compound. The non-ionic morpholino-based oligomeric compounds are less likely to have undesired interactions with cellular proteins. Morpholino-based polynucleotides are non-ionic mimics of oligonucleotides which are less likely to form undesired interactions with cellular proteins (Dwaine A. Braasch and David R. Corey, Biochemistry, 2002, 41(14), 4503-4510). Morpholino-based polynucleotides are disclosed in U.S. Pat. No. 5,034,506, the disclosure of which is incorporated herein by reference in its entirety. A variety of compounds within the morpholino class of polynucleotides have been prepared, having a variety of different linking groups joining the monomeric subunits.

A further class of polynucleotide mimetic is referred to as cyclohexenyl nucleic acids (CeNA). The furanose ring normally present in a DNA/RNA molecule is replaced with a cyclohexenyl ring. CeNA DMT protected phosphoramidite monomers have been prepared and used for oligomeric compound synthesis following classical phosphoramidite chemistry. Fully modified CeNA oligomeric compounds and oligonucleotides having specific positions modified with CeNA have been prepared and studied (see Wang et al., J. Am. Chem. Soc., 2000, 122, 8595-8602, the disclosure of which is incorporated herein by reference in its entirety). In general the incorporation of CeNA monomers into a DNA chain increases its stability of a DNA/RNA hybrid. CeNA oligoadenylates formed complexes with RNA and DNA complements with similar stability to the native complexes. The study of incorporating CeNA structures into natural nucleic acid structures was shown by NMR and circular dichroism to proceed with easy conformational adaptation.

A further modification includes Locked Nucleic Acids (LNAs) in which the 2′-hydroxyl group is linked to the 4′ carbon atom of the sugar ring thereby forming a 2′-C,4′-C-oxymethylene linkage thereby forming a bicyclic sugar moiety. The linkage can be a methylene (—CH₂—), group bridging the 2′ oxygen atom and the 4′ carbon atom wherein n is 1 or 2 (Singh et al., Chem. Commun., 1998, 4, 455-456, the disclosure of which is incorporated herein by reference in its entirety). LNA and LNA analogs display very high duplex thermal stabilities with complementary DNA and RNA (Tm=+3 to +10° C.), stability towards 3′-exonucleolytic degradation and good solubility properties. Potent and nontoxic antisense oligonucleotides containing LNAs have been described (e.g., Wahlestedt et al., Proc. Natl. Acad. Sci. U.S.A., 2000, 97, 5633-5638, the disclosure of which is incorporated herein by reference in its entirety).

The synthesis and preparation of the LNA monomers adenine, cytosine, guanine, 5-methyl-cytosine, thymine and uracil, along with their oligomerization, and nucleic acid recognition properties have been described (e.g., Koshkin et al., Tetrahedron, 1998, 54, 3607-3630, the disclosure of which is incorporated herein by reference in its entirety). LNAs and preparation thereof are also described in WO 98/39352 and WO 99/14226, as well as U.S. applications 20120165514, 20100216983, 20090041809, 20060117410, 20040014959, 20020094555, and 20020086998, the disclosures of which are incorporated herein by reference in their entirety.

### Modified Sugar Moieties

A subject nucleic acid (e.g., a guide RNA) can also include one or more substituted sugar moieties. Suitable polynucleotides comprise a sugar substituent group selected from: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly suitable are O((CH₂)ₙO)ₘCH₃, O(CH₂)ₙOCH₃, O(CH₂)ₙNH₂, O(CH₂)ₙCH₃, O(CH₂)ₙONH₂, and O(CH₂)ₙON((CH₂)ₙCH₃)₂, where n and m are from 1 to about 10. Other suitable polynucleotides comprise a sugar substituent group selected from: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A suitable modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504, the disclosure of which is incorporated herein by reference in its entirety) i.e., an alkoxyalkoxy group. A further suitable modification includes 2′-dimethylaminooxyethoxy, i.e., a O(CH₂)₂ON(CH₃)₂ group, also known as 2′-DMAOE, as described in examples hereinbelow, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃)₂.

Other suitable sugar substituent groups include methoxy (—O—CH₃), aminopropoxy CH₂CH₂CH₂NH₂), allyl (—CH₂—CH═CH₂), —O-allyl CH₂—CH═CH₂) and fluoro (F). 2′-sugar substituent groups may be in the arabino (up) position or ribo (down) position. A suitable 2′-arabino modification is 2′-F. Similar modifications may also be made at other positions on the oligomeric compound, particularly the 3′ position of the sugar on the 3′ terminal nucleoside or in 2′-5′ linked oligonucleotides and the 5′ position of 5′ terminal nucleotide. Oligomeric compounds may also have sugar mimetics such as cyclobutyl moieties in place of the pentofuranosyl sugar. Base modifications and substitutions

A subject nucleic acid (e.g., a guide RNA) may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-aminoadenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine(1H-pyrimido(5,4-b)(1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g. 9-(2-aminoethoxy)-H-pyrimido(5,4-(b) (1,4)benzoxazin-2(3H)-one), carbazole cytidine (2H-pyrimido(4,5-b)indol-2-one), pyridoindole cytidine (H-pyrido(3′,2′:4,5)pyrrolo(2,3-d)pyrimidin-2-one).

Heterocyclic base moieties may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993; the disclosures of which are incorporated herein by reference in their entirety. Certain of these nucleobases are useful for increasing the binding affinity of an oligomeric compound. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi et al., eds., Antisense Research and Applications, CRC Press, Boca Raton, 1993, pp. 276-278; the disclosure of which is incorporated herein by reference in its entirety) and are suitable base substitutions, e.g., when combined with 2′-O-methoxyethyl sugar modifications.

### Conjugates

Another possible modification of a subject nucleic acid involves chemically linking to the polynucleotide one or more moieties or conjugates which enhance the activity, cellular distribution or cellular uptake of the oligonucleotide. These moieties or conjugates can include conjugate groups covalently bound to functional groups such as primary or secondary hydroxyl groups. Conjugate groups include, but are not limited to, intercalators, reporter molecules, polyamines, polyamides, polyethylene glycols, polyethers, groups that enhance the pharmacodynamic properties of oligomers, and groups that enhance the pharmacokinetic properties of oligomers. Suitable conjugate groups include, but are not limited to, cholesterols, lipids, phospholipids, biotin, phenazine, folate, phenanthridine, anthraquinone, acridine, fluoresceins, rhodamines, coumarins, and dyes. Groups that enhance the pharmacodynamic properties include groups that improve uptake, enhance resistance to degradation, and/or strengthen sequence-specific hybridization with the target nucleic acid. Groups that enhance the pharmacokinetic properties include groups that improve uptake, distribution, metabolism or excretion of a subject nucleic acid.

Conjugate moieties include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. N.Y Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937).

A conjugate may include a “Protein Transduction Domain” or PTD (also known as a CPP—cell penetrating peptide), which may refer to a polypeptide, polynucleotide, carbohydrate, or organic or inorganic compound that facilitates traversing a lipid bilayer, micelle, cell membrane, organelle membrane, or vesicle membrane. A PTD attached to another molecule, which can range from a small polar molecule to a large macromolecule and/or a nanoparticle, facilitates the molecule traversing a membrane, for example going from extracellular space to intracellular space, or cytosol to within an organelle (e.g., the nucleus). In some embodiments, a PTD is covalently linked to the 3′ end of an exogenous polynucleotide. In some embodiments, a PTD is covalently linked to the 5′ end of an exogenous polynucleotide. Exemplary PTDs include but are not limited to a minimal undecapeptide protein transduction domain (corresponding to residues 47-57 of HIV-1 TAT comprising YGRKKRRQRRR; SEQ ID NO:854); a polyarginine sequence comprising a number of arginines sufficient to direct entry into a cell (e.g., 3, 4, 5, 6, 7, 8, 9, 10, or 10-50 arginines); a VP22 domain (Zender et al. (2002) Cancer Gene Ther. 9(6):489-96); an Drosophila antennapedia protein transduction domain (Noguchi et al. (2003) Diabetes 52(7):1732-1737); a truncated human calcitonin peptide (Trehin et al. (2004) Pharm. Research 21:1248-1256); polylysine (Wender et al. (2000) Proc. Natl. Acad. Sci. USA 97:13003-13008); RRQRRTSKLMKR SEQ ID NO:855); Transportan GWTLNSAGYLLGKINLKALAALAKKIL SEQ ID NO:856); KALAWEAKLAKALAKALAKHLAKALAKALKCEA SEQ ID NO:857); and RQIKIWFQNRRMKWKK SEQ ID NO:853). Exemplary PTDs include but are not limited to, YGRKKRRQRRR SEQ ID NO:854), RKKRRQRRR SEQ ID NO:858); an arginine homopolymer of from 3 arginine residues to 50 arginine residues; Exemplary PTD domain amino acid sequences include, but are not limited to, any of the following: YGRKKRRQRRR SEQ ID NO:854); RKKRRQRR SEQ ID NO:859); YARAAARQARA SEQ ID NO:860); THRLPRRRRRR SEQ ID NO:861); and GGRRARRRRRR SEQ ID NO:862). In some embodiments, the PTD is an activatable CPP (ACPP) (Aguilera et al. (2009) Integr Biol (Camb) June; 1(5-6): 371-381). ACPPs comprise a polycationic CPP (e.g., Arg9 or “R9”) connected via a cleavable linker to a matching polyanion (e.g., Glu9 or “E9”), which reduces the net charge to nearly zero and thereby inhibits adhesion and uptake into cells. Upon cleavage of the linker, the polyanion is released, locally unmasking the polyarginine and its inherent adhesiveness, thus “activating” the ACPP to traverse the membrane.

Introducing Components into a Target Cell

A guide RNA (or a nucleic acid comprising a nucleotide sequence encoding same) and/or an RNA-guided effector polypeptide of the present disclosure (or a nucleic acid comprising a nucleotide sequence encoding same) and/or a fusion polypeptide of the present disclosure (or a nucleic acid that includes a nucleotide sequence encoding a fusion polypeptide of the present disclosure) and/or a donor polynucleotide (donor template) can be introduced into a host cell by any of a variety of well-known methods.

Any of a variety of compounds and methods can be used to deliver to a target cell a system of the present disclosure (e.g., where a system of the present disclosure comprises: a) a an RNA-guided effector polypeptide of the present disclosure and a guide RNA; b) a fusion polypeptide of the present disclosure and a guide RNA; c) a fusion polypeptide of the present disclosure, a guide RNA, and a donor template nucleic acid; d) an mRNA encoding an RNA-guided effector polypeptide of the present disclosure; and a guide RNA; e) an mRNA encoding a fusion polypeptide of the present disclosure; and a guide RNA; f) an mRNA encoding a fusion polypeptide of the present disclosure, a guide RNA, and a donor template nucleic acid; g) a recombinant expression vector comprising a nucleotide sequence encoding an RNA-guided effector polypeptide of the present disclosure and a nucleotide sequence encoding a guide RNA; h) a recombinant expression vector comprising a nucleotide sequence encoding a fusion polypeptide of the present disclosure and a nucleotide sequence encoding a guide RNA; i) a recombinant expression vector comprising a nucleotide sequence encoding a fusion polypeptide of the present disclosure, a nucleotide sequence encoding a guide RNA, and a nucleotide sequence encoding a donor template nucleic acid; j) a first recombinant expression vector comprising a nucleotide sequence encoding an RNA-guided effector polypeptide of the present disclosure, and a second recombinant expression vector comprising a nucleotide sequence encoding a guide RNA; k) a first recombinant expression vector comprising a nucleotide sequence encoding a fusion polypeptide of the present disclosure, and a second recombinant expression vector comprising a nucleotide sequence encoding a guide RNA; l) a first recombinant expression vector comprising a nucleotide sequence encoding a fusion polypeptide of the present disclosure, and a second recombinant expression vector comprising a nucleotide sequence encoding a guide RNA; and a donor template nucleic acid; m) a recombinant expression vector comprising a nucleotide sequence encoding an RNA-guided effector polypeptide of the present disclosure, a nucleotide sequence encoding a first guide RNA, and a nucleotide sequence encoding a second guide RNA; or n) a recombinant expression vector comprising a nucleotide sequence encoding a fusion polypeptide of the present disclosure, a nucleotide sequence encoding a first guide RNA, and a nucleotide sequence encoding a second guide RNA; or some variation of one of (a) through (n). As a non-limiting example, a system of the present disclosure can be combined with a lipid. As another non-limiting example, a system of the present disclosure can be combined with a particle, or formulated into a particle.

Methods of introducing a nucleic acid into a host cell are known in the art, and any convenient method can be used to introduce a subject nucleic acid (e.g., an expression construct/vector) into a target cell (e.g., prokaryotic cell, eukaryotic cell, plant cell, animal cell, mammalian cell, human cell, and the like). Suitable methods include, e.g., viral infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery (see, e.g., Panyam et., al Adv Drug Deliv Rev. 2012 Sep. 13. pii: 50169-409X(12)00283-9. doi: 10.1016/j.addr.2012.09.023), and the like.

In some cases, an RNA-guided effector polypeptide of the present disclosure is provided as a nucleic acid (e.g., an mRNA, a DNA, a plasmid, an expression vector, a viral vector, etc.) that encodes the RNA-guided effector polypeptide. In some cases, the RNA-guided effector polypeptide of the present disclosure is provided directly as a protein (e.g., without an associated guide RNA or with an associate guide RNA, i.e., as a ribonucleoprotein complex). An RNA-guided effector polypeptide of the present disclosure can be introduced into a cell (provided to the cell) by any convenient method; such methods are known to those of ordinary skill in the art. As an illustrative example, an RNA-guided effector polypeptide of the present disclosure can be injected directly into a cell (e.g., with or without a guide RNA or nucleic acid encoding a guide RNA, and with or without a donor polynucleotide). As another example, a preformed complex of an RNA-guided effector polypeptide of the present disclosure and a guide RNA (an RNP) can be introduced into a cell (e.g., eukaryotic cell) (e.g., via injection, via nucleofection; via a protein transduction domain (PTD) conjugated to one or more components, e.g., conjugated to the RNA-guided effector polypeptide protein, conjugated to a guide RNA, conjugated to an RNA-guided effector polypeptide of the present disclosure and a guide RNA; etc.).

In some cases, a fusion polypeptide (an RNA-guided effector polypeptide fused to a heterologous fusion partner) of the present disclosure is provided as a nucleic acid (e.g., an mRNA, a DNA, a plasmid, an expression vector, a viral vector, etc.) that encodes the fusion polypeptide. In some cases, a fusion polypeptide of the present disclosure is provided directly as a protein (e.g., without an associated guide RNA or with an associate guide RNA, i.e., as a ribonucleoprotein complex). A fusion polypeptide of the present disclosure can be introduced into a cell (provided to the cell) by any convenient method; such methods are known to those of ordinary skill in the art. As an illustrative example, a fusion polypeptide of the present disclosure can be injected directly into a cell (e.g., with or without nucleic acid encoding a guide RNA and with or without a donor polynucleotide). As another example, a preformed complex of a fusion polypeptide of the present disclosure and a guide RNA (an RNP) can be introduced into a cell (e.g., via injection, via nucleofection; via a protein transduction domain (PTD) conjugated to one or more components, e.g., conjugated to the fusion protein, conjugated to a guide RNA, conjugated to a fusion polypeptide of the present disclosure and a guide RNA; etc.).

In some cases, a nucleic acid (e.g., a guide RNA; a nucleic acid comprising a nucleotide sequence encoding an RNA-guided effector polypeptide of the present disclosure; a nucleic acid comprising a nucleotide sequence encoding a fusion polypeptide of the present disclosure; etc.) is delivered to a cell (e.g., a target host cell) and/or a polypeptide (e.g., an RNA-guided effector polypeptide; a fusion polypeptide) in a particle, or associated with a particle. In some cases, a system of the present disclosure is delivered to a cell in a particle, or associated with a particle. The terms “particle” and “nanoparticle” can be used interchangeable, as appropriate. A recombinant expression vector comprising a nucleotide sequence encoding an RNA-guided effector polypeptide of the present disclosure or a fusion polypeptide of the present disclosure and/or a guide RNA, an mRNA comprising a nucleotide sequence encoding an RNA-guided effector polypeptide of the present disclosure or a fusion polypeptide of the present disclosure, and guide RNA may be delivered simultaneously using particles or lipid envelopes; for instance, an RNA-guided effector polypeptide of the present disclosure polypeptide and a guide RNA, or a fusion polypeptide of the present disclosure and a guide RNA, e.g., as a complex (e.g., a ribonucleoprotein (RNP) complex), can be delivered via a particle, e.g., a delivery particle comprising lipid or lipidoid and hydrophilic polymer, e.g., a cationic lipid and a hydrophilic polymer, for instance wherein the cationic lipid comprises 1,2-dioleoyl-3-trimethylammonium-propane (DOTAP) or 1,2-ditetradecanoyl-sn-glycero-3-phosphocholine (DMPC) and/or wherein the hydrophilic polymer comprises ethylene glycol or polyethylene glycol (PEG); and/or wherein the particle further comprises cholesterol (e.g., particle from formulation 1=DOTAP 100, DMPC 0, PEG 0, Cholesterol 0; formulation number 2=DOTAP 90, DMPC 0, PEG 10, Cholesterol 0; formulation number 3=DOTAP 90, DMPC 0, PEG 5, Cholesterol 5). For example, a particle can be formed using a multistep process in which an RNA-guided effector polypeptide of the present disclosure and a guide RNA, or a fusion polypeptide of the present disclosure and a guide RNA, are mixed together, e.g., at a 1:1 molar ratio, e.g., at room temperature, e.g., for 30 minutes, e.g., in sterile, nuclease free 1× phosphate-buffered saline (PBS); and separately, DOTAP, DMPC, PEG, and cholesterol as applicable for the formulation are dissolved in alcohol, e.g., 100% ethanol; and, the two solutions are mixed together to form particles containing the complexes).

A RNA-guided effector polypeptide of the present disclosure polypeptide (or an mRNA comprising a nucleotide sequence encoding an RNA-guided effector polypeptide of the present disclosure polypeptide; or a recombinant expression vector comprising a nucleotide sequence encoding an RNA-guided effector polypeptide of the present disclosure polypeptide) and/or guide RNA (or a nucleic acid such as one or more expression vectors encoding the guide RNA), or a fusion polypeptide of the present disclosure (or an mRNA comprising a nucleotide sequence encoding a fusion polypeptide of the present disclosure; or a recombinant expression vector comprising a nucleotide sequence encoding a fusion polypeptide of the present disclosure polypeptide) and/or a guide RNA may be delivered simultaneously using particles or lipid envelopes. For example, a biodegradable core-shell structured nanoparticle with a poly (β-amino ester) (PBAE) core enveloped by a phospholipid bilayer shell can be used. In some cases, particles/nanoparticles based on self assembling bioadhesive polymers are used; such particles/nanoparticles may be applied to oral delivery of peptides, intravenous delivery of peptides and nasal delivery of peptides, e.g., to the brain. Other embodiments, such as oral absorption and ocular delivery of hydrophobic drugs are also contemplated. A molecular envelope technology, which involves an engineered polymer envelope which is protected and delivered to the site of the disease, can be used. Doses of about 5 mg/kg can be used, with single or multiple doses, depending on various factors, e.g., the target tissue.

Lipidoid compounds (e.g., as described in US patent application 20110293703) are also useful in the administration of polynucleotides, and can be used to deliver an RNA-guided effector polypeptide of the present disclosure, a fusion polypeptide of the present disclosure, an RNP of the present disclosure, a nucleic acid of the present disclosure, or a system of the present disclosure (e.g., where a system of the present disclosure comprises: a) a an RNA-guided effector polypeptide of the present disclosure and a guide RNA; b) a fusion polypeptide of the present disclosure and a guide RNA; c) a fusion polypeptide of the present disclosure, a guide RNA, and a donor template nucleic acid; d) an mRNA encoding an RNA-guided effector polypeptide of the present disclosure; and a guide RNA; e) an mRNA encoding a fusion polypeptide of the present disclosure; and a guide RNA; f) an mRNA encoding a fusion polypeptide of the present disclosure, a guide RNA, and a donor template nucleic acid; g) a recombinant expression vector comprising a nucleotide sequence encoding an RNA-guided effector polypeptide of the present disclosure and a nucleotide sequence encoding a guide RNA; h) a recombinant expression vector comprising a nucleotide sequence encoding a fusion polypeptide of the present disclosure and a nucleotide sequence encoding a guide RNA; i) a recombinant expression vector comprising a nucleotide sequence encoding a fusion polypeptide of the present disclosure, a nucleotide sequence encoding a guide RNA, and a nucleotide sequence encoding a donor template nucleic acid; j) a first recombinant expression vector comprising a nucleotide sequence encoding an RNA-guided effector polypeptide of the present disclosure, and a second recombinant expression vector comprising a nucleotide sequence encoding a guide RNA; k) a first recombinant expression vector comprising a nucleotide sequence encoding a fusion polypeptide of the present disclosure, and a second recombinant expression vector comprising a nucleotide sequence encoding a guide RNA; l) a first recombinant expression vector comprising a nucleotide sequence encoding a fusion polypeptide of the present disclosure, and a second recombinant expression vector comprising a nucleotide sequence encoding a guide RNA; and a donor template nucleic acid; m) a recombinant expression vector comprising a nucleotide sequence encoding an RNA-guided effector polypeptide of the present disclosure, a nucleotide sequence encoding a first guide RNA, and a nucleotide sequence encoding a second guide RNA; or n) a recombinant expression vector comprising a nucleotide sequence encoding a fusion polypeptide of the present disclosure, a nucleotide sequence encoding a first guide RNA, and a nucleotide sequence encoding a second guide RNA; or some variation of one of (a) through (n). In one aspect, the aminoalcohol lipidoid compounds are combined with an agent to be delivered to a cell or a subject to form microparticles, nanoparticles, liposomes, or micelles. The aminoalcohol lipidoid compounds may be combined with other aminoalcohol lipidoid compounds, polymers (synthetic or natural), surfactants, cholesterol, carbohydrates, proteins, lipids, etc. to form the particles. These particles may then optionally be combined with a pharmaceutical excipient to form a pharmaceutical composition.

A poly(beta-amino alcohol) (PBAA) can be used to deliver an RNA-guided effector polypeptide of the present disclosure, a fusion polypeptide of the present disclosure, an RNP of the present disclosure, a nucleic acid of the present disclosure, or a system of the present disclosure, to a target cell. US Patent Publication No. 20130302401 relates to a class of poly(beta-amino alcohols) (PBAAs) that has been prepared using combinatorial polymerization.

Sugar-based particles may be used, for example GalNAc, as described with reference to WO2014118272 (incorporated herein by reference) and Nair, J K et al., 2014, Journal of the American Chemical Society 136 (49), 16958-16961) can be used to deliver an RNA-guided effector polypeptide of the present disclosure, a fusion polypeptide of the present disclosure, an RNP of the present disclosure, a nucleic acid of the present disclosure, or a system of the present disclosure, to a target cell.

In some cases, lipid nanoparticles (LNPs) are used to deliver an RNA-guided effector polypeptide of the present disclosure, a fusion polypeptide of the present disclosure, an RNP of the present disclosure, a nucleic acid of the present disclosure, or a system of the present disclosure, to a target cell. Negatively charged polymers such as RNA may be loaded into LNPs at low pH values (e.g., pH 4) where the ionizable lipids display a positive charge. However, at physiological pH values, the LNPs exhibit a low surface charge compatible with longer circulation times. Four species of ionizable cationic lipids have been focused upon, namely 1,2-dilineoyl-3-dimethylammonium-propane (DLinDAP), 1,2-dilinoleyloxy-3-N,N-dimethylaminopropane (DLinDMA), 1,2-dilinoleyloxy-keto-N,N-dimethyl-3-aminopropane (DLinKDMA), and 1,2-dilinoleyl-4-(2-dimethylaminoethyl)[1,3]-dioxolane (DLinKC2-DMA). Preparation of LNPs and is described in, e.g., Rosin et al. (2011) Molecular Therapy 19:1286-2200). The cationic lipids 1,2-dilineoyl-3-dimethylammonium-propane (DLinDAP), 1,2-dilinoleyloxy-3-N,N-dimethylaminopropane (DLinDMA), 1,2-dilinoleyloxyketo-N,N-dimethyl-3-aminopropane (DLinK-DMA), 1,2-dilinoleyl-4-(2-dimethylaminoethyl)[1,3]-dioxolane (DLinKC2-DMA), (3-o-[2″-(methoxypolyethyleneglycol 2000) succinoyl]-1,2-dimyristoyl-sn-glycol (PEG-S-DMG), and R-3-[(omega-methoxy-poly(ethylene glycol)2000) carbamoyl]-1,2-dimyristyloxlpropyl-3-amine (PEG-C-DOMG) may be used. A nucleic acid (e.g., a guide RNA; a nucleic acid of the present disclosure; etc.) may be encapsulated in LNPs containing DLinDAP, DLinDMA, DLinK-DMA, and DLinKC2-DMA (cationic lipid:DSPC:CHOL:PEGS-DMG or PEG-C-DOMG at 40:10:40:10 molar ratios). In some cases, 0.2% SP-DiOC18 is incorporated.

Spherical Nucleic Acid (SNA™) constructs and other nanoparticles (particularly gold nanoparticles) can be used to deliver an RNA-guided effector polypeptide of the present disclosure, a fusion polypeptide of the present disclosure, an RNP of the present disclosure, a nucleic acid of the present disclosure, or a system of the present disclosure, to a target cell. See, e.g., Cutler et al., J. Am. Chem. Soc. 2011 133:9254-9257, Hao et al., Small. 2011 7:3158-3162, Zhang et al., ACS Nano. 2011 5:6962-6970, Cutler et al., J. Am. Chem. Soc. 2012 134:1376-1391, Young et al., Nano Lett. 2012 12:3867-71, Zheng et al., Proc. Natl. Acad. Sci. USA. 2012 109:11975-80, Mirkin, Nanomedicine 2012 7:635-638 Zhang et al., J. Am. Chem. Soc. 2012 134:16488-1691, Weintraub, Nature 2013 495:S14-S16, Choi et al., Proc. Natl. Acad. Sci. USA. 2013 110(19): 7625-7630, Jensen et al., Sci. Transl. Med. 5, 209ra152 (2013) and Mirkin, et al., Small, 10:186-192.

Self-assembling nanoparticles with RNA may be constructed with polyethyleneimine (PEI) that is PEGylated with an Arg-Gly-Asp (RGD) peptide ligand attached at the distal end of the polyethylene glycol (PEG).

In general, a “nanoparticle” refers to any particle having a diameter of less than 1000 nm. In some cases, nanoparticles suitable for use in delivering an RNA-guided effector polypeptide of the present disclosure, a fusion polypeptide of the present disclosure, an RNP of the present disclosure, a nucleic acid of the present disclosure, or a system of the present disclosure, to a target cell have a diameter of 500 nm or less, e.g., from 25 nm to 35 nm, from 35 nm to 50 nm, from 50 nm to 75 nm, from 75 nm to 100 nm, from 100 nm to 150 nm, from 150 nm to 200 nm, from 200 nm to 300 nm, from 300 nm to 400 nm, or from 400 nm to 500 nm. In some cases, nanoparticles suitable for use in delivering an RNA-guided effector polypeptide of the present disclosure, a fusion polypeptide of the present disclosure, an RNP of the present disclosure, a nucleic acid of the present disclosure, or a system of the present disclosure, to a target cell have a diameter of from 25 nm to 200 nm. In some cases, nanoparticles suitable for use in delivering an RNA-guided effector polypeptide of the present disclosure, a fusion polypeptide of the present disclosure, an RNP of the present disclosure, a nucleic acid of the present disclosure, or a system of the present disclosure, to a target cell have a diameter of 100 nm or less In some cases, nanoparticles suitable for use in delivering an RNA-guided effector polypeptide of the present disclosure, a fusion polypeptide of the present disclosure, an RNP of the present disclosure, a nucleic acid of the present disclosure, or a system of the present disclosure, to a target cell have a diameter of from 35 nm to 60 nm.

Nanoparticles suitable for use in delivering an RNA-guided effector polypeptide of the present disclosure, a fusion polypeptide of the present disclosure, an RNP of the present disclosure, a nucleic acid of the present disclosure, or a system of the present disclosure, to a target cell may be provided in different forms, e.g., as solid nanoparticles (e.g., metal such as silver, gold, iron, titanium), non-metal, lipid-based solids, polymers), suspensions of nanoparticles, or combinations thereof. Metal, dielectric, and semiconductor nanoparticles may be prepared, as well as hybrid structures (e.g., core-shell nanoparticles). Nanoparticles made of semiconducting material may also be labeled quantum dots if they are small enough (typically below 10 nm) that quantization of electronic energy levels occurs. Such nanoscale particles are used in biomedical applications as drug carriers or imaging agents and may be adapted for similar purposes in the present disclosure.

Semi-solid and soft nanoparticles are also suitable for use in delivering polypeptide of the present disclosure, a fusion polypeptide of the present disclosure, an RNP of the present disclosure, a nucleic acid of the present disclosure, or a system of the present disclosure, to a target cell. A prototype nanoparticle of semi-solid nature is the liposome.

In some cases, an exosome is used to deliver an RNA-guided effector polypeptide of the present disclosure, a fusion polypeptide of the present disclosure, an RNP of the present disclosure, a nucleic acid of the present disclosure, or a system of the present disclosure, to a target cell. Exosomes are endogenous nano-vesicles that transport RNAs and proteins, and which can deliver RNA to the brain and other target organs.

In some cases, a liposome is used to deliver an RNA-guided effector polypeptide of the present disclosure, a fusion polypeptide of the present disclosure, an RNP of the present disclosure, a nucleic acid of the present disclosure, or a system of the present disclosure, to a target cell. Liposomes are spherical vesicle structures composed of a uni- or multilamellar lipid bilayer surrounding internal aqueous compartments and a relatively impermeable outer lipophilic phospholipid bilayer. Liposomes can be made from several different types of lipids; however, phospholipids are most commonly used to generate liposomes. Although liposome formation is spontaneous when a lipid film is mixed with an aqueous solution, it can also be expedited by applying force in the form of shaking by using a homogenizer, sonicator, or an extrusion apparatus. Several other additives may be added to liposomes in order to modify their structure and properties. For instance, either cholesterol or sphingomyelin may be added to the liposomal mixture in order to help stabilize the liposomal structure and to prevent the leakage of the liposomal inner cargo. A liposome formulation may be mainly comprised of natural phospholipids and lipids such as 1,2-distearoryl-sn-glycero-3-phosphatidyl choline (DSPC), sphingomyelin, egg phosphatidylcholines and monosialoganglioside.

A stable nucleic-acid-lipid particle (SNALP) can be used to deliver an RNA-guided effector polypeptide of the present disclosure, a fusion polypeptide of the present disclosure, an RNP of the present disclosure, a nucleic acid of the present disclosure, or a system of the present disclosure, to a target cell. The SNALP formulation may contain the lipids 3-N-Rmethoxypoly(ethylene glycol) 2000) carbamoyl]-1,2-dimyristyloxy-propylamine (PEG-C-DMA), 1,2-dilinoleyloxy-N,N-dimethyl-3-aminopropane (DLinDMA), 1,2-distearoyl-sn-glycero-3-phosphocholine (DSPC) and cholesterol, in a 2:40:10:48 molar percent ratio. The SNALP liposomes may be prepared by formulating D-Lin-DMA and PEG-C-DMA with distearoylphosphatidylcholine (DSPC), Cholesterol and siRNA using a 25:1 lipid/siRNA ratio and a 48/40/10/2 molar ratio of Cholesterol/D-Lin-DMA/DSPC/PEG-C-DMA. The resulting SNALP liposomes can be about 80-100 nm in size. A SNALP may comprise synthetic cholesterol (Sigma-Aldrich, St Louis, Mo., USA), dipalmitoylphosphatidylcholine (Avanti Polar Lipids, Alabaster, Ala., USA), 3-N-[(w-methoxy poly(ethylene glycol)2000)carbamoyl]-1,2-dimyrestyloxypropylamine, and cationic 1,2-dilinoleyloxy-3-N,N-dimethylaminopropane. A SNALP may comprise synthetic cholesterol (Sigma-Aldrich), 1,2-distearoyl-sn-glycero-3-phosphocholine (DSPC; Avanti Polar Lipids Inc.), PEG-cDMA, and 1,2-dilinoleyloxy-3-(N;N-dimethyl)aminopropane (DLinDMA).

Other cationic lipids, such as amino lipid 2,2-dilinoleyl-4-dimethylaminoethyl-[1,3]-dioxolane (DLin-KC2-DMA) can be used to deliver an RNA-guided effector polypeptide of the present disclosure, a fusion polypeptide of the present disclosure, an RNP of the present disclosure, a nucleic acid of the present disclosure, or a system of the present disclosure, to a target cell. A preformed vesicle with the following lipid composition may be contemplated: amino lipid, distearoylphosphatidylcholine (DSPC), cholesterol and (R)-2,3-bis(octadecyloxy) propyl-1-(methoxy poly(ethylene glycol)2000)propylcarbamate (PEG-lipid) in the molar ratio 40/10/40/10, respectively, and a FVII siRNA/total lipid ratio of approximately 0.05 (w/w). To ensure a narrow particle size distribution in the range of 70-90 nm and a low polydispersity index of 0.11.+−.0.04 (n=56), the particles may be extruded up to three times through 80 nm membranes prior to adding the guide RNA. Particles containing the highly potent amino lipid 16 may be used, in which the molar ratio of the four lipid components 16, DSPC, cholesterol and PEG-lipid (50/10/38.5/1.5) which may be further optimized to enhance in vivo activity.

Lipids may be formulated with a system of the present disclosure or component(s) thereof or nucleic acids encoding the same to form lipid nanoparticles (LNPs). Suitable lipids include, but are not limited to, DLin-KC2-DMA4, C12-200 and colipids disteroylphosphatidyl choline, cholesterol, and PEG-DMG may be formulated with a system, or component thereof, of the present disclosure, using a spontaneous vesicle formation procedure. The component molar ratio may be about 50/10/38.5/1.5 (DLin-KC2-DMA or C12-200/disteroylphosphatidyl choline/cholesterol/PEG-DMG).

A system of the present disclosure, or a component thereof, may be delivered encapsulated in PLGA microspheres such as that further described in US published applications 20130252281 and 20130245107 and 20130244279.

Supercharged proteins can be used to deliver an RNA-guided effector polypeptide of the present disclosure, a fusion polypeptide of the present disclosure, an RNP of the present disclosure, a nucleic acid of the present disclosure, or a system of the present disclosure, to a target cell. Supercharged proteins are a class of engineered or naturally occurring proteins with unusually high positive or negative net theoretical charge. Both supernegatively and superpositively charged proteins exhibit the ability to withstand thermally or chemically induced aggregation. Superpositively charged proteins are also able to penetrate mammalian cells. Associating cargo with these proteins, such as plasmid DNA, RNA, or other proteins, can facilitate the functional delivery of these macromolecules into mammalian cells both in vitro and in vivo.

Cell Penetrating Peptides (CPPs) can be used to deliver an RNA-guided effector polypeptide of the present disclosure, a fusion polypeptide of the present disclosure, an RNP of the present disclosure, a nucleic acid of the present disclosure, or a system of the present disclosure, to a target cell. CPPs typically have an amino acid composition that either contains a high relative abundance of positively charged amino acids such as lysine or arginine or has sequences that contain an alternating pattern of polar/charged amino acids and non-polar, hydrophobic amino acids.

An implantable device can be used to deliver an RNA-guided effector polypeptide of the present disclosure, a fusion polypeptide of the present disclosure, an RNP of the present disclosure, a nucleic acid of the present disclosure (e.g., a guide RNA, a nucleic acid encoding a guide RNA, a nucleic acid encoding an RNA-guided effector polypeptide, a donor template, and the like), or a system of the present disclosure, to a target cell (e.g., a target cell in vivo, where the target cell is a target cell in circulation, a target cell in a tissue, a target cell in an organ, etc.). An implantable device suitable for use in delivering an RNA-guided effector polypeptide of the present disclosure, a fusion polypeptide of the present disclosure, an RNP of the present disclosure, a nucleic acid of the present disclosure, or a system of the present disclosure, to a target cell (e.g., a target cell in vivo, where the target cell is a target cell in circulation, a target cell in a tissue, a target cell in an organ, etc.) can include a container (e.g., a reservoir, a matrix, etc.) that comprises the RNA-guided effector polypeptide, the fusion polypeptide, the RNP, or the system (or component thereof, e.g., a nucleic acid of the present disclosure).

A suitable implantable device can comprise a polymeric substrate, such as a matrix for example, that is used as the device body, and in some cases additional scaffolding materials, such as metals or additional polymers, and materials to enhance visibility and imaging. An implantable delivery device can be advantageous in providing release locally and over a prolonged period, where the polypeptide and/or nucleic acid to be delivered is released directly to a target site, e.g., the extracellular matrix (ECM), the vasculature surrounding a tumor, a diseased tissue, etc. Suitable implantable delivery devices include devices suitable for use in delivering to a cavity such as the abdominal cavity and/or any other type of administration in which the drug delivery system is not anchored or attached, comprising a biostable and/or degradable and/or bioabsorbable polymeric substrate, which may for example optionally be a matrix. In some cases, a suitable implantable drug delivery device comprises degradable polymers, wherein the main release mechanism is bulk erosion. In some cases, a suitable implantable drug delivery device comprises non degradable, or slowly degraded polymers, wherein the main release mechanism is diffusion rather than bulk erosion, so that the outer part functions as membrane, and its internal part functions as a drug reservoir, which practically is not affected by the surroundings for an extended period (for example from about a week to about a few months). Combinations of different polymers with different release mechanisms may also optionally be used. The concentration gradient at the can be maintained effectively constant during a significant period of the total releasing period, and therefore the diffusion rate is effectively constant (termed “zero mode” diffusion). By the term “constant” it is meant a diffusion rate that is maintained above the lower threshold of therapeutic effectiveness, but which may still optionally feature an initial burst and/or may fluctuate, for example increasing and decreasing to a certain degree. The diffusion rate can be so maintained for a prolonged period, and it can be considered constant to a certain level to optimize the therapeutically effective period, for example the effective silencing period.

In some cases, the implantable delivery system is designed to shield the nucleotide based therapeutic agent from degradation, whether chemical in nature or due to attack from enzymes and other factors in the body of the subject.

The site for implantation of the device, or target site, can be selected for maximum therapeutic efficacy. For example, a delivery device can be implanted within or in the proximity of a tumor environment, or the blood supply associated with a tumor. The target location can be, e.g.: 1) the brain at degenerative sites like in Parkinson or Alzheimer disease at the basal ganglia, white and gray matter; 2) the spine, as in the case of amyotrophic lateral sclerosis (ALS); 3) uterine cervix; 4) active and chronic inflammatory joints; 5) dermis as in the case of psoriasis; 7) sympathetic and sensoric nervous sites for analgesic effect; 7) a bone; 8) a site of acute or chronic infection; 9) Intra vaginal; 10) Inner ear—auditory system, labyrinth of the inner ear, vestibular system; 11) Intra tracheal; 12) Intra-cardiac; coronary, epicardiac; 13) urinary tract or bladder; 14) biliary system; 15) parenchymal tissue including and not limited to the kidney, liver, spleen; 16) lymph nodes; 17) salivary glands; 18) dental gums; 19) Intra-articular (into joints); 20) Intra-ocular; 21) Brain tissue; 22) Brain ventricles; 23) Cavities, including abdominal cavity (for example but without limitation, for ovary cancer); 24) Intra esophageal; and 25) Intra rectal; and 26) into the vasculature.

The method of insertion, such as implantation, may optionally already be used for other types of tissue implantation and/or for insertions and/or for sampling tissues, optionally without modifications, or alternatively optionally only with non-major modifications in such methods. Such methods optionally include but are not limited to brachytherapy methods, biopsy, endoscopy with and/or without ultrasound, such as stereotactic methods into the brain tissue, laparoscopy, including implantation with a laparoscope into joints, abdominal organs, the bladder wall and body cavities.

### Modified Host Cells

The present disclosure provides a modified cell comprising an RNA-guided effector polypeptide of the present disclosure and/or a nucleic acid comprising a nucleotide sequence encoding an RNA-guided effector polypeptide of the present disclosure. The present disclosure provides a modified cell comprising an RNA-guided effector polypeptide of the present disclosure, where the modified cell is a cell that does not normally comprise the RNA-guided effector polypeptide. The present disclosure provides a modified cell (e.g., a genetically modified cell) comprising nucleic acid comprising a nucleotide sequence encoding an RNA-guided effector polypeptide of the present disclosure. The present disclosure provides a genetically modified cell that is genetically modified with an mRNA comprising a nucleotide sequence encoding an RNA-guided effector polypeptide of the present disclosure. The present disclosure provides a genetically modified cell that is genetically modified with a recombinant expression vector comprising a nucleotide sequence encoding an RNA-guided effector polypeptide of the present disclosure. The present disclosure provides a genetically modified cell that is genetically modified with a recombinant expression vector comprising: a) a nucleotide sequence encoding an RNA-guided effector polypeptide of the present disclosure; and b) a nucleotide sequence encoding a guide RNA of the present disclosure. The present disclosure provides a genetically modified cell that is genetically modified with a recombinant expression vector comprising: a) a nucleotide sequence encoding an RNA-guided effector polypeptide of the present disclosure; b) a nucleotide sequence encoding a guide RNA of the present disclosure; and c) a nucleotide sequence encoding a donor template.

The present disclosure provides a modified cell comprising a fusion polypeptide of the present disclosure and/or a nucleic acid comprising a nucleotide sequence encoding a fusion polypeptide of the present disclosure. The present disclosure provides a modified cell comprising a fusion polypeptide of the present disclosure, where the modified cell is a cell that does not normally comprise the fusion polypeptide. The present disclosure provides a modified cell (e.g., a genetically modified cell) comprising nucleic acid comprising a nucleotide sequence encoding a fusion polypeptide of the present disclosure. The present disclosure provides a genetically modified cell that is genetically modified with an mRNA comprising a nucleotide sequence encoding a fusion polypeptide of the present disclosure. The present disclosure provides a genetically modified cell that is genetically modified with a recombinant expression vector comprising a nucleotide sequence encoding a fusion polypeptide of the present disclosure. The present disclosure provides a genetically modified cell that is genetically modified with a recombinant expression vector comprising: a) a nucleotide sequence encoding a fusion polypeptide of the present disclosure; and b) a nucleotide sequence encoding a guide RNA of the present disclosure. The present disclosure provides a genetically modified cell that is genetically modified with a recombinant expression vector comprising: a) a nucleotide sequence encoding a fusion polypeptide of the present disclosure; b) a nucleotide sequence encoding a guide RNA of the present disclosure; and c) a nucleotide sequence encoding a donor template.

A cell that serves as a recipient for an RNA-guided effector polypeptide of the present disclosure and/or a nucleic acid comprising a nucleotide sequence encoding an RNA-guided effector polypeptide of the present disclosure and/or a guide RNA of the present disclosure, or that serves as a recipient for a fusion polypeptide of the present disclosure and/or a nucleic acid comprising a nucleotide sequence encoding a fusion polypeptide of the present disclosure and/or a guide RNA of the present disclosure, can be any of a variety of cells, including, e.g., in vitro cells; in vivo cells; ex vivo cells; primary cells; cancer cells; animal cells; plant cells; algal cells; fungal cells; etc. A cell that serves as a recipient for an RNA-guided effector polypeptide of the present disclosure and/or a nucleic acid comprising a nucleotide sequence encoding an RNA-guided effector polypeptide of the present disclosure and/or a guide RNA of the present disclosure, or that serves as a recipient for a fusion polypeptide of the present disclosure and/or a nucleic acid comprising a nucleotide sequence encoding a fusion polypeptide of the present disclosure and/or a guide RNA of the present disclosure, is referred to as a “host cell” or a “target cell.” A host cell or a target cell can be a recipient of a system of the present disclosure. A host cell or a target cell can be a recipient of an RNP of the present disclosure. A host cell or a target cell can be a recipient of a single component of a system of the present disclosure.

Non-limiting examples of cells (target cells) include: a prokaryotic cell, eukaryotic cell, a bacterial cell, an archaeal cell, a cell of a single-cell eukaryotic organism, a protozoa cell, a cell from a plant (e.g., cells from plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, angiosperms, ferns, clubmosses, hornworts, liverworts, mosses, dicotyledons, monocotyledons, etc.), an algal cell, (e.g., Botryococcus braunii, Chlamydomonas reinhardtii, Nannochloropsis gaditana, Chlorella pyrenoidosa, Sargassum patens, C. agardh, and the like), seaweeds (e.g. kelp) a fungal cell (e.g., a yeast cell, a cell from a mushroom), an animal cell, a cell from an invertebrate animal (e.g., fruit fly, cnidarian, echinoderm, nematode, etc.), a cell from a vertebrate animal (e.g., fish, amphibian, reptile, bird, mammal), a cell from a mammal (e.g., an ungulate (e.g., a pig, a cow, a goat, a sheep); a rodent (e.g., a rat, a mouse); a non-human primate; a human; a feline (e.g., a cat); a canine (e.g., a dog); etc.), and the like. In some cases, the cell is a cell that does not originate from a natural organism (e.g., the cell can be a synthetically made cell; also referred to as an artificial cell).

A cell can be an in vitro cell (e.g., established cultured cell line). A cell can be an ex vivo cell (cultured cell from an individual). A cell can be and in vivo cell (e.g., a cell in an individual). A cell can be an isolated cell. A cell can be a cell inside of an organism. A cell can be an organism. A cell can be a cell in a cell culture (e.g., in vitro cell culture). A cell can be one of a collection of cells. A cell can be a prokaryotic cell or derived from a prokaryotic cell. A cell can be a bacterial cell or can be derived from a bacterial cell. A cell can be an archaeal cell or derived from an archaeal cell. A cell can be a eukaryotic cell or derived from a eukaryotic cell. A cell can be a plant cell or derived from a plant cell. A cell can be an animal cell or derived from an animal cell. A cell can be an invertebrate cell or derived from an invertebrate cell. A cell can be a vertebrate cell or derived from a vertebrate cell. A cell can be a mammalian cell or derived from a mammalian cell. A cell can be a rodent cell or derived from a rodent cell. A cell can be a human cell or derived from a human cell. A cell can be a microbe cell or derived from a microbe cell. A cell can be a fungi cell or derived from a fungi cell. A cell can be an insect cell. A cell can be an arthropod cell. A cell can be a protozoan cell. A cell can be a helminth cell.

Suitable cells include a stem cell (e.g. an embryonic stem (ES) cell, an induced pluripotent stem (iPS) cell; a germ cell (e.g., an oocyte, a sperm, an oogonia, a spermatogonia, etc.); a somatic cell, e.g. a fibroblast, an oligodendrocyte, a glial cell, a hematopoietic cell, a neuron, a muscle cell, a bone cell, a hepatocyte, a pancreatic cell, etc.

Suitable cells include human embryonic stem cells, fetal cardiomyocytes, myofibroblasts, mesenchymal stem cells cardiomyocytes, adipocytes, totipotent cells, pluripotent cells, blood stem cells, myoblasts, adult stem cells, bone marrow cells, mesenchymal cells, embryonic stem cells, parenchymal cells, epithelial cells, endothelial cells, mesothelial cells, fibroblasts, osteoblasts, chondrocytes, exogenous cells, endogenous cells, stem cells, hematopoietic stem cells, bone-marrow derived progenitor cells, myocardial cells, skeletal cells, fetal cells, undifferentiated cells, multi-potent progenitor cells, unipotent progenitor cells, monocytes, cardiac myoblasts, skeletal myoblasts, macrophages, capillary endothelial cells, xenogenic cells, allogenic cells, and post-natal stem cells.

In some cases, the cell is an immune cell, a neuron, an epithelial cell, and endothelial cell, or a stem cell. In some cases, the immune cell is a T cell, a B cell, a monocyte, a natural killer cell, a dendritic cell, or a macrophage. In some cases, the immune cell is a cytotoxic T cell. In some cases, the immune cell is a helper T cell. In some cases, the immune cell is a regulatory T cell (Treg). In some cases, the immune cell is a T effector cell.

In some cases, the cell is a stem cell. Stem cells include adult stem cells. Adult stem cells are also referred to as somatic stem cells.

Adult stem cells are resident in differentiated tissue, but retain the properties of self-renewal and ability to give rise to multiple cell types, usually cell types typical of the tissue in which the stem cells are found. Numerous examples of somatic stem cells are known to those of skill in the art, including muscle stem cells; hematopoietic stem cells; epithelial stem cells; neural stem cells; mesenchymal stem cells; mammary stem cells; intestinal stem cells; mesodermal stem cells; endothelial stem cells; olfactory stem cells; neural crest stem cells; and the like.

Stem cells of interest include mammalian stem cells, where the term “mammalian” refers to any animal classified as a mammal, including humans; non-human primates; domestic and farm animals; and zoo, laboratory, sports, or pet animals, such as dogs, horses, cats, cows, mice, rats, rabbits, etc. In some cases, the stem cell is a human stem cell. In some cases, the stem cell is a rodent (e.g., a mouse; a rat) stem cell. In some cases, the stem cell is a non-human primate stem cell.

Stem cells can express one or more stem cell markers, e.g., SOX9, KRT19, KRT7, LGR5, CA9, FXYD2, CDH6, CLDN18, TSPAN8, BPIFB1, OLFM4, CDH17, and PPARGC1A.

In some embodiments, the stem cell is a hematopoietic stem cell (HSC). HSCs are mesoderm-derived cells that can be isolated from bone marrow, blood, cord blood, fetal liver and yolk sac. HSCs are characterized as CD34⁺ and CD3⁻. HSCs can repopulate the erythroid, neutrophil-macrophage, megakaryocyte and lymphoid hematopoietic cell lineages in vivo. In vitro, HSCs can be induced to undergo at least some self-renewing cell divisions and can be induced to differentiate to the same lineages as is seen in vivo. As such, HSCs can be induced to differentiate into one or more of erythroid cells, megakaryocytes, neutrophils, macrophages, and lymphoid cells.

In other embodiments, the stem cell is a neural stem cell (NSC). Neural stem cells (NSCs) are capable of differentiating into neurons, and glia (including oligodendrocytes, and astrocytes). A neural stem cell is a multipotent stem cell which is capable of multiple divisions, and under specific conditions can produce daughter cells which are neural stem cells, or neural progenitor cells that can be neuroblasts or glioblasts, e.g., cells committed to become one or more types of neurons and glial cells respectively. Methods of obtaining NSCs are known in the art.

In other embodiments, the stem cell is a mesenchymal stem cell (MSC). MSCs originally derived from the embryonal mesoderm and isolated from adult bone marrow, can differentiate to form muscle, bone, cartilage, fat, marrow stroma, and tendon. Methods of isolating MSC are known in the art; and any known method can be used to obtain MSC. See, e.g., U.S. Pat. No. 5,736,396, which describes isolation of human MSC.

A cell is in some cases a plant cell. A plant cell can be a cell of a monocotyledon. A cell can be a cell of a dicotyledon.

In some cases, the cell is a plant cell. For example, the cell can be a cell of a major agricultural plant, e.g., Barley, Beans (Dry Edible), Canola, Corn, Cotton (Pima), Cotton (Upland), Flaxseed, Hay (Alfalfa), Hay (Non-Alfalfa), Oats, Peanuts, Rice, Sorghum, Soybeans, Sugarbeets, Sugarcane, Sunflowers (Oil), Sunflowers (Non-Oil), Sweet Potatoes, Tobacco (Burley), Tobacco (Flue-cured), Tomatoes, Wheat (Durum), Wheat (Spring), Wheat (Winter), and the like. As another example, the cell is a cell of a vegetable crops which include but are not limited to, e.g., alfalfa sprouts, aloe leaves, arrow root, arrowhead, artichokes, asparagus, bamboo shoots, banana flowers, bean sprouts, beans, beet tops, beets, bittermelon, bok choy, broccoli, broccoli rabe (rappini), brussels sprouts, cabbage, cabbage sprouts, cactus leaf (nopales), calabaza, cardoon, carrots, cauliflower, celery, chayote, chinese artichoke (crosnes), chinese cabbage, chinese celery, chinese chives, choy sum, chrysanthemum leaves (tung ho), collard greens, corn stalks, corn-sweet, cucumbers, daikon, dandelion greens, dasheen, dau mue (pea tips), donqua (winter melon), eggplant, endive, escarole, fiddle head ferns, field cress, frisee, gai choy (chinese mustard), gailon, galanga (siam, thai ginger), garlic, ginger root, gobo, greens, hanover salad greens, huauzontle, jerusalem artichokes, jicama, kale greens, kohlrabi, lamb's quarters (quilete), lettuce (bibb), lettuce (boston), lettuce (boston red), lettuce (green leaf), lettuce (iceberg), lettuce (lolla rossa), lettuce (oak leaf—green), lettuce (oak leaf—red), lettuce (processed), lettuce (red leaf), lettuce (romaine), lettuce (ruby romaine), lettuce (russian red mustard), linkok, lo bok, long beans, lotus root, mache, maguey (agave) leaves, malanga, mesculin mix, mizuna, moap (smooth luffa), moo, moqua (fuzzy squash), mushrooms, mustard, nagaimo, okra, ong choy, onions green, opo (long squash), ornamental corn, ornamental gourds, parsley, parsnips, peas, peppers (bell type), peppers, pumpkins, radicchio, radish sprouts, radishes, rape greens, rape greens, rhubarb, romaine (baby red), rutabagas, salicornia (sea bean), sinqua (angled/ridged luffa), spinach, squash, straw bales, sugarcane, sweet potatoes, swiss chard, tamarindo, taro, taro leaf, taro shoots, tatsoi, tepeguaje (guaje), tindora, tomatillos, tomatoes, tomatoes (cherry), tomatoes (grape type), tomatoes (plum type), tumeric, turnip tops greens, turnips, water chestnuts, yampi, yams (names), yu choy, yuca (cassava), and the like.

A cell is in some cases an arthropod cell. For example, the cell can be a cell of a sub-order, a family, a sub-family, a group, a sub-group, or a species of, e.g., Chelicerata, Myriapodia, Hexipodia, Arachnida, Insecta, Archaeognatha, Thysanura, Palaeoptera, Ephemeroptera, Odonata, Anisoptera, Zygoptera, Neoptera, Exopter ygota, Plecoptera, Embioptera, Orthoptera, Zoraptera, Dermaptera, Dictyoptera, Notoptera, Grylloblattidae, Mantophasmatidae, Phasmatodea, Blattaria, Isoptera, Mantodea, Parapneuroptera, Psocoptera, Thysanoptera, Phthiraptera, Hemiptera, Endopterygota or Holometabola, Hymenoptera, Coleoptera, Strepsiptera, Raphidioptera, Megaloptera, Neuroptera, Mecoptera, Siphonaptera, Diptera, Trichoptera, or Lepidoptera.

A cell is in some cases an insect cell. For example, in some cases, the cell is a cell of a mosquito, a grasshopper, a true bug, a fly, a flea, a bee, a wasp, an ant, a louse, a moth, or a beetle.

### Kits

The present disclosure provides a kit comprising a system of the present disclosure, or a component of a system of the present disclosure.

A kit of the present disclosure can comprise: a) a an RNA-guided effector polypeptide of the present disclosure and a guide RNA; b) a fusion polypeptide of the present disclosure and a guide RNA; c) a fusion polypeptide of the present disclosure, a guide RNA, and a donor template nucleic acid; d) an mRNA encoding an RNA-guided effector polypeptide of the present disclosure; and a guide RNA; e) an mRNA encoding a fusion polypeptide of the present disclosure; and a guide RNA; f) an mRNA encoding a fusion polypeptide of the present disclosure, a guide RNA, and a donor template nucleic acid; g) a recombinant expression vector comprising a nucleotide sequence encoding an RNA-guided effector polypeptide of the present disclosure and a nucleotide sequence encoding a guide RNA; h) a recombinant expression vector comprising a nucleotide sequence encoding a fusion polypeptide of the present disclosure and a nucleotide sequence encoding a guide RNA; i) a recombinant expression vector comprising a nucleotide sequence encoding a fusion polypeptide of the present disclosure, a nucleotide sequence encoding a guide RNA, and a nucleotide sequence encoding a donor template nucleic acid; j) a first recombinant expression vector comprising a nucleotide sequence encoding an RNA-guided effector polypeptide of the present disclosure, and a second recombinant expression vector comprising a nucleotide sequence encoding a guide RNA; k) a first recombinant expression vector comprising a nucleotide sequence encoding a fusion polypeptide of the present disclosure, and a second recombinant expression vector comprising a nucleotide sequence encoding a guide RNA; l) a first recombinant expression vector comprising a nucleotide sequence encoding a fusion polypeptide of the present disclosure, and a second recombinant expression vector comprising a nucleotide sequence encoding a guide RNA; and a donor template nucleic acid; m) a recombinant expression vector comprising a nucleotide sequence encoding an RNA-guided effector polypeptide of the present disclosure, a nucleotide sequence encoding a first guide RNA, and a nucleotide sequence encoding a second guide RNA; or n) a recombinant expression vector comprising a nucleotide sequence encoding a fusion polypeptide of the present disclosure, a nucleotide sequence encoding a first guide RNA, and a nucleotide sequence encoding a second guide RNA; or some variation of one of (a) through (n).

In some cases, a kit of the present disclosure comprises: a) a RNA-guided effector polypeptide of the present disclosure, or the fusion polypeptide of the present disclosure; and b) a guide RNA, or a nucleic acid encoding the guide RNA, wherein the guide RNA comprises a guide sequence that is complementary to a target sequence of a target nucleic acid, and comprises a region that can bind to the RNA-guided effector polypeptide. In some cases, the guide RNA is a single-molecule guide RNA. In some cases, the RNA-guided effector polypeptide and the guide RNA are in separate containers. In some cases, the RNA-guided effector polypeptide and the guide RNA are together in a single container. In some cases, the guide RNA comprises one or more of: a modified nucleobase, a modified backbone or non-natural internucleoside linkage, a modified sugar moiety, a Locked Nucleic Acid, a Peptide Nucleic Acid, and a deoxyribonucleotide. In some cases, the kit further comprises a donor nucleic acid template.

A kit of the present disclosure can comprise: a) a component, as described above, of a system of the present disclosure, or can comprise a system of the present disclosure; and b) one or more additional reagents, e.g., i) a buffer; ii) a protease inhibitor; iii) a nuclease inhibitor; iv) a reagent required to develop or visualize a detectable label; v) a positive and/or negative control target DNA; vi) a positive and/or negative control guide RNA; and the like. A kit of the present disclosure can comprise: a) a component, as described above, of a system of the present disclosure, or can comprise a system of the present disclosure; and b) a therapeutic agent.

A kit of the present disclosure can comprise a recombinant expression vector comprising: a) an insertion site for inserting a nucleic acid comprising a nucleotide sequence encoding a portion of a guide RNA that hybridizes to a target nucleotide sequence in a target nucleic acid; and b) a nucleotide sequence encoding the RNA-guided effector protein-binding portion of a guide RNA. A kit of the present disclosure can comprise a recombinant expression vector comprising: a) an insertion site for inserting a nucleic acid comprising a nucleotide sequence encoding a portion of a guide RNA that hybridizes to a target nucleotide sequence in a target nucleic acid; b) a nucleotide sequence encoding the RNA-guided effector protein-binding portion of a guide RNA; and c) a nucleotide sequence encoding RNA-guided effector polypeptide of the present disclosure.

A kit of the present disclosure can comprise a recombinant expression vector comprising: i) a nucleotide sequence encoding an RNA-guided effector polypeptide of the present disclosure; and ii) an insertion site for a nucleic acid comprising a nucleotide sequence encoding a heterologous fusion partner of interest. The insertion site can be located relative to the nucleotide sequence encoding the RNA-guided effector polypeptide, such that, after insertion of a nucleic acid comprising a nucleotide sequence encoding a heterologous fusion partner of interest, the resulting fusion polypeptide would comprise, in order from N-terminus to C-terminus: i) the RNA-guided effector polypeptide; and ii) the fusion partner. The insertion site can be located relative to the nucleotide sequence encoding the RNA-guided effector polypeptide, such that, after insertion of a nucleic acid comprising a nucleotide sequence encoding a heterologous fusion partner of interest, the resulting fusion polypeptide would comprise, in order from N-terminus to C-terminus: i) the fusion partner; and ii) the RNA-guided effector polypeptide. A kit of the present disclosure can comprise a recombinant expression vector comprising: i) a nucleotide sequence encoding an RNA-guided effector polypeptide of the present disclosure; ii) an insertion site for a nucleic acid comprising a nucleotide sequence encoding a heterologous fusion partner of interest; iii) an insertion site for inserting a nucleic acid comprising a nucleotide sequence encoding a portion of a guide RNA that hybridizes to a target nucleotide sequence in a target nucleic acid; and iv) a nucleotide sequence encoding the RNA-guided effector protein-binding portion of a guide RNA

### Utility

An RNA-guided effector polypeptide of the present disclosure, or a fusion polypeptide of the present disclosure, finds use in a variety of methods (e.g., in combination with a guide RNA and in some cases further in combination with a donor template). An RNA-guided effector polypeptide of the present disclosure can be used to bind to a target nucleic acid. For example, a fusion polypeptide of the present disclosure can be used to (i) modify (e.g., cleave, e.g., nick; methylate; etc.) target nucleic acid (DNA or RNA; single stranded or double stranded); (ii) modulate transcription of a target nucleic acid; (iii) label a target nucleic acid; (iv) bind a target nucleic acid (e.g., for purposes of isolation, labeling, imaging, tracking, etc.); (v) modify a polypeptide (e.g., a histone) associated with a target nucleic acid; and the like. Thus, the present disclosure provides a method of modifying a target nucleic acid. In some cases, a method of the present disclosure for modifying a target nucleic acid comprises contacting the target nucleic acid with: a) a fusion polypeptide of the present disclosure; and b) one or more (e.g., two) guide RNAs. In some cases, a method of the present disclosure for modifying a target nucleic acid comprises contacting the target nucleic acid with: a) a fusion polypeptide of the present disclosure; b) a guide RNA; and c) a donor nucleic acid (e.g., a donor template). In some cases, the contacting step is carried out in a cell in vitro. In some cases, the contacting step is carried out in a cell in vivo. In some cases, the contacting step is carried out in a cell ex vivo.

In some cases, a method of the present disclosure is a method of binding to a target nucleic acid. Because a method that uses an RNA-guided effector polypeptide includes binding of the RNA-guided effector polypeptide to a particular region in a target nucleic acid (by virtue of being targeted there by an associated guide RNA), the methods are generally referred to herein as methods of binding (e.g., a method of binding a target nucleic acid). However, it is to be understood that in some cases, while a method of binding may result in nothing more than binding of the target nucleic acid, in other cases, the method can have different final results (e.g., the method can result in modification of the target nucleic acid, e.g., cleavage/methylation/etc., modulation of transcription from the target nucleic acid; modulation of translation of the target nucleic acid; genome editing; modulation of a protein associated with the target nucleic acid; isolation of the target nucleic acid; etc.).

The present disclosure provides a method of binding and/or modifying a target nucleic acid and/or modifying a polypeptide that binds to a target nucleic acid, the method comprising contacting the target nucleic acid with: i) an RNA-guided effector polypeptide of the present disclosure; or ii) a fusion polypeptide of the present disclosure. In some cases, the target nucleic acid is selected from: double stranded DNA, single stranded DNA, RNA, genomic DNA, and extrachromosomal DNA. In some cases, the contacting results in genome editing. In some cases, the contacting takes place outside of a bacterial cell and outside of an archaeal cell. In some cases, the contacting takes place in vitro outside of a cell. In some cases, the contacting takes place inside of a target cell. In some cases, the target cell is a eukaryotic cell. In some cases, the target cell is in vivo. In some cases, the target cell is ex vivo. In some cases, the eukaryotic cell is selected from the group consisting of: a plant cell, a fungal cell, a single cell eukaryotic organism, a mammalian cell, a reptile cell, an insect cell, an avian cell, a fish cell, a parasite cell, an arthropod cell, a cell of an invertebrate, a cell of a vertebrate, a rodent cell, a mouse cell, a rat cell, a primate cell, a non-human primate cell, and a human cell. In some cases, the method further comprises introducing a DNA donor template into the target cell.

For examples of suitable methods, see, for example, Jinek et al., Science. 2012 Aug. 17; 337(6096):816-21; Chylinski et al., RNA Biol. 2013 May; 10(5):726-37; Ma et al., Biomed Res Int. 2013; 2013:270805; Hou et al., Proc Natl Acad Sci USA. 2013 Sep. 24; 110(39):15644-9; Jinek et al., Elife. 2013; 2:e00471; Pattanayak et al., Nat Biotechnol. 2013 September; 31(9):839-43; Qi et al, Cell. 2013 Feb. 28; 152(5):1173-83; Wang et al., Cell. 2013 May 9; 153(4):910-8; Auer et al., Genome Res. 2013 Oct. 31; Chen et al., Nucleic Acids Res. 2013 Nov. 1; 41(20):e19; Cheng et al., Cell Res. 2013 October; 23(10):1163-71; Cho et al., Genetics. 2013 November; 195(3):1177-80; DiCarlo et al., Nucleic Acids Res. 2013 April; 41(7):4336-43; Dickinson et al., Nat Methods. 2013 October; 10(10):1028-34; Ebina et al., Sci Rep. 2013; 3:2510; Fujii et al, Nucleic Acids Res. 2013 Nov. 1; 41(20):e187; Hu et al., Cell Res. 2013 November; 23(11):1322-5; Jiang et al., Nucleic Acids Res. 2013 Nov. 1; 41(20):e188; Larson et al., Nat Protoc. 2013 November; 8(11):2180-96; Mali et. at., Nat Methods. 2013 October; 10(10):957-63; Nakayama et al., Genesis. 2013 December; 51(12):835-43; Ran et al., Nat Protoc. 2013 November; 8(11):2281-308; Ran et al., Cell. 2013 Sep. 12; 154(6):1380-9; Upadhyay et al., G3 (Bethesda). 2013 Dec. 9; 3(12):2233-8; Walsh et al., Proc Natl Acad Sci USA. 2013 Sep. 24; 110(39):15514-5; Xie et al., Mol Plant. 2013 Oct. 9; Yang et al., Cell. 2013 Sep. 12; 154(6):1370-9; and U.S. patents and patent applications: U.S. Pat. Nos. 8,906,616; 8,895,308; 8,889,418; 8,889,356; 8,871,445; 8,865,406; 8,795,965; 8,771,945; 8,697,359; 20140068797; 20140170753; 20140179006; 20140179770; 20140186843; 20140186919; 20140186958; 20140189896; 20140227787; 20140234972; 20140242664; 20140242699; 20140242700; 20140242702; 20140248702; 20140256046; 20140273037; 20140273226; 20140273230; 20140273231; 20140273232; 20140273233; 20140273234; 20140273235; 20140287938; 20140295556; 20140295557; 20140298547; 20140304853; 20140309487; 20140310828; 20140310830; 20140315985; 20140335063; 20140335620; 20140342456; 20140342457; 20140342458; 20140349400; 20140349405; 20140356867; 20140356956; 20140356958; 20140356959; 20140357523; 20140357530; 20140364333; and 20140377868; each of which is hereby incorporated by reference in its entirety.

For example, where a method of the present disclosure comprises contacting a target nucleic acid with a fusion polypeptide of the present disclosure complexed with a guide RNA, the present disclosure provides (but is not limited to) methods of cleaving a target nucleic acid; methods of editing a target nucleic acid; methods of modulating transcription from a target nucleic acid; methods of isolating a target nucleic acid, methods of binding a target nucleic acid, methods of imaging a target nucleic acid, methods of modifying a target nucleic acid, and the like.

As used herein, the terms/phrases “contact a target nucleic acid” and “contacting a target nucleic acid”, for example, with an RNA-guided effector polypeptide or with a fusion polypeptide, etc., encompass all methods for contacting the target nucleic acid. For example, an RNA-guided effector polypeptide can be provided to a cell as protein, RNA (encoding the RNA-guided effector polypeptide), or DNA (encoding the RNA-guided effector polypeptide); while a guide RNA can be provided as a guide RNA or as a nucleic acid encoding the guide RNA. As another example, a fusion polypeptide can be provided to a cell as protein, RNA (encoding the fusion polypeptide), or DNA (encoding the fusion polypeptide); while a guide RNA can be provided as a guide RNA or as a nucleic acid encoding the guide RNA. As such, when, for example, performing a method in a cell (e.g., inside of a cell in vitro, inside of a cell in vivo, inside of a cell ex vivo), a method that includes contacting the target nucleic acid encompasses the introduction into the cell of any or all of the components in their active/final state (e.g., in the form of a protein(s) for an RNA-guided effector polypeptide; in the form of a protein for a fusion polypeptide; in the form of an RNA in some cases for the guide RNA), and also encompasses the introduction into the cell of one or more nucleic acids encoding one or more of the components (e.g., nucleic acid(s) comprising nucleotide sequence(s) encoding RNA-guided effector polypeptide or a fusion polypeptide, nucleic acid(s) comprising nucleotide sequence(s) encoding guide RNA(s), nucleic acid comprising a nucleotide sequence encoding a donor template, and the like). Because the methods can also be performed in vitro outside of a cell, a method that includes contacting a target nucleic acid, (unless otherwise specified) encompasses contacting outside of a cell in vitro, inside of a cell in vitro, inside of a cell in vivo, inside of a cell ex vivo, etc.

In some cases, a method of the present disclosure for modifying a target nucleic acid comprises contacting a target nucleic acid with RNA-guided effector polypeptide of the present disclosure, or with a fusion polypeptide of the present disclosure. In some cases, a method of the present disclosure for modifying a target nucleic acid comprises contacting the target nucleic acid with a fusion polypeptide of the present disclosure and a guide RNA. In some cases, a method of the present disclosure for binding a target nucleic acid comprises contacting the target nucleic acid with an RNA-guided effector polypeptide of the present disclosure, and a guide RNA.

### Target Nucleic Acids and Target Cells of Interest

An RNA-guided effector polypeptide of the present disclosure, or a fusion polypeptide of the present disclosure, when bound to a guide RNA, can bind to a target nucleic acid (e.g., in the case of an RNA-guided effector polypeptide of the present disclosure), and in some cases, can bind to and modify a target nucleic acid (e.g., in the case of a fusion polypeptide of the present disclosure). A target nucleic acid can be any nucleic acid (e.g., DNA, RNA), can be double stranded or single stranded, can be any type of nucleic acid (e.g., a chromosome (genomic DNA), derived from a chromosome, chromosomal DNA, plasmid, viral, extracellular, intracellular, mitochondrial, chloroplast, linear, circular, etc.) and can be from any organism (e.g., as long as the guide RNA comprises a nucleotide sequence that hybridizes to a target sequence in a target nucleic acid, such that the target nucleic acid can be targeted).

A target nucleic acid can be DNA or RNA. A target nucleic acid can be double stranded (e.g., dsDNA, dsRNA) or single stranded (e.g., ssRNA, ssDNA). In some cases, a target nucleic acid is single stranded. In some cases, a target nucleic acid is a single stranded RNA (ssRNA). In some cases, a target ssRNA (e.g., a target cell ssRNA, a viral ssRNA, etc.) is selected from: mRNA, rRNA, tRNA, non-coding RNA (ncRNA), long non-coding RNA (lncRNA), and microRNA (miRNA). In some cases, a target nucleic acid is a single stranded DNA (ssDNA) (e.g., a viral DNA). As noted above, in some cases, a target nucleic acid is single stranded.

A target nucleic acid can be located anywhere, for example, outside of a cell in vitro, inside of a cell in vitro, inside of a cell in vivo, inside of a cell ex vivo. Suitable target cells (which can comprise target nucleic acids such as genomic DNA) include, but are not limited to: a bacterial cell; an archaeal cell; a cell of a single-cell eukaryotic organism; a plant cell; an algal cell, e.g., Botryococcus braunii, Chlamydomonas reinhardtii, Nannochloropsis gaditana, Chlorella pyrenoidosa, Sargassum patens, C. agardh, and the like; a fungal cell (e.g., a yeast cell); an animal cell; a cell from an invertebrate animal (e.g. fruit fly, a cnidarian, an echinoderm, a nematode, etc.); a cell of an insect (e.g., a mosquito; a bee; an agricultural pest; etc.); a cell of an arachnid (e.g., a spider; a tick; etc.); a cell from a vertebrate animal (e.g., a fish, an amphibian, a reptile, a bird, a mammal); a cell from a mammal (e.g., a cell from a rodent; a cell from a human; a cell of a non-human mammal; a cell of a rodent (e.g., a mouse, a rat); a cell of a lagomorph (e.g., a rabbit); a cell of an ungulate (e.g., a cow, a horse, a camel, a llama, a vicuña, a sheep, a goat, etc.); a cell of a marine mammal (e.g., a whale, a seal, an elephant seal, a dolphin, a sea lion; etc.) and the like. Any type of cell may be of interest (e.g. a stem cell, e.g. an embryonic stem (ES) cell, an induced pluripotent stem (iPS) cell, a germ cell (e.g., an oocyte, a sperm, an oogonia, a spermatogonia, etc.), an adult stem cell, a somatic cell, e.g. a fibroblast, a hematopoietic cell, a neuron, a muscle cell, a bone cell, a hepatocyte, a pancreatic cell; an in vitro or in vivo embryonic cell of an embryo at any stage, e.g., a 1-cell, 2-cell, 4-cell, 8-cell, etc. stage zebrafish embryo; etc.).

Cells may be from established cell lines or they may be primary cells, where “primary cells”, “primary cell lines”, and “primary cultures” are used interchangeably herein to refer to cells and cells cultures that have been derived from a subject and allowed to grow in vitro for a limited number of passages, i.e. splittings, of the culture. For example, primary cultures are cultures that may have been passaged 0 times, 1 time, 2 times, 4 times, 5 times, 10 times, or 15 times, but not enough times go through the crisis stage. Typically, the primary cell lines are maintained for fewer than 10 passages in vitro. Target cells can be unicellular organisms and/or can be grown in culture. If the cells are primary cells, they may be harvest from an individual by any convenient method. For example, leukocytes may be conveniently harvested by apheresis, leukocytapheresis, density gradient separation, etc., while cells from tissues such as skin, muscle, bone marrow, spleen, liver, pancreas, lung, intestine, stomach, etc. can be conveniently harvested by biopsy.

In some of the above applications, the subject methods may be employed to induce target nucleic acid cleavage, target nucleic acid modification, and/or to bind target nucleic acids (e.g., for visualization, for collecting and/or analyzing, etc.) in mitotic or post-mitotic cells in vivo and/or ex vivo and/or in vitro (e.g., to disrupt production of a protein encoded by a targeted mRNA, to cleave or otherwise modify target DNA, to genetically modify a target cell, and the like). Because the guide RNA provides specificity by hybridizing to target nucleic acid, a mitotic and/or post-mitotic cell of interest in the disclosed methods may include a cell from any organism (e.g. a bacterial cell, an archaeal cell, a cell of a single-cell eukaryotic organism, a plant cell, an algal cell, e.g., Botryococcus braunii, Chlamydomonas reinhardtii, Nannochloropsis gaditana, Chlorella pyrenoidosa, Sargassum patens, C. agardh, and the like, a fungal cell (e.g., a yeast cell), an animal cell, a cell from an invertebrate animal (e.g. fruit fly, cnidarian, echinoderm, nematode, etc.), a cell from a vertebrate animal (e.g., fish, amphibian, reptile, bird, mammal), a cell from a mammal, a cell from a rodent, a cell from a human, etc.). In some cases, a subject RNA-guided effector protein (and/or nucleic acid encoding the protein such as DNA and/or RNA), and/or guide RNA (and/or a DNA encoding the guide RNA), and/or donor template, or a fusion polypeptide of the present disclosure (or a nucleic acid encoding the fusion polypeptide) and/or guide RNA, and/or RNP and/or system can be introduced into an individual (i.e., the target cell can be in vivo) (e.g., a mammal, a rat, a mouse, a pig, a primate, a non-human primate, a human, etc.). In some case, such an administration can be for the purpose of treating and/or preventing a disease, e.g., by editing the genome of targeted cells.

Plant cells include cells of a monocotyledon, and cells of a dicotyledon. The cells can be root cells, leaf cells, cells of the xylem, cells of the phloem, cells of the cambium, apical meristem cells, parenchyma cells, collenchyma cells, sclerenchyma cells, and the like. Plant cells include cells of agricultural crops such as wheat, corn, rice, sorghum, millet, soybean, etc. Plant cells include cells of agricultural fruit and nut plants, e.g., plant that produce apricots, oranges, lemons, apples, plums, pears, almonds, etc.

Additional examples of target cells are listed above in the section titled “Modified cells.” Non-limiting examples of cells (target cells) include: a prokaryotic cell, eukaryotic cell, a bacterial cell, an archaeal cell, a cell of a single-cell eukaryotic organism, a protozoa cell, a cell from a plant (e.g., cells from plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, angiosperms, ferns, clubmosses, hornworts, liverworts, mosses, dicotyledons, monocotyledons, etc.), an algal cell, (e.g., Botryococcus braunii, Chlamydomonas reinhardtii, Nannochloropsis gaditana, Chlorella pyrenoidosa, Sargassum patens, C. agardh, and the like), seaweeds (e.g. kelp) a fungal cell (e.g., a yeast cell, a cell from a mushroom), an animal cell, a cell from an invertebrate animal (e.g., fruit fly, cnidarian, echinoderm, nematode, etc.), a cell from a vertebrate animal (e.g., fish, amphibian, reptile, bird, mammal), a cell from a mammal (e.g., an ungulate (e.g., a pig, a cow, a goat, a sheep); a rodent (e.g., a rat, a mouse); a non-human primate; a human; a feline (e.g., a cat); a canine (e.g., a dog); etc.), and the like. In some cases, the cell is a cell that does not originate from a natural organism (e.g., the cell can be a synthetically made cell; also referred to as an artificial cell).

A cell can be an in vitro cell (e.g., established cultured cell line). A cell can be an ex vivo cell (cultured cell from an individual). A cell can be an in vivo cell (e.g., a cell in an individual). A cell can be an isolated cell. A cell can be a cell inside of an organism. A cell can be an organism. A cell can be a cell in a cell culture (e.g., in vitro cell culture). A cell can be one of a collection of cells. A cell can be a prokaryotic cell or derived from a prokaryotic cell. A cell can be a bacterial cell or can be derived from a bacterial cell. A cell can be an archaeal cell or derived from an archaeal cell. A cell can be a eukaryotic cell or derived from a eukaryotic cell. A cell can be a plant cell or derived from a plant cell. A cell can be an animal cell or derived from an animal cell. A cell can be an invertebrate cell or derived from an invertebrate cell. A cell can be a vertebrate cell or derived from a vertebrate cell. A cell can be a mammalian cell or derived from a mammalian cell. A cell can be a rodent cell or derived from a rodent cell. A cell can be a human cell or derived from a human cell. A cell can be a microbe cell or derived from a microbe cell. A cell can be a fungi cell or derived from a fungi cell. A cell can be an insect cell. A cell can be an arthropod cell. A cell can be a protozoan cell. A cell can be a helminth cell.

Suitable cells include a stem cell (e.g. an embryonic stem (ES) cell, an induced pluripotent stem (iPS) cell; a germ cell (e.g., an oocyte, a sperm, an oogonia, a spermatogonia, etc.); a somatic cell, e.g. a fibroblast, an oligodendrocyte, a glial cell, a hematopoietic cell, a neuron, a muscle cell, a bone cell, a hepatocyte, a pancreatic cell, etc.

Suitable cells include human embryonic stem cells, fetal cardiomyocytes, myofibroblasts, mesenchymal stem cells, cardiomyocytes, adipocytes, totipotent cells, pluripotent cells, blood stem cells, myoblasts, adult stem cells, bone marrow cells, mesenchymal cells, embryonic stem cells, parenchymal cells, epithelial cells, endothelial cells, mesothelial cells, fibroblasts, osteoblasts, chondrocytes, exogenous cells, endogenous cells, stem cells, hematopoietic stem cells, bone-marrow derived progenitor cells, myocardial cells, skeletal cells, fetal cells, undifferentiated cells, multi-potent progenitor cells, unipotent progenitor cells, monocytes, cardiac myoblasts, skeletal myoblasts, macrophages, capillary endothelial cells, xenogenic cells, allogenic cells, and post-natal stem cells.

In some cases, the cell is an immune cell, a neuron, an epithelial cell, and endothelial cell, or a stem cell. In some cases, the immune cell is a T cell, a B cell, a monocyte, a natural killer cell, a dendritic cell, or a macrophage. In some cases, the immune cell is a cytotoxic T cell. In some cases, the immune cell is a helper T cell. In some cases, the immune cell is a regulatory T cell (Treg).

In some cases, the cell is a stem cell. Stem cells include adult stem cells. Adult stem cells are also referred to as somatic stem cells.

Adult stem cells are resident in differentiated tissue, but retain the properties of self-renewal and ability to give rise to multiple cell types, usually cell types typical of the tissue in which the stem cells are found. Numerous examples of somatic stem cells are known to those of skill in the art, including muscle stem cells; hematopoietic stem cells; epithelial stem cells; neural stem cells; mesenchymal stem cells; mammary stem cells; intestinal stem cells; mesodermal stem cells; endothelial stem cells; olfactory stem cells; neural crest stem cells; and the like.

Stem cells of interest include mammalian stem cells, where the term “mammalian” refers to any animal classified as a mammal, including humans; non-human primates; domestic and farm animals; and zoo, laboratory, sports, or pet animals, such as dogs, horses, cats, cows, mice, rats, rabbits, etc. In some cases, the stem cell is a human stem cell. In some cases, the stem cell is a rodent (e.g., a mouse; a rat) stem cell. In some cases, the stem cell is a non-human primate stem cell.

Stem cells can express one or more stem cell markers, e.g., SOX9, KRT19, KRT7, LGR5, CA9, FXYD2, CDH6, CLDN18, TSPAN8, BPIFB1, OLFM4, CDH17, and PPARGC1A.

In some embodiments, the stem cell is a hematopoietic stem cell (HSC). HSCs are mesoderm-derived cells that can be isolated from bone marrow, blood, cord blood, fetal liver and yolk sac. HSCs are characterized as CD34+ and CD3-. HSCs can repopulate the erythroid, neutrophil-macrophage, megakaryocyte and lymphoid hematopoietic cell lineages in vivo. In vitro, HSCs can be induced to undergo at least some self-renewing cell divisions and can be induced to differentiate to the same lineages as is seen in vivo. As such, HSCs can be induced to differentiate into one or more of erythroid cells, megakaryocytes, neutrophils, macrophages, and lymphoid cells.

In other embodiments, the stem cell is a neural stem cell (NSC). Neural stem cells (NSCs) are capable of differentiating into neurons, and glia (including oligodendrocytes, and astrocytes). A neural stem cell is a multipotent stem cell which is capable of multiple divisions, and under specific conditions can produce daughter cells which are neural stem cells, or neural progenitor cells that can be neuroblasts or glioblasts, e.g., cells committed to become one or more types of neurons and glial cells respectively. Methods of obtaining NSCs are known in the art.

In other embodiments, the stem cell is a mesenchymal stem cell (MSC). MSCs originally derived from the embryonal mesoderm and isolated from adult bone marrow, can differentiate to form muscle, bone, cartilage, fat, marrow stroma, and tendon. Methods of isolating MSC are known in the art; and any known method can be used to obtain MSC. See, e.g., U.S. Pat. No. 5,736,396, which describes isolation of human MSC.

A cell is in some cases a plant cell. A plant cell can be a cell of a monocotyledon. A cell can be a cell of a dicotyledon.

In some cases, the cell is a plant cell. For example, the cell can be a cell of a major agricultural plant, e.g., Barley, Beans (Dry Edible), Canola, Corn, Cotton (Pima), Cotton (Upland), Flaxseed, Hay (Alfalfa), Hay (Non-Alfalfa), Oats, Peanuts, Rice, Sorghum, Soybeans, Sugarbeets, Sugarcane, Sunflowers (Oil), Sunflowers (Non-Oil), Sweet Potatoes, Tobacco (Burley), Tobacco (Flue-cured), Tomatoes, Wheat (Durum), Wheat (Spring), Wheat (Winter), and the like. As another example, the cell is a cell of a vegetable crops which include but are not limited to, e.g., alfalfa sprouts, aloe leaves, arrow root, arrowhead, artichokes, asparagus, bamboo shoots, banana flowers, bean sprouts, beans, beet tops, beets, bittermelon, bok choy, broccoli, broccoli rabe (rappini), brussels sprouts, cabbage, cabbage sprouts, cactus leaf (nopales), calabaza, cardoon, carrots, cauliflower, celery, chayote, chinese artichoke (crosnes), chinese cabbage, chinese celery, chinese chives, choy sum, chrysanthemum leaves (tung ho), collard greens, corn stalks, corn-sweet, cucumbers, daikon, dandelion greens, dasheen, dau mue (pea tips), donqua (winter melon), eggplant, endive, escarole, fiddle head ferns, field cress, frisee, gai choy (chinese mustard), gailon, galanga (siam, thai ginger), garlic, ginger root, gobo, greens, hanover salad greens, huauzontle, jerusalem artichokes, jicama, kale greens, kohlrabi, lamb's quarters (quilete), lettuce (bibb), lettuce (boston), lettuce (boston red), lettuce (green leaf), lettuce (iceberg), lettuce (lolla rossa), lettuce (oak leaf—green), lettuce (oak leaf—red), lettuce (processed), lettuce (red leaf), lettuce (romaine), lettuce (ruby romaine), lettuce (russian red mustard), linkok, lo bok, long beans, lotus root, mache, maguey (agave) leaves, malanga, mesculin mix, mizuna, moap (smooth luffa), moo, moqua (fuzzy squash), mushrooms, mustard, nagaimo, okra, ong choy, onions green, opo (long squash), ornamental corn, ornamental gourds, parsley, parsnips, peas, peppers (bell type), peppers, pumpkins, radicchio, radish sprouts, radishes, rape greens, rape greens, rhubarb, romaine (baby red), rutabagas, salicornia (sea bean), sinqua (angled/ridged luffa), spinach, squash, straw bales, sugarcane, sweet potatoes, swiss chard, tamarindo, taro, taro leaf, taro shoots, tatsoi, tepeguaje (guaje), tindora, tomatillos, tomatoes, tomatoes (cherry), tomatoes (grape type), tomatoes (plum type), tumeric, turnip tops greens, turnips, water chestnuts, yampi, yams (names), yu choy, yuca (cassava), and the like.

A cell is in some cases an arthropod cell. For example, the cell can be a cell of a sub-order, a family, a sub-family, a group, a sub-group, or a species of, e.g., Chelicerata, Myriapodia, Hexipodia, Arachnida, Insecta, Archaeognatha, Thysanura, Palaeoptera, Ephemeroptera, Odonata, Anisoptera, Zygoptera, Neoptera, Exopterygota, Plecoptera, Embioptera, Orthoptera, Zoraptera, Dermaptera, Dictyoptera, Notoptera, Grylloblattidae, Mantophasmatidae, Phasmatodea, Blattaria, Isoptera, Mantodea, Parapneuroptera, Psocoptera, Thysanoptera, Phthiraptera, Hemiptera, Endopterygota or Holometabola, Hymenoptera, Coleoptera, Strepsiptera, Raphidioptera, Megaloptera, Neuroptera, Mecoptera, Siphonaptera, Diptera, Trichoptera, or Lepidoptera.

A cell is in some cases an insect cell. For example, in some cases, the cell is a cell of a mosquito, a grasshopper, a true bug, a fly, a flea, a bee, a wasp, an ant, a louse, a moth, or a beetle.

Introducing Components into a Target Cell

A Cas9 guide RNA (or a nucleic acid comprising a nucleotide sequence encoding same), and/or a Cas9 fusion polypeptide (or a nucleic acid comprising a nucleotide sequence encoding same) and/or a donor polynucleotide can be introduced into a host cell by any of a variety of well-known methods.

Methods of introducing a nucleic acid into a cell are known in the art, and any convenient method can be used to introduce a nucleic acid (e.g., an expression construct) into a target cell (e.g., eukaryotic cell, human cell, stem cell, progenitor cell, and the like). Suitable methods are described in more detail elsewhere herein and include e.g., viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery (see, e.g., Panyam et., al Adv Drug Deliv Rev. 2012 Sep. 13. pii: 50169-409X(12)00283-9. doi: 10.1016/j.addr.2012.09.023), and the like. Any or all of the components can be introduced into a cell as a composition (e.g., including any convenient combination of: an RNA-guided effector polypeptide, a fusion polypeptide, a guide RNA, a donor polynucleotide, etc.) using known methods, e.g., such as nucleofection.

### Donor Polynucleotide (Donor Template)

Guided by a dual or single guide RNA, a fusion protein in some cases generates site-specific double strand breaks (DSBs) or single strand breaks (SSBs) within double-stranded DNA (dsDNA) target nucleic acids, which are repaired either by non-homologous end joining (NHEJ) or homology-directed recombination (HDR).

In some cases, contacting a target DNA (with fusion protein and a guide RNA) occurs under conditions that are permissive for nonhomologous end joining or homology-directed repair. Thus, in some cases, a subject method includes contacting the target DNA with a donor polynucleotide (e.g., by introducing the donor polynucleotide into a cell), wherein the donor polynucleotide, a portion of the donor polynucleotide, a copy of the donor polynucleotide, or a portion of a copy of the donor polynucleotide integrates into the target DNA. In some cases, the method does not comprise contacting a cell with a donor polynucleotide, and the target DNA is modified such that nucleotides within the target DNA are deleted.

In applications in which it is desirable to insert a polynucleotide sequence into the genome where a target sequence is cleaved, a donor polynucleotide (a nucleic acid comprising a donor sequence) can also be provided to the cell. By a “donor sequence” or “donor polynucleotide” or “donor template” it is meant a nucleic acid sequence to be inserted at the site cleaved by the fusion protein (e.g., after dsDNA cleavage, after nicking a target DNA, after dual nicking a target DNA, and the like). The donor polynucleotide can contain sufficient homology to a genomic sequence at the target site, e.g. 70%, 80%, 85%, 90%, 95%, or 100% homology with the nucleotide sequences flanking the target site, e.g. within about 50 bases or less of the target site, e.g. within about 30 bases, within about 15 bases, within about 10 bases, within about 5 bases, or immediately flanking the target site, to support homology-directed repair between it and the genomic sequence to which it bears homology. Approximately 25, 50, 100, or 200 nucleotides, or more than 200 nucleotides, of sequence homology between a donor and a genomic sequence (or any integral value between 10 and 200 nucleotides, or more) can support homology-directed repair. Donor polynucleotides can be of any length, e.g. 10 nucleotides or more, 50 nucleotides or more, 100 nucleotides or more, 250 nucleotides or more, 500 nucleotides or more, 1000 nucleotides or more, 5000 nucleotides or more, etc.

The donor sequence is typically not identical to the genomic sequence that it replaces. Rather, the donor sequence may contain at least one or more single base changes, insertions, deletions, inversions or rearrangements with respect to the genomic sequence, so long as sufficient homology is present to support homology-directed repair (e.g., for gene correction, e.g., to convert a disease-causing base pair of a non disease-causing base pair). In some embodiments, the donor sequence comprises a non-homologous sequence flanked by two regions of homology, such that homology-directed repair between the target DNA region and the two flanking sequences results in insertion of the non-homologous sequence at the target region. Donor sequences may also comprise a vector backbone containing sequences that are not homologous to the DNA region of interest and that are not intended for insertion into the DNA region of interest. Generally, the homologous region(s) of a donor sequence will have at least 50% sequence identity to a genomic sequence with which recombination is desired. In certain embodiments, 60%, 70%, 80%, 90%, 95%, 98%, 99%, or 99.9% sequence identity is present. Any value between 1% and 100% sequence identity can be present, depending upon the length of the donor polynucleotide.

The donor sequence may comprise certain sequence differences as compared to the genomic sequence, e.g. restriction sites, nucleotide polymorphisms, selectable markers (e.g., drug resistance genes, fluorescent proteins, enzymes etc.), etc., which may be used to assess for successful insertion of the donor sequence at the cleavage site or in some cases may be used for other purposes (e.g., to signify expression at the targeted genomic locus). In some cases, if located in a coding region, such nucleotide sequence differences will not change the amino acid sequence, or will make silent amino acid changes (i.e., changes which do not affect the structure or function of the protein). Alternatively, these sequences differences may include flanking recombination sequences such as FLPs, loxP sequences, or the like, that can be activated at a later time for removal of the marker sequence.

In some cases, the donor sequence is provided to the cell as single-stranded DNA. In some cases, the donor sequence is provided to the cell as double-stranded DNA. It may be introduced into a cell in linear or circular form. If introduced in linear form, the ends of the donor sequence may be protected (e.g., from exonucleolytic degradation) by any convenient method and such methods are known to those of skill in the art. For example, one or more dideoxynucleotide residues can be added to the 3′ terminus of a linear molecule and/or self-complementary oligonucleotides can be ligated to one or both ends. See, for example, Chang et al. (1987) Proc. Natl. Acad Sci USA 84:4959-4963; Nehls et al. (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues. As an alternative to protecting the termini of a linear donor sequence, additional lengths of sequence may be included outside of the regions of homology that can be degraded without impacting recombination. A donor sequence can be introduced into a cell as part of a vector molecule having additional sequences such as, for example, replication origins, promoters and genes encoding antibiotic resistance. Moreover, donor sequences can be introduced as naked nucleic acid, as nucleic acid complexed with an agent such as a liposome or poloxamer, or can be delivered by viruses (e.g., adenovirus, AAV), as described elsewhere herein for nucleic acids encoding a guide RNA and/or a fusion polypeptide and/or donor polynucleotide.

### Transgenic, Non-Human Organisms

As described above, in some cases, a nucleic acid (e.g., a recombinant expression vector) of the present disclosure (e.g., a nucleic acid comprising a nucleotide sequence encoding an RNA-guided effector polypeptide of the present disclosure; a nucleic acid comprising a nucleotide sequence encoding a fusion polypeptide of the present disclosure; etc.), is used as a transgene to generate a transgenic non-human organism that produces an RNA-guided effector polypeptide, or a fusion polypeptide, of the present disclosure. The present disclosure provides a transgenic-non-human organism comprising a nucleotide sequence encoding an RNA-guided effector polypeptide, or a fusion polypeptide, of the present disclosure.

### Transgenic, Non-Human Animals

The present disclosure provides a transgenic non-human animal, which animal comprises a transgene comprising a nucleic acid comprising a nucleotide sequence encoding an RNA-guided effector polypeptide or a fusion polypeptide. In some embodiments, the genome of the transgenic non-human animal comprises a nucleotide sequence encoding an RNA-guided effector polypeptide, or a fusion polypeptide, of the present disclosure. In some cases, the transgenic non-human animal is homozygous for the genetic modification. In some cases, the transgenic non-human animal is heterozygous for the genetic modification. In some embodiments, the transgenic non-human animal is a vertebrate, for example, a fish (e.g., salmon, trout, zebra fish, gold fish, puffer fish, cave fish, etc.), an amphibian (frog, newt, salamander, etc.), a bird (e.g., chicken, turkey, etc.), a reptile (e.g., snake, lizard, etc.), a non-human mammal (e.g., an ungulate, e.g., a pig, a cow, a goat, a sheep, etc.; a lagomorph (e.g., a rabbit); a rodent (e.g., a rat, a mouse); a non-human primate; etc.), etc. In some cases, the transgenic non-human animal is an invertebrate. In some cases, the transgenic non-human animal is an insect (e.g., a mosquito; an agricultural pest; etc.). In some cases, the transgenic non-human animal is an arachnid.

Nucleotide sequences encoding an RNA-guided effector polypeptide, or a fusion polypeptide, of the present disclosure can be under the control of (i.e., operably linked to) an unknown promoter (e.g., when the nucleic acid randomly integrates into a host cell genome) or can be under the control of (i.e., operably linked to) a known promoter. Suitable known promoters can be any known promoter and include constitutively active promoters (e.g., CMV promoter), inducible promoters (e.g., heat shock promoter, tetracycline-regulated promoter, steroid-regulated promoter, metal-regulated promoter, estrogen receptor-regulated promoter, etc.), spatially restricted and/or temporally restricted promoters (e.g., a tissue specific promoter, a cell type specific promoter, etc.), etc.

### Transgenic Plants

As described above, in some cases, a nucleic acid (e.g., a recombinant expression vector) of the present disclosure (e.g., a nucleic acid comprising a nucleotide sequence encoding an RNA-guided effector polypeptide of the present disclosure; a nucleic acid comprising a nucleotide sequence encoding a fusion polypeptide of the present disclosure; etc.), is used as a transgene to generate a transgenic plant that produces an RNA-guided effector polypeptide, or a fusion polypeptide, of the present disclosure. The present disclosure provides a transgenic plant comprising a nucleotide sequence encoding an RNA-guided effector polypeptide, or a fusion polypeptide, of the present disclosure. In some embodiments, the genome of the transgenic plant comprises a subject nucleic acid. In some embodiments, the transgenic plant is homozygous for the genetic modification. In some embodiments, the transgenic plant is heterozygous for the genetic modification.

Methods of introducing exogenous nucleic acids into plant cells are well known in the art. Such plant cells are considered “transformed,” as defined above. Suitable methods include viral infection (such as double stranded DNA viruses), transfection, conjugation, protoplast fusion, electroporation, particle gun technology, calcium phosphate precipitation, direct microinjection, silicon carbide whiskers technology, Agrobacterium-mediated transformation and the like. The choice of method is generally dependent on the type of cell being transformed and the circumstances under which the transformation is taking place (i.e. in vitro, ex vivo, or in vivo).

Transformation methods based upon the soil bacterium Agrobacterium tumefaciens are particularly useful for introducing an exogenous nucleic acid molecule into a vascular plant. The wild type form of Agrobacterium contains a Ti (tumor-inducing) plasmid that directs production of tumorigenic crown gall growth on host plants. Transfer of the tumor-inducing T-DNA region of the Ti plasmid to a plant genome requires the Ti plasmid-encoded virulence genes as well as T-DNA borders, which are a set of direct DNA repeats that delineate the region to be transferred. An Agrobacterium-based vector is a modified form of a Ti plasmid, in which the tumor inducing functions are replaced by the nucleic acid sequence of interest to be introduced into the plant host.

Agrobacterium-mediated transformation generally employs cointegrate vectors or binary vector systems, in which the components of the Ti plasmid are divided between a helper vector, which resides permanently in the Agrobacterium host and carries the virulence genes, and a shuttle vector, which contains the gene of interest bounded by T-DNA sequences. A variety of binary vectors is well known in the art and are commercially available, for example, from Clontech (Palo Alto, Calif.). Methods of coculturing Agrobacterium with cultured plant cells or wounded tissue such as leaf tissue, root explants, hypocotyledons, stem pieces or tubers, for example, also are well known in the art. See, e.g., Glick and Thompson, (eds.), Methods in Plant Molecular Biology and Biotechnology, Boca Raton, Fla.: CRC Press (1993).

Microprojectile-mediated transformation also can be used to produce a subject transgenic plant. This method, first described by Klein et al. (Nature 327:70-73 (1987)), relies on microprojectiles such as gold or tungsten that are coated with the desired nucleic acid molecule by precipitation with calcium chloride, spermidine or polyethylene glycol. The microprojectile particles are accelerated at high speed into an angiosperm tissue using a device such as the BIOLISTIC PD-1000 (Biorad; Hercules Calif.).

A nucleic acid of the present disclosure (e.g., a nucleic acid (e.g., a recombinant expression vector) comprising a nucleotide sequence encoding an RNA-guided effector polypeptide, or a fusion polypeptide, of the present disclosure) may be introduced into a plant in a manner such that the nucleic acid is able to enter a plant cell(s), e.g., via an in vivo or ex vivo protocol. By “in vivo,” it is meant in the nucleic acid is administered to a living body of a plant e.g. infiltration. By “ex vivo” it is meant that cells or explants are modified outside of the plant, and then such cells or organs are regenerated to a plant. A number of vectors suitable for stable transformation of plant cells or for the establishment of transgenic plants have been described, including those described in Weissbach and Weissbach, (1989) Methods for Plant Molecular Biology Academic Press, and Gelvin et al., (1990) Plant Molecular Biology Manual, Kluwer Academic Publishers. Specific examples include those derived from a Ti plasmid of Agrobacterium tumefaciens, as well as those disclosed by Herrera-Estrella et al. (1983) Nature 303: 209, Bevan (1984) Nucl Acid Res. 12: 8711-8721, Klee (1985) Bio/Technolo 3: 637-642. Alternatively, non-Ti vectors can be used to transfer the DNA into plants and cells by using free DNA delivery techniques. By using these methods transgenic plants such as wheat, rice (Christou (1991) Bio/Technology 9:957-9 and 4462) and corn (Gordon-Kamm (1990) Plant Cell 2: 603-618) can be produced. An immature embryo can also be a good target tissue for monocots for direct DNA delivery techniques by using the particle gun (Weeks et al. (1993) Plant Physiol 102: 1077-1084; Vasil (1993) Bio/Technolo 10: 667-674; Wan and Lemeaux (1994) Plant Physiol 104: 37-48 and for Agrobacterium-mediated DNA transfer (Ishida et al. (1996) Nature Biotech 14: 745-750). Exemplary methods for introduction of DNA into chloroplasts are biolistic bombardment, polyethylene glycol transformation of protoplasts, and microinjection (Danieli et al Nat. Biotechnol 16:345-348, 1998; Staub et al Nat. Biotechnol 18: 333-338, 2000; O'Neill et al Plant J. 3:729-738, 1993; Knoblauch et al Nat. Biotechnol 17: 906-909; U.S. Pat. Nos. 5,451,513, 5,545,817, 5,545,818, and 5,576,198; in Intl. Application No. WO 95/16783; and in Boynton et al., Methods in Enzymology 217: 510-536 (1993), Svab et al., Proc. Natl. Acad. Sci. USA 90: 913-917 (1993), and McBride et al., Proc. Natl. Acad. Sci. USA 91: 7301-7305 (1994)). Any vector suitable for the methods of biolistic bombardment, polyethylene glycol transformation of protoplasts and microinjection will be suitable as a targeting vector for chloroplast transformation. Any double stranded DNA vector may be used as a transformation vector, especially when the method of introduction does not utilize Agrobacterium.

Plants which can be genetically modified include grains, forage crops, fruits, vegetables, oil seed crops, palms, forestry, and vines. Specific examples of plants which can be modified follow: maize, banana, peanut, field peas, sunflower, tomato, canola, tobacco, wheat, barley, oats, potato, soybeans, cotton, carnations, sorghum, lupin and rice.

The present disclosure provides transformed plant cells, tissues, plants and products that contain the transformed plant cells. A feature of the subject transformed cells, and tissues and products that include the same is the presence of a subject nucleic acid integrated into the genome, and production by plant cells of an RNA-guided effector polypeptide, or a fusion polypeptide, of the present disclosure. Recombinant plant cells of the present invention are useful as populations of recombinant cells, or as a tissue, seed, whole plant, stem, fruit, leaf, root, flower, stem, tuber, grain, animal feed, a field of plants, and the like.

Nucleotide sequences encoding an RNA-guided effector polypeptide, or a fusion polypeptide, of the present disclosure can be under the control of (i.e., operably linked to) an unknown promoter (e.g., when the nucleic acid randomly integrates into a host cell genome) or can be under the control of (i.e., operably linked to) a known promoter. Suitable known promoters can be any known promoter and include constitutively active promoters, inducible promoters, spatially restricted and/or temporally restricted promoters, etc.

### Compositions, Ribonucleoprotein Complexes, and Systems

The present disclosure provides a ribonucleoprotein (RNP) complex comprising: a) a fusion polypeptide of the present disclosure; and b) a guide RNA, or a nucleic acid encoding the guide RNA, wherein the guide RNA comprises a guide sequence that is complementary to a target sequence of a target nucleic acid, and comprises a region that can bind to the RNA-guided effector polypeptide component of the fusion polypeptide. The present disclosure provides an RNP complex comprising: a) an RNA-guided effector polypeptide of the present disclosure; and b) a guide RNA, or a nucleic acid encoding the guide RNA, wherein the guide RNA comprises a guide sequence that is complementary to a target sequence of a target nucleic acid, and comprises a region that can bind to the RNA-guided effector polypeptide. In some cases, the guide RNA is a single-molecule guide RNA.

The present disclosure provides a composition comprising: a) an RNP complex of the present disclosure; and b) at least one additional component, where the at least one additional component is selected from a salt, a buffer, a protease inhibitor, a nuclease inhibitor, a lipid, and the like. The present disclosure provides a composition comprising: a) an RNP complex of the present disclosure; and b) a pharmaceutically acceptable excipient. In some cases, a composition of the present disclosure comprises a lipid. In some cases, a composition of the present disclosure comprises a liposome, a hydrogel, a microparticle, a nanoparticle, or a block copolymer micelle.

The present disclosure provides a system comprising an RNA-guided effector polypeptide of the present disclosure, or a fusion polypeptide of the present disclosure. The present disclosure provides a system comprising a nucleic acid comprising a nucleotide sequence encoding an RNA-guided effector polypeptide of the present disclosure, or a nucleic acid comprising a nucleotide sequence encoding a fusion polypeptide of the present disclosure. In some cases, a system of the present disclosure further comprises a guide RNA.

A system of the present disclosure can comprise: a) an RNA-guided effector polypeptide of the present disclosure and a guide RNA; b) a fusion polypeptide of the present disclosure and a guide RNA; c) a fusion polypeptide of the present disclosure, a guide RNA, and a donor template nucleic acid; d) an mRNA encoding an RNA-guided effector polypeptide of the present disclosure; and a guide RNA; e) an mRNA encoding a fusion polypeptide of the present disclosure; and a guide RNA; f) an mRNA encoding a fusion polypeptide of the present disclosure, a guide RNA, and a donor template nucleic acid; g) a recombinant expression vector comprising a nucleotide sequence encoding an RNA-guided effector polypeptide of the present disclosure and a nucleotide sequence encoding a guide RNA; h) a recombinant expression vector comprising a nucleotide sequence encoding a fusion polypeptide of the present disclosure and a nucleotide sequence encoding a guide RNA; i) a recombinant expression vector comprising a nucleotide sequence encoding a fusion polypeptide of the present disclosure, a nucleotide sequence encoding a guide RNA, and a nucleotide sequence encoding a donor template nucleic acid; j) a first recombinant expression vector comprising a nucleotide sequence encoding an RNA-guided effector polypeptide of the present disclosure, and a second recombinant expression vector comprising a nucleotide sequence encoding a guide RNA; k) a first recombinant expression vector comprising a nucleotide sequence encoding a fusion polypeptide of the present disclosure, and a second recombinant expression vector comprising a nucleotide sequence encoding a guide RNA; l) a first recombinant expression vector comprising a nucleotide sequence encoding a fusion polypeptide of the present disclosure, and a second recombinant expression vector comprising a nucleotide sequence encoding a guide RNA; and a donor template nucleic acid; m) a recombinant expression vector comprising a nucleotide sequence encoding an RNA-guided effector polypeptide of the present disclosure, a nucleotide sequence encoding a first guide RNA, and a nucleotide sequence encoding a second guide RNA; or n) a recombinant expression vector comprising a nucleotide sequence encoding a fusion polypeptide of the present disclosure, a nucleotide sequence encoding a first guide RNA, and a nucleotide sequence encoding a second guide RNA; or some variation of one of (a) through (n).

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-77 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. An RNA-guided effector polypeptide having a length of from about 610 amino acids to about 1310 amino acids, wherein the RNA-guided effector polypeptide comprises one or more of:

i) a deletion of from 5 contiguous amino acids to 158 contiguous amino acids of amino acids 166-324;

ii) a deletion of from 30 contiguous amino acids to 255 contiguous amino acids of amino acids 484-739;

iii) a deletion of from 4 contiguous amino acids to 220 contiguous amino acids of amino acids 761-981; and

d) a deletion of from 20 contiguous amino acids to 118 contiguous amino acids of amino acids 994-1112,

wherein the amino acid numbering is based on SEQ ID NO:5, or corresponding amino acid positions of an amino acid sequence set forth in any one of SEQ ID NOs:6-816.

Aspect 2. The RNA-guided effector polypeptide of aspect 1, wherein the RNA-guided effector polypeptide comprises a deletion of from 5 contiguous amino acids to 158 contiguous amino acids of amino acids 166-324.

Aspect 3. The RNA-guided effector polypeptide of aspect 1, wherein the RNA-guided effector polypeptide comprises a deletion of from 30 contiguous amino acids to 255 contiguous amino acids of amino acids 484-739.

Aspect 4. The RNA-guided effector polypeptide of aspect 1, wherein the RNA-guided effector polypeptide comprises a deletion of from 4 contiguous amino acids to 220 contiguous amino acids of amino acids 761-981.

Aspect 5. The RNA-guided effector polypeptide of aspect 1, wherein the RNA-guided effector polypeptide comprises a deletion of from 20 contiguous amino acids to 118 contiguous amino acids of amino acids 994-1112.

Aspect 6. The RNA-guided effector polypeptide of aspect 1, wherein the RNA-guided effector polypeptide comprises:

i) a deletion of from 5 contiguous amino acids to 158 contiguous amino acids of amino acids 166-324; and

ii) a deletion of from 30 contiguous amino acids to 255 contiguous amino acids of amino acids 484-739.

Aspect 7. The RNA-guided effector polypeptide of aspect 1, wherein the RNA-guided effector polypeptide comprises:

i) a deletion of from 5 contiguous amino acids to 158 contiguous amino acids of amino acids 166-324; and

ii) a deletion of from 4 contiguous amino acids to 220 contiguous amino acids of amino acids 761-981.

Aspect 8. The RNA-guided effector polypeptide of aspect 1, wherein the RNA-guided effector polypeptide comprises:

i) a deletion of from 5 amino acids to 158 amino acids of amino acids 166-324; and

ii) a deletion of from 20 amino acids to 118 amino acids of amino acids 994-1112.

Aspect 9. The RNA-guided effector polypeptide of aspect 1, wherein the RNA-guided effector polypeptide comprises:

i) a deletion of from 5 contiguous amino acids to 158 contiguous amino acids of amino acids 166-324;

ii) a deletion of from 30 contiguous amino acids to 255 contiguous amino acids of amino acids 484-739; and

iii) a deletion of from 4 contiguous amino acids to 220 contiguous amino acids of amino acids 761-981.

Aspect 10. The RNA-guided effector polypeptide of aspect 1, wherein the RNA-guided effector polypeptide comprises:

i) a deletion of from 5 contiguous amino acids to 158 contiguous amino acids of amino acids 166-324;

ii) a deletion of from 4 contiguous amino acids to 220 contiguous amino acids of amino acids 761-981; and

iii) a deletion of from 20 contiguous amino acids to 118 contiguous amino acids of amino acids 994-1112.

Aspect 11. The RNA-guided effector polypeptide of aspect 1, wherein the RNA-guided effector polypeptide comprises:

i) a deletion of from 5 contiguous amino acids to 158 contiguous amino acids of amino acids 166-324;

ii) a deletion of from 30 contiguous amino acids to 255 contiguous amino acids of amino acids 484-739;

iii) a deletion of from 4 contiguous amino acids to 220 contiguous amino acids of amino acids 761-981; and

iv) a deletion of from 20 contiguous amino acids to 118 contiguous amino acids of amino acids 994-1112.

Aspect 12. The RNA-guided effector polypeptide of aspect 1, wherein the RNA-guided effector polypeptide comprises:

i) a deletion of from 30 contiguous amino acids to 255 contiguous amino acids of amino acids 484-739; and

ii) a deletion of from 4 contiguous amino acids to 220 contiguous amino acids of amino acids 761-981.

Aspect 13. The RNA-guided effector polypeptide of aspect 1, wherein the RNA-guided effector polypeptide comprises:

i) a deletion of from 30 contiguous amino acids to 255 contiguous amino acids of amino acids 484-739; and

ii) a deletion of from 20 contiguous amino acids to 118 contiguous amino acids of amino acids 994-1112.

Aspect 14. The RNA-guided effector polypeptide of aspect 1, wherein the RNA-guided effector polypeptide comprises:

i) a deletion of from 30 contiguous amino acids to 255 contiguous amino acids of amino acids 484-739;

ii) a deletion of from 4 contiguous amino acids to 220 contiguous amino acids of amino acids 761-981; and

iii) a deletion of from 20 contiguous amino acids to 118 contiguous amino acids of amino acids 994-1112.

Aspect 15. The RNA-guided effector polypeptide of aspect 1, wherein the RNA-guided effector polypeptide comprises:

i) a deletion of from 4 contiguous amino acids to 220 contiguous amino acids of amino acids 761-981; and

ii) a deletion of from 20 contiguous amino acids to 118 contiguous amino acids of amino acids 994-1112.

Aspect 16. The RNA-guided effector polypeptide of aspect 1, wherein the RNA-guided effector polypeptide comprises a deletion of amino acids 179-296 based on the number of SEQ ID NO:5.

Aspect 17. The RNA-guided effector polypeptide of aspect 1, wherein the RNA-guided effector polypeptide comprises a deletion of amino acids 503-708 based on the number of SEQ ID NO:5.

Aspect 18. The RNA-guided effector polypeptide of aspect 1, wherein the RNA-guided effector polypeptide comprises a deletion of amino acids 792-897 based on the number of SEQ ID NO:5.

Aspect 19. The RNA-guided effector polypeptide of aspect 1, wherein the RNA-guided effector polypeptide comprises a deletion of amino acids 1010-1081 based on the number of SEQ ID NO:5.

Aspect 20. The RNA-guided effector polypeptide of aspect 1, wherein the RNA-guided effector polypeptide comprises:

i) a deletion of amino acids 503-708 based on the number of SEQ ID NO:5;

ii) a deletion of amino acids 792-897 based on the number of SEQ ID NO:5; and

iii) a deletion of amino acids 1010-1081 based on the number of SEQ ID NO:5.

Aspect 21. The RNA-guided effector polypeptide of aspect 1, wherein the RNA-guided effector polypeptide comprises:

i) a deletion of amino acids 179-296 based on the number of SEQ ID NO:5;

ii) a deletion of amino acids 503-708 based on the number of SEQ ID NO:5;

iii) a deletion of amino acids 792-897 based on the number of SEQ ID NO:5; and

iv) a deletion of amino acids 1010-1081 based on the number of SEQ ID NO:5.

Aspect 22. The RNA-guided effector polypeptide of aspect 1, wherein the polypeptide comprises an amino acid sequence having at least 85% amino acid sequence identity to the amino acid sequence depicted in FIG. 2, wherein the RNA-guided effector polypeptide is 990 amino acids in length.

Aspect 23. The RNA-guided effector polypeptide of aspect 1, wherein the polypeptide comprises an amino acid sequence having at least 85% amino acid sequence identity to the amino acid sequence depicted in FIG. 3, wherein the RNA-guided effector polypeptide is 874 amino acids in length.

Aspect 24. A nucleic acid comprising a nucleotide sequence encoding the RNA-guided effector polypeptide of any one of aspects 1-23.

Aspect 25. The nucleic acid of aspect 24, wherein the nucleotide sequence is operably linked to a transcriptional control element.

Aspect 26. The nucleic acid of aspect 25, wherein the transcriptional control element is a promoter.

Aspect 27. The nucleic acid of aspect 26, wherein the promoter is a regulatable promoter.

Aspect 28. The nucleic acid of aspect 27, wherein the promoter is functional in a eukaryotic cell.

Aspect 29. The nucleic acid of any one of aspects 24-28, further comprising a nucleotide sequence encoding a guide RNA, wherein the guide RNA comprises a guide sequence that is complementary to a target sequence of a target nucleic acid, and comprises a region that can bind to the RNA-guided effector polypeptide.

Aspect 30. A recombinant expression vector comprising the nucleic acid of any one of aspects 24-29.

Aspect 31. The recombinant expression vector of aspect 30, wherein the recombinant expression vector is recombinant viral expression vector.

Aspect 32. A fusion polypeptide comprising:

a) the RNA-guided effector polypeptide of any one of aspects 1-23; and

b) a heterologous fusion partner.

Aspect 33. The fusion polypeptide of aspect 32, wherein the heterologous fusion partner provides for control of gene expression.

Aspect 34. The fusion polypeptide of aspect 32, wherein the heterologous fusion partner is a protein-modifying enzyme.

Aspect 35. The fusion polypeptide of aspect 32, wherein the heterologous fusion partner is a modifying enzyme is a DNA-modifying enzyme.

Aspect 36. The fusion polypeptide of aspect 32, wherein the heterologous fusion partner exhibits nuclease activity.

Aspect 37. The fusion polypeptide of aspect 36, wherein the nuclease activity is double-stranded DNA cleavage activity.

Aspect 38. The fusion polypeptide of aspect 37, wherein the heterologous fusion partner comprises an amino acid sequence having at least 85% amino acid sequence identity to the catalytic domain of the amino acid sequence depicted in FIG. 2A and has a length of no more than 200 amino acids.

Aspect 39. The fusion polypeptide of any one of aspects 32-38, wherein the fusion partner has a length of no more than 400 amino acids.

Aspect 40. The fusion polypeptide of aspect 35, wherein the heterologous fusion partner is a base editor.

Aspect 41. The fusion polypeptide of aspect 40, wherein the base editor is a cytidine deaminase.

Aspect 42. The fusion polypeptide of aspect 40, wherein the base editor is an adenosine deaminase.

Aspect 43. The fusion polypeptide of any one of aspects 32-42, further comprising one or more nuclear localization sequences (NLS).

Aspect 44. The fusion polypeptide of aspect 43, wherein the fusion polypeptide comprises a single NLS.

Aspect 45. The fusion polypeptide of aspect 43, wherein the fusion polypeptide comprises 2 or more NLSs.

Aspect 46. The fusion polypeptide of aspect 43, wherein the fusion polypeptide comprises an NLS at the C-terminus, at the N-terminus, or at the C-terminus and the N-terminus, of the fusion polypeptide.

Aspect 47. The fusion polypeptide of any one of aspects 32-42, further comprising an endosomolytic peptide.

Aspect 48. A nucleic acid comprising a nucleotide sequence encoding the fusion polypeptide of any one of aspects 32-47.

Aspect 49. The nucleic acid of aspect 48, wherein the nucleotide sequence is operably linked to a transcriptional control element.

Aspect 50. The nucleic acid of aspect 49, wherein the transcriptional control element is a promoter.

Aspect 51. The nucleic acid of aspect 50, wherein the promoter is a regulatable promoter.

Aspect 52. The nucleic acid of aspect 50 or aspect 51, wherein the promoter is functional in a eukaryotic cell.

Aspect 53. The nucleic acid of any one of aspects 48-52, further comprising a nucleotide sequence encoding a guide RNA, wherein the guide RNA comprises a guide sequence that is complementary to a target sequence of a target nucleic acid, and comprises a region that can bind to the RNA-guided effector polypeptide.

Aspect 54. A recombinant expression vector comprising the nucleic acid of any one of aspects 48-53.

Aspect 55. The recombinant expression vector of aspect 54, wherein the recombinant expression vector is recombinant viral expression vector.

Aspect 56. A ribonucleoprotein (RNP) complex comprising:

a) the fusion polypeptide of any one of aspects 32-47; and

b) a guide RNA, or a nucleic acid encoding the guide RNA, wherein the guide RNA comprises a guide sequence that is complementary to a target sequence of a target nucleic acid, and comprises a region that can bind to the RNA-guided effector polypeptide.

Aspect 57. The RNP complex of aspect 56, wherein the guide RNA is a single-molecule guide RNA.

Aspect 58. A composition comprising:

a) the RNP complex of aspect 56 or aspect 57; and

b) a pharmaceutically acceptable excipient.

Aspect 59. The composition of aspect 58, comprising a lipid.

Aspect 60. The composition of aspect 58, comprising a liposome, a hydrogel, a microparticle, a nanoparticle, or a block copolymer micelle.

Aspect 61. A kit comprising:

a) the RNA-guided effector polypeptide of any one of aspects 1-23, or the fusion polypeptide of any one of aspects 32-47; and

b) a guide RNA, or a nucleic acid encoding the guide RNA, wherein the guide RNA comprises a guide sequence that is complementary to a target sequence of a target nucleic acid, and comprises a region that can bind to the RNA-guided effector polypeptide.

Aspect 62. The kit of aspect 61, wherein the guide RNA is a single-molecule guide RNA.

Aspect 63. The kit of aspect 61 or aspect 62, wherein the RNA-guided effector polypeptide and the guide RNA are in separate containers.

Aspect 64. The kit of aspect 61 or aspect 62, wherein the RNA-guided effector polypeptide and the guide RNA are together in a single container.

Aspect 65. The kit of any one of aspects 61-64, wherein the guide RNA comprises one or more of: a modified nucleobase, a modified backbone or non-natural internucleoside linkage, a modified sugar moiety, a Locked Nucleic Acid, a Peptide Nucleic Acid, and a deoxyribonucleotide.

Aspect 66. The kit of any one of aspects 61-65, further comprising a donor nucleic acid template.

Aspect 67. A method of binding and/or modifying a target nucleic acid and/or modifying a polypeptide that binds to a target nucleic acid, the method comprising contacting the target nucleic acid with:

i) the RNA-guided effector polypeptide of any one of aspects 1-23; or

ii) the fusion polypeptide of any one of aspects 32-47.

Aspect 68. The method of aspect 67, wherein the target nucleic acid is selected from: double stranded DNA, single stranded DNA, RNA, genomic DNA, and extrachromosomal DNA.

Aspect 69. The method of aspect 67, wherein contacting results in genome editing.

Aspect 70. The method of aspect 67 or 68, wherein said contacting takes place outside of a bacterial cell and outside of an archaeal cell.

Aspect 71. The method of any one of aspects 67-69, wherein said contacting takes place in vitro outside of a cell.

Aspect 72. The method of any one of aspects 67-69, wherein said contacting takes place inside of a target cell.

Aspect 73. The method of aspect 72, wherein the target cell is a eukaryotic cell.

Aspect 74. The method of aspect 72 or aspect 73, wherein the target cell is in vivo.

Aspect 75. The method of aspect 72 or aspect 73, wherein the target cell is ex vivo.

Aspect 76. The method of aspect 72 or aspect 73, wherein the eukaryotic cell is selected from the group consisting of: a plant cell, a fungal cell, a single cell eukaryotic organism, a mammalian cell, a reptile cell, an insect cell, an avian cell, a fish cell, a parasite cell, an arthropod cell, a cell of an invertebrate, a cell of a vertebrate, a rodent cell, a mouse cell, a rat cell, a primate cell, a non-human primate cell, and a human cell.

Aspect 77. The method of any one of aspects 72-76, wherein said contacting further comprises: introducing a DNA donor template into the target cell.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1: Generation of Deletion Variants that Retain Target Nucleic Acid Binding

### MISER Protocol

1. A gene of interest is cloned into a suitable shuttle plasmid (e.g. pUC19).

2. Primers are designed such that they contain homology to 60 bp or more of the gene of interest, and a Type II restriction site (e.g. XbaI) is inserted into the middle of this primer. This process is then repeated for every single amino acid position of the protein such that, in the end, there are tiled primers capable of annealing at every amino acid position of the protein.

3. This process is repeated and a different, complementary restriction site (e.g. for XbaI, SpeI is suitable) is introduced into a second set of designed primers.

4. Primers can be synthesized via standard commercial synthesis. Olio Library Synthesis (OLS) primers (LeProust E M, et al. (2010) Nucleic Acids Res 38: 2522-2540; Kosuri et al. (2010) Nat. Biotechnol. 28:1295) were used; the OLS primers were obtained from Agilent.

5. Using recombineering (Higgins S A, Ouonkap S V Y, Savage D F. 2017. Rapid and Programmable Protein Mutagenesis Using Plasmid Recombineering. ACS Synth Biol 6: 1825-1833.), these primers can be used to mutagenize the shuttle plasmid in a cell (e.g., in an Escherichia coli cell in vitro). This process systematically inserts the restriction site designed in 2 and 3, above, into the gene of interest once per gene but does so at every site of the gene across the population of plasmids.

6. This can be done in a variety of configurations but, briefly, in a typical work flow, recombineering would be carried out first for XbaI-containing primers, the plasmids would be miniprepped, treated with XbaI to confirm they contain a single XbaI site, and then recombineering would be repeated a second time with SpeI. At the end of recombineering, the library is a diverse series of plasmids in which a single plasmid contains just one XbaI and one SpeI randomly distributed throughout the gene. All possible positions are represented across the library of plasmids,

7. The plasmid library is digested with XbaI and SpeI and plasmids are size-selected for electrophoresis or similar technique to select for desired deletion sizes. Recovered plasmids are re-ligated and transformed. (Cut XbaI and SpeI sites can ligated together).

8. This library now contains targeted deletions across the gene, where average deletion size is controllable by size-selection in step 7.

9. The protein library is assayed for function via a selection or screening assay. In the case of Cas9, one example of how this can be done is via a DNA-binding screen, e.g., as described in Oakes B L, Nadler D C, Savage D F. 2014. “Protein engineering of Cas9 for enhanced function.” Methods in Enzymology 546: 491-511. For example, catalytically inactive Cas9 (Cas9 lacking nuclease activity; “dCas9”) with a guide sequence of 5′-AACTTTCAGTTTAGCGGTCT-3′ (SEQ ID NO:897) can target and repress a genome-encoded red fluorescent protein (RFP) while avoiding repression of a genome-encoded upstream green fluorescent protein (GFP). In a screening context, this provides a simple output for assaying dCas9 target binding functionality (i.e. RFP knock-down) while correcting for extrinsic noise in the population by monitoring GFP. Briefly, cells containing functional dCas9 (dCas9 that, when complexed with a guide RNA, retains target nucleic acid binding) will repress RFP and express GFP while those with non-functional dCas9 will express both fluorescent proteins.

10. Recovered libraries can be deep sequenced, the deletion site identified via bioinformatics, and compared to the initial library to quantitatively assess the functionality of a given deletion.

11. This entire process gives all possible single contiguous deletions. Functional single deletions can be subjected to a second (or third, etc.) round to further investigate how deletions can be stacked together.

The above “MISER” protocol was used to generate deletions in Cas9 that retain the ability, when complexed with a guide RNA, to bind a target nucleic acid. Example data are shown in FIG. 8.

### Example 2: Mammalian CRISPR Interference Assay to Assess Function of Deletion Variants

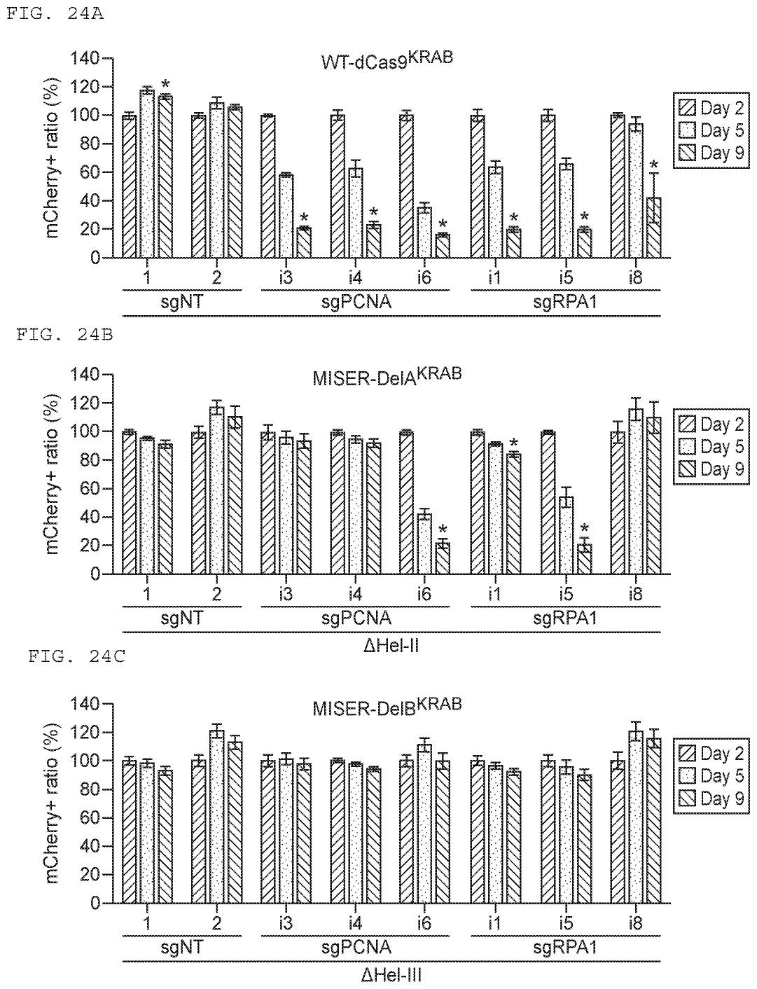

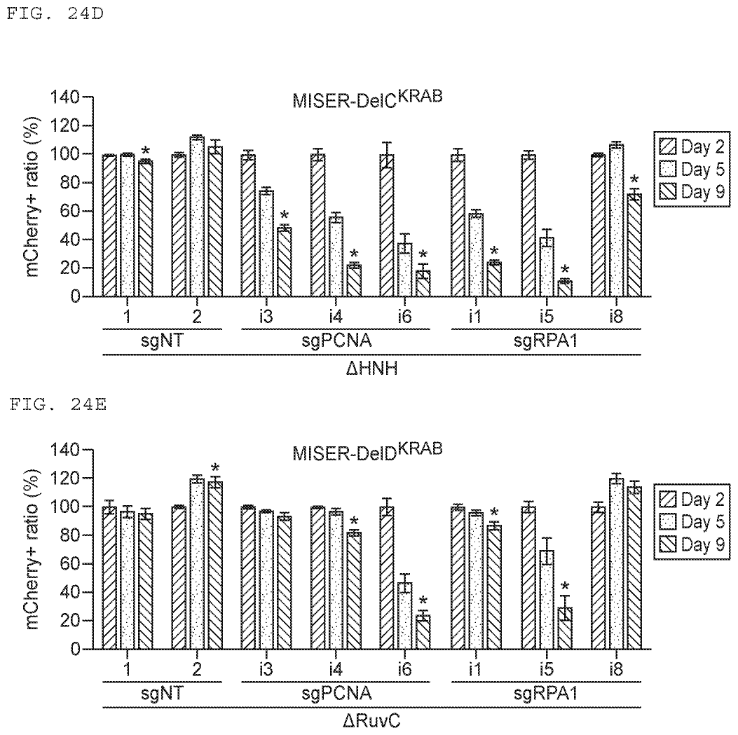

CRISPR interference (CRISPRi) assays (FIGS. 24A-24E) were used to assess the function of deletion variants in mammalian cells. Specifically, using a CRISPRi-based competitive proliferation assay, human U-251 glioblastoma cells were stably transduced with lentiviral vectors (pSC066) expressing MISER-dCas9 (MISER-DelA^{KRAB}, MISER DelB^{KRAB}, MISER-DelC^{KRAB}, MISER-DelD^{KRAB}) or WT-dCas9 KRAB fusion proteins, followed by selection on puromycin. The positions of the DelA, DelB, DelC, and DelD deletions of the of the dCas9 polypeptide present in the dCas9-DelA^{KRAB}, dCas9-DelB^{KRAB}, dCas9-DelC^{KRAB}, and dCas9-DelD^{KRAB} fusions are as follows (amino acid numbering based on SEQ ID NO:5): delA: deletion of amino acids 180-297; delB: deletion of amino acids 503-708; delC: deletion of amino acids 792-897; and delD: deletion of amino acids 1010-1081. The respective cell lines were then transduced with a secondary lentiviral vector (pCF221) expressing mCherry fluorescent protein and either CRISPRi sgRNAs targeting essential genes proliferating cell nuclear antigen (PCNA) or replicating protein A1 (RPA1) (sgPCNA (sgRNAs i3, i4, and i6); or sgRPA1 (sgRNAs i1, i5, or i8) or non-targeting controls (sgNT (sgRNAs 1 and 2)). After mixing with the respective parental population (at approximately an 80:20 ratio of transduced to non-transduced cells), the percentage of mCherry positive cells was monitored by flow cytometry over several days to assess the effect of CRISPRi with the given Cas9-variant on cell proliferation. A functional CRISPRi variant is expected to inhibit essential gene expression, thus lowering the observed mCherry signal. Error bars indicate the standard deviation of triplicates. Significance in cell depletion was assessed by comparing samples to their respective day two controls using unpaired, two-tailed t-tests (alpha=0.01).

FIG. 24A-24E depicts the percentage of mCherry positive mammalian cells in a CRISPR interference assay to assess the function in mammalian cells (human U-251 glioblastoma) of dCas9 variants with deletions. FIG. 24A depicts the results using the WT (not deleted)-dCas9 construct fused to a KRAB repression domain. Sets of guide RNAs (CRISPRi sgRNAs) targeting three essential genes, proliferating cell nuclear antigen (PCNA) (sgPCNA: sgRNAs i3, i4, and i6) or replicating protein A1 (RPA1) (sgRPA1: sgRNAs i1, i5, or i8) or non-targeting controls (sgNT (sgRNAs 1 and 2)) where the target guide RNAs are also co-expressed with the mCherry fluorescent protein reporter Cells were analyzed on days 2, 5, 9. The results demonstrate that the WT-cCas9 KRAB construct is able to repress mCherry signal. FIG. 24B depicts the same experiment using the MISER-DelA-KRAB fusion in place of the WT construct, and demonstrates that the amount of repression is less that with the WT cCas9 construct while others demonstrated repression. The DelA construct comprises deletions in the dCas9 helix I and II regions. FIG. 24C similarly depicts the results with the MISER-DelB-KRAB construct where none of the samples exhibited significant repression. The DelB construct comprises deletions in the dCas9 helix II regions. FIG. 24D depicts the results with the MISER-DelC-KRAB construct and demonstrates that all of the samples demonstrated significant repression at day 9 as compared to day 2. The DelC construct comprises deletions in the dCas9 HNH nuclease domain FIG. 24E depicts the results with the MISER0DelD-KRAB construct and demonstrated that many of the samples shows significant expression. The DelD construct comprises deletions in the dCas9 RuvC nuclease domain. (*) means significant repression compared to day 2 results of the grouping.

As shown in FIG. 24A, the wild-type (non-deleted) dCas9-KRAB fusion protein effectively inhibited PCNA expression and RPA1 expression. As shown in FIG. 24B, the DelA-dCas9-KRAB fusion protein (MISER-DelA′), combined with the i6 sgRNA, was effective in inhibiting PCNA gene expression. Similarly, as shown in FIG. 24B, the DelA-dCas9-KRAB fusion protein (MISER-DelA′), combined with the i5 sgRNA, was effective in inhibiting RPA1 gene expression. As shown in FIG. 24D, the DelC-dCas9-KRAB fusion protein (MISER-DelC^{KRAB}) (in combination with i3, i4, or i6 sgRNAs) effectively inhibited expression of PCNA; and (in combination with it or i5 sgRNAs) effectively inhibited expression of RPA1. As shown in FIG. 24E, the DelD-dCas9-KRAB fusion protein (MISER-DelD^{KRAB}) (in combination with i6 sgRNA) effectively inhibited expression of PCNA; and (in combination with i5 sgRNA) effectively inhibited expression of RPA1.

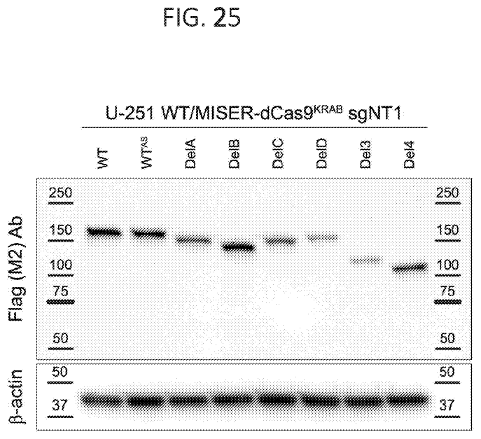

FIG. 25 provides an immunoblotting assay for Flag-tagged MISER-dCas9 or WT-dCas9 KRAB fusion proteins stably expressed in U-251 cells co-expressing a non-targeting guide (sgNT1). The indicated MISER deletions resulted in reduction of protein size. Beta-actin (ACTB) was used as loading control. Protein ladders indicated reference molecular weight markers in kDa.

### Example 3: In Vitro Binding of WT dCas9 and Variants Δ3CE, and Δ4CE

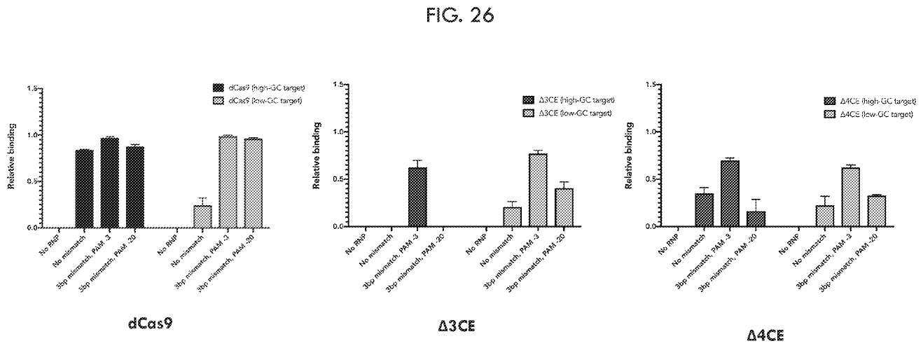

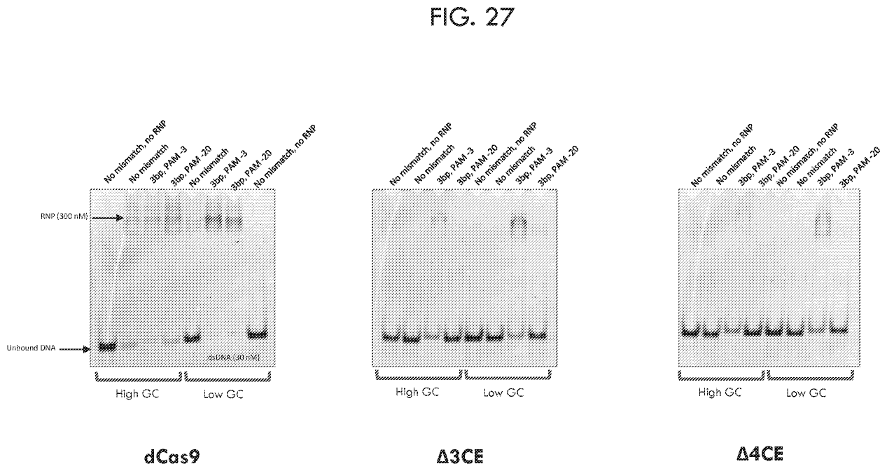

In vitro function of WT-dCas9 and variants Δ3CE and Δ4CE was assessed using an RNP-based gel shift assay (FIG. 26). WT dCas9 and stacked variants Δ3CE and Δ4CE were expressed and purified to homogeneity from E. coli. Oligonucleotide-based target dsDNAs were designed possessing either a low (˜30%, 298.2 tdTomato and DYRK1-BS14) or high (70%, B9) GC-content in the PAM proximal region. dsDNA was labeled on the 5′ end of the non-target strand using either FAM or Cy5 fluorophores. RNP was formed following the protocol in Lin et al. (Lin et al. (2014) eLife; 3: e04766) with sgRNA against the respective target sequence. For gel shift, 300 nM RNP was incubated with 30 nM dsDNA target. The results were analyzed using gel electrophoresis and quantified using fluorescence of the gel-shifted fluorescently labeled dsDNA.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. An RNA-guided effector polypeptide having a length of from about 610 amino acids to about 1310 amino acids, wherein the RNA-guided effector polypeptide comprises one or more of:
i) a deletion of from 5 contiguous amino acids to 158 contiguous amino acids of amino acids 166-324;
ii) a deletion of from 30 contiguous amino acids to 255 contiguous amino acids of amino acids 484-739;
iii) a deletion of from 4 contiguous amino acids to 220 contiguous amino acids of amino acids 761-981; and
d) a deletion of from 20 contiguous amino acids to 118 contiguous amino acids of amino acids 994-1112,
wherein the amino acid numbering is based on SEQ ID NO:5.

2. The RNA-guided effector polypeptide of claim 1, wherein the RNA-guided effector polypeptide comprises a deletion of from 5 contiguous amino acids to 158 contiguous amino acids of amino acids 166-324.

3. The RNA-guided effector polypeptide of claim 1, wherein the RNA-guided effector polypeptide comprises a deletion of from 30 contiguous amino acids to 255 contiguous amino acids of amino acids 484-739.

4. The RNA-guided effector polypeptide of claim 1, wherein the RNA-guided effector polypeptide comprises a deletion of from 4 contiguous amino acids to 220 contiguous amino acids of amino acids 761-981.

5. The RNA-guided effector polypeptide of claim 1, wherein the RNA-guided effector polypeptide comprises a deletion of from 20 contiguous amino acids to 118 contiguous amino acids of amino acids 994-1112.

6. The RNA-guided effector polypeptide of claim 1, wherein the RNA-guided effector polypeptide comprises:
i) a deletion of from 5 contiguous amino acids to 158 contiguous amino acids of amino acids 166-324; and
ii) a deletion of from 30 contiguous amino acids to 255 contiguous amino acids of amino acids 484-739.

7. The RNA-guided effector polypeptide of claim 1, wherein the RNA-guided effector polypeptide comprises:
i) a deletion of from 5 contiguous amino acids to 158 contiguous amino acids of amino acids 166-324; and
ii) a deletion of from 4 contiguous amino acids to 220 contiguous amino acids of amino acids 761-981.

8. The RNA-guided effector polypeptide of claim 1, wherein the RNA-guided effector polypeptide comprises:
i) a deletion of from 5 amino acids to 158 amino acids of amino acids 166-324; and
ii) a deletion of from 20 amino acids to 118 amino acids of amino acids 994-1112.

9. The RNA-guided effector polypeptide of claim 1, wherein the RNA-guided effector polypeptide comprises:
i) a deletion of from 5 contiguous amino acids to 158 contiguous amino acids of amino acids 166-324;
ii) a deletion of from 30 contiguous amino acids to 255 contiguous amino acids of amino acids 484-739; and
iii) a deletion of from 4 contiguous amino acids to 220 contiguous amino acids of amino acids 761-981.

10. The RNA-guided effector polypeptide of claim 1, wherein the RNA-guided effector polypeptide comprises:
i) a deletion of from 5 contiguous amino acids to 158 contiguous amino acids of amino acids 166-324;
ii) a deletion of from 30 contiguous amino acids to 255 contiguous amino acids of amino acids 484-739; and
iii) a deletion of from 20 contiguous amino acids to 118 contiguous amino acids of amino acids 994-1112.

11. The RNA-guided effector polypeptide of claim 1, wherein the RNA-guided effector polypeptide comprises:
i) a deletion of from 5 contiguous amino acids to 158 contiguous amino acids of amino acids 166-324;
ii) a deletion of from 30 contiguous amino acids to 255 contiguous amino acids of amino acids 484-739;
iii) a deletion of from 4 contiguous amino acids to 220 contiguous amino acids of amino acids 761-981; and
iv) a deletion of from 20 contiguous amino acids to 118 contiguous amino acids of amino acids 994-1112.

12. The RNA-guided effector polypeptide of claim 1, wherein the RNA-guided effector polypeptide comprises:
i) a deletion of from 30 contiguous amino acids to 255 contiguous amino acids of amino acids 484-739; and
ii) a deletion of from 4 contiguous amino acids to 220 contiguous amino acids of amino acids 761-981.

13. The RNA-guided effector polypeptide of claim 1, wherein the RNA-guided effector polypeptide comprises:
i) a deletion of from 30 contiguous amino acids to 255 contiguous amino acids of amino acids 484-739; and
ii) a deletion of from 20 contiguous amino acids to 118 contiguous amino acids of amino acids 994-1112.

14. The RNA-guided effector polypeptide of claim 1, wherein the RNA-guided effector polypeptide comprises:
i) a deletion of from 30 contiguous amino acids to 255 contiguous amino acids of amino acids 484-739;
ii) a deletion of from 4 contiguous amino acids to 220 contiguous amino acids of amino acids 761-981; and
iii) a deletion of from 20 contiguous amino acids to 118 contiguous amino acids of amino acids 994-1112.

15. The RNA-guided effector polypeptide of claim 1, wherein the RNA-guided effector polypeptide comprises:
i) a deletion of from 4 contiguous amino acids to 220 contiguous amino acids of amino acids 761-981; and
ii) a deletion of from 20 contiguous amino acids to 118 contiguous amino acids of amino acids 994-1112.

16. The RNA-guided effector polypeptide of claim 1, wherein the RNA-guided effector polypeptide comprises a deletion of amino acids 179-296 based on the number of SEQ ID NO:5.

17. The RNA-guided effector polypeptide of claim 1, wherein the RNA-guided effector polypeptide comprises a deletion of amino acids 503-708 based on the number of SEQ ID NO:5.

18. The RNA-guided effector polypeptide of claim 1, wherein the RNA-guided effector polypeptide comprises a deletion of amino acids 792-897 based on the number of SEQ ID NO:5.

19. The RNA-guided effector polypeptide of claim 1, wherein the RNA-guided effector polypeptide comprises a deletion of amino acids 1010-1081 based on the number of SEQ ID NO:5.

20. The RNA-guided effector polypeptide of claim 1, wherein the RNA-guided effector polypeptide comprises:
i) a deletion of amino acids 503-708 based on the number of SEQ ID NO:5;
ii) a deletion of amino acids 792-897 based on the number of SEQ ID NO:5; and
iii) a deletion of amino acids 1010-1081 based on the number of SEQ ID NO:5.

21. The RNA-guided effector polypeptide of claim 1, wherein the RNA-guided effector polypeptide comprises:
i) a deletion of amino acids 179-296 based on the number of SEQ ID NO:5;
ii) a deletion of amino acids 503-708 based on the number of SEQ ID NO:5;
iii) a deletion of amino acids 792-897 based on the number of SEQ ID NO:5; and
iv) a deletion of amino acids 1010-1081 based on the number of SEQ ID NO:5.

22. The RNA-guided effector polypeptide of claim 1, wherein the polypeptide comprises an amino acid sequence having at least 85% amino acid sequence identity to the amino acid sequence depicted in FIG. 2, wherein the RNA-guided effector polypeptide is 990 amino acids in length.

23. The RNA-guided effector polypeptide of claim 1, wherein the polypeptide comprises an amino acid sequence having at least 85% amino acid sequence identity to the amino acid sequence depicted in FIG. 3, wherein the RNA-guided effector polypeptide is 874 amino acids in length.

24. A nucleic acid comprising a nucleotide sequence encoding the RNA-guided effector polypeptide of any one of claims 1-23.

25. The nucleic acid of claim 24, wherein the nucleotide sequence is operably linked to a transcriptional control element.

26. The nucleic acid of claim 25, wherein the transcriptional control element is a promoter.

27. The nucleic acid of claim 26, wherein the promoter is a regulatable promoter.

28. The nucleic acid of claim 27, wherein the promoter is functional in a eukaryotic cell.

29. The nucleic acid of any one of claims 24-28, further comprising a nucleotide sequence encoding a guide RNA, wherein the guide RNA comprises a guide sequence that is complementary to a target sequence of a target nucleic acid, and comprises a region that can bind to the RNA-guided effector polypeptide.

30. A recombinant expression vector comprising the nucleic acid of any one of claims 24-29.

31. The recombinant expression vector of claim 30, wherein the recombinant expression vector is recombinant viral expression vector.

32. A fusion polypeptide comprising:
a) the RNA-guided effector polypeptide of any one of claims 1-23; and
b) a heterologous fusion partner.

33. The fusion polypeptide of claim 32, wherein the heterologous fusion partner provides for control of gene expression.

34. The fusion polypeptide of claim 32, wherein the heterologous fusion partner is a protein-modifying enzyme.

35. The fusion polypeptide of claim 32, wherein the heterologous fusion partner is a modifying enzyme is a DNA-modifying enzyme.

36. The fusion polypeptide of claim 32, wherein the heterologous fusion partner exhibits nuclease activity.

37. The fusion polypeptide of claim 36, wherein the nuclease activity is double-stranded DNA cleavage activity.

38. The fusion polypeptide of claim 37, wherein the heterologous fusion partner comprises an amino acid sequence having at least 85% amino acid sequence identity to the catalytic domain of the amino acid sequence depicted in FIG. 2A and has a length of no more than 200 amino acids.

39. The fusion polypeptide of any one of claims 32-38, wherein the fusion partner has a length of no more than 400 amino acids.

40. The fusion polypeptide of claim 35, wherein the heterologous fusion partner is a base editor.

41. The fusion polypeptide of claim 40, wherein the base editor is a cytidine deaminase.

42. The fusion polypeptide of claim 40, wherein the base editor is an adenosine deaminase.

43. The fusion polypeptide of claim 32, further comprising one or more nuclear localization sequences (NLS).

44. The fusion polypeptide of claim 43, wherein the fusion polypeptide comprises a single NLS.

45. The fusion polypeptide of claim 43, wherein the fusion polypeptide comprises 2 or more NLSs.

46. The fusion polypeptide of claim 43, wherein the fusion polypeptide comprises an NLS at the C-terminus, at the N-terminus, or at the C-terminus and the N-terminus, of the fusion polypeptide.

47. The fusion polypeptide of claim 32, further comprising an endosomolytic peptide.

48. A nucleic acid comprising a nucleotide sequence encoding the fusion polypeptide of any one of claims 32-47.

49. The nucleic acid of claim 48, wherein the nucleotide sequence is operably linked to a transcriptional control element.

50. The nucleic acid of claim 49, wherein the transcriptional control element is a promoter.

51. The nucleic acid of claim 50, wherein the promoter is a regulatable promoter.

52. The nucleic acid of claim 50 or claim 51, wherein the promoter is functional in a eukaryotic cell.

53. The nucleic acid of any one of claims 48-52, further comprising a nucleotide sequence encoding a guide RNA, wherein the guide RNA comprises a guide sequence that is complementary to a target sequence of a target nucleic acid, and comprises a region that can bind to the RNA-guided effector polypeptide.

54. A recombinant expression vector comprising the nucleic acid of any one of claims 48-53.

55. The recombinant expression vector of claim 54, wherein the recombinant expression vector is recombinant viral expression vector.

56. A ribonucleoprotein (RNP) complex comprising:
a) the fusion polypeptide of claim 32; and
b) a guide RNA, or a nucleic acid encoding the guide RNA, wherein the guide RNA comprises a guide sequence that is complementary to a target sequence of a target nucleic acid, and comprises a region that can bind to the RNA-guided effector polypeptide.

57. The RNP complex of claim 56, wherein the guide RNA is a single-molecule guide RNA.

58. A composition comprising:
a) the RNP complex of claim 56 or claim 57; and
b) a pharmaceutically acceptable excipient.

59. The composition of claim 58, comprising a lipid.

60. The composition of claim 58, comprising a liposome, a hydrogel, a microparticle, a nanoparticle, or a block copolymer micelle.

61. A kit comprising:
a) the RNA-guided effector polypeptide of claim 1, or the fusion polypeptide of claim 32; and
b) a guide RNA, or a nucleic acid encoding the guide RNA, wherein the guide RNA comprises a guide sequence that is complementary to a target sequence of a target nucleic acid, and comprises a region that can bind to the RNA-guided effector polypeptide.

62. The kit of claim 61, wherein the guide RNA is a single-molecule guide RNA.

63. The kit of claim 61 or 62, wherein the RNA-guided effector polypeptide and the guide RNA are in separate containers.

64. The kit of claim 61 or 62, wherein the RNA-guided effector polypeptide and the guide RNA are together in a single container.

65. The kit of any one of claims 61-64, wherein the guide RNA comprises one or more of: a modified nucleobase, a modified backbone or non-natural internucleoside linkage, a modified sugar moiety, a Locked Nucleic Acid, a Peptide Nucleic Acid, and a deoxyribonucleotide.

66. The kit of any one of claims 61-65, further comprising a donor nucleic acid template.

67. A method of binding and/or modifying a target nucleic acid and/or modifying a polypeptide that binds to a target nucleic acid, the method comprising contacting the target nucleic acid with:
i) the RNA-guided effector polypeptide of any one of claims 1-23; or
ii) the fusion polypeptide of any one of claims 32-47.

68. The method of claim 67, wherein the target nucleic acid is selected from: double stranded DNA, single stranded DNA, RNA, genomic DNA, and extrachromosomal DNA.

69. The method of claim 67, wherein contacting results in genome editing.

70. The method of claim 67 or 68, wherein said contacting takes place outside of a bacterial cell and outside of an archaeal cell.

71. The method of any one of claims 67-69, wherein said contacting takes place in vitro outside of a cell.

72. The method of any one of claims 67-69, wherein said contacting takes place inside of a target cell.

73. The method of claim 72, wherein the target cell is a eukaryotic cell.

74. The method of claim 72 or 73, wherein the target cell is in vivo.

75. The method of claim 72 or 73, wherein the target cell is ex vivo.

76. The method of claim 72 or 73, wherein the eukaryotic cell is selected from the group consisting of: a plant cell, a fungal cell, a single cell eukaryotic organism, a mammalian cell, a reptile cell, an insect cell, an avian cell, a fish cell, a parasite cell, an arthropod cell, a cell of an invertebrate, a cell of a vertebrate, a rodent cell, a mouse cell, a rat cell, a primate cell, a non-human primate cell, and a human cell.

77. The method of any one of claims 72-76, wherein said contacting further comprises: introducing a DNA donor template into the target cell.
